(19) United States
(12) Patent Application Publication
(10) Pub. No.: US 2024/0110194 A1
(43) Pub. Date: Apr. 4, 2024

(54) COMPOSITIONS AND METHODS FOR TRANSFORMATION OF EMBRYO EXPLANT POPULATIONS

(71) Applicant: Monsanto Technology LLC, St. Louis, MO (US)

(72) Inventors: Brent Brower-Toland, St. Louis, MO (US); David Vincent Butruille, Chesterfield, MO (US); Edward J. Cargill, Chesterfield, MO (US); Yurong Chen, Chesterfield, MO (US); Megan Elizabeth Hassebrock, O'Fallon, MO (US); Thomas Ream, Wildwood, MO (US); Jennifer Rinehart, Spring Green, WI (US); Mary Ann Saltarikos, O'Fallon, MO (US); Michelle Folta Valentine, Troy, MO (US)

(21) Appl. No.: 18/377,421

(22) Filed: Oct. 6, 2023

Related U.S. Application Data
(63) Continuation-in-part of application No. PCT/US2023/065349, filed on Apr. 5, 2023.
(60) Provisional application No. 63/492,279, filed on Mar. 27, 2023.
(60) Provisional application No. 63/441,369, filed on Jan. 26, 2023.
(60) Provisional application No. 63/328,567, filed on Apr. 7, 2022.

(51) Int. Cl. C12N 15/82; C12N 9/22; C12N 15/11

(52) U.S. Cl. CPC C12N 15/8205; C12N 9/22; C12N 15/11

(57) ABSTRACT

The present disclosure provides novel compositions and methods for collectively transforming or genetically modifying a population of distinct germplasm of different germplasms or having different genotypes. The compositions of the present disclosure may include a population of distinct germplasm, such as embryo explants, and a heterologous polynucleotide molecule, a ribonucleoprotein, or a site-specific nuclease. The methods of the present disclosure may include one or more steps of explant preparation, explant rehydration, Rhizobiales bacterium inoculation and co-culture or particle bombardment, bud induction, extended bud induction, and/or regeneration or development of genetically modified plants or plant parts. The methods provided herein may include transforming at least one plant cell of the embryo explants with a heterologous polynucleotide. The methods provided herein also include methods of regenerating or growing a plurality of genetically modified plants or plant parts from the population of transformed or edited plant cells or explants and comparing, selecting or screening one or more genotypes having an improved phenotype or other culturing characteristic.

### CROSS-REFERENCE TO RELATED APPLICATIONS

This application is a continuation-in-part of International App. No. PCT/US23/65349, which application claims the priority of U.S. Provisional Appl. Ser. No. 63/328,567, filed Apr. 7, 2022, U.S. Provisional Appl. Ser. No. 63/441,369, filed Jan. 26, 2023, and U.S. Provisional Appl. Ser. No. 63/492,279, filed Mar. 27, 2023, the entire disclosure of each of which is incorporated herein by reference.

### FIELD OF THE INVENTION

The present disclosure relates to compositions and methods for genetically modifying a population of germplasm having distinct genotypes.

### BACKGROUND

Crop plants, such as corn, wheat, rice, barley, sorghum, soybean, cotton, and canola are important crops and are primary food sources in many areas of the world. Genetic modification of embryo explants has been used to produce such crop plants which have improved traits or characteristics. There is, however, a continuing need in the art for improved methods of genetically modifying a plurality of different plant genotypes at once, which reduce or eliminate the use of costly and time consuming plant breeding techniques.

The disclosure provides novel compositions and methods for genetic modification of a population of embryo explants having different plant genotypes and the regeneration of genetically modified plants or plant parts therefrom, which reduce or eliminate the use of plant breeding or introgression techniques to overcome many of the challenges and limitations in the art.

### SUMMARY

In one aspect the present disclosure provides a composition comprising a population of distinct germplasm and at least one heterologous polynucleotide molecule, wherein the population of distinct germplasm comprises at least two different plant genotypes. In another aspect, the present disclosure provides a composition comprising a population of distinct germplasm and a ribonucleoprotein or a site-specific nuclease, wherein the population of distinct germplasm comprises least two different plant genotypes. In a specific embodiment, the population is defined as a population of distinct monocot or dicot germplasm. In another embodiment, the population is defined as a population of distinct corn, wheat, rice, barely, sorghum, turfgrass, soybean, cotton, or canola germplasm. In yet another embodiment, the population is defined as a population of distinct dicot or monocot embryo explants. In still yet another embodiment, the population is defined as a population of distinct corn, wheat, rice, barely, sorghum, turfgrass, soybean, cotton, or canola embryo explants. In one embodiment, the present disclosure provides a composition comprising a field, wherein the field comprises plants having a population of distinct germplasm and, wherein at least one plant of each distinct germplasm comprises a heterologous polynucleotide molecule.

Compositions can also include a mix of heterologous nucleic acid molecules with the populations of distinct germplasm. One of ordinary skill in the art will appreciate that using a population of germplasm and heterologous nucleic acid sequences allows for the identification of an optimum combination of germplasm and expression elements, such as promoters, untranslated regions, terminators, and the like. The heterologous polynucleotide molecule, in one embodiment, comprises an expression cassette and the expression cassette comprises a selectable marker sequence, a screenable marker sequence, a coding region for a protein of interest, a nucleotide sequence encoding a site-specific nuclease, an untranslated region (UTR), a morphogenic regulator, a nucleotide sequence encoding a guide RNA molecule and combinations thereof. The heterologous polynucleotide molecule, in another embodiment, comprises a guide RNA molecule. The ribonucleoprotein, in yet another embodiment, comprises a site-specific nuclease and a guide RNA molecule. In still yet another embodiment, the composition may comprise a Rhizobiales bacterium. In one embodiment, the Rhizobiales bacterium may be selected from the group consisting of: a) a Rhizobiaceae, a Phyllobacteriaceae, a Brucellaceae, a Bradyrhizobiaceae, and a Xanthobacteraceae bacterium; or an Agrobacterium, a Rhizobium, a Sinorhizobium, a Mesorhizobium, a Phyllobacterium, an Ochrobactrum, a Bradyrhizobium, and an Azorhizobium bacterium. The composition, in one embodiment, may further comprise an inoculation medium.

In some embodiments, the distinct germplasm of the at least two different plant genotypes comprise germplasm of a first genotype and germplasm of a second genotype, and wherein the distinct germplasm of the first genotype and the distinct germplasm of the second genotype are present in the population in a predetermined ratio. In one embodiment, the predetermined ratio is determined based upon at least one culturing characteristic associated with the first genotype, associated with the second genotype, or associated with the first genotype and the second genotype. In another embodiment, the at least one culturing characteristic is selected from the group consisting of: explant excision efficiency, regeneration efficiency, shoot generation efficiency, genetic modification efficiency, transformation efficiency, and ability to regenerate into a genetically modified plant or plant part. The predetermined ratio of the distinct germplasm of the first genotype and the second genotype, in yet another embodiment, comprises an approximately equal number of germplasm of the first genotype and of the second genotype.

In another aspect, the present disclosure provides a method of producing a population of distinct genetically modified germplasm, comprising: a) contacting a population of distinct germplasm with at least one heterologous polynucleotide molecule, wherein the population of distinct germplasm comprises germplasm of at least two different plant genotypes; b) genetically modifying the population of distinct germplasm to be genetically modified; and c) collecting the population of distinct genetically modified germplasm. In one embodiment, the population of distinct genetically modified germplasm comprises germplasm of at least two different plant genotypes. In another embodiment, the method may further comprise identifying a genotype of at least one germplasm of the population of distinct genetically modified germplasm. The identifying the genotype, in yet another embodiment, may comprise detecting at least one genetic marker in the at least germplasm, wherein the at least one genetic marker comprises a polynucleotide sequence that is characteristic of the genotype. In still yet another embodiment, the polynucleotide sequence is exclusively characteristic of the genotype. Identifying the genotype, in one embodiment, may comprise detecting at least 2, at least 3, at least 4, at least 5, at least 6, at least 7, at least 8, at least 9, at least 10, at least 15, at least 20, or at least 25 genetic markers in the at least one germplasm, wherein each of the genetic markers comprises a polynucleotide sequence that is characteristic of the genotype. In another embodiment, each of the genetic markers comprises a polynucleotide sequence that is exclusively characteristic of the genotype. Identifying the genotype, in yet another embodiment, comprises identifying at least one genotype of a plurality of germplasms of the population of distinct genetically modified germplasm. In still yet another embodiment, identifying the genotype comprises identifying a plurality of genotypes of the plurality of germplasms of the population of distinct genetically modified germplasms. In one embodiment, the methods of the present disclosure may further comprise identifying at least one genetic modification present in at least one germplasm of the population of distinct genetically modified germplasm. In another embodiment, the methods of the present disclosure may further comprise identifying at least one genetic modification present in a plurality of germplasms of the population of distinct genetically modified germplasm.

In yet another aspect the present disclosure provides a method of genetically modifying a population of plant embryo explants, comprising: collectively introducing a heterologous polynucleotide molecule into at least two embryo explants of the population, the at least two plant embryo explants each comprising meristematic tissue, wherein the population comprises embryo explants of at least two different plant genotypes. In some embodiments, the population is defined as a population of monocot embryo explants. The population of monocot embryo explants, in further embodiments, may be defined as a population of corn, wheat, rice, barley, sorghum or turfgrass embryo explants. In particular embodiments, the population is defined as a population of dicot embryo explants. The population of dicot embryo explants, in further embodiments, may be defined as a population of soybean, cotton, or canola embryo explants. In certain embodiments, collectively introducing the heterologous polynucleotide molecule comprises introducing the heterologous polynucleotide molecule into the at least two explants of the population via bacterial-mediated transformation. In other embodiments, collectively introducing the heterologous polynucleotide molecule comprises introducing the heterologous polynucleotide molecule into the at least two explants of the population via Rhizobiales bacterium mediated transformation. The Rhizobiales bacterium, in some embodiments, is selected from the group consisting of: a) a Rhizobiaceae, a Phyllobacteriaceae, a Brucellaceae, a Bradyrhizobiaceae, and a Xanthobacteraceae bacterium; or b) an Agrobacterium, a Rhizobium, a Sinorhizobium, a Mesorhizobium, a Phyllobacterium, an Ochrobactrum, a Bradyrhizobium, and an Azorhizobium bacterium. In further embodiments, collectively introducing the heterologous polynucleotide molecule comprises introducing the heterologous polynucleotide molecule via Agrobacterium-mediated transformation. In particular embodiments, collectively introducing the heterologous polynucleotide molecule comprises inoculating the at least two embryo explants with an inoculation medium comprising a Rhizobiales bacterium competent to transform the at least two embryo explants with the heterologous polynucleotide molecule. In certain embodiments, a force treatment is applied to the population in contact with the inoculation medium. In other embodiments, a force treatment is applied prior to collectively introducing the heterologous polynucleotide molecule. In particular embodiments, the force treatment comprises a gravitational force treatment within a range from about 3,000×g to about 6,000×g, about 3,500×g to about 5,000×g, or about 3,500×g to about 4,500×g. In some embodiments, the method may further comprise co-culturing the at least two embryo explants with the Rhizobiales bacterium in contact with a co-culture medium. In certain embodiments, the population of embryo explants is a population of monocot seed embryo explants, and the method further comprises co-culturing the at least two embryo explants of the population in contact with the co-culture medium at a density less than or equal to about 9.1 embryo explants per square centimeter (cm²) of co-culture surface area. In some embodiment, the population of embryo explants is a population of monocot seed embryo explants, and the method further comprises co-culturing the at least two embryo explants of the population in contact with the co-culture medium for a time period in a range from about 6 days to about 8 days. In additional embodiments, collectively introducing the heterologous polynucleotide molecule comprises introducing the heterologous polynucleotide molecule into the at least two explants of the population via by particle bombardment. In certain embodiments, the methods provided by the present disclosure may further comprise collectively introducing a site-specific nuclease into the at least two embryo explants of the population. In particular embodiments, the heterologous polynucleotide molecule comprises a guide RNA molecule, and the collectively introducing comprises introducing a site-specific nuclease into the at least two embryo explants. The site-specific nuclease, in some embodiments, is a ribonucleoprotein and the ribonucleoprotein comprises the site-specific nuclease and the guide RNA molecule.

In further embodiments, the heterologous polynucleotide molecule may comprise a first expression cassette and a second expression cassette. The first expression cassette, in certain embodiments, may comprise a first selectable marker gene, a first screenable marker gene, a first gene of interest, a nucleotide sequence encoding a first site-specific nuclease, or a nucleotide sequence encoding a first guide RNA; and the second expression cassette, in particular embodiments, may comprise a second selectable gene, a second screenable marker gene, a second gene of interest, a nucleotide sequence encoding a second site-specific nuclease, or a nucleotide sequence encoding a second guide RNA.

In certain embodiments, collectively introducing the heterologous polynucleotide molecule comprises introducing at least two heterologous polynucleotides into the at least two explants of the population, wherein the at least two heterologous polynucleotides are different. In further embodiments, the at least two heterologous polynucleotides comprise a first heterologous polynucleotide comprising a first expression cassette and a second heterologous polynucleotide comprising a second expression cassette. The first expression cassette, in some embodiments, may comprise a first selectable marker gene, a first screenable marker gene, a first gene of interest, a nucleotide sequence encoding a first site-specific nuclease, or a nucleotide sequence encoding a first guide RNA; and the second expression cassette, in certain embodiments, may comprise a second selectable marker gene, a second screenable marker gene, a second gene of interest, a nucleotide sequence encoding a second site-specific nuclease, or a nucleotide sequence encoding a second guide RNA.

In particular embodiments, the method of genetically modifying a population of plant embryo explants provided by the present disclosure may further comprise culturing the at least two embryo explants in contact with a first bud induction medium comprising a first auxin and a first cytokinin. The first bud induction medium, in certain embodiments, comprises a high cytokinin to auxin ratio. In some embodiments, the first auxin in the first bud induction medium is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). The concentration of the first auxin in the first bud induction medium, in further embodiments, is from about 0.02 mg/L to about 25 mg/L or is from about 1 mg/L to about 2 mg/L. In certain embodiments, the first cytokinin in the first bud induction medium is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the first cytokinin in the first bud induction medium, in particular embodiments, is in a range from about 0.1 mg/L to about 50 mg/L. In some embodiments, the population of embryo explants is a population of monocot seed embryo explants and the method further comprises culturing the at least two embryo explants in contact with the first bud induction medium at a density less than or equal to about 3.9 embryo explants per square centimeter (cm²) of first bud induction surface area.

In certain embodiments, the method of genetically modifying a population of plant embryo explants provided by the present disclosure may further comprise culturing the at least two embryo explants in contact with a second bud induction medium comprising the first auxin or a second auxin and the first cytokinin or a second cytokinin. The embryo explant is cultured in contact with the second bud induction medium, in some embodiments, at a temperature in a range from about 20° C. to about 32° C., from about 25° C. to about 29° C., or from about 27° C. to about 28° C. In particular embodiments, the second bud induction medium comprises a high cytokinin to auxin ratio. In specific embodiments, the second bud induction medium comprises: a) the first auxin and the first cytokinin; b) the first auxin and the second cytokinin; c) the second auxin and the first cytokinin; or d) the second auxin and the second cytokinin. In certain embodiments, the population of embryo explants is a population of monocot seed embryo explants and the method further comprises culturing the at least two embryo explants in contact with the second bud induction medium at a density less than or equal to about 2.6 embryo explants per square centimeter (cm²) of second bud induction surface area. In some embodiments, the first auxin or the second auxin in the second bud induction medium is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). The first cytokinin or the second cytokinin in the second bud induction medium, in some embodiments, is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the first cytokinin or the second cytokinin in the second bud induction medium, in specific embodiments, is in a range from about 0.1 mg/L to about 1 mg/L, about 0.1 mg/L to about 5 mg/L, about 0.1 mg/L to about 50 mg/L, from about 0.1 mg/L to about 25 mg/L, about 0.5 mg/L to about 25 mg/L, or from about 2 mg/L to about 10 mg/L. The concentration of the first auxin or the second auxin in the second bud induction medium, in certain embodiments, is about 0.01 mg/L to about 25 mg/L, about 0.02 mg/L to about 10 mg/L, or about 1 mg/L to about 2 mg/L. In particular embodiments, the heterologous polynucleotide molecule comprises a selectable marker gene, the second bud induction medium comprises a selection agent, and the selectable marker gene provides resistance in a plant to the selection agent.

In specific embodiments, the method of genetically modifying a population of plant embryo explants provided by the present disclosure may further comprise regenerating or growing a plurality of genetically modified plants or plant parts in contact with a regeneration medium from the at least two embryo explants or any progeny generation of a cell thereof. In some embodiments, the population is a population of monocot seed embryo explants and the method comprises regenerating or growing the plurality of genetically modified plants or plant parts from the at least two embryo explants in contact with the regeneration medium at a density less than or equal to about 2.6 embryo explants per square centimeter (cm²) of regeneration surface area. In still yet other embodiments, the methods of the present disclosure may further comprise regenerating a cultured population of monocot seed embryo explants in contact with a first regeneration medium, transferring the cultured population of monocot seed embryo explants, or a subset of the cultured population of monocot seed embryo explants, to a second regeneration medium, and regenerating the plurality of genetically modified monocot plants or plant parts in contact with the second regeneration medium. The regeneration, the first regeneration medium, or the second regeneration medium, in some embodiments, has a low salt concentration. In some embodiments, the heterologous polynucleotide molecule comprises a selectable marker gene, the regeneration medium comprises a selection agent, and the selectable marker gene provides resistance in a plant to the selection agent. In certain embodiments, the genetically modified plants or plant parts comprise at least one genetic modification. The genetic modification may comprise, in particular embodiments, an integration or insertion of the heterologous polynucleotide molecule or a fragment thereof into the genome of the plurality of genetically modified plants or plant parts, wherein the integration or insertion comprises at least one expression cassette or at least one transgene. The genetic modification may comprise, in some embodiments, an edit introduced into the genome of the plurality of genetically modified plants or plant parts by a genome editing technique with a site-specific nuclease and/or a guide RNA molecule. In certain embodiments, the heterologous polynucleotide molecule comprises at least one expression cassette, and the at least one expression cassette encodes the site-specific nuclease or the guide RNA molecule, or the heterologous polynucleotide molecule comprises at least two expression cassettes comprising a first expression cassette encoding the site-specific nuclease and a second expression cassette encoding the guide RNA molecule. In particular embodiments, the genetically modified plant parts comprise shoots or roots. In certain embodiments, the genetically modified plant parts comprise seeds. In further embodiments, the genetically modified plants or plant parts are non-chimeric. The genetically modified plants or plant parts, in particular embodiments, are cultured and/or regenerated without generating a callus tissue culture.

In further embodiments, the method of genetically modifying a population of plant embryo explants provided by the present disclosure may further comprise regenerating or growing a plurality of genetically modified plants or plant parts in contact with a regeneration medium from at least two embryo explants or any progeny generation of a cell thereof, wherein each of the plurality of genetically modified plants or plant parts comprises at least one genetic modification. In certain embodiments, the methods provided by the present disclosure may additionally comprise: selecting a genetically modified plant comprising at least one genetic modification; and crossing the plant with itself or a second plant to obtain a progeny plant or seed. The selecting, in some embodiments, comprises identifying a genotype of the genetically modified plant and selecting the plant comprising the genotype. In further embodiments, the methods provided by the present disclosure may further comprise: selecting a first genetically modified plant comprising at least a first genetic modification and a second genetically modified plant comprising at least a second genetic modification; crossing the first genetically modified plant with itself or a first different plant to obtain a first progeny plant or seed; and crossing the second genetically modified plant with either itself, the first genetically modified plant, or a second different plant to obtain a second progeny plant or seed. The first different plant and/or the second different plant may, in certain embodiments, have a different genotype than the first genetically modified plant and/or the second genetically modified plant. Selecting, in particular embodiments, comprises identifying a first genotype of the first genetically modified plant and selecting the first genetically modified plant comprising the first genotype; and identifying a second genotype of the second genetically modified plant and selecting the second genetically modified plant comprising the second genotype.

In particular embodiments, the population of embryo explants provided by the present disclosure comprises embryo explants having an internal moisture content in a range from about 3% to about 25% prior to introducing the heterologous polynucleotide molecule. In specific embodiments, the population comprises embryo explants comprising an apical portion of an embryo axis lacking the radical, and wherein remaining portions of the seeds from which the embryo explants have been prepared have been substantially removed from the embryo explants. The population, in certain embodiments, is defined as a population of dry, dry excised, wet excised or wet seed embryo explants. The population, in other embodiments, is defined as a population of mature or immature seed embryo explants. In further embodiments, the population comprising embryo explants is prepared from a population of monocot seeds under conditions wherein the embryo explants do not germinate and remain viable and competent for genetic modification. The population, in certain embodiments, comprises embryo explants having at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. The at least two embryo explants of the population, in particular embodiments, comprise at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 600, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. The population, in some embodiments, comprises at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 20,000, at least 30,000, at least 40,000, or at least 50,000 embryo explants, or from about 2 to about 50,000, about 1,000 to about 50,000, about 1,000 to about 40,000, about 1,000 to about 30,000, about 1,000 to about 20,000, about 1,000 to about 10,000, about 1,000 to about 9,000, about 1,000 to about 8,000, about 1,000 to about 7,000, about 1,000 to about 6,000, about 1,000 to about 5,000, about 1,000 to about 4,000, about 1,000 to about 3,000, about 5,000 to about 50,000, about 5,000 to about 40,000, about 5,000 to about 30,000, about 5,000 to about 20,000, about 5,000 to about 10,000, about 2 to about 1000, about 5 to about 900, about 5 to about 800, about 5 to about 700, about 5 to about 600, about 5 to about 500, about 10 to about 500, from about 15 to about 400, from about 20 to about 300, from about 25 to about 200, from about 10 to about 150, from about 10 to about 100, from about 10 to about 90, from about 10 to about 80, from about 10 to about 70, from about 10 to about 60, from about 10 to about 50, from about 10 to about 40, from about 10 to about 30, from about 10 to about 20, from 2 to about 50, from 2 to about 40, from 2 to about 30, from 2 to about 20, from 2 to about 15, or from 2 to about 10 embryo explants. In some embodiments, the population comprises at least one embryo explant having at least one unidentified genotype prior to collectively introducing the heterologous polynucleotide molecule. In other embodiments, the at least two different genotypes of the embryo explants are known. In further embodiments, each embryo explant of the population is an embryo explant of a known genotype.

In certain embodiments, the heterologous polynucleotide is collectively introduced into embryo explants having at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 600, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. In further embodiments, the heterologous polynucleotide molecule is introduced into embryo explants and a plurality of genetically modified plants or plant parts is regenerated therefrom, wherein the plurality of genetically modified plants or plant parts comprises at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 600, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. In additional embodiments, the plurality of genetically modified plants or plant parts comprise at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 600, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes.

In particular embodiments, the method of genetically modifying a population of plant embryo explants provided by the present disclosure may further comprise identifying a genotype of at least one of the embryo explants of the population or at least one of the genetically modified plants or plant parts. Identifying the genotype, in some embodiments, comprises detecting at least one genetic marker in the at least one embryo explant or the at least one genetically modified plant or plant part, wherein the at least one genetic marker comprises a polynucleotide sequence that is characteristic of the genotype. In specific embodiments, the polynucleotide sequence is exclusively characteristic of the genotype. Identifying the genotype, in further embodiments, comprises detecting at least 2, at least 3, at least 4, at least 5, at least 6, at least 7, at least 8, at least 9, at least 10, at least 15, at least 20, at least 25, or from about 2 to about 26, about 5 to about 26, about 10 to about 26, about 15 to about 26, or about 20 to about 26 genetic markers in the at least one embryo explant or the at least one genetically modified plant or plant part, wherein each of the genetic markers comprises a polynucleotide sequence that is characteristic of the genotype. In certain embodiments, each of the genetic markers comprises a polynucleotide sequence that is exclusively characteristic of the genotype. In some embodiments, identifying a genotype comprises identifying the genotype before or after the heterologous polynucleotide molecule is introduced into the at least two embryo explants of the population; identifying the genotype before or after co-culturing the at least two embryo explants of the population; identifying the genotype before or after culturing the at least two embryo explants of the population in contact with the first bud induction medium; identifying the genotype before or after culturing the at least two embryo explants of the population in contact with the second bud induction medium; or identifying the genotype before or after regenerating or growing the plurality of genetically modified plants or plant parts from the at least two embryo explants of the population or any progeny generation of a cell thereof. Identifying the genotype, in particular embodiments, comprises performing genetic sequencing on a sample comprising a polynucleotide molecule from or derived from the at least one embryo explant or the at least one genetically modified plant or plant part; and detecting at least 1, at least 2, at least 3, at least 4, at least 5, at least 6, at least 7, at least 8, at least 9, at least 10, at least 15, or at least 20 genetic markers in the sample, wherein the polynucleotide molecule is a genomic DNA molecule or a fragment thereof or a mRNA molecule or a fragment thereof or wherein the polynucleotide molecule is derived from a genomic DNA molecule or a fragment thereof or a mRNA molecule or a fragment thereof, and wherein each of the genetic markers comprises a polynucleotide sequence that is characteristic of the genotype. In other embodiments, identifying the genotype comprises contacting a sample comprising a polynucleotide molecule from or derived from the at least one embryo explant or the at least one genetically modified plant or plant part with at least 1, at least 2, at least 3, at least 4, at least 5, at least 6, at least 7, at least 8, at least 9, at least 10, at least 15, or at least 20 polynucleotide probe(s), wherein each of the polynucleotide probe(s) is specific for one genetic marker; subjecting the sample and the polynucleotide probe(s) to stringent hybridization conditions; and detecting the hybridization of the polynucleotide probe(s) to at least 1, at least 2, at least 3, at least 4, at least 5, at least 6, at least 7, at least 8, at least 9, at least 10, at least 15, or at least 20 genetic markers in the sample, wherein the polynucleotide molecule is a genomic DNA molecule or a fragment thereof or a mRNA molecule or a fragment thereof or wherein the polynucleotide molecule is derived from a genomic DNA molecule or a fragment thereof or a mRNA molecule or a fragment thereof, and wherein each of the genetic markers comprises a polynucleotide sequence that is characteristic of the genotype. In various embodiments, identifying the genotype comprises amplification of a polynucleotide molecule or restriction mapping. In specific embodiments, identifying the genotype comprises identifying a genotype of a plurality of the embryo explants or a plurality of the genetically modified plants or plant parts. Identifying the genotype, in certain embodiments, comprises identifying a plurality of genotypes of at least one of the embryo explants of the population or at least one of the genetically modified plants or plant parts. In some embodiments, identifying the genotype comprises identifying a plurality of genotypes of a plurality of the embryo explants or a plurality of the genetically modified plants or plant parts.

In certain embodiments, identifying the genotype comprises detecting at least two genetic markers in at least two of the embryo explants of the population or at least two of the genetically modified plants or plant parts. The at least two genetic markers, in further embodiments, comprise a first genetic marker and a second genetic marker, wherein the first genetic marker comprises a first polynucleotide sequence that is characteristic of a first genotype and the second genetic marker comprises a second polynucleotide sequence that is characteristic of a second genotype. In particular embodiments, the first polynucleotide sequence is exclusively characteristic of the first genotype or the second polynucleotide sequence is exclusively characteristic of the second genotype. In some embodiments, identifying comprises detecting at least 3, at least 4, at least 5, at least 6, at least 7, at least 8, at least 9, at least 10, at least 15, or at least 20 genetic markers in at least two embryo explants or at least two genetically modified plants or plant parts, wherein at least two of the genetic markers comprise a polynucleotide sequence that is characteristic of the first genotype and at least one of the genetic markers comprises a polynucleotide sequence that is characteristic of the second genotype. Identifying comprises, in further embodiments, performing genetic sequencing on at least a first sample and a second sample, wherein the first sample comprises a first polynucleotide molecule from or derived from a first embryo explant or a first genetically modified plant or plant part, and the second sample comprises a second polynucleotide molecule from or derived from a second embryo explant or a second genetically modified plant or plant part; and detecting at least 2, at least 3, at least 4, at least 5, at least 6, at least 7, at least 8, at least 9, at least 10, at least 15, or at least 20 genetic markers in the first sample and the second sample, wherein the first polynucleotide molecule is a genomic DNA molecule or a fragment thereof or a mRNA molecule or a fragment thereof or is derived from a genomic DNA molecule or a fragment thereof or a mRNA molecule or a fragment thereof, or wherein the second polynucleotide molecule is a genomic DNA molecule or a fragment thereof or a mRNA molecule or a fragment thereof or is derived from a genomic DNA molecule or a fragment thereof or a mRNA molecule or a fragment thereof, and wherein each of the genetic markers comprises a polynucleotide sequence that is characteristic of the genotype, or the genetic markers comprise a first genetic marker and a second genetic marker, the first genetic marker comprising a first polynucleotide sequence that is characteristic of a first genotype and the second genetic marker comprising a second polynucleotide sequence that is characteristic of a second genotype.

In certain embodiments, the method of genetically modifying a population of plant embryo explants provided by the present disclosure may further comprise identifying a genetic modification present in the at least one embryo explant of the population or the at least one genetically modified plant or plant part; and selecting an embryo explant of the population or a genetically modified plant or plant part comprising the genetic modification, wherein the selected embryo explant or the selected genetically modified plant or plant part further comprises the at least one genetic marker characteristic of the genotype, or wherein the selected embryo explant or the selected genetically modified plant or plant part does not further comprise the at least one genetic marker characteristic of the genotype. In some embodiments, the method of genetically modifying a population of plant embryo explants provided by the present disclosure may further comprise regenerating or growing a regenerated genetically modified plant or plant part from the selected embryo explant or any progeny generation of a cell thereof; or crossing the selected genetically modified plant with itself or a different plant to obtain a progeny plant or seed. In these embodiments, the method may further comprise identifying a genetic modification present in the at least two embryo explants of the population or the at least two genetically modified plants or plant parts; and selecting a first embryo explant of the population or a first genetically modified plant or plant part comprising the genetic modification and a second embryo explant of the population or a second genetically modified plant or plant part comprising the genetic modification, wherein the first selected embryo explant or the first selected genetically modified plant or plant part further comprises the first genetic marker and/or the second genetic marker, or wherein the first selected embryo explant or the first selected genetically modified plant or plant part does not further comprise the first genetic marker and/or the second genetic marker, and wherein the second selected embryo explant or the second selected genetically modified plant or plant part further comprises the first genetic marker and/or the second genetic marker, or wherein the second selected embryo explant or the second selected genetically modified plant or plant part does not further comprise the first genetic marker and/or the second genetic marker. In particular embodiments, the methods provided by the present disclosure may further comprise: identifying at least two genetic modifications present in the at least two embryo explants of the population or the at least two genetically modified plants or plant parts, the at least two genetic modifications comprising a first genetic modification and a second genetic modification; and selecting a first embryo explant of the population or a first genetically modified plant or plant part comprising the first genetic modification and a second embryo explant of the population or a second genetically modified plant or plant part comprising the second genetic modification, wherein the first selected embryo explant or the first selected genetically modified plant or plant part further comprises the first genetic marker and/or the second genetic marker, or wherein the first selected embryo explant or the first selected genetically modified plant or plant part does not further comprise the first genetic marker and/or the second genetic marker, and wherein the second selected embryo explant or the second selected genetically modified plant or plant part further comprises the first genetic marker and/or the second genetic marker, or wherein the second selected embryo explant or the second selected genetically modified plant or plant part does not further comprise the first genetic marker and/or the second genetic marker.

In some embodiments, the method of genetically modifying a population of plant embryo explants provided by the present disclosure may further comprise regenerating or growing a first regenerated genetically modified plant or plant part from the first selected embryo explant or any progeny generation of a cell thereof, or regenerating or growing a second regenerated genetically modified plant or plant part from the second selected embryo explant or any progeny generation of a cell thereof. In some embodiments, the present disclosure provides methods of genetically modifying a population of plant embryo explants further comprising observing a culturing characteristic of the first selected embryo explant and of the second selected embryo explant; observing a phenotype of the first regenerated genetically modified plant or plant part and of the second regenerated genetically modified plant or plant part; or observing a phenotype of the first selected genetically modified plant or plant part and of the second selected genetically modified plant or plant part. The methods of genetically modifying a population of plant embryo explants provided by the present disclosure, in some embodiments, may further comprise comparing the culturing characteristic of the first selected embryo explant and the second selected embryo explant and determining whether the culturing characteristic of the first selected embryo explant or the second selected embryo explant is superior; comparing the phenotype of the first regenerated genetically modified plant or plant part and of the second regenerated genetically modified plant or plant part and determining whether the phenotype of the first regenerated genetically modified plant or plant part or the second regenerated genetically modified plant or plant part is superior; or comparing the phenotype of the first selected genetically modified plant or plant part and of the second selected genetically modified plant or plant part and determining whether the phenotype of the first selected genetically modified plant or plant part or the second selected genetically modified plant or plant part is superior. In certain embodiments, these methods may further comprise regenerating or growing a first regenerated genetically modified plant or plant part from the first selected embryo explant or any progeny generation of a cell thereof, or regenerating or growing a second regenerated genetically modified plant or plant part from the second selected embryo explant or any progeny generation of a cell thereof based on the culturing characteristic of the first selected embryo explant and the second selected embryo explant. In particular embodiments, the methods provided by the present disclosure may further comprise crossing the first selected genetically modified plant with itself or a different plant to obtain a first progeny plant or seed; crossing the second selected genetically modified plant with itself or a different plant to obtain a second progeny plant or seed; crossing the first regenerated genetically modified plant with itself or a different plant to obtain a third progeny plant or seed; or crossing the second regenerated genetically modified plant with itself or a different plant to obtain a fourth progeny plant or seed.

In certain embodiments, the present disclosure provides methods of genetically modifying a population of plant embryo explants further comprising introducing a second heterologous polynucleotide molecule into at least one explant of a second population of embryo explants, wherein the at least one embryo explant of the second population has the same genotype or has a different genotype compared to: the at least one embryo explant of the population; the least one genetically modified plant or plant part; the selected embryo explant; or the selected genetically modified plant or plant part. The methods provided by the present disclosure, in particular embodiments, may comprise detecting a genetic modification of at least one of the embryo explants of the population or at least one of the genetically modified plants or plant parts.

In further embodiments, the method of genetically modifying a population of plant embryo explants provided by the present disclosure may further comprise identifying a genotype of at least one of the embryo explants of the population or at least one of the genetically modified plants or plant parts; and associating the genotype with at least one culturing characteristic or phenotype. Identifying, in particular embodiments, may comprise identifying a genetic marker or a quantitative trait locus (QTL) associated with the at least one culturing characteristic or phenotype. In certain embodiments, the at least one culturing characteristic or phenotype is selected from the group consisting of: explant excision efficiency, regeneration efficiency, genetic modification efficiency, transformation efficiency, shoot generation, and ability to regenerate into a genetically modified plant or plant part. In particular embodiments, the phenotype is an observable plant trait or results from the expression of a selectable or screenable marker. Plant traits may be observed using any method known in the art. Non-limiting examples of observable plant traits include plant height, ear height, brace root color, internode direction, internode length, leaf color, leaf length, leaf width, leaf sheath pubescence, leaf marginal waves, tassel length, anther color, glume color, silk color, silk position, husk opening, husk color, cob diameter, kernel row number, kernel number per row, endosperm type, endosperm color, relative maturity, flower color, hilum color, seed coat color, seed shape, leaf shape and growth habit. Selectable marker genes that may be used include, but are in no way limited to, aroA, EPSPS, aadA, pat, bar, hph (hygromycin B phosphotransferase), DMO (dicamba monooxygenase) CAT and NPT II. Although a plant selectable marker gene is generally used to confer tolerance to a selection agent, additional screenable or scorable marker gene(s) may also be used in addition to the selectable marker, perhaps also along with a gene of agronomic interest. Such screenable marker genes may include, for example, uidA for β-glucuronidase (GUS; e.g., as described in U.S. Pat. No. 5,599,670, which is hereby incorporated by reference) or gfp for green fluorescent protein and variants thereof (GFP described in U.S. Pat. Nos. 5,491,084 and 6,146,826, all of which are hereby incorporated by reference) or crtB for phytoene synthase (e.g., as described in U.S. Pat. Nos. 8,237,016 and 10,240,165, all of which are hereby incorporated by reference. Additional examples of screenable markers may include secretable markers whose expression causes secretion of a molecule(s) that can be detected as a means for identifying transformed cells.

In additional embodiments, the phenotype is the phenotype of a genetic modification, wherein the genetic modification results from an integration of the heterologous polynucleotide molecule or a fragment thereof into the genome of the at least one genetically modified plant or plant part, wherein the heterologous polynucleotide molecule comprises an expression cassette encoding a gene of interest, a site-specific nuclease, or a guide RNA molecule. In some embodiments, the method provided by the present disclosure may further comprise introgressing the chromosomal segment conferring the at least one culturing characteristic or the at least one phenotype into a plant having a plant genotype that lacks the culturing characteristic or the phenotype in the absence of the chromosomal segment; or crossing a genetically modified plant comprising the chromosomal segment conferring the at least one culturing characteristic or the at least one phenotype with itself or a second plant to produce a progeny plant or seed comprising the chromosomal segment.

In some embodiments, the present disclosure provides a method of genetically modifying a population of plant embryo explants, comprising: collectively introducing a heterologous polynucleotide molecule into at least two embryo explants of the population, wherein the embryo explants of the at least two different genotypes comprise embryo explants of a first genotype and embryo explants of a second genotype, and wherein the embryo explants of the first genotype and the embryo explants of the second genotype are present in the population at a predetermined ratio. The predetermined ratio is determined, in specific embodiments, based upon at least one culturing characteristic associated with the first genotype, with the second genotype, or with the first genotype and the second genotype. The at least one culturing characteristics, in certain embodiments, is selected from the group consisting of: explant excision efficiency, regeneration efficiency, shoot generation efficiency, genetic modification efficiency, transformation efficiency, and ability to regenerate into a genetically modified plant or plant part. In some embodiments, the predetermined ratio of the embryo explants of the first genotype and the second genotype comprises approximately an equal number of embryo explants of the first genotype and the second genotype. In further embodiments, the predetermined ratio of the embryo explants of the first genotype and the second genotype results in an approximately equivalent number of regenerated genetically modified plants or plant parts of the first genotype and the second genotype. In some embodiments, the first genotype is associated with a preferred culturing characteristic relative to the second genotype and the method comprises modifying the predetermined ratio to include an increased number of embryo explants of the second genotype compared to the first genotype in the population; or the second genotype is associated with the preferred culturing characteristic relative to the first genotype and the method comprises modifying the predetermined ratio to include an increased number of embryo explants of the first genotype compared to the second genotype in the population. In certain embodiments, the predetermined ratio of the embryo explants of the first genotype and the second genotype results in an approximately equivalent number of regenerated genetically modified plants or plant parts of the first genotype and the second genotype. The preferred culturing characteristic may, in certain embodiments, result in an increase in explant excision efficiency, regeneration efficiency, shoot generation efficiency, genetic modification efficiency, transformation efficiency, or ability to regenerate into a genetically modified plant or part.

In specific embodiments, the method of genetically modifying a population of plant embryo explants provided by the present disclosure may further comprise regenerating or growing a plurality of genetically modified plants or plant parts in contact with a regeneration medium from at least two embryo explants or any progeny generation of a cell thereof; and observing at least one culturing characteristic or phenotype of at least one of the embryo explants of the population or at least one of the genetically modified plants or plant parts. The at least one culturing characteristic or phenotype, in some embodiments, is associated with a genetic modification of the at least one embryo explant or the at least one genetically modified plant or plant part. In further embodiments, the methods provided by the present disclosure may further comprise observing a first culturing characteristic or phenotype of at least one embryo explant of a first genotype or at least one genetically modified plant or plant part of the first genotype; and observing a second culturing characteristic or phenotype of at least one embryo explant of a second genotype or at least one genetically modified plant or plant part of the second genotype. The first culturing characteristic or phenotype and the second culturing characteristic or phenotype, in particular embodiments, are the same. The first culturing characteristic or phenotype and the second culturing characteristic or phenotype, in additional embodiments, are different. The methods provided by the present disclosure may further, in some embodiments, comprise evaluating the at least one embryo explant or the at least one genetically modified plant or plant part of the first genotype and the at least one embryo explant or the at least one genetically modified plant or plant part of the second genotype by comparing the first culturing characteristic or phenotype and the second culturing characteristic or phenotype. In specific embodiments, each of the plurality of genetically modified plants or plant parts comprises at least one genetic modification. The methods provided by the present disclosure may, in particular embodiments, further comprise selecting a genetically modified plant comprising the at least one genetic modification, and crossing the genetically modified plant with itself or a second plant to obtain a progeny plant or seed, wherein the second plant has the same genotype or a different genotype as the genetically modified plant. In certain embodiments, the selecting comprises identifying a genotype of the genetically modified plant and selecting the genetically modified plant comprising the genotype.

The present disclosure provides, in certain embodiments, a method of genetically modifying a population of plant embryo explants, comprising: collectively introducing a heterologous polynucleotide molecule into at least two embryo explants of the population. In particular embodiments, the collectively introducing comprises site-directed integration of the heterologous polynucleotide or a fragment thereof. In some embodiments, the heterologous polynucleotide molecule comprises at least one expression cassette, wherein the at least one expression cassette comprises a selectable marker gene, a screenable marker gene, a gene of interest, a nucleotide sequence encoding a guide RNA molecule, or a nucleotide sequence encoding a site-specific nuclease. In certain embodiments, the heterologous polynucleotide molecule comprises or encodes a guide RNA. In further embodiments, the collectively introducing comprises introducing the heterologous polynucleotide molecule into the population contemporaneously. In particular embodiments, collectively introducing comprises introducing the heterologous polynucleotide molecule into the population of embryo explants while the population is present together in a single container.

In some embodiments, the present disclosure provides a method of genetically modifying a population of plant embryo explants, comprising: collectively introducing a heterologous polynucleotide molecule into at least two embryo explants of the population, wherein the population comprises embryo explants of at least two different plant genotypes, and wherein the population is defined as a population of dicot dry embryo explants. In particular embodiments, the population is defined as a population of soybean, cotton, or canola embryo explants.

In some embodiments, the present disclosure provides, a method of genetically modifying a population of plant embryo explants, the method comprising: collectively introducing a ribonucleoprotein or a site-specific nuclease into at least two plant embryo explants of the population, the at least two plant embryo explants each comprising meristematic tissue, wherein the population comprises plant embryo explants of at least two different plant genotypes. In certain embodiments, the ribonucleoprotein comprises a site-specific nuclease and a guide RNA molecule.

In certain embodiments, the methods provided by the present disclosure may further comprise excising the population of embryo explants from a population of plant seeds, wherein the excising is preformed prior to collectively introducing the heterologous polynucleotide molecule, a ribonucleoprotein, or a site-specific nuclease, wherein the population of plant seeds comprises plant seeds of at least two different plant genotypes. The methods provided by the present disclosure may, in certain embodiments, further comprise sorting the population of plant seeds into at least two batches of plant seeds according to a plant seed size, a plant seed shape, or a combination thereof prior to excising the population of embryo explants. In further embodiments, the at least two batches of plant seeds comprise a first batch of plant seeds and a second batch of plant seeds, wherein the population of plant embryo explants comprises a first batch of embryo explants and a second batch of embryo explants, and the excising comprises: excising the first batch of embryo explants from the first batch of plant seeds and the second batch of embryo explants from the second batch of plant seeds using the same excision method; or excising the first batch of embryo explants from the first batch plant seeds and the second batch of embryo explants from the second batch of plant seeds using different excision methods.

In some embodiments, the present disclosure provided a method further comprising: sorting a first population of plant seeds having a first genotype into at least two batches of plant seeds comprising a first batch of plant seeds and a second batch of plant seeds, wherein the first batch of plant seeds comprises a first plant seed size, a first plant seed shape, or a combination thereof, and the second batch of plant seeds comprises a second plant seed size, a second plant seed shape, or a combination thereof, and sorting a second population of plant seeds having a second genotype into at least two batches of plant seeds comprising a first batch of plant seeds and a second batch of plant seeds, wherein the first batch of plant seeds comprises the first plant seed size, the first plant seed shape, or a combination thereof, and the second batch of plant seeds comprises the second plant seed size, the second plant seed shape, or a combination thereof, wherein the population of plant seeds comprises: a first predetermined ratio of plant seeds from the first batch of plant seeds of the first genotype and the second batch of plant seeds of the first genotype, and a second predetermined ratio of plant seeds from the first batch of plant seeds of the second genotype and the second batch of plant seeds of the second genotype. Is some embodiments, the first predetermined ratio of plant seeds of the first genotype and the second predetermined ratio of plant seeds of the second genotype are approximately equal. In particular embodiments, the number of plant seeds from the first batch of plant seeds of the first genotype is approximately equal to the number of plant seeds from the first batch of plant seeds of the second genotype, and/or wherein the number of plant seeds from the second batch of plant seeds of the first genotype is approximately equal to the number of plant seeds from the second batch of plant seeds of the second genotype. In some embodiments, the at least two different genotypes comprise a first genotype and a second genotype and the excising results in the excision of an approximately equivalent number of embryo explants of the first genotype and the second genotype.

The plant seeds of the at least two different genotypes, in certain embodiments, may comprise plant seeds of a first genotype and plant seeds of a second genotype, wherein the plant seeds of the first genotype and the plant seeds of the second genotype are present in the population at a predetermined ratio. In certain embodiments, the predetermined ratio is determined based upon at least one culturing characteristic associated with the first genotype or with the second genotype. Non-limiting examples of such culturing characteristics include explant excision efficiency, regeneration efficiency, shoot generation efficiency, genetic modification efficiency, transformation efficiency, and ability to regenerate into a genetically modified plant or plant part. In some embodiments, the predetermined ratio of the plant seeds of the first genotype and the second genotype results in an approximately equivalent number of regenerated genetically modified plants or plant parts of the first genotype and the second genotype. In certain embodiments, the first genotype is associated with a preferred culturing characteristic and the method comprises modifying the predetermined ratio to include an increased number of plant seeds of the second genotype compared to the first genotype in the population; or the second genotype is associated with the preferred culturing characteristic and the method comprises modifying the predetermined ratio to include an increased number of plant seeds of the first genotype compared to the second genotype in the population, wherein the predetermined ratio of the plant seeds of the first genotype and the second genotype results in an approximately equivalent number of regenerated genetically modified plants or plant parts of the first genotype and the second genotype. Non-limiting examples of such preferred culturing characteristics include an increase in explant excision efficiency, regeneration efficiency, shoot generation efficiency, genetic modification efficiency, transformation efficiency, or ability to regenerate into a genetically modified plant or part.

In some embodiments, the population is defined as a population of dicot embryo explants, and the co-culture medium comprises at least one cytokinin or lipoic acid. Non-limiting examples of cytokinins that may be present in the co-culture medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). In certain embodiments, the concentration of the cytokinin in the co-culture medium is about 0.1 mg/L to about 50 mg/L. In specific embodiments, the at least one cytokinin is thidiazuron (TDZ) and the concentration of thidiazuron (TDZ) in the co-culture medium is about 0.1 mg/L to about 10 mg/L. In particular embodiments, the at least one cytokinin is 6-benzylaminopurine (BAP) and the concentration of the 6-benzylaminopurine (BAP) in the co-culture medium is about 0.1 mg/L to about 15 mg/L. The concentration of the lipoic acid in the co-culture medium, in some embodiments, is about 0.1 mg/L to about 500 mg/L.

In particular embodiments, the population is defined as a population of dicot embryo explants. In some embodiments, the heterologous polynucleotide molecule comprises a selectable marker gene, the regeneration medium comprises a selection agent, and the selectable marker gene provides resistance in a plant to the selection agent. In certain embodiments, the plurality of genetically modified plants or plant parts comprise at least one genetic modification. The at least one genetic modification, in particular embodiments, comprises an integration or insertion of the heterologous polynucleotide molecule or a fragment thereof into the genome of the plurality of genetically modified plants or plant parts, wherein the integration or insertion comprises at least one expression cassette or at least one transgene. In some embodiments, the at least one genetic modification comprises an edit introduced into the genome of the plurality of genetically modified plants or plant parts by a genome editing technique with a site-specific nuclease or a guide RNA molecule. The heterologous polynucleotide molecule, in certain embodiments, comprises at least one expression cassette, and the at least one expression cassette encodes the site-specific nuclease or the guide RNA molecule, or the heterologous polynucleotide molecule comprises at least two expression cassettes comprising a first expression cassette encoding the site-specific nuclease, and a second expression cassette encoding the guide RNA molecule. In certain embodiments, the regeneration medium comprises at least one cytokinin. Non-limiting examples of cytokinins that may be present in the regeneration medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). In some embodiments, the concentration of the cytokinin in the regeneration medium is about 0.1 mg/L to about 50 mg/L. The at least one cytokinin, in some embodiments, is zeatin or 6-benzylaminopurine (BAP) and the concentration of the zeatin or 6-benzylaminopurine (BAP) in the regeneration medium is about 0.1 mg/L to about 15 mg/L. In certain embodiments, the methods of the present disclosure may comprise regenerating or growing the plurality of genetically modified plants or plant parts in contact with the regeneration medium at about 15° C. to about 40° C. In certain embodiments, the population of embryo explants is a population of cotton embryo explants, and the method comprises regenerating or growing the plurality of genetically modified plants or plant parts in contact with the regeneration medium at about 30° C. to about 40° C. for a first regeneration period. The first regeneration period, in some embodiments, is about 1 hour to about 14 days. In particular embodiments, the method may further comprise regenerating or growing the plurality of genetically modified cotton plants or cotton plant parts in contact with the regeneration medium at about 20° C. to about 33° C. for a second regeneration period. The second regeneration period, in certain embodiments, is about 7 days to about 56 days. In particular embodiments, the method comprises regenerating or growing the plurality of genetically modified plants or plant parts in contact with the regeneration medium for about 5 days to about 70 days or about 14 days to about 50 days. In one embodiment, the population of embryo explants is a population of soybean embryo explants, and the method comprises regenerating or growing the plurality of genetically modified plants or plant parts in contact with the regeneration medium for about 5 days to about 70 days. In another embodiment, the population of embryo explants is a population of cotton embryo explants, and the method comprises regenerating or growing the plurality of genetically modified plants or plant parts in contact with the regeneration medium for about 14 days to about 70 days. In yet another embodiment, the population of embryo explants is a population of canola embryo explants, and the method comprises regenerating or growing the plurality of genetically modified plants or plant parts in contact with the regeneration medium for about 14 days to about 70 days.

In certain aspects, the population of embryo explants is a population of dicot embryo explants and the methods of the present disclosure further comprise regenerating or growing the plurality of genetically modified plants or plant parts in contact with a second regeneration medium for an extended regeneration period. In some embodiments, the second regeneration medium comprises at least one auxin, at least one cytokinin, or at least one auxin and at least one cytokinin. Non-limiting examples of auxins that may be present in the second regeneration medium include 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). Non-limiting examples of cytokinins that may be present in the second regeneration medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the auxin in the second regeneration medium, in particular embodiments, is about 0.1 mg/L to about 15 mg/L. The concentration of the at least one cytokinin in the second regeneration medium, in certain embodiments, is about 0.1 mg/L to about 50 mg/L. In some embodiments, the method comprises regenerating or growing the plurality of genetically modified plants or plant parts in contact with the second regeneration medium at about 15° C. to about 40° C. In certain embodiments, the extended regeneration period is about 7 days to about 56 days. In particular embodiments, the population of embryo explants is a population of cotton embryo explants, and the method further comprises transferring a selected portion of the plurality of genetically modified cotton plants or cotton plant parts to the second regeneration medium prior to regenerating or growing the plurality of genetically modified cotton plants or cotton plant parts in contact with the second regeneration medium.

In particular aspects, the population of embryo explants is a population of dicot embryo explants and the methods of the present disclosure may further comprise regenerating or growing the plurality of genetically modified plants or plant parts in contact with a first elongation medium for a first elongation period. In some embodiments, the first elongation medium comprises at least one auxin, at least one cytokinin, or at least one auxin and at least one cytokinin. In particular embodiments, the population of embryo explants is a population of canola embryo explants. Non-limiting examples of auxins that may be present in the first elongation medium include 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). Non-limiting examples of cytokinins that may be present in the first elongation medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the at least one auxin in the first elongation medium, in some embodiments, is about 0.1 mg/L to about 15 mg/L. The concentration of the at least one cytokinin in the first elongation medium, in certain embodiments, is about 0.1 mg/L to about 50 mg/L. In some embodiments, the methods of the present disclosure may comprise regenerating or growing the plurality of genetically modified plants or plant parts in contact with the first elongation medium at about 15° C. to about 40° C. The first elongation period, in particular embodiments, is about 7 days to about 56 days.

In some aspects, the population of embryo explants is a population of dicot embryo explants and the methods of the present disclosure may further comprise regenerating or growing the plurality of genetically modified plants or plant parts in contact with a second elongation medium for a second elongation period. The second elongation medium, in certain embodiments, comprises at least one auxin, at least one cytokinin, or at least one auxin and at least one cytokinin. Non-limiting examples of auxins that may be present in the second elongation medium include 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). Non-limiting examples of cytokinins that may be present in the second elongation medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the at least one auxin in the second elongation medium, in certain embodiments, is about 0.1 mg/L to about 15 mg/L. The concentration of the at least one cytokinin in the second elongation medium, in particular embodiments, is about 0.1 mg/L to about 50 mg/L. In some embodiments, the methods of the present disclosure comprise regenerating or growing the plurality of genetically modified plants or plant parts in contact with the second elongation medium at about 15° C. to about 40° C. In particular embodiments, the second elongation period is about 7 days to about 56 days.

In certain embodiments, the present disclosure provides a combination of a robust genomic platform, a genome-editing toolbox, and a high throughput, genotype-independent transformation system and enables genome editing at any target site in any genotype and species.

The present disclosure also provides a method of simultaneously transforming and editing numerous genotypes in a single experiment under identical conditions.

In addition, the present disclosure provides certain embodiments for bulk transformation of mixed germplasm, which demonstrates genotype-flexible regeneration and genome editing of up to 100 elite genotypes via seed embryo explants-based meristem transformation in soybean and maize. In specific embodiments, in soybean, over 800 distinct edits were recovered at a conserved target site, such as near a gene locus of interest, with individuals representing nearly every transformed germplasm.

In additional embodiments in maize, although more inbred to inbred variations were observed, transformants from 23 of 40 female inbreds were recovered. The present disclosure therefore describes accelerated implementation and deployment of genome-editing strategies in breeding and product development for precision breeding.

Any embodiment or aspect of the present disclosure may be used in combination with any other embodiment or aspect described herein.

### BRIEF DESCRIPTION OF THE DRAWINGS

FIG. 1 shows a graphical representation of the similarity scores of soybean lines sampled following bulk transformation during genotyping.

FIG. 2 shows a comparison of transformation and editing frequencies by maturity group in a soybean bulk. FIG. 2A shows a comparison of the frequency of transformation events in each maturity group to the proportion of the bulk consisting of that maturity group. FIG. 2B shows the total number of edited versus non-edited events by maturity group.

FIG. 3 shows the proportion of heritable edit reads by T-DNA copy number following bulk soybean transformation.

### DETAILED DESCRIPTION

The following is a detailed description provided to aid those skilled in the art in practicing the embodiments of the present disclosure. Modifications and variations to the embodiments described herein can be made without departing from the spirit or scope of the present disclosure. Compositions and methods are provided for collectively transforming or genetically modifying a population of embryo explants, which may include one or more steps of explant preparation, explant rehydration, Rhizobiales bacterium inoculation and co-culture, particle bombardment, bud induction, extended bud induction, and/or regeneration or development of genetically modified plants or plant parts as described herein. The present disclosure further provides methods for identifying and selecting individual members of the population which comprise a desired genotype, germplasm, genetic modification, culturing characteristic, and/or phenotype.

The present disclosure provides compositions that include a population of distinct germplasm and at least one heterologous polynucleotide molecule, a ribonucleoprotein, morphogenic regulator, or a site specific nuclease, wherein the population of germplasm comprises at least two different plant genotypes. The present disclosure further provides methods for collectively introducing a heterologous polynucleotide into a population of germplasm, such as embryo explants, having different genotypes to introduce one or more genetic modification(s). As described herein, a genetic modification may comprise a transgene or site-directed integration of a DNA segment or transgene, and/or a genetic modification may comprise a mutation or edit. The present disclosure represents a substantial advance in the art, as it provides compositions and methods for producing a plurality of genetically modified plants having different genotypes at once or collectively, without the use of time consuming, costly, and inefficient plant breeding or introgression techniques. The present disclosure further provides methods and compositions for investigating or screening the variable phenotypes produced when introducing the same genetic modification into different plant germplasms or genetic backgrounds, which may be described as “Germplasm x Transgene/Edit (or Trait) Interactions.” Prior to the present disclosure, analysis of such Germplasm x Transgene/Edit (or Trait) Interactions typically involved the creation of a transgenic event, edit, and/or trait in a single germplasm followed by crossing or introgressing the event, edit, and/or trait into other germplasms, a process which is time consuming, costly, and inefficient. Prior to the present disclosure any genetic background or germplasm dependent differences in phenotype for a given transgenic event, edit, and/or trait could not be tested, observed, screened, or selected until the event, edit, and/or trait has been crossed or introgressed into multiple germplasms over several generations. The present disclosure further provides methods and compositions for investigating or screening the variable characteristics of different plant germplasms or genetic backgrounds when collectively introducing a heterologous polynucleotide and/or genetic modification(s) into the different plant germplasms or genetic backgrounds.

One of ordinary skill in the art will appreciate that there are a number of transformation methods used for a variety of different plant types. These known methods can start with a variety of different plant tissues and those known methods and associated compositions can be modified to be used with the methods and compositions described herein.

### A. Explant Population Preparation

In accordance with embodiments of the present disclosure, populations of seed embryo explants can be produced from plant seeds for production of genetically modified plants or plant parts. Populations of embryo explants may be produced from seeds by applying mechanical force, for example by cutting, grinding, scraping, crushing, or wounding, the seeds. Seeds for use according to the present disclosure may be harvested from plants grown in a field or greenhouse, and may be mature or immature seeds, but may preferably be mature seeds. Examples of seeds for use according to the present disclosure include, but are not limited to, monocot seeds, dicot seeds, corn seeds, wheat seeds, barley seeds, rice seeds, oat seeds, sorghum seeds, rye seeds, millet seeds, soybean seeds, cotton seeds, canola seeds, and Brassica seeds. Examples of embryo explants for use according to the present disclosure include, but are not limited to, mature embryo explants, immature embryo explants, monocot embryo explants, dicot embryo explants, corn embryo explants, wheat embryo explants, barley embryo explants, rice embryo explants, oat embryo explants, sorghum explants, rye embryo explants, millet embryo explants, soybean embryo explants, cotton embryo explants, canola embryo explants, and Brassica embryo explants. Use of mature seeds may provide the benefits or advantages of improved seed storage, explant preparation, and/or culturing. Examples of monocot plants, seeds, or explants that may be used according to present embodiments include those derived from any plant species within the Poaceae or Gramineae family of monocot or cereal plants and grasses, which may include any Zea genus corn or maize species, such as Zea mays, any Oryza genus rice species, such as Oryza sativa, any Triticum genus wheat species, such as Triticum aestivum or Triticum turgidum var durum, any Hordeum genus barley species, such as Hordeum vulgare, any Avena genus oat species, such as Avena sativa, any Sorghum genus sorghum species, such as Sorghum bicolor or Sorghum vulgare, any Secale genus rye species, such as Secale cereale, any Saccharum sugarcane species, or any Setaria, Pennisetum, Eleusine, Echinochloa, or Panicum genus millet species, such as Setaria virdis, Setaria italica, Pennisetum glaucum, Eleusine coracana, Echinochloa frumentacea, Panicum sumatrense, or Panicum miliaceum. Examples of dicot plants, seeds, and explants that may be used according to the present embodiments include those derived from any plant species within the family Fabaceae, Malvaceae, or Brassicaceae, which may include any Glycine genus species, such as Glycine max, any Gossypium genus species, such as Gossypium arboretum, Gossypium herbaceum, Gossypium raimondii, Gossypium thurberi, Gossypium barbadense, Gossypium hirsutum, Gossypium darwinii, Gossypium mustelinum, Gossypium tomentosum, Gossypioides brevilanatum, or Gossypioides kirkii, or any Brassica genus species, such as Brassica napus, Brassica rapa, or Brassica juncea.

According to some embodiments, methods and compositions are provided for preparing, culturing, selecting, and using a population of explants, as well as the population of explants or cultured explants produced thereby. As used herein, the term “explant” or “seed embryo explant” refers to a plant part or plant tissue that is capable of being genetically modified and subsequently generated/regenerated into a genetically modified plant or plant part. An “explant” or “seed embryo explant” may refer to a plant seed or any part of a plant seed, which comprises at least a portion of a plant seed embryo in the case of a seed embryo explant. An “explant” or “seed embryo explant” may comprise an embryo explant excised from a plant seed that may comprise at least a part of an embryo meristem tissue. Alternatively, an “explant” or “seed embryo explant” may refer to a whole or intact plant seed, or a crushed, deformed or partially opened plant seed that may be produced by any suitable mechanical process. As used herein the term “distinct embryo explant” refers to an explant comprising genomic features that are identifiable and correlated to true breeding characteristics. Distinct embryo explants are characterized by their ability to be identified at the genomic level and distinguished from other embryo explants included within the same bulk transformation pool. Various methods for preparing plant seed explants and plant seed embryo explants are known in the art. As used in reference to an explant or seed embryo explant, “partially opened” refers to an altered state of a plant seed that has one or more openings or fissures in the plant seed. Such openings or fissures may be introduced by a mechanical force, such as squeezing, crushing, rolling, pressing, or extruding. An explant or seed embryo explant that is a whole or intact plant seed or a crushed, deformed or partially opened plant seed may in many cases have its seed coat removed. An explant may be defined, in one aspect or embodiment, as comprising meristematic tissue or embryonic meristem tissue, which contains plant cells that can differentiate or develop to produce multiple plant structures including, but not limited to, stem, roots, leaves, germ line tissue, and seeds. In certain embodiments, an embryo explant may be defined as comprising all or part of a seed embryo removed from other non-embryonic seed tissues and further comprising all or part of a meristematic tissue or embryonic meristem tissue. In some embodiments, the present disclosure provides embryo explants comprised of the apical portion of the embryo axis lacking the radical, wherein remaining portions of the seeds have been substantially removed from the embryo explant. In further embodiments, the present disclosure provides embryo explants which do not germinate and remain viable and competent for genetic modification. As used herein, the term “cultured embryo explant” refers to an embryo explant that is in culture but has not yet regenerated into a plant or plant part. In specific embodiments, the cultured embryo explant may be a genetically modified embryo explant. A cultured embryo explant may be cultured, in some embodiments, in contact with co-culture medium, bud induction medium, extended bud induction medium, or regeneration medium. A cultured embryo explant from a monocot seed may be cultured, in some embodiments, following any inoculation step in contact with a co-culture medium, a bud induction medium, an extended bud induction medium, and a regeneration medium, and a cultured embryo explant from a dicot seed may be cultured, in some embodiments, following any inoculation step in contact with a co-culture medium and a regeneration medium. As used herein, a “population of embryo explants” refers to a group of explants from the same plant species. The population of explants, in some embodiments, may include explants having the same or different genotypes, germplasms, and/or genetic backgrounds. In certain embodiments, the genotype of the explants within the population may be known or may be unknown. In specific embodiments, the population of embryo explants may refer to a group of embryo explants which includes embryo explants of at least two different plant genotypes. In specific embodiments, the present disclosure provides a population of embryo explants or distinct embryo explants having at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. In certain embodiments, the present disclosure provides a population of embryo explants or distinct embryo explants comprising at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 10,000, at least 15,000, at least 20,000, at least 30,000, at least 40,000, at least 50,000, at least 60,000, at least 70,000, at least 80,000, at least 90,000, at least 100,000, at least 150,000, or at least 200,000 embryo explants, or from about 2 to about 1000, about 5 to about 900, about 5 to about 800, about 5 to about 700, about 5 to about 600, about 5 to about 500, about 10 to about 500, from about 15 to about 400, from about 20 to about 300, from about 25 to about 200, from about 10 to about 150, from about 10 to about 100, from about 10 to about 90, from about 10 to about 80, from about 10 to about 70, from about 10 to about 60, from about 10 to about 50, from about 10 to about 40, from about 10 to about 30, from about 10 to about 20, from 2 to about 50, from 2 to about 40, from 2 to about 30, from 2 to about 20, from 2 to about 15, or from 2 to about 10 embryo explants. In certain embodiments, explants according to this disclosure may be produced manually or using an automated process. For example, seed tissues may be removed from a seed by cutting, grinding, scraping, crushing, wounding, or any other similar process. Manual or automated methods for removal of unnecessary seed parts may also be carried out. A fluid, non-limiting examples of which include compressed air, other gases, and liquids, can be used to separate explants from debris during explant purification.

In particular embodiments provided by the present disclosure, embryo explants or distinct embryo explants may be excised from a population of plant seeds, wherein the population of plant seeds comprise plant seeds of at least two different plant genotypes. In specific embodiments, the population of plant seeds from which the population of embryo explants or distinct embryo explants is excised may have at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes. In certain embodiments, the present disclosure provides a population of plant seeds from which embryo explants or distinct embryo explants may be excised comprising at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 10,000, at least 15,000, at least 20,000, at least 30,000, at least 40,000, at least 50,000, at least 60,000, at least 70,000, at least 80,000, at least 90,000, at least 100,000, at least 150,000, or at least 200,000 plant seeds, or from about 2 to about 1000, about 5 to about 900, about 5 to about 800, about 5 to about 700, about 5 to about 600, about 5 to about 500, about 10 to about 500, from about 15 to about 400, from about 20 to about 300, from about 25 to about 200, from about 10 to about 150, from about 10 to about 100, from about 10 to about 90, from about 10 to about 80, from about 10 to about 70, from about 10 to about 60, from about 10 to about 50, from about 10 to about 40, from about 10 to about 30, from about 10 to about 20, from 2 to about 50, from 2 to about 40, from 2 to about 30, from 2 to about 20, from 2 to about 15, or from 2 to about 10 plant seeds.

In some embodiments, the present disclosure provides methods which include sorting a population of plant seeds into at least two batches of plant seeds according to a plant seed size and/or a plant seed shape prior to excising a population of embryo explants or distinct embryo explants. Sorting a population of seeds according to seed size and/or seed shape prior to excision may, in some embodiments, result in an approximately equivalent number of explants excised from the seeds of each genotype present in the population. Different batches of seeds sorted according to seed size and/or seed shape may be excised, in certain embodiments, using different excision methods and/or settings in order maximize explant excision efficiency. In particular embodiments, different batches of seeds sorted according to seed size and/or seed shape may be excised using the same excision method and/or settings. In some embodiments, two or more different batches of seeds sorted according to seed size and/or shape may be combined prior to explant excision. In some embodiments, two or more different batches of seeds sorted according to seed size and/or shape may be combined prior to explant excision in equal numbers or in different proportions or percentages to account for their relative transformation, culturing, and/or regeneration efficiency. For example, a batch of seeds whose explants have a greater transformation, culturing, and/or regeneration frequency may be added to a population of seeds for explant excision at a lower percentage than another batch of seeds whose explants have a lower transformation, culturing, and/or regeneration frequency. Alternatively, for example, explants may be excised from different batches of seeds separately and then combined prior to subsequent rehydration, culturing, and/or transformation steps in equal numbers or in different proportions or percentages to account for their relative transformation, culturing, and/or regeneration efficiency.

Embryo explants may be excised from dry, dried, or wet seeds. Mature plant seeds may become drier as part of their normal maturation process, although seeds may be further dried prior to explant excision and/or explants may be dried following excision from seeds. Dry or dried excised plant embryo explants may be immediately used for genetic modification or may be stored for a period of time for later use. Explant preparation may further comprise drying the seed and/or explant to a desired moisture content. Drying the seed and/or explant to such a desired moisture content may improve excision, storage, and/or use of the seed and/or explant, depending upon the initial moisture content of the seed or explant. Following excision, the explant may be purified or separated from other seed material and debris by rinsing, flotation, or other methods known in the art. In certain embodiments, the present disclosure provides a seed or explant having an internal moisture of about 3% to about 25%, about 3% to about 20%, about 3% to about 15%, about 3% to about 10%, about 3% to about 11%, about 4% to about 16%, about 4% to about 12%, about 5% to about 10%, including about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or about 25% internal moisture, including all values and ranges derivable therebetween. An explant may be produced from a mature seed having a moisture content as described herein. In particular embodiments, the moisture content of the seed or explant may be measured prior to or after explant excision, prior to or after explant storage, during explant storage, prior to explant rehydration, and/or prior to genetic modification or transformation. Seed embryo explants may be defined, in one aspect or embodiment, as comprising meristematic tissue or embryonic meristem tissue, which contains plant cells that can differentiate or develop to produce multiple plant structures including, but not limited to, stem, roots, leaves, germ line tissue, and seeds. Indeed, an embryo explant may be defined as comprising all or part of a seed embryo removed from other non-embryonic seed tissues and further comprising all or part of a meristematic tissue or embryonic meristem tissue.

In one aspect, any embryo explant may be prepared or used according to the embodiments of the present disclosure. In particular embodiments, the embryo explant may be a mature embryo, or an immature embryo, and/or may comprise meristematic tissue, callus tissue, or any other tissue that is transformable and regenerable. In particular embodiments, the embryo explant is defined as a distinct embryo explant. In some embodiments, the mature embryo explant is a dry excised explant. Dry excised explants may be taken from seeds and used almost directly as targets for transformation or genetic modification. In some embodiments, dry excised explants or dried explants may be taken from mature dry seeds and used as targets for transformation or genetic modification with perhaps only minimal wetting, hydration, or pre-culturing steps. In further embodiments, wet, dried wet, or wet excised embryo explants may be used as a target for transformation or genetic modification. In further embodiments, immature or mature embryo explants or excised embryo explants may be dried prior to transformation or genetic transformation. As used herein, “wet” embryo explants refer to dry excised explants subjected to wetting, hydration, imbibition, or other minimal culturing steps prior to transformation or genetic modification. As used herein, “dried” embryo explants refer to excised explants subjected to drying steps prior to transformation or genetic modification. As used herein, “dried wet” embryo explants refer to embryo explants which are primed for germination by wetting and then dried to arrest germination. As used herein, “wet excised” explants refer to explants excised from imbibed or hydrated seeds. A wet embryo explant is hydrated or imbibed after excision from a seed, whereas a wet excised embryo explant is excised from an already hydrated or imbibed seed. As used herein, a “callus” refers to a proliferating mass of unorganized, undifferentiated and/or dedifferentiated plant cells or tissue.

According to present embodiments, the explants of the population may be defined, in some embodiments, as comprising meristematic tissue or embryonic meristem tissue, which contains plant cells that can differentiate or develop to produce multiple plant structures including, but not limited to, stem, roots, leaves, germ line tissue, and seeds. In particular embodiments, the embryo explants of the population may be defined as comprising all or part of a seed embryo removed from other non-embryonic seed tissues and further comprising all or part of a meristematic tissue or embryonic meristem tissue.

Explants for use according to the present disclosure may be genetically modified at various times after isolation, excision, and/or removal from the seed. In one embodiment, explants may have been removed from seeds for less than a day, for example, from about 1 to about 24 hours, such as about 1, 2, 3, 5, 7, 8, 9, 10, 11, 12, 13, 14, 15, 16, 17, 18, 19, 20, 21, 22, 23 or 24 hours prior to use. In further embodiments, explants may be stored for longer periods, including days, weeks, months, or years prior to use. Methods and parameters for drying, storing, transforming, regenerating, and/or germinating seeds or seed embryo explants are known in the art (see e.g., U.S. Pat. Nos. 7,402,734, 8,044,260, 8,030,544, 8,362,317, U.S. 2022/0340916, and U.S. 2022/0340925, specifically incorporated herein by reference in their entirety; Senaratna et al., 1983, Pl. Physiol. 72:620-624, 1983; Vertucci and Roos, 1990, Pl. Physiol. 90:1019-1023, 1990; Chai et al., 1998, Seed Science Research 8 (Supplement 1):23-28, 1998). Any conditions may be used as desired, including incubation or storage at temperatures, for example, of about −80° C. to about 60° C. If explants or seeds are stored in the freezer, they can be thawed prior to use in subsequent steps, wherein such a thawing step may be for a time period between about 20 minutes and about 4 hours or longer, or between about 30 minutes and about 2 hours, depending on the storage temperature and number of seeds or explants to be thawed and brought to room temperature for explant excision or use in transformation.

The disclosure may in certain aspects involve sterilization of seeds or explants. Sterilization can include contacting seed or explant material with various liquids or gases that serve to reduce or eliminate the presence of viable bacterial or fungal contaminants that could otherwise interfere with seed or embryo viability. Sterilization by application of liquid may also hydrate or partially hydrate the plant seeds, explants, embryos, or tissues and serve the purpose of priming the seeds, explants, embryos, or tissues. Methods for sterilization include, but are not limited to, the use of chlorine gas, ozone, solutions of bleach or alcohol, ultraviolet light, temperatures of −20° C. or lower, and exposure to a temperature higher than 40° C. In some embodiments, a sterilization medium may comprise polyethylene glycol and/or an antifungal or antimicrobial agent.

In one aspect of the present disclosure, explants may be rehydrated prior to transformation or genetic modification. Rehydration media or solutions are known in the art and may comprise, for example, water, basal salts, macronutrients, micronutrients, and/or vitamins. In certain embodiments, the rehydration solution may comprise polyethylene glycol, an antimicrobial agent, and/or an antifungal agent. In some embodiments, the rehydration medium may be water. In some embodiments, the rehydration medium may be an inoculation medium. The rehydration medium will typically not contain any plant hormones, such as an auxin or cytokinin. It may be important to have an optimal time period for rehydration. If the explants are in a rehydration medium/media for too long, the explants can become “mushy” and not regenerable or viable, but if the explants are in a rehydration medium/media for a time period that is too short, the explants may not rehydrate fully, and thus may not become transformed or may be transformed less efficiently. Embryo explants of some plant species are more or less sensitive to rehydration, which may depend on their relative size or composition. Embryo explants that are more sensitive to rehydration may be placed in a rehydration medium for a shorter period of time but may tolerate up to about 1 hour to about 2 hours of rehydration. In one embodiment, rehydrating seed embryo explants may be carried out for a period of time in a range from about 15 minutes to about 24 hours, or about 15 minutes to about 12 hours, or about 15 minutes to about 6 hours, or about 15 minutes to about 4 hours, or about 30 minutes to about 2 hours prior to transformation or genetic modification or any length of time within such ranges, such as for about 15 minutes, about 20 minutes, about 30 minutes, about 1 hour, about 1.5 hours, about 2 hours, about 2.5 hours, about 3.5 hours, or about 4 hours, or less than or equal to about 4 hours, or less than or equal to about 3 hours, or less than or equal to about 2.5 hours, or less than or equal to about 2 hours, or in a range of about 20 minutes to about 4 hours, or in a range of about 20 minutes to about 3 hours, or in a range of about 20 minutes to about 2 hours, or in a range of about 20 minutes to about 1.5 hours, or in a range of about 1 hour to about 3 hours, or in a range of about 1 hour to about 2.5 hours, or in range of about 1 hour to about 2.0 hours, or in a range of about 1.5 hours to about 2.5 hours, including all values and ranges derivable therebetween. Rehydration of embryo explants prior to transformation or genetic modification may improve transformation or editing frequency or the recovery of transformed or edited plants, as compared to explants that were not rehydrated. In some embodiments, the rehydration medium may be shaken or rocked, such as on a shaker or rocker, or otherwise physically or mechanically agitated, moved, or inverted during this step to improve rehydration and/or reduce time for rehydration of the explants. In particular embodiments, the embryo explants may be monocot embryo explants and rehydrating seed embryo explants may be for at least about 2 hours prior to transformation or genetic modification, which may improve transformation or editing frequency or the recovery of transformed or edited plants, as compared to explants rehydrated for about 1 hour or less. In particular embodiments, the embryo explants may be dicot embryo explants and rehydrating embryo explants may be carried out for about 1 hour or from about 15 minutes to about 4 hours. In certain embodiments, embryo explants may be rinsed to remove rehydration medium prior to subsequent steps.

In certain embodiments, embryo explants may be rinsed to remove rehydration medium prior to subsequent steps. In certain embodiments, embryo explants may be rinsed for about 20 seconds to about 10 minutes, about 20 seconds to about 9 minutes, about 20 seconds to about 8 minutes, about 20 seconds to about 7 minutes, about 20 seconds to about 6 minutes, about 20 seconds to about 5 minutes, about 20 seconds to about 4 minutes, about 20 seconds to about 3 minutes, about 20 seconds to about 2 minutes, about 20 seconds to about 1 minute, about 1 minute to about 10 minutes, about 1 minutes to about 6 minutes, about 2 minutes to about 6 minutes, or about 3 minutes to about 5 minutes, including all values and ranges derivable therebetween. The embryo explants may be subjected to, in certain embodiments, about 1 round to about 10 rounds, about 1 round to about 9 rounds, about 1 round to about 8 rounds, about 1 round to about 7 rounds, about 1 round to about 6 rounds, about 1 round to about 5 rounds, about 1 round to about 4 rounds, about 1 round to about 3 rounds, about 1 round to about 2 rounds, about 2 rounds to about 6 rounds, or about 3 rounds to about 5 rounds of rinsing, including all values and ranges derivable therebetween. In particular embodiments, embryo explants may be rinsed in a container or by placement in a strainer, fishnet, or the like and then allowing rinse solution to flow over the explants. In particular embodiments, embryo explants may be rinsed in a container by removing, decanting, and/or aspirating the rehydration solution or liquid, and then adding the rinse solution or liquid. The first volume of rinse solution or liquid may be removed, decanted, and/or aspirated and then replaced with new rinse solution or liquid, which may be repeated one or more times (i.e., for two or more rounds of rinsing in total). In some embodiments, the rehydration solution or liquid and the rinsing solution or liquid are the same solution or liquid. In certain embodiments, the rehydration solution or liquid and the rinsing solution or liquid are different solutions or liquids. In some embodiments, the rinse solution is water. After rehydration and/or rinsing of the explants, the rehydration and/or rinsing medium may be removed, such as by decanting, pipetting, vacuuming, and/or aspirating the medium, and the explants may be dried by blotting, wicking, or other method, such as with filter paper or other absorbent material in contact with the rehydration or rinsing solution or liquid, to remove at least an excess amount of rehydration or rinsing solution or liquid. Blotting and the like, may be particularly useful or necessary for explants that are more sensitive to excessive rehydration.

As used herein, a “genetically modified” germplasm, plant, plant part, plant tissue, explant, or plant cell comprises a genetic modification, such as a mutation, edit, or transgene introduced into the genome of the germplasm, plant, plant part, plant tissue, explant, or plant cell through genetic engineering, which may be via a genetic transformation, mutagenesis, or a genome editing technique. As used herein, a “genetic modification” refers to one or more transgenic event(s), mutation(s), and/or edit(s) introduced into the genome of a plant, plant part or plant cell using a transformation, mutagenesis, or genome editing technique. Apart from a genome editing technique, a mutagenesis technique may include any chemical, physical, radiological, or biological (e.g., transposon-mediated) mutagenesis technique or mutagen. As used herein, a “transgenic” plant, plant part, plant tissue, explant or plant cell has an exogenous nucleic acid sequence, polynucleotide, expression cassette, or transgene integrated into the genome of the plant, plant part, plant tissue, explant, or plant cell. A genetically modified plant, plant part, plant tissue, explant, or plant cell may comprise, in certain embodiments, a heritable edit or a non-heritable edit. A heritable edit or a non-heritable edit may be identified, in some embodiments, through genetic sequencing. When genetic sequencing is performed on a sample comprising a polynucleotide molecule from or derived from a genetically modified plant, plant part, plant tissue, explant, or plant cell and >10% of the sequencing reads contain the expected edit, then the edit is likely heritable and thus may be described as a “heritable edit.” When genetic sequencing is performed on a sample comprising a polynucleotide molecule from or derived from a genetically modified plant, plant part, plant tissue, explant, or plant cell and 1-10%, or possibly 0.1%-10%, 0.1%-5%, or 0.1%-1%, or greater than 0%, of sequencing reads contain the expected edit, then the edit may not be heritable and thus may be described as a “non-heritable edit”.

Transformation or editing of embryo explants or plants may be measured, in some embodiments of the present disclosure, by genotyping, # shoots generated (or # shoots regenerated) after Agrobacterium-mediated inoculation, % shoots generated (or % shoots regenerated) after Agrobacterium-mediated inoculation, relative shoot presence, # transformants, % transformants, relative transformation rate, # edited, % edited, relative editing rate, # edited (heritable edits), % edited (heritable edits), relative editing rate (heritable), # edited (non-heritable edits), % edited (non-heritable edits), or relative editing rate (non-heritable), as described herein.

### B. Introduction of a Heterologous Polynucleotide Molecule, Ribonucleoprotein, or Nuclease

Methods and compositions are provided herein for collective genetic transformation or modification of a population of embryo explants. In specific embodiments, a heterologous polynucleotide molecule, a ribonucleoprotein, and/or a site-specific nuclease is collectively introduced into at least two embryo explants of the population. As used herein, the term “collectively introducing” refers to introducing the heterologous polynucleotide molecule, a ribonucleoprotein, and/or a site-specific nuclease into one or more explants of the population at approximately the same time and/or when the explants of the population are within the same or approximately the same area or within one or more containers. In some embodiments, a step of collectively introducing a heterologous polynucleotide molecule, a ribonucleoprotein, and/or a site-specific nuclease may be included in any transformation protocol known in the art. Non-limiting examples of such transformation protocols include protocols for protoplast transformation, plastid transformation, callus related transformation, dry embryo explant transformation, bacterium related transformation, and non-bacterium related transformation, including electroporation, microprojectile or particle bombardment, microinjection, PEG-mediated transformation, and other modes of direct DNA uptake. Heterologous polynucleotides and recombinant nucleic acid sequences can also encode morphogenic regulators such as, a Wuschel (WUS) gene (e.g., from blackberry and/or cherry), Baby boom (BBM, e.g., from blackberry and/or cherry), Knotted 1 (KN1) gene (e.g., from maize, blackberry, and/or cherry), isopentenyl transferase (ipt) gene (e.g., from Agrobacterium tumefaciens, blackberry, and/or cherry), CLAVATA3 (CLV3) mutant, miR156 (e.g., from citrus), AtGRF5 (e.g., from Arabidopsis thaliana), NTT (e.g., from Arabidopsis thaliana, blackberry, and/or cherry), HDZipII (e.g., from Arabidopsis thaliana, blackberry, and/or cherry), and orthologs or polypeptides thereof. One of ordinary skill in the art will appreciate that morphogenic regulators can be introduced into transformation methods as either a protein or encoded in recombinant nucleic acid sequences. The heterologous polynucleotide molecule, the ribonucleoprotein and/or the site-specific nuclease may, in some embodiments, be collectively introduced into the explants of the population contemporaneously, simultaneously, or approximately simultaneously. In specific embodiments, the population of embryo explants may comprise one or more groups or batches of embryo explants. The groups or batches of the population may be present, in certain embodiments, in separate containers, which may have the same approximate cross-sectional area or volume, and the heterologous polynucleotide molecule, the ribonucleoprotein, and/or the site-specific nuclease may be collectively introduced into the groups or batches of the population at approximately the same time. In further embodiments, the heterologous polynucleotide molecule, ribonucleoprotein, and/or site-specific nuclease may be collectively introduced into the population of embryo explants while the individual explants of the population are present together within a single container.

Embodiments of the present disclosure may include genetically transforming or modifying at least one cell of each of at least two embryo explants of the population by collectively introducing a heterologous polynucleotide molecule, ribonucleoprotein, and/or site-specific nuclease by any suitable method or technique known in the art, such as electroporation, microprojectile or particle bombardment, microinjection, PEG-mediated transformation, Rhizobiales- or Agrobacterium-mediated transformation, and other modes of direct DNA uptake. All or part of the heterologous polynucleotide may then be transformed or incorporated into the genome of the plant cell, expressed into one or more editing molecules or tools (such as a guide RNA and/or site-specific nuclease), and/or provide a template for editing or site-directed integration. According to many embodiments, the heterologous polynucleotide is introduced into the population via Rhizobiales- or Agrobacterium-mediated transformation.

In particular embodiments, the Rhizobiales bacterium or the Agrobacterium may be cultured in a medium comprising optionally one or more cytokinins and/or lipoic acid prior to Rhizobiales- or Agrobacterium-mediated transformation. Alternatively, the medium for culturing the Rhizobiales bacterium or the Agrobacterium may not contain or comprise a cytokinin and/or lipoic acid. Such culturing medium may comprise, for example, water, basal salts, macronutrients, micronutrients, and/or vitamins. In certain embodiments, the culturing medium may comprise polyethylene glycol and/or an antimicrobial agent and/or an antifungal agent. According to some embodiments, the culturing medium comprises an inoculation medium. Non-limiting examples of cytokinins that may be used in the bacterial culture medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the cytokinin in the bacterial culture medium may be, in some embodiments, in a range from about 0.1 mg/L to about 50 mg/L, about 0.1 mg/L to about 45 mg/L, about 0.1 mg/L to about 40 mg/L, about 0.1 mg/L to about 35 mg/L, about 0.1 mg/L to about 30 mg/L, about 0.1 mg/L to about 25 mg/L, about 0.1 mg/L to about 20 mg/L, about 0.1 mg/L to about 15 mg/L, about 0.1 mg/L to about 10 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 5 mg/L, about 0.5 mg/L to about 4 mg/L, about 0.5 mg/L to about 3 mg/L, about 1 mg/L to about 5 mg/L, about 1 mg/L to about 4 mg/L, about 1 mg/L to about 3 mg/L, about 5 mg/L to about 40 mg/L, about 5 mg/L to about 30 mg/L, about 10 mg/L to about 30 mg/L, or about 20 mg/L to about 30 mg/L, or about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all values and ranges derivable therebetween. In certain embodiments, lipoic acid may be present in the bacterial culture medium at a concentration in a range of about 0.1 mg/L to about 500 mg/L, about 0.1 mg/L to about 400 mg/L, about 0.1 mg/L to about 300 mg/L, about 0.1 mg/L to about 200 mg/L, about 10 mg/L to about 200 mg/L, about 10 mg/L to about 180 mg/L, about 10 mg/L to about 160 mg/L, about 10 mg/L to about 140 mg/L, about 10 mg/L to about 120 mg/L, about 10 mg/L to about 100 mg/L, about 20 mg/L to about 80 mg/L, about 40 mg/L to about 60 mg/L, about 50 mg/L to about 60 mg/L, or about 50 mg/L to about 55 mg/L, or about 5 mg/L, about 10 mg/L, about 15 mg/L, about 20 mg/L, about 25 mg/L, about 30 mg/L, about 35 mg/L, about 40 mg/L, about 45 mg/L, about 50 mg/L, about 55 mg/L, about 60 mg/L, about 65 mg/L, about 70 mg/L, about 75 mg/L, about 80 mg/L, about 85 mg/L, about 90 mg/L, about 95 mg/L, about 100 mg/L, about 105 mg/L, about 110 mg/L, about 115 mg/L, about 120 mg/L, about 125 mg/L, about 130 mg/L, about 135 mg/L, about 140 mg/L, about 145 mg/L, about 150 mg/L, about 155 mg/L, about 160 mg/L, about 165 mg/L, about 170 mg/L, about 175 mg/L, about 180 mg/L, about 185 mg/L, about 190 mg/L, about 195 mg/L, about 200 mg/L, about 210 mg/L, about 220 mg/L, about 230 mg/L, about 240 mg/L, about 250 mg/L, about 260 mg/L, about 270 mg/L, about 280 mg/L, about 290 mg/L, about 300 mg/L, 310 mg/L, 320 mg/L, 330 mg/L, 340 mg/L, 350 mg/L, 360 mg/L, 370 mg/L, 380 mg/L, 390 mg/L, 400 mg/L, about 410 mg/L, about 420 mg/L, about 430 mg/L, about 440 mg/L, about 450 mg/L, about 460 mg/L, about 470 mg/L, about 480 mg/L, about 490 mg/L, or about 500 mg/L, including all ranges and values derivable therebetween. The inclusion of one or more cytokinins and/or lipoic acid in the bacterial culture medium may, in particular embodiments, improve viability, regenerability, and/or transformation/editing frequency following Rhizobiales- or Agrobacterium-mediated transformation. In certain embodiments, the inclusion of lipoic acid in the bacterial culture medium reduces stress during Rhizobiales- or Agrobacterium-mediated transformation. In some embodiments, the inclusion of one or more cytokinins and/or lipoic acid in the bacterial culture medium improves the viability, regenerability, and/or transformation/editing frequency of dicot embryo explants. The inclusion of one or more cytokinins and/or lipoic acid in the bacterial culture medium, in particular embodiments, improves the viability, regenerability, and/or transformation/editing frequency of soybean embryo explants.

In some embodiments, thidiazuron (TDZ) may be included in the bacterial culture medium at a concentration in a range from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.25 mg/L to about 1.75 mg/L, about 0.5 mg/L to about 1.5 mg/L, about 0.1 mg/L to about 5 mg/L, about 0.5 mg/L to about 4 mg/L, about 0.5 mg/L to about 3 mg/L, about 0.5 mg/L to about 2 mg/L, about 0.5 mg/L to about 1.5 mg/L, about 1 mg/L to about 5 mg/L, about 1 mg/L to about 4 mg/L, about 1 mg/L to about 3 mg/L, or about 0.1 mg/L, about 0.25 mg/L, about 0.5 mg/L, about 0.75 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween. In some embodiments, a cytokinin other than thidiazuron (TDZ), such as 6-benzylaminopurine (BAP), kinetin, zeatin, diphenyl urea (DPU), 6-(gamma,gamma-dimethylallylamino)purine (2iP), and 6-(3-hydroxybenzylamino)purine (meta-topolin), may be included in the bacterial culture medium at a concentration adjusted and set according to its relative activity. For example, 6-benzylaminopurine (BAP), kinetin, zeatin, and/or 6-(3-hydroxybenzylamino)purine (meta-topolin) may be present in the bacterial culture medium at a concentration from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 1 mg/L to about 10 mg/L, about 2 mg/L to about 10 mg/L, about 2 mg/L to about 8 mg/L, about 2.5 mg/L to about 7.5 mg/L, about 3 mg/L to about 10 mg/L, about 4 mg/L to about 6 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween, which may improve the viability, regenerability, and/or transformation/editing frequency of embryo explants, such as dicot embryo explants or soybean, cotton or canola embryo explants, following Rhizobiales- or Agrobacterium-mediated transformation. In some embodiments, 6-(gamma,gamma-dimethylallylamino)purine (2iP) may be present in the bacterial culture medium at a concentration in a range from about 5 mg/L to about 50 mg/L, about 10 mg/L to about 50 mg/L, about 10 mg/L to about 40 mg/L, about 15 mg/L to about 40 mg/L, about 15 mg/L to about 35 mg/L about 15 mg/L to about 30 mg/L, about 20 mg/L to about 30 mg/L, about 22.5 mg/L to about 30 mg/L, about 22.5 mg/L to about 27.5 mg/L, or of about 5 mg/L, about 7.5 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all ranges and values derivable therebetween, which may improve the viability, regenerability and/or transformation/editing frequency of soybean embryo explants following Rhizobiales- or Agrobacterium-mediated transformation.

Embodiments of the present disclosure may comprise genetically transforming or genetically modifying embryo explants, such as at least one cell of each of at least two embryo explants of a population, by collectively introducing a heterologous polynucleotide molecule via Rhizobiales- or Agrobacterium-mediated transformation, which will generally involve contacting the explants with the Rhizobiales bacterium or Agrobacterium containing the heterologous polynucleotide. Such introducing step may comprise the explants or population of explants being present in, added to and/or in contact with, an inoculation medium containing the Rhizobiales bacterium or Agrobacterium and allowing the inoculation step to occur for a period of time before removing the Rhizobiales bacterium or Agrobacterium from the explants. The inoculation medium may comprise, for example, water, basal salts, macronutrients, micronutrients, and/or vitamins. In certain embodiments, the inoculation may comprise polyethylene glycol and/or an antimicrobial agent and/or an antifungal agent. The concentration of the Rhizobiales bacterium or Agrobacterium in the inoculation medium can be measured and/or defined in terms of optical density (OD). Higher concentrations of Agrobacterium in the inoculation and/or co-culture mediums can improve or increase shoot frequency and plugging frequency, and thus transformation frequency. The OD concentration of the Rhizobiales bacterium or Agrobacterium in the inoculation medium can be in a range from about 0.1 to about 2.0, from about 0.1 to about 1.0, from about 0.1 to about 0.75, from about 0.1 to about 0.5, from about 0.2 to about 2.0, from about 0.2 to about 1.0, from about 0.2 to about 0.5, from about 0.25 to about 2.0, from about 0.25 to about 1.0, from about 0.25 to about 0.5, from about 0.5 to about 2.0, from about 0.5 to about 1.5, from about 0.75 to about 1.5, or from about 0.75 to about 1.25, or about 0.1, about 0.2, about 0.25, about 0.3, about 0.4, about 0.5, about 0.6, about 0.7, about 0.75, about 0.8, about 0.9, about 1.0, about 1.25, about 1.5, or about 2.0, including all ranges and values derivable therebetween. According to some embodiments, transformation may be improved with some monocot or corn germplasms, including explants from male corn lines, by lowering the OD concentration of the Rhizobiales bacterium or Agrobacterium in the inoculation medium, such as in a range from about 0.25 to about 1.0 or from about 0.25 to about 0.5, or about 0.1, about 0.2, about 0.25, about 0.3, about 0.4, about 0.5, about 0.6, about 0.7, about 0.75, about 0.8, about 0.9, or about 1.0, including all ranges and values derivable therebetween.

In certain embodiments, the inoculation medium may optionally comprise thiabedazole (TBZ) and/or nystatin, which may be used as an antimicrobial or antifungal agent. The concentration of TBZ may be in a range between about 0 g/L and about 100 g/L or more preferably between about 5 g/L and about 15 g/L, or about 10 g/L, and/or the concentration of nystatin may be in a range between about 0 g/L and about 300 g/L, or more preferably between about 25 g/L and about 75 g/L, or about 50 g/L. In certain embodiments, the inoculation medium may comprise Rhizobiales or Agrobacterium culturing medium described above, which may be transferred at a certain volume to contact the explants. In certain embodiments, the inoculation medium may optionally contain or comprise a cytokinin and/or lipoic acid. According to some embodiments, the explants (or population or plurality of explants) may be transferred or placed into a tube or container and a certain quantity of Rhizobiales or Agrobacterium culturing medium may be added to the tube or container. The amount of inoculum (or inoculation medium containing the Rhizobiales or Agrobacterium) may be added to the tube or container in an amount or volume sufficient to cover and/or submerge the explants.

According to some embodiments, the tube or container containing the explants in the inoculation medium may be optionally sonicated, vortexed, or otherwise physically or mechanically agitated. Such agitation treatments may help improve transformation and/or delivery or introduction of the heterologous polynucleotide into a cell(s) of the explants, such as by wounding and/or by increasing the permeation or penetration of the Rhizobiales bacterium into meristematic or explant tissues. According to some embodiments, the explants may be sonicated or agitated for a time period ranging from about 1 second to about 10 minutes, or from about 5 seconds to about 5 minutes, or from about 10 seconds to about 4 minutes, or from about 10 seconds to about 3 minutes, or of about 10 seconds, about 20 seconds, about 30 seconds, about 40 seconds, about 50 seconds, about 1 minute, about 1.5 minutes, about 2 minutes, about 2.5 minutes, about 3 minutes, or about 4 minutes, including all ranges and values derivable therebetween. With or without the physical or mechanical agitation step, the explants may be (subsequently) agitated, moved, or inverted more gently (than the prior more vigorous agitation or wounding) as part of this inoculation step, such as by rocking or shaking, to improve transformation and/or delivery or introduction of the heterologous polynucleotide into cell(s) of the explants. This gentle movement may be for a time period ranging from about 5 second to about 2 hours, or from about 5 seconds to about 20 minutes, or from about 10 seconds to about 15 minutes, or from about 1 minute to about 15 minutes, or from about 5 minutes to about 15 minutes, or from about 7.5 minutes to about 12.5 minutes, or from about 1 minute to about 2 hours, or from about 5 minutes to about 2 hours, or from about 10 minutes to about 2 hours, or from about 20 minutes to about 1.5 hours, or from about 30 minutes to about 1.5 hours, or from about 45 minutes to about 1.25 hours, or from about 1 minute to about 1 hour, or from about 5 minutes to about 45 minutes, or from about 15 minutes to about 45 minutes, or from about 20 minutes to about 40 minutes, or of about 10 seconds, about 20 seconds, about 30 seconds, about 40 seconds, about 50 seconds, about 1 minute, about 1.5 minutes, about 2 minutes, about 2.5 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 40 minutes, about 50 minutes, about 1 hour, about 1.5 hours, or about 2 hours, including all ranges and values derivable therebetween.

In some embodiments, the introducing or inoculation step may be carried out under ambient lighting conditions. In some embodiments, the introducing or inoculation step, such as for monocot embryo explants, may include subjecting the population of embryo explants to a force treatment, such as a centrifugation and/or pressure treatment(s). According to some embodiments, the heterologous polynucleotide molecule, the ribonucleoprotein, and/or the site-specific nuclease, is introduced into the population via particle bombardment.

According to embodiments of the present disclosure, a force treatment is applied to the population of seed embryo explants either prior to or during inoculation, or prior to and during inoculation, of the population with a Rhizobiales or Agrobacterium bacterium comprising the heterologous polynucleotide molecule. In certain embodiments, the force treatment is applied during and/or after rehydration of the seed embryo explants. The force treatment, in particular embodiments, can be applied during the inoculation step while the population is in contact with the inoculation medium. In one embodiment, explants “in contact with” a medium may be positioned completely or partially in or on a medium. Non-limiting examples of medium in which an explant may be in contact with include a liquid medium, a solid medium, and a substrate comprising a medium. The population may be submerged in a volume of the inoculation medium when the force treatment is applied. Alternatively, the force treatment may be applied to the embryo explants of the population after an excess amount of the inoculation medium has been removed. The inoculation medium, for example, may be decanted, poured, aspirated or blotted from the explants prior to application of the force treatment. If the force treatment is applied during the inoculation step, then the inoculation medium may not be entirely absent from contacting the explants of the population, even if an amount or volume of the inoculation medium is removed from the explants before the force treatment.

As used herein, the term “heterologous polynucleotide molecule” refers to a polynucleotide molecule that is not naturally present, or is not naturally present in the same form or structure, in the cell being transformed or modified, without human intervention. For example, a heterologous polynucleotide molecule may not naturally occur in the plant species being transformed or modified, or may be expressed in a manner or genomic context that differs from the natural expression pattern or genomic context found in the species being transformed or modified, for example, in some embodiments the heterologous polynucleotide molecule may be overexpressed. In particular embodiments, the heterologous polynucleotide molecule may be the combination of two or more polynucleotide molecules, wherein such a combination is not normally found in nature. The two polynucleotide molecules may, in certain embodiments, be derived from different species or may be derived from different genes, such as, different genes from the same species or the same genes from different species. In some embodiments, a heterologous polynucleotide molecule may comprise two polynucleotide sequences that are not found juxtaposed or operably linked in any naturally occurring polynucleotide molecule. The heterologous polynucleotide molecule, in further embodiments, may comprise a promoter or other regulatory sequence operably linked to a transcribable polynucleotide sequence, wherein the promoter or other regulatory sequence and the transcribable polynucleotide sequence are not operably linked in any naturally occurring polynucleotide molecule. As used herein, the term “polynucleotide molecule” refers to a linear or circular single-stranded or double-stranded DNA or RNA polynucleotide molecule or sequence, which may be derived from any source. For example, a polynucleotide molecule may comprise a polynucleotide sequence in which one or more nucleic acid sequences have been linked together in a functionally operative manner. As used herein, the term “nucleic acid sequence” refers to deoxyribonucleic acid (DNA) or ribonucleic acid (RNA) sequence.

As used herein, the term “ribonucleoprotein” refers to a protein which may interact with a nucleic acid or polynucleotide molecule. A ribonucleoprotein may be, for example, a site-specific nuclease known in the art to be associated with a nucleic acid or polynucleotide molecule. Non-limiting examples of site-specific nucleases that may be a ribonucleoprotein include RNA-guided endonucleases, such as those of the CRISPR/Cas systems (see, for example, U.S. Pat. Nos. 8,697,359, 8,771,945 and 9,790,490 and U.S. Pat. Appl. Pub. No. 2014/0068797) and CRISPR-associated transposases or CAST (see, for example US Patent Application Pub. No. 2020/0190487), the entire contents and disclosures of which are incorporated herein by reference. In some embodiments, the polynucleotide molecule, the heterologous polynucleotide molecule, or the ribonucleoprotein may be a recombinant polynucleotide molecule or a recombinant protein. In some embodiments, the polynucleotide molecule or heterologous polynucleotide molecule may be a recombinant polynucleotide molecule. As used herein, the term “recombinant” when used in reference to a polynucleotide (DNA or RNA) molecule, protein, construct, vector, or the like, refers to a polynucleotide or protein molecule or sequence that is not naturally present, or is not naturally present in the same form, and/or structure without human intervention. In particular embodiments, a recombinant polynucleotide (DNA or RNA) molecule, protein, construct, vector, or the like may comprise, for example, a combination of two or more polynucleotide or protein sequences that do not naturally occur together in the same manner, such as a polynucleotide molecule, protein, construct, or the like, comprising at least two polynucleotide or protein sequences that are operably linked but heterologous with respect to each other. In additional embodiments, a recombinant polynucleotide (DNA or RNA) molecule, protein, construct, vector, or the like may comprise, for example, any combination of two or more polynucleotide or protein sequences in the same molecule (e.g., a plasmid, construct, vector, chromosome, protein, or the like) where such a combination is man-made and not normally found in nature. As used herein, the phrase “not normally found in nature” means not found in nature without human intervention. A recombinant polynucleotide or protein molecule, construct, or the like, may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, or the like, may also refer to a polynucleotide or protein molecule or sequence that has been genetically engineered and/or constructed outside of a cell. For example, a recombinant polynucleotide molecule may comprise any engineered or man-made plasmid, vector, or the like, and may include a linear or circular polynucleotide molecule. Such plasmids, vectors, or the like, may contain various maintenance elements including, for example, a prokaryotic origin of replication and selectable marker, as well as one or more transgenes or expression cassettes perhaps in addition to a plant selectable marker gene.

To improve transformation or editing of a population of explants, a variety of different force treatments may be used or applied to the population before and/or during the inoculation step, such as a centrifugal force treatment, a gravitational force treatment, a vacuum treatment, a sonication treatment, a vortexing treatment, a shearing treatment, a mechanical force treatment, a pressure treatment, or any combination thereof. In some embodiments, a force treatment may comprise a pressure treatment and/or a gravitational (or centrifugal) force treatment. In specific embodiments, a force treatment may comprise a pressure treatment. In further embodiments, a force treatment may comprise a gravitational (or centrifugal) force treatment. In certain embodiments, the methods described herein may further comprise applying a mechanical force treatment, a vortexing treatment, a shaking or shearing treatment, a sonication treatment, and/or a vacuum treatment, in addition to a pressure treatment and/or a gravitational (or centrifugal) force treatment. Without being bound by theory, application of a force treatment prior to or during inoculation may improve transformation by increasing the contact and attachment of the Rhizobiales bacterium to the explants of the population, by wounding the explants, and/or by increasing the permeation or penetration of the Rhizobiales bacterium into meristematic or explant tissues.

In some embodiments, the force treatment may comprise applying a pressure force or treatment in a range from about 100 pounds per square inch (psi) to about 20,000 psi, about 100 psi to about 18,000 psi, about 100 psi to about 16,000 psi, about 100 to about 14,000 psi, about 100 to about 12,000 psi, about 100 to about 10,000 psi, about 100 to about 8,000 psi, about 100 to about 6,000 psi, about 100 to about 4,000 psi, about 100 to about 2,000 psi, about 100 to about 1,000 psi, or about 100 psi to about 500 psi, such as about 100 psi, about 150 psi, about 200 psi, about 250 psi, about 300 psi, about 350 psi, about 400 psi, or about 500 psi, of pressure to the population of embryo explants, including all values and ranges derivable therebetween. Other units for pressure are also known in the art. Methods for converting pressure in psi to other units, for example, standard atmospheres (atm) and Newtons (N) per square meter (N/m²) are known in the art. Pressure in atm can be accurately calculated using the following formula: atm=pressure (psi)/14.6959488, and 1 psi equals about 6894.76 N/m². Therefore, 100 psi is equal to about 6.80 atm, and 20,000 psi is equal to about 1360.9 atm. The pressure treatment can also be converted to an amount of force when the surface area is known or fixed. For example, the surface area of piston/cell cavity of the French Press 40K pressure cell (Thermo® IEC, FA-032) used in the Examples herein is about 0.88 in². Therefore, 3,334 psi applied using the French Press 40K pressure cell is equal to about 13,000 N [(3,334 psi×0.88 in²)]/[0.225 pounds/N] The pressure treatment, in some embodiments, may be applied from about 10 seconds to about 10 minutes, from about 15 seconds to about 8 minutes, from about 30 seconds to about 6 minutes, from about 2 minutes to about 4 minutes, or for about 3 minutes, including all values and ranges derivable therebetween.

The methods described herein comprise applying a gravitational or centrifugal force in a range from about 100×g to about 10,000×g, about 100×g to about 5,000×g, about 250×g to about 5,000×g, about 500×g to about 5,000×g, about 500×g to about 3,000×g, about 600×g to about 2,700×g, such as about 500×g, about 550×g, about 600×g, about 650×g, about 700×g, about 750×g, about 800×g, about 850×g, about 900×g, about 950×g, about 1000×g, about 1500×g, about 2000×g, about 2500×g, about 3000×g, about 3500×g, or about 4000×g, may be applied to the population of embryo explants, including all values and ranges derivable therebetween. A non-limiting example of a gravitational force treatment which may be applied to the population includes a centrifugal force or relative centrifugal force, which may be applied using an appropriate centrifuge. Methods for converting gravitational or centrifugal force, such as the relative centrifugal force (RCF) created by a centrifuge, to other units, such as revolutions per minute (rpm) and newton (N), are known in the art. Relative centrifugal force can be calculated based on the rpm and known dimensions of the device using the following formula: rpm=√[RCF/(r×1.118)×1×10⁵], wherein r=the rotational radius in centimeters. For the Sorvall™ RC3BP centrifuge (Thermo Fisher Scientific, Waltham, MA, USA) used in the Examples described herein, the rotational radius is about 24.67 cm. Therefore, 2620×g is equal to about 3,082 rpm [√[2620/(24.67×1.118)]×1×10⁵]. Similarly, centrifugal force in Newtons can be accurately estimated using the following formula: Force (N)=RCF×mass of the contents of the centrifugation tube (kg)×9.82 m/s². In particular embodiments, if the mass of contents of the centrifugation tube may be about 0.05 kg, then 2620×g would be equal to about 1286 N [(2620×g)×0.05 kg×9.82 m/s²]. The gravitational or centrifugal force treatment may be applied, in some embodiments, in a range from about 1 minute to about 2 hours, about 2 minutes to about 110 minutes, about 5 minutes to about 90 minutes, about 10 minutes to about 90 minutes, about 10 minutes to about 80 minutes, about 10 minutes to about 70 minutes, about 10 minutes to about 60 minutes, about 10 minutes to about 50 minutes, about 15 minutes to about 45 minutes, or about 20 minutes to about 40 minutes, such as about 10 minutes, about 15 minutes, about 20 minutes, about 25 minutes, about 30 minutes about 35 minutes, about 40 minutes, about 45 minutes, about 50 minutes, about 55 minutes, or about 60 minutes (1 hour), including all values and ranges derivable therebetween.

According to some embodiments, methods described herein comprise applying a higher gravitational or centrifugal force or relative centrifugal force (RCF) in a range from about 2,500×g to about 10,000×g, about 2,500×g to about 5,000×g, about 2,500×g to about 4,500×g, about 2,800×g to about 5,000×g, about 2,800×g to about 4,500×g, about 3,000×g to about 8,000×g, about 3,000×g to about 7,000×g, about 3,000×g to about 6,000×g, about 3,000×g to about 5,000×g, about 3,500×g to about 5,000×g, or about 3,500×g to about 4,500×g, such as about 2,500×g, about 3,000×g, about 3,500×g, about 3,600×g, about 3,700×g, about 3,800×g, about 3,900×g, about 4,000×g, about 4,100×g, about 4,200×g, about 4,300×g, about 4,400×g, about 4,500×g, about 4,600×g, about 4,700×g, about 4,800×g, about 4,900×g, about 5,000×g, about 6,000×g, about 7,000×g, about 8,000×g, about 9,000×g, or about 10,000×g to the monocot seed embryo explant, including all ranges and values derivable therebetween. According to some embodiments, applying a higher gravitational or centrifugal force or relative centrifugal force (RCF) may improve transformation and/or regeneration of genetically modified plants of monocot or corn embryo explants or certain monocot or corn lines that are more resistant to efficient transformation and/or regeneration of genetically modified plants, such as certain male germplasm corn lines.

The force treatment, such as the gravitational (or centrifugal) and/or pressure treatment(s), may be applied at a temperature of about 0.5° C. to about 28° C., about 2° C. to about 28° C., about 4° C. to about 28° C., about 10° C. to about 28° C., about 10° C. to about 25° C., or about 15° C. to about 23° C., including all values and ranges derivable therebetween.

In one aspect of the methods provided herein, the force treatment may comprise applying both a pressure treatment and a gravitational force treatment to the population of embryo explants. The pressure treatment and/or the gravitational force treatment may be applied prior to, during, or prior to and during inoculation of the population with a bacterium from the order Rhizobiales, wherein the Rhizobiales bacterium comprises a heterologous polynucleotide for transforming, editing or genetically modifying at least one plant cell of the explants of the population. In some embodiments, the pressure treatment is applied prior to applying the gravitational force treatment. In other embodiments, the gravitational force treatment is applied prior to the pressure treatment. The order of application of a pressure treatment and a gravitational force treatment may be preferred based on improved transformation or editing efficiency or frequency or based on ease of handling. In some embodiments, when a combination of pressure and gravitational force treatments are applied to the population, the pressure treatment may be applied before the gravitational force treatment, which may be due at least in part, to the ability to apply the force treatment more evenly prior to pelleting the explants with the gravitational or centrifugal treatment. Alternatively, the centrifuged or pelleted explants could be resuspended prior to a subsequent pressure treatment, or the pressure treatment could be applied to the centrifuged or pelleted explants without resuspension. In an aspect of the present disclosure, applying a pressure treatment and a gravitational force treatment either prior to, during, or prior to and during inoculation may improve transformation or editing of plants, as compared to applying only the pressure treatment or only the gravitational force treatment.

In another aspect, the methods described herein may further comprise applying a vacuum treatment to the population of embryo explants. The vacuum treatment may comprise, for example, submerging the population in a liquid inoculation medium comprising a Rhizobiales bacterium and subjecting the population to decreased pressure followed by rapid or gradual repressurization. Alternatively, a vacuum treatment may be applied to a population of embryo explants that is not submerged in a liquid inoculation medium. The vacuum treatment, in some embodiments, may be applied before the force treatment is applied, after the force treatment is applied, before the gravitational force treatment is applied, after the gravitational force treatment is applied, before the pressure treatment is applied, and/or after the pressure treatment is applied. In particular embodiments, where the force treatment comprises applying a pressure treatment and a gravitational force treatment, a vacuum treatment may be applied between applying the pressure treatment and applying the gravitational force treatment, regardless as to whether the gravitational force treatment or the pressure treatment is applied first. In one embodiment, the population may be subjected to a vacuum treatment of about 0.05 atm to about 0.50 atm, about 0.05 atm to about 0.40 atm, about 0.05 atm to about 0.30 atm, about 0.05 atm to about 0.20 atm, about 0.05 atm to about 0.10 atm, about 0.10 atm to about 0.50 atm, about 0.10 atm to about 0.40 atm, about 0.10 to about 0.30 atm of pressure, or about 0.10 atm to about 0.20 atm of pressure, including all values and ranges derivable therebetween.

After inoculation of a population of embryo explants with a Rhizobiales or Agrobacterium to introduce a heterologous polynucleotide into at least one cell of the explants, the inoculation medium containing the Rhizobiales or Agrobacterium, or most of the inoculation medium or at least an excess amount of the inoculation medium, may be removed before any subsequent transformation or culturing steps. The inoculation medium may be removed by any combination of decanting (perhaps with strainer or net), filtering, aspirating, pipetting, vacuuming and/or blotting or wicking away the inoculation medium. The blotting or wicking of inoculation medium may be done by contacting the medium with filter paper or other absorbent material. The removal of inoculation medium may be performed before the subsequent co-culturing step.

### C. Co-Culture of Embryo Explants

Following inoculation of a population of embryo explants with a Rhizobiales or Agrobacterium comprising a heterologous polynucleotide to introduce the heterologous polynucleotide into at least one cell of the explants, and possibly following removal of the inoculation medium, the explants may be co-cultured in contact with a co-culture medium. According to present embodiments, the seed embryo explant(s) may be transferred to, or become in contact with, a co-culture medium, or the seed embryo explant(s) may be transferred to one or more (co-culture) plate(s) containing a co-culture medium, or to which a co-culture medium is added after the explants are transferred. The co-culture medium may comprise, for example, water, basal salts, macronutrients, micronutrients, and/or vitamins. According to some embodiments, the co-culture medium may not contain or comprise any plant hormones, such as an auxin and/or cytokinin, and/or any surfactant or wetting agent, or alternatively the co-culture medium may contain or comprise a plant hormone, such as an auxin and/or cytokinin, and/or a surfactant or wetting agent. In particular embodiments, the co-culture medium comprises one or more cytokinins. Non-limiting examples of cytokinins that may be used in co-culture medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). According to some embodiments, the co-culture medium may optionally comprise an antimicrobial or antifungal agent, such as nystatin and/or thiabendazole (TBZ), which may be present at the concentration(s) described above for the bacterial culture and/or inoculation medium. The inclusion of one or more cytokinins and/or lipoic acid in the co-culture medium may, in particular embodiments, improve viability, regenerability, and/or transformation/editing frequency following Rhizobiales- or Agrobacterium-mediated transformation. In certain embodiments, the inclusion of lipoic acid in the co-culture medium reduces stress during Rhizobiales- or Agrobacterium-mediated transformation (e.g., during the inoculation and co-culture steps).

The concentration of cytokinin in the co-culture medium may be at or within the concentration(s) described above for the bacterial culture and/or inoculation medium. The concentration of the cytokinin in the co-culture medium may be, in some embodiments, about 0.1 mg/L to about 50 mg/L, about 0.1 mg/L to about 45 mg/L, about 0.1 mg/L to about 40 mg/L, about 0.1 mg/L to about 35 mg/L, about 0.1 mg/L to about 30 mg/L, about 0.1 mg/L to about 25 mg/L, about 0.1 mg/L to about 20 mg/L, about 0.1 mg/L to about 15 mg/L, about 0.1 mg/L to about 10 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 5 mg/L, about 0.5 mg/L to about 4 mg/L, about 0.5 mg/L to about 3 mg/L, about 1 mg/L to about 5 mg/L, about 1 mg/L to about 4 mg/L, about 1 mg/L to about 3 mg/L, about 5 mg/L to about 40 mg/L, about 5 mg/L to about 30 mg/L, about 10 mg/L to about 30 mg/L, or about 20 mg/L to about 30 mg/L, or about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all values and ranges derivable therebetween.

In certain embodiments, lipoic acid may be present in the co-culture medium at a concentration of about 0.1 mg/L to about 500 mg/L, about 0.1 mg/L to about 400 mg/L, about 0.1 mg/L to about 300 mg/L, about 0.1 mg/L to about 200 mg/L, about 10 mg/L to about 200 mg/L, about 10 mg/L to about 180 mg/L, about 10 mg/L to about 160 mg/L, about 10 mg/L to about 140 mg/L, about 10 mg/L to about 120 mg/L, about 10 mg/L to about 100 mg/L, about 20 mg/L to about 80 mg/L, about 40 mg/L to about 60 mg/L, about 50 mg/L to about 60 mg/L, or about 50 mg/L to about 55 mg/L, or about 5 mg/L, about 10 mg/L, about 15 mg/L, about 20 mg/L, about 25 mg/L, about 30 mg/L, about 35 mg/L, about 40 mg/L, about 45 mg/L, about 50 mg/L, about 55 mg/L, about 60 mg/L, about 65 mg/L, about 70 mg/L, about 75 mg/L, about 80 mg/L, about 85 mg/L, about 90 mg/L, about 95 mg/L, about 100 mg/L, about 105 mg/L, about 110 mg/L, about 115 mg/L, about 120 mg/L, about 125 mg/L, about 130 mg/L, about 135 mg/L, about 140 mg/L, about 145 mg/L, about 150 mg/L, about 155 mg/L, about 160 mg/L, about 165 mg/L, about 170 mg/L, about 175 mg/L, about 180 mg/L, about 185 mg/L, about 190 mg/L, about 195 mg/L, about 200 mg/L, about 210 mg/L, about 220 mg/L, about 230 mg/L, about 240 mg/L, about 250 mg/L, about 260 mg/L, about 270 mg/L, about 280 mg/L, about 290 mg/L, about 300 mg/L, 310 mg/L, 320 mg/L, 330 mg/L, 340 mg/L, 350 mg/L, 360 mg/L, 370 mg/L, 380 mg/L, 390 mg/L, 400 mg/L, about 410 mg/L, about 420 mg/L, about 430 mg/L, about 440 mg/L, about 450 mg/L, about 460 mg/L, about 470 mg/L, about 480 mg/L, about 490 mg/L, or about 500 mg/L, including all ranges and values derivable therebetween.

In some embodiments, thidiazuron (TDZ) may be included in the co-culture medium at a concentration in a range from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.25 mg/L to about 1.75 mg/L, about 0.5 mg/L to about 1.5 mg/L, about 0.1 mg/L to about 5 mg/L, about 0.5 mg/L to about 4 mg/L, about 0.5 mg/L to about 3 mg/L, about 0.5 mg/L to about 2 mg/L, about 0.5 mg/L to about 1.5 mg/L, about 1 mg/L to about 5 mg/L, about 1 mg/L to about 4 mg/L, about 1 mg/L to about 3 mg/L, or about 0.1 mg/L, about 0.25 mg/L, about 0.5 mg/L, about 0.75 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween. The inclusion of thidiazuron (TDZ) in the co-culture medium may improve the viability, regenerability, and/or transformation/editing frequency following Rhizobiales- or Agrobacterium-mediated transformation. In some embodiments, the inclusion of one or more cytokinins and/or lipoic acid in the co-culture medium improves the viability, regenerability, and/or transformation/editing frequency of dicot embryo explants. The inclusion of one or more cytokinins and/or lipoic acid in the co-culture medium, in particular embodiments, improves the viability, regenerability, and/or transformation/editing frequency of soybean embryo explants, cotton embryo explants or canola embryo explants.

In some embodiments, a cytokinin other than thidiazuron (TDZ), such as 6-benzylaminopurine (BAP), kinetin, zeatin, diphenyl urea (DPU), 6-(gamma,gamma-dimethylallylamino)purine (2iP), and 6-(3-hydroxybenzylamino)purine (meta-topolin), may be included in the co-culture medium at a concentration adjusted and set according to its relative activity. For example, 6-benzylaminopurine (BAP), kinetin, zeatin, and/or 6-(3-hydroxybenzylamino)purine (meta-topolin) may be present in the bacterial culture medium at a concentration from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 1 mg/L to about 10 mg/L, about 2 mg/L to about 10 mg/L, about 2 mg/L to about 8 mg/L, about 2.5 mg/L to about 7.5 mg/L, about 3 mg/L to about 10 mg/L, about 4 mg/L to about 6 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween, which may improve the viability, regenerability, and/or transformation/editing frequency of embryo explants, such as dicot embryo explants or soybean, cotton or canola embryo explants, following Rhizobiales- or Agrobacterium-mediated transformation.

In some embodiments, 6-(gamma,gamma-dimethylallylamino)purine (2iP) may be present in the co-culture medium at a concentration in a range from about 5 mg/L to about 50 mg/L, about 10 mg/L to about 50 mg/L, about 10 mg/L to about 40 mg/L, about 15 mg/L to about 40 mg/L, about 15 mg/L to about 35 mg/L about 15 mg/L to about 30 mg/L, about 20 mg/L to about 30 mg/L, about 22.5 mg/L to about 30 mg/L, about 22.5 mg/L to about 27.5 mg/L, or of about 5 mg/L, about 7.5 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all ranges and values derivable therebetween, which may improve the viability, regenerability, and/or transformation/editing frequency of embryo explants, such as dicot embryo explants or soybean, cotton or canola embryo explants, following Rhizobiales- or Agrobacterium-mediated transformation.

In certain embodiments, the co-culture medium may comprise a surfactant, which may be particularly applicable for transformation or editing of monocot embryo explants and may include any suitable surfactant or combination of surfactants known in the art, for example a detergent, a wetting agent, an emulsifier, a foaming agent, or a dispersant. In some embodiments, the surfactant may be Silwet® or a similar surfactant.

According to some embodiments, methods described herein comprise transferring monocot seed embryo explant(s) to co-culture plate(s) or container(s) at a lower density of visible or total explants per container or plate. According to some embodiments, a lower density of visible or total explants per co-culture plate may improve transformation and/or regeneration of genetically modified plants of monocot or corn embryo explants or certain monocot or corn lines that are more resistant to efficient transformation and/or regeneration of genetically modified plants, such as certain male germplasm corn lines. As described herein, transformation of male corn lines, and possibly other monocot germplasms, may often be difficult or less efficient as compared to female corn lines or other monocot germplasms, which may be due to variable or lower germination rates, lower Agrobacterium infection rate, crowding and tissue death on culturing and selection media especially at higher densities, and/or minimal shoot regeneration. These differences may be due to different genetics and characteristics of male corn lines or other germplasms and embryo explants from seeds of male corn lines or other germplasms, as compared to female corn lines or other germplasms. Given that seed embryo explants of certain male germplasms or lines or other monocot germplasms may have a lower regeneration rate, which may be related to reduced, lowered or decreased viability and/or regenerability, a higher number of visible (or total) explants may be needed to have a similar number of regenerable explants and produce a similar number of regenerated explants, plantlets or plants as compared to female corn lines or other monocot germplasms having a relatively higher or increased regeneration rate (or higher or increased viability and/or regenerability). However, if the higher number of visible (or total) explants are placed onto the same number of co-culture plate(s) or container(s), then the co-culture plate(s) or container(s) will become too crowded, which can cause or further lead to a reduced, lowered or decreased regeneration rate (or reduced, lowered or decreased viability and/or regenerability). In some embodiments, crowding may result in decreased desiccation of the embryo explants, which may in turn result in decreased delivery of the heterologous polynucleotide molecule to the at least one cell of the embryo explant. If delivery of the heterologous polynucleotide molecule is decreased this may lead, in some embodiments, to a reduced, lowered, or decreased regeneration rate (or reduced, lowered, or decreased viability and/or regenerability) if the co-culture medium, bud induction medium, extended bud induction medium, and/or regeneration medium includes a selection agent. Thus, increasing the number of co-culture plate(s) of a given size and/or decreasing, lowering or reducing the number of visible (or total) explants per plate or co-culture plate area may lead to a relatively higher or increased regeneration rate (or higher or increased viability and/or regenerability), or a relatively higher or increased number of regenerable explants, per number of visible (or total) explants in the co-culture plate(s) or container(s), and/or per number of inoculated explants in contact with the co-culture medium. In some embodiments, the number of visible explants may be defined as the total number of explants, including regenerable and non-regenerable explants. In certain embodiments, the percent of regenerable explants may be calculated as the number of regenerable explants per gram, or the number of viable explants capable of germinating prior to transformation, divided by the total number of visible explants per gram, multiplied by 100. According to some embodiments, a given number of visible (or total) monocot seed embryo explants, such as in a range from about 500 to about 50,000, about 500 to about 25,000, about 500 to about 20,000, about 500 to about 15,000, about 500 to about 14,000, about 500 to about 13,000, about 500 to about 12,000, about 500 to about 11,000, about 500 to about 10,000, about 500 to about 9,000, about 500 to about 8,000, about 500 to about 7,000, about 500 to about 6,000, about 500 to about 5,000, about 500 to about 4,000, about 500 to about 3,000, about 500 to about 2,500, about 500 to about 2,000, about 500 to about 1,500, about 1,000 to about 3,000, about 1,000 to about 2,500, about 1,000 to about 2,500, about 1,000 to about 2,000, or about 1,000 to about 1,500, including all ranges and values derivable therebetween, may be transferred from an inoculation medium to a relatively greater or higher number of co-culture plate(s) or container(s), such as 5 or more, 6 or more, 7 or more, 8 or more, 9 or more, or 10 or more co-culture plate(s) or container(s).

According to some embodiments, a certain number of visible monocot seed embryo explants of a given germplasm or line, corresponding to a target number of regenerable seed embryo explants based on a known or theorized regeneration rate for such germplasm or line, are added or transferred to a greater number of co-culture plate(s) or container(s), or to a greater total surface area of co-culture plate(s) or container(s), to achieve a lower density of visible (or total) explants per plate or container, or per total surface area of the co-culture plate(s) or container(s). According to some embodiments, the number of visible (or total) monocot seed embryo explants transferred or added to co-culture plate(s) or container(s) may be at a density of less than or equal to about 800 seed embryo explants per plate, less than or equal to about 700 seed embryo explants per plate, or less than or equal to about 600 seed embryo explants per plate, or in a range of densities from about 50 to about 800 seed embryo explants per plate, about 50 to about 700 seed embryo explants per plate, about 50 to about 600 seed embryo explants per plate, about 50 to about 500 seed embryo explants per plate, about 50 to about 400 seed embryo explants per plate, about 100 to about 700 seed embryo explants per plate, about 100 to about 600 seed embryo explants per plate, about 100 to about 500 seed embryo explants per plate, about 100 to about 400 seed embryo explants per plate, about 150 to about 700 seed embryo explants per plate, about 150 to about 600 seed embryo explants per plate, about 150 to about 500 seed embryo explants per plate, about 150 to about 400 seed embryo explants per plate, about 200 to about 500 seed embryo explants per plate, about 200 to about 400 seed embryo explants per plate, about 300 to about 700 seed embryo explants per plate, about 400 to about 700 seed embryo explants per plate, or about 500 to about 700 seed embryo explants per plate, or at about 100, about 150, about 175, about 200, about 225, about 250, about 255, about 260, about 265, about 270, about 275, about 280, about 285, about 290, about 295, about 300, about 325, about 350, about 375, about 400, about 425, about 450, about 475, about 500, about 525, about 550, about 570, about 575, about 600, about 650, about 700, about 750, or about 800 seed embryo explants per plate, including all ranges and values derivable therebetween. For these density values and ranges, the surface area of each co-culture plate is approximately 11.9 square inches (in²) or 76.8 square centimeters (cm²). Thus, all of the above density values and ranges for seed embryo explants per plate can be readily converted into density values and ranges of seed embryo explants per co-culture surface area (for example, a density of 100 seed embryo explants per plate can be divided by the surface area per plate to provide a density of seed embryo explants per co-culture surface area of about 8.4 seed embryo explants/square inch (in²) or about 1.3 seed embryo explants/square centimeter (cm²), and similar conversions can be readily made for other density values and ranges). Density values and ranges of seed embryo explants per co-culture surface area is a more universal definition for density of seed embryo explants in a variety of different co-culture plate(s) or container(s) that may each have different surface areas.

In some embodiments, the number of visible (or total) monocot seed embryo explants transferred or added to the co-culture plate(s) or container(s) may be at a density of less than or equal to about 11.0, about 10.9, about 10.8, about 10.7, about 10.6, about 10.5, about 10.4, about 10.3, about 10.2, about 10.1, about 10.0, about 9.9, about 9.8, about 9.7, about 9.6, about 9.5, about 9.4, about 9.3, about 9.2, about 9.1, about 9.0, about 8.9, about 8.8, about 8.7, about 8.6, about 8.5, about 8.4, about 8.3, about 8.2, about 8.1, about 8.0, about 7.9, about 7.8, about 7.7, about 7.6, about 7.5, about 7.4, about 7.3, about 7.2, about 7.1, about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 embryo explants per square centimeter (cm²) of co-culture surface area, including all ranges and values derivable therebetween. In certain embodiments, the number of visible (or total) monocot seed embryo explants transferred or added to the co-culture plate(s) or container(s) may be at a density in a range from about 0.1 to about 11.0, about 0.1 to about 10.5, about 0.1 to about 10.0, about 0.1 to about 9.5, about 0.1 to about 9.0, about 0.1 to about 8.5, about 0.1 to about 8.0, about 0.1 to about 7.5, about 0.1 to about 7.0, about 0.1 to about 6.5, about 0.1 to about 6.0, about 0.1 to about 5.5, about 0.1 to about 5.0, about 0.1 to about 4.5, about 0.1 to about 4.0, about 0.1 to about 3.5, about 0.1 to about 3.0, about 0.1 to about 2.5, about 0.1 to about 2.0, about 0.1 to about 1.5, about 0.1 to about 1.0, about 0.1 to about 0.5, about 0.2 to about 11.0, about 0.2 to about 10.5, about 0.2 to about 10.0, about 0.2 to about 9.5, about 0.2 to about 9.0, about 0.2 to about 8.5, about 0.2 to about 8.0, about 0.2 to about 7.5, about 0.2 to about 7.0, about 0.2 to about 6.5, about 0.2 to about 6.0, about 0.2 to about 5.5, about 0.2 to about 5.0, about 0.2 to about 4.5, about 0.2 to about 4.0, about 0.2 to about 3.5, about 0.2 to about 3.0, about 0.2 to about 2.5, about 0.2 to about 2.0, about 0.2 to about 1.5, about 0.2 to about 1.0, about 0.2 to about 0.5, about 0.3 to about 11.0, about 0.3 to about 10.5, about 0.3 to about 10.0, about 0.3 to about 9.5, about 0.3 to about 9.0, about 0.3 to about 8.5, about 0.3 to about 8.0, about 0.3 to about 7.5, about 0.3 to about 7.0, about 0.3 to about 6.5, about 0.3 to about 6.0, about 0.3 to about 5.5, about 0.3 to about 5.0, about 0.3 to about 4.5, about 0.3 to about 4.0, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.4 to about 11.0, about 0.4 to about 10.5, about 0.4 to about 10.0, about 0.4 to about 9.5, about 0.4 to about 9.0, about 0.4 to about 8.5, about 0.4 to about 8.0, about 0.4 to about 7.5, about 0.4 to about 7.0, about 0.4 to about 6.5, about 0.4 to about 6.0, about 0.4 to about 5.5, about 0.4 to about 5.0, about 0.4 to about 4.5, about 0.4 to about 4.0, about 0.4 to about 3.5, about 0.4 to about 3.0, about 0.4 to about 2.5, about 0.4 to about 2.0, about 0.4 to about 1.5, about 0.4 to about 1.0, about 0.4 to about 0.5, about 0.5 to about 11.0, about 0.5 to about 10.5, about 0.5 to about 10.0, about 0.5 to about 9.5, about 0.5 to about 9.0, about 0.5 to about 8.5, about 0.5 to about 8.0, about 0.5 to about 7.5, about 0.5 to about 7.0, about 0.5 to about 6.5, about 0.5 to about 6.0, about 0.5 to about 5.5, about 0.5 to about 5.0, about 0.5 to about 4.5, about 0.5 to about 4.0, about 0.5 to about 3.5, about 0.5 to about 3.0, about 0.5 to about 2.5, about 0.5 to about 2.0, about 0.5 to about 1.5, about 0.5 to about 1.0, about 0.6 to about 11.0, about 0.6 to about 10.5, about 0.6 to about 10.0, about 0.6 to about 9.5, about 0.6 to about 9.0, about 0.6 to about 8.5, about 0.6 to about 8.0, about 0.6 to about 7.5, about 0.6 to about 7.0, about 0.6 to about 6.5, about 0.6 to about 6.0, about 0.6 to about 5.5, about 0.6 to about 5.0, about 0.6 to about 4.5, about 0.6 to about 4.0, about 0.6 to about 3.5, about 0.6 to about 3.0, about 0.6 to about 2.5, about 0.6 to about 2.0, about 0.6 to about 1.5, about 0.6 to about 1.0, about 0.7 to about 11.0, about 0.7 to about 10.5, about 0.7 to about 10.0, about 0.7 to about 9.5, about 0.7 to about 9.0, about 0.7 to about 8.5, about 0.7 to about 8.0, about 0.7 to about 7.5, about 0.7 to about 7.0, about 0.7 to about 6.5, about 0.7 to about 6.0, about 0.7 to about 5.5, about 0.7 to about 5.0, about 0.7 to about 4.5, about 0.7 to about 4.0, about 0.7 to about 3.5, about 0.7 to about 3.0, about 0.7 to about 2.5, about 0.7 to about 2.0, about 0.7 to about 1.5, about 0.7 to about 1.0, about 0.8 to about 11.0, about 0.8 to about 10.5, about 0.8 to about 10.0, about 0.8 to about 9.5, about 0.8 to about 9.0, about 0.8 to about 8.5, about 0.8 to about 8.0, about 0.8 to about 7.5, about 0.8 to about 7.0, about 0.8 to about 6.5, about 0.8 to about 6.0, about 0.8 to about 5.5, about 0.8 to about 5.0, about 0.8 to about 4.5, about 0.8 to about 4.0, about 0.8 to about 3.5, about 0.8 to about 3.0, about 0.8 to about 2.5, about 0.8 to about 2.0, about 0.8 to about 1.5, about 0.8 to about 1.0, about 0.9 to about 11.0, about 0.9 to about 10.5, about 0.9 to about 10.0, about 0.9 to about 9.5, about 0.9 to about 9.0, about 0.9 to about 8.5, about 0.9 to about 8.0, about 0.9 to about 7.5, about 0.9 to about 7.0, about 0.9 to about 6.5, about 0.9 to about 6.0, about 0.9 to about 5.5, about 0.9 to about 5.0, about 0.9 to about 4.5, about 0.9 to about 4.0, about 0.9 to about 3.5, about 0.9 to about 3.0, about 0.9 to about 2.5, about 0.9 to about 2.0, about 0.9 to about 1.5, about 0.9 to about 1.0, about 1.0 to about 11.0, about 1.0 to about 10.5, about 1.0 to about 10.0, about 1.0 to about 9.5, about 1.0 to about 9.0, about 1.0 to about 8.5, about 1.0 to about 8.0, about 1.0 to about 7.5, about 1.0 to about 7.0, about 1.0 to about 6.5, about 1.0 to about 6.0, about 1.0 to about 5.5, about 1.0 to about 5.0, about 1.0 to about 4.5, about 1.0 to about 4.0, about 1.0 to about 3.5, about 1.0 to about 3.0, about 1.0 to about 2.5, about 1.0 to about 2.0, about 1.0 to about 1.5, about 1.5 to about 11.0, about 1.5 to about 10.5, about 1.5 to about 10.0, about 1.5 to about 9.5, about 1.5 to about 9.0, about 1.5 to about 8.5, about 1.5 to about 8.0, about 1.5 to about 7.5, about 1.5 to about 7.0, about 1.5 to about 6.5, about 1.5 to about 6.0, about 1.5 to about 5.5, about 1.5 to about 5.0, about 1.5 to about 4.5, about 1.5 to about 4.0, about 1.5 to about 3.5, about 1.5 to about 3.0, about 1.5 to about 2.5, about 1.5 to about 2.0, about 2.0 to about 11.0, about 2.0 to about 10.5, about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 2.0 to about 6.5, about 2.0 to about 6.0, about 2.0 to about 5.5, about 2.0 to about 5.0, about 2.0 to about 4.5, about 2.0 to about 4.0, about 2.0 to about 3.5, about 2.0 to about 3.0, about 2.0 to about 2.5, about 2.5 to about 11.0, about 2.5 to about 10.5, about 2.5 to about 10.0, about 2.5 to about 9.5, about 2.5 to about 9.0, about 2.5 to about 8.5, about 2.5 to about 8.0, about 2.5 to about 7.5, about 2.5 to about 7.0, about 2.5 to about 6.5, about 2.5 to about 6.0, about 2.5 to about 5.5, about 2.5 to about 5.0, about 2.5 to about 4.5, about 2.5 to about 4.0, about 2.5 to about 3.5, about 2.5 to about 3.0, about 3.0 to about 11.0, about 3.0 to about 10.5, about 3.0 to about 10.0, about 3.0 to about 9.5, about 3.0 to about 9.0, about 3.0 to about 8.5, about 3.0 to about 8.0, about 3.0 to about 7.5, about 3.0 to about 7.0, about 3.0 to about 6.5, about 3.0 to about 6.0, about 3.0 to about 5.5, about 3.0 to about 5.0, about 3.0 to about 4.5, about 3.0 to about 4.0, about 3.0 to about 3.5, about 3.5 to about 11.0, about 3.5 to about 10.5, about 3.5 to about 10.0, about 3.5 to about 9.5, about 3.5 to about 9.0, about 3.5 to about 8.5, about 3.5 to about 8.0, about 3.5 to about 7.5, about 3.5 to about 7.0, about 3.5 to about 6.5, about 3.5 to about 6.0, about 3.5 to about 5.5, about 3.5 to about 5.0, about 3.5 to about 4.5, about 3.5 to about 4.0, about 4.0 to about 11.0, about 4.0 to about 10.5, about 4.0 to about 10.0, about 4.0 to about 9.5, about 4.0 to about 9.0, about 4.0 to about 8.5, about 4.0 to about 8.0, about 4.0 to about 7.5, about 4.0 to about 7.0, about 4.0 to about 6.5, about 4.0 to about 6.0, about 4.0 to about 5.5, about 4.0 to about 5.0, about 4.0 to about 4.5, about 4.5 to about 11.0, about 4.5 to about 10.5, about 4.5 to about 10.0, about 4.5 to about 9.5, about 4.5 to about 9.0, about 4.5 to about 8.5, about 4.5 to about 8.0, about 4.5 to about 7.5, about 4.5 to about 7.0, about 4.5 to about 6.5, about 4.5 to about 6.0, about 4.5 to about 5.5, about 4.5 to about 5.0 embryo, about 5.0 to about 11.0, about 5.0 to about 10.5, about 5.0 to about 10.0, about 5.0 to about 9.5, about 5.0 to about 9.0, about 5.0 to about 8.5, about 5.0 to about 8.0, about 5.0 to about 7.5, about 5.0 to about 7.0, about 5.0 to about 6.5, about 5.0 to about 6.0, about 5.0 to about 5.5, about 5.5 to about 11.0, about 5.5 to about 10.5, about 5.5 to about 10.0, about 5.5 to about 9.5, about 5.5 to about 9.0, about 5.5 to about 8.5, about 5.5 to about 8.0, about 5.5 to about 7.5, about 5.5 to about 7.0, about 5.5 to about 6.5, about 5.5 to about 6.0, about 6.0 to about 11.0, about 6.0 to about 10.5, about 6.0 to about 10.0, about 6.0 to about 9.5, about 6.0 to about 9.0, about 6.0 to about 8.5, about 6.0 to about 8.0, about 6.0 to about 7.5, about 6.0 to about 7.0, about 6.0 to about 6.5, about 6.5 to about 11.0, about 6.5 to about 10.5, about 6.5 to about 10.0, about 6.5 to about 9.5, about 6.5 to about 9.0, about 6.5 to about 8.5, about 6.5 to about 8.0, about 6.5 to about 7.5, about 6.5 to about 7.0, about 7.0 to about 11.0, about 7.0 to about 10.5, about 7.0 to about 10.0, about 7.0 to about 9.5, about 7.0 to about 9.0, about 7.0 to about 8.5, about 7.0 to about 8.0, about 7.0 to about 7.5, about 7.5 to about 11.0, about 7.5 to about 10.5, about 7.5 to about 10.0, about 7.5 to about 9.5, about 7.5 to about 9.0, about 7.5 to about 8.5, about 7.5 to about 8.0, about 8.0 to about 11.0, about 8.0 to about 10.5, about 8.0 to about 10.0, about 8.0 to about 9.5, about 8.0 to about 9.0, about 8.0 to about 8.5, about 8.5 to about 11.0, about 8.5 to about 10.5, about 8.5 to about 10.0, about 8.5 to about 9.5, or about 8.5 to about 9.0, explants per square centimeter (cm²) of co-culture surface area, including all ranges and values derivable therebetween.

In particular embodiments, the embryo explants may be in contact with the co-culture medium at a temperature in a range from about 15° C. to about 25° C., or from about 17° C. to about 23° C., or from about 18° C. to about 20° C., or at a temperature of about 15° C., about 16° C., about 17° C., about 18° C., about 19° C., about 20° C., about 21° C., about 22° C., about 23° C., about 24° C., or about 25° C. The explants may be in contact with the co-culture medium, in some embodiments, for a time period ranging from about 1 day to about 14 days, from about 1 day to about 13 days, from about 1 day to about 12 days, from about 1 day to about 11 days, from about 1 day to about 10 days, from about 1 day to about 9 days, from about 1 day to about 8 days, from about 1 day to about 7 days, from about 1 day to about 6 days, from about 1 day to about 5 days, from about 1 day to about 4 days, from about 1 day to about 3 days, from about 1 day to about 2 days, from about 2 days to about 10 days, from about 2 days to about 8 days, from about 2 days to about 4 days, from about 2 days to about 5 days, from about 3 days to about 8 days, from about 4 days to about 8 days, from about 4 days to about 5 days, from about 5 days to about 7 days, such as for about 1 day, about 2 days, about 3 days, about 4 days, about 5 days, about 6 days, about 7 days, about 8 days, about 9 days, about 10 days, about 11 days, about 12 days, about 13 days, or about 14 days, including all ranges and values derivable therebetween. The explants, in further embodiments, may be in contact with the co-culture medium for at least 5 days or at least 6 days. In present embodiments, the co-culture medium in contact with the explants may be a solid, liquid or semi-solid medium.

According to some embodiments, the monocot seed embryo explant(s) may be in contact with the co-culture medium for a time period ranging from about 1 day to about 10 days, or from about 2 days to about 10 days, or from about 2 days to about 8 days, or from about 3 days to about 8 days, or from about 4 days to about 8 days, or from about 5 days to about 7 days, such as for about 2 days, about 3 days, about 4 days, about 5 days, about 6 days, about 7 days, or about 8 days, including all ranges and vales derivable therebetween. According to some embodiments, the monocot seed embryo explant(s) may be in contact with the co-culture medium for a longer time period to improve transformation, shoot and/or regeneration frequency, which may be particularly useful for seed embryo explants of certain male germplasms or lines or other monocot germplasms having a lower transformation, shoot and/or regeneration frequency. According to some embodiments, the monocot seed embryo explant(s) may be in contact with the co-culture medium for a time period ranging from about 5 day to about 10 days, or from about 5 days to about 9 days, or from about 5 days to about 8 days, or from about 5 days to about 7 days, or from about 5 days to about 6 days, or from about 6 days to about 7 days, such as for about 5 days, about 6 days, about 7 days, about 8 days, about 9 days, or about 10 days, including all ranges and values derivable therebetween.

According to some embodiments, the co-culture medium may comprise the Rhizobiales bacterium or Agrobacterium competent to transform at least one cell of the explant with the heterologous polynucleotide molecule. Higher concentrations of Agrobacterium in the inoculation and/or co-culture mediums can improve or increase shoot frequency and plugging frequency, and thus transformation frequency. The OD₆₆₀ concentration of the Rhizobiales bacterium or Agrobacterium in the inoculation and/or co-culture medium can be in a range from about 0.1 to about 2.0, from about 0.1 to about 1.0, from about 0.1 to about 0.75, from about 0.1 to about 0.5, from about 0.2 to about 2.0, from about 0.2 to about 1.0, from about 0.2 to about 0.5, from about 0.25 to about 2.0, from about 0.5 to about 2.0, from about 0.5 to about 1.5, from about 0.75 to about 1.5, or from about 0.75 to about 1.25, or about 0.1, 0.2, 0.25, 0.5. 0.75, 1.0, 1.25, 1.5, or 2.0, including all ranges and values derivable therebetween. However, in some cases, which may depend on the monocot plant germplasm or genetic line being transformed, a lower OD₆₆₀ concentration, such as in a range from about 0.1 to about 1.0 or from about 0.25 to about 0.5, or of about 0.1, about 0.2, about 0.25, about 0.3, about 0.4, about 0.5, about 0.6, about 0.7, about 0.75, about 0.8, about 0.9, or about 1.0, including all ranges and values derivable therebetween, may improve viability and/or regenerability of explants.

The explants, in certain embodiments, may be in contact with a matrix, paper or mesh material or substrate, such as a Whatman or other filter paper, that is wetted, filled or soaked with a liquid co-culture medium. In particular embodiments, the explants may be in contact with, but not submerged in, the co-culture medium. In certain embodiments, the explants may be co-cultured at a relative humidity of about 20% to about 90%, about 25% to about 65%, about 30% to about 60%, about 35% to about 55%, about 40% to about 50%, about 25%, about 30%, about 35%, about 40%, about 45%, about 50%, about 55%, about 60%, or about 65%, including all ranges and values derivable therebetween.

The co-culturing step may also be carried out under a variety of lighting conditions. While some degree of lighting may generally be used, all or part of the co-culture step may alternatively be performed in the dark. The lighting treatments may be quantified in terms of the light/dark cycle and light intensity, which may be expressed as the Photosynthetic Photon Flux Density (PPFD) in units of μE/m²·s. In some embodiments, the co-culturing step may be carried out with an average or set light intensity of Photosynthetic Active Radiation (PAR) in a range from about 0 μ/m²·s to about 200 μE/m²·s, 20 μE/m²·s to about 200 μE/m²·s, 20 μE/m²·s to about 180 μE/m²·s, 30 μE/m²·s to about 180 μE/m²·s, 30 μE/m²·s to about 150 μE/m²·s, 30 μE/m²·s to about 120 μE/m²·s, 60 μE/m²·s to about 120 μE/m²·s, 70 μE/m²·s to about 110 μE/m²·s, or 80 μE/m²·s to about 100 μE/m²·s. In certain embodiments, the co-culturing step may be carried out with an average or set light intensity of Photosynthetic Active Radiation (PAR) at about 0 μE/m²·s, about 10 μE/m²·s, about 20 μE/m²·s, about 30 μE/m²·s, about 40 μE/m²·s, about 50 μE/m²·s, about 60 μE/m²·s, about 70 μE/m²·s, about 80 μE/m²·s, about 90 μE/m²·s, about 100 μE/m²·s, about 110 μE/m²·s, about 120 μE/m²·s, about 130 μE/m²·s, about 140 μE/m²·s, about 150 μE/m²·s, about 160 μE/m²·s, about 170 μE/m²·s, about 180 μE/m²·s, about 190 μE/m²·s, or about 200 μE/m²·s. Different amounts of light and dark cycles, in some embodiments, may be used during the co-culture step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length. According to some embodiments, the amounts of light and dark cycles during the co-culture step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

After the introducing and/or inoculation step and any co-culture step, the embryo explants from dicot plant species, in particular embodiments, can be transferred to a regeneration medium to regenerate a plant or part thereof. However, embryo explants from monocot plant species may instead, in some embodiments, be transferred to a bud induction medium to carry out a bud induction and possibly extended bud induction steps following the introducing and/or inoculation step and any co-culture step. After the bud induction and possibly extended bud induction steps, the monocot embryo explants may then be transferred to a regeneration medium to regenerate a plant or part thereof as described further below.

### D. Bud Induction and Extended Bud Induction

According to present embodiments, a population of monocot embryo explants that have been transformed or edited by introducing a heterologous polynucleotide molecule into at least one cell of the embryo explants may be cultured in contact with at least a first bud induction medium comprising an auxin and a cytokinin. The monocot embryo explants may have been inoculated with an inoculation medium comprising a Rhizobiales or Agrobacterium that comprises the heterologous polynucleotide molecule, and the monocot seed embryo explant may also have been co-cultured in contact with a co-culture medium, prior to the bud induction step.

As provided herein, the monocot embryo explants may be further cultured in contact with a second or extended bud induction medium comprising an auxin and a cytokinin and then cultured in contact with a regeneration medium to produce a genetically modified plant or plant part. In some embodiments, the methods described herein comprise culturing the monocot embryo explants in contact with a second bud induction medium after the monocot embryo explants are cultured in contact with the bud induction medium (or first bud induction medium) and before regenerating or growing the genetically modified monocot plant or plant part from the cultured monocot embryo explants in contact with a regeneration medium. In further embodiments, the (first) bud induction medium and/or the second (or extended) bud induction medium may each comprise a high cytokinin to auxin ratio.

In certain embodiments, the bud induction medium (or first bud induction medium) and the second bud induction medium (or extended bud induction medium) may each comprise a variety of standard culture media or solution ingredients or components, such as for example, basal salts, macronutrients, micronutrients, sugars, antibiotics and/or vitamins. The bud induction medium (or first bud induction medium) and the second bud induction medium (or extended bud induction medium) may each comprise an auxin and a cytokinin. The bud induction medium (or first bud induction medium) and the second bud induction medium (or extended bud induction medium) may each comprise one or more selection agent(s), although according to many embodiments, a selection agent is absent in the first bud induction medium. The absence of the selection agent in the first bud induction medium may allow the first bud induction medium to function as a delay medium. The identity of the selection agent will typically depend on the selectable marker gene present in the heterologous polynucleotide molecule introduced into the population of monocot embryo explants. The bud induction medium (or first bud induction medium) and/or the second bud induction medium (or extended bud induction medium) may each be a solid, semi-solid or liquid medium, although each of these media may typically be a solid medium. A solid medium may comprise a gelling or polymeric agent or ingredient, such as agarose or similar, that can solidify and form the solid medium.

As used herein, a “high cytokinin to auxin ratio” generally refers to a condition where the level of cytokinin activity is relatively high in comparison to the level of auxin activity present in the medium, which may typically be a cytokinin:auxin ratio of at least about 1:1 or higher in terms of weight/volume provided. The exact cytokinin:auxin ratio, however, will depend on the exact chemical identities of the auxin and cytokinin since different auxins and cytokinins can have different activities and/or modes of action, as known in the art. The levels of cytokinin and auxin in a medium having a high cytokinin to auxin ratio may be present in the medium (measured in terms of weight/volume), for example, at a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1, including all values and ranges derivable therebetween.

The levels of cytokinin and auxin in a culture medium having a high cytokinin to auxin ratio may be, for example, greater than or equal to about 1:1 or at least about 1:1 or higher, greater than or equal to about 1.5:1 or at least about 1.5:1 or higher, greater than or equal to about 2:1 or at least about 2:1 or higher, greater than or equal to about 2.5:1 or at least about 2.5:1 or higher, greater than or equal to about 3:1 or at least about 3:1 or higher, greater than or equal to about 3.5:1 or at least about 3.5:1 or higher, greater than or equal to about 4:1 or at least about 4:1 or higher, greater than or equal to about 4.5:1 or at least about 4.5:1 or higher, greater than or equal to about 5:1 or at least about 5:1 or higher, greater than or equal to about 5.5:1 or at least about 5.5:1 or higher, greater than or equal to about 6:1 or at least about 6:1 or higher, greater than or equal to about 6.5:1 or at least about 6.5:1 or higher, greater than or equal to about 7:1 or at least about 7:1 or higher, greater than or equal to about 7.5:1 or at least about 7.5:1 or higher, greater than or equal to about 8:1 or at least about 8:1 or higher, greater than or equal to about 8.5:1 or at least about 8.5:1 or higher, greater than or equal to about 9:1 or at least about 9:1 or higher, greater than or equal to about 9.5:1 or at least about 9.5:1 or higher, greater than or equal to about 10:1 or at least about 10:1 or higher, greater than or equal to about 10.5:1 or at least about 10.5:1 or higher, greater than or equal to about 11:1 or at least about 11:1 or higher, greater than or equal to about 11.5:1 or at least about 11.5:1 or higher, or greater than or equal to about 12:1 or at least about 12:1 or higher, including all values and ranges derivable therebetween.

The levels of cytokinin and auxin in a culture medium having a high cytokinin to auxin ratio may be, for example, in a range between about 1:1 and about 12:1, about 2:1 and about 12:1, about 4:1 and about 12:1, about 6:1 and about 12:1, about 8:1 and about 12:1, about 1:1 and about 10:1, about 2:1 and about 10:1, about 4:1 and about 10:1, about 6:1 and about 10:1, about 8:1 and about 10:1, about 1:1 and about 8:1, about 2:1 and about 8:1, about 4:1 and about 8:1, about 6:1 and about 8:1, about 1:1 and about 6:1, about 2:1 and about 6:1, about 4:1 and about 6:1, about 1:1 and about 5:1, about 2:1 and about 5:1, about 3:1 and about 5:1, about 1:1 and about 4:1, about 2:1 and about 4:1, about 3:1 and about 4:1, about 1:1 and about 3:1, or about 1:1 and about 2:1, including all values and ranges derivable therebetween.

Non-limiting examples of cytokinins that may be used in the accordance with the present disclosure may include, but are not limited to: 6-benzylaminopurine (BAP), thidiazuron (TDZ), N-(2-chloro-4-pyridyl)-N-phenylurea (4-CPPU), kinetin, zeatin, diphenyl urea (DPU), 6-(gamma,gamma-dimethylallylamino)purine (2iP), and 6-(3-hydroxybenzylamino)purine (meta-topolin). Auxins which may be used in accordance with the present disclosure may include, but are not limited to: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

In present embodiments, the bud induction medium (or first bud induction medium) may comprise the same or a different auxin and/or the same or a different cytokinin than the second bud induction medium (or extended bud induction medium). The bud induction medium (or first bud induction medium) may comprise a first auxin and a first cytokinin, and the second bud induction medium (or extended bud induction medium) may comprise the first auxin or a second auxin and the first cytokinin or a second cytokinin. In some embodiments, the second bud induction medium (or extended bud induction medium) may comprise the same auxin or a different auxin as the bud induction medium (or the first bud induction medium). In further embodiments, the second bud induction (or extended bud induction medium) may comprise the same cytokinin or a different cytokinin as the bud induction medium (or first bud induction medium).

In certain embodiments, the concentration of the cytokinin (or two or more cytokinins) or the total cytokinin concentration in the first bud induction medium and/or the second (or extended) bud induction medium is in a range from about 0.1 mg/L to about 100.0 mg/L, 1 mg/L to about 90.0 mg/L, 1 mg/L to about 80.0 mg/L, 1 mg/L to about 75.0 mg/L, 2 mg/L to about 90.0 mg/L, 2 mg/L to about 80.0 mg/L, 2 mg/L to about 75.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, or about 1.0 mg/L to about 3.0 mg/L, including all values and ranges derivable therebetween. In some embodiments, the concentration of the cytokinin in the first bud induction medium or the second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all values and ranges derivable therebetween. The cytokinin in the first and second bud induction media may be the same or different, and each of these bud induction media may comprise one or more cytokinins.

In some embodiments, the concentration of the auxin (or two or more auxins) or the total auxin concentration in the first bud induction medium and/or the second (or extended) bud induction medium is in the range from about 0.01 mg/L to about 25.0 mg/L, about 0.05 mg/L to about 25 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, about 9.0 mg/L to about 11.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 0.05 mg/L to about 7.5 mg/L, about 0.02 mg/L to about 5 mg/L, or about 0.75 mg/L to about 2.5 mg/L, including all values and ranges derivable therebetween. In some embodiments, the concentration of the auxin in the first bud induction or second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween. The auxins in the first and second bud induction media may be the same or different, and each of these media may comprise one or more auxins.

As used herein, the “total cytokinin concentration” of a medium is the total concentration of all cytokinins present in the medium. For example, if the concentration of TDZ in a medium is 1 mg/L and the concentration of BAP in the medium is 2 mg/L, and no other cytokinins are present in the medium, then the total cytokinin concentration in the medium is 3 mg/L. As used herein, the “total auxin concentration” of a medium is the total concentration of all auxins present in the medium. For example, if the concentration of 2,4-D in a medium is 0.5 mg/L and the concentration of IAA in the medium is 1 mg/L, and no other auxins are present in the medium, then the total auxin concentration in the medium is 1.5 mg/L. For clarity, if only one cytokinin is present in a medium, then the total cytokinin concentration of the medium would equal the concentration of the one cytokinin in the medium, and if only one auxin is present in a medium, then the total auxin concentration of the medium would equal the concentration of the one auxin in the medium.

As used herein, “co-culture surface area”, “bud induction surface area”, “first bud induction surface area”, “extended bud induction surface area”, “second bud induction surface area”, and “regeneration surface area” each refer to the top surface area of a solid or semi-solid medium that is a co-culture medium, bud induction medium, first bud induction medium, extended bud induction medium, second bud induction medium, or regeneration medium, respectively.

In certain embodiments, the first bud induction medium and/or the second (or extended) bud induction medium may comprise a cytokinin, wherein the cytokinin is 6-benzylaminopurine (BAP). In some embodiments, the concentration of BAP in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all values and ranges derivable therebetween. In some embodiments, the concentration of BAP in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

In particular embodiments, the first bud induction medium and/or the second (or extended) bud induction medium may comprise a cytokinin, wherein the cytokinin is thidiazuron (TDZ). In some embodiments, the concentration of TDZ in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. In some embodiments, the concentration of TDZ in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In some embodiments, the first bud induction medium and/or the second (or extended) bud induction medium may comprise a cytokinin, wherein the cytokinin is N-(2-chloro-4-pyridyl)-N-phenylurea (4-CPPU). In specific embodiments, the concentration of 4-CPPU in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. In further embodiments, the concentration of 4-CPPU in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In specific embodiments, the first bud induction medium and/or the second (or extended) bud induction medium may comprise a cytokinin, wherein the cytokinin is kinetin. In some embodiments, the concentration of kinetin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all values and ranges derivable therebetween. In further embodiments, the concentration of kinetin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

In particular embodiments, the first bud induction medium and/or the second (or extended) bud induction medium may comprise a cytokinin, wherein the cytokinin is zeatin. In some embodiments, the concentration of zeatin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all values and ranges derivable therebetween. In further embodiments, the concentration of zeatin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

In certain embodiments, the first bud induction medium and/or the second (or extended) bud induction medium may comprise a cytokinin, wherein the cytokinin is 6-(gamma,gamma-dimethylallylamino)purine (2iP). In some embodiments, the concentration of 2iP in the first bud induction medium and/or the second (or extended) bud induction medium may be in the range from 5 mg/L to about 100.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 100.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 100.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 100.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 100.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 100.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, including all values and ranges derivable therebetween. In further embodiments, the concentration of 2iP in the first bud induction medium and/or the second (or extended) bud induction medium may be about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all values and ranges derivable therebetween.

In particular embodiments, the first bud induction medium and/or the second (or extended) bud induction medium may comprise a cytokinin, wherein the cytokinin is 6-(3-hydroxybenzylamino)purine (meta-topolin). In some embodiments, the concentration of meta-topolin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all values and ranges derivable therebetween. In further embodiments, the concentration of meta-topolin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

According to some embodiments, the second (or extended) bud induction medium may have a lower concentration of cytokinin(s) to improve transformation, shoot and/or regeneration frequency, which may be particularly useful for monocot seed embryo explants of certain male germplasms or lines or other monocot germplasms having a lower transformation, shoot and/or regeneration frequency. According to some embodiments, a cytokinin (or two or more cytokinins) may be present in the second (or extended) bud induction medium at a lower concentration, or the total cytokinin concentration in the second (or extended) bud induction medium may be lower, in a range from about 0.1 mg/L to about 20 mg/L, about 0.1 mg/L to about 15 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.2 mg/L to about 20 mg/L, about 0.2 mg/L to about 15 mg/L, about 0.2 mg/L to about 10 mg/L, about 0.2 mg/L to about 9 mg/L, about 0.2 mg/L to about 8 mg/L, about 0.2 mg/L to about 7 mg/L, about 0.2 mg/L to about 6 mg/L, about 0.2 mg/L to about 5 mg/L, about 0.2 mg/L to about 4 mg/L, about 0.2 mg/L to about 3 mg/L, about 0.2 mg/L to about 2 mg/L, about 0.2 mg/L to about 1 mg/L, about 0.5 mg/L to about 20 mg/L, about 0.5 mg/L to about 15 mg/L, about 0.5 mg/L to about 10 mg/L, about 0.5 mg/L to about 9 mg/L, about 0.5 mg/L to about 8 mg/L, about 0.5 mg/L to about 7 mg/L, about 0.5 mg/L to about 6 mg/L, about 0.5 mg/L to about 5 mg/L, about 0.5 mg/L to about 4 mg/L, about 0.5 mg/L to about 3 mg/L, about 0.5 mg/L to about 2 mg/L, or about 0.5 mg/L to about 1 mg/L, including all ranges and values derivable therebetween. In some embodiments, the lower concentration of the cytokinin in the second (or extended) bud induction medium, or the lower total cytokinin concentration in the second (or extended) bud induction medium, may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all ranges and values derivable therebetween.

In some embodiments, the second (or extended) bud induction medium may comprise a cytokinin at a lower concentration, wherein the cytokinin is 6-benzylaminopurine (BAP), kinetin, zeatin, or 6-(3-hydroxybenzylamino)purine (meta-topolin). In some embodiments, the lower concentration of BAP, kinetin, zeatin, or 6-(3-hydroxybenzylamino)purine (meta-topolin) in the second (or extended) bud induction medium may be in the range from about 0.1 mg/L to about 20 mg/L, about 0.1 mg/L to about 15 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 20 mg/L, about 0.5 mg/L to about 15 mg/L, about 0.5 mg/L to about 10 mg/L, about 0.5 mg/L to about 9 mg/L, about 0.5 mg/L to about 8 mg/L, about 0.5 mg/L to about 7 mg/L, about 0.5 mg/L to about 6 mg/L, about 0.5 mg/L to about 5 mg/L, about 0.5 mg/L to about 4 mg/L, about 0.5 mg/L to about 3 mg/L, about 0.5 mg/L to about 2 mg/L, about 0.5 mg/L to about 1 mg/L, about 1 mg/L to about 20 mg/L, about 1 mg/L to about 15 mg/L, about 1 mg/L to about 10 mg/L, about 1 mg/L to about 9 mg/L, about 1 mg/L to about 8 mg/L, about 1 mg/L to about 7 mg/L, about 1 mg/L to about 6 mg/L, about 1 mg/L to about 5 mg/L, about 1 mg/L to about 4 mg/L, about 1 mg/L to about 3 mg/L, about 1 mg/L to about 2 mg/L, about 2 mg/L to about 20 mg/L, about 2 mg/L to about 15 mg/L, about 2 mg/L to about 10 mg/L, about 2 mg/L to about 9 mg/L, about 2 mg/L to about 8 mg/L, about 2 mg/L to about 7 mg/L, about 2 mg/L to about 6 mg/L, about 2 mg/L to about 5 mg/L, about 2 mg/L to about 4 mg/L, or about 2 mg/L to about 3 mg/L, or at about 0.5 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, or about 15 mg/L, including all ranges and values derivable therebetween.

In some embodiments, the second (or extended) bud induction medium may comprise a cytokinin at a lower concentration, wherein the cytokinin is thidiazuron (TDZ). In some embodiments, the lower concentration of TDZ in the second (or extended) bud induction medium may be in the range from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 10 mg/L, about 0.5 mg/L to about 9 mg/L, about 0.5 mg/L to about 8 mg/L, about 0.5 mg/L to about 7 mg/L, about 0.5 mg/L to about 6 mg/L, about 0.5 mg/L to about 5 mg/L, about 0.5 mg/L to about 4 mg/L, about 0.5 mg/L to about 3 mg/L, about 0.5 mg/L to about 2 mg/L, about 0.5 mg/L to about 1 mg/L, about 1 mg/L to about 10 mg/L, about 1 mg/L to about 9 mg/L, about 1 mg/L to about 8 mg/L, about 1 mg/L to about 7 mg/L, about 1 mg/L to about 6 mg/L, about 1 mg/L to about 5 mg/L, about 1 mg/L to about 4 mg/L, about 1 mg/L to about 3 mg/L, about 1 mg/L to about 2 mg/L, about 2 mg/L to about 10 mg/L, about 2 mg/L to about 9 mg/L, about 2 mg/L to about 8 mg/L, about 2 mg/L to about 7 mg/L, about 2 mg/L to about 6 mg/L, about 2 mg/L to about 5 mg/L, about 2 mg/L to about 4 mg/L, or about 2 mg/L to about 3 mg/L, or at about 0.5 mg/L, about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween.

In some embodiments, the second (or extended) bud induction medium may comprise a cytokinin at a lower concentration, wherein the cytokinin is 6-(gamma,gamma-dimethylallylamino)purine (2iP). In some embodiments, the lower concentration of 2iP in the second (or extended) bud induction medium may be in the range from about 0.5 mg/L to about 40 mg/L, 0.5 mg/L to about 30 mg/L, 0.5 mg/L to about 25 mg/L, 0.5 mg/L to about 20 mg/L, about 0.5 mg/L to about 15 mg/L, about 0.5 mg/L to about 10 mg/L, about 0.5 mg/L to about 5 mg/L, about 1 mg/L to about 40 mg/L, 1 mg/L to about 30 mg/L, 1 mg/L to about 25 mg/L, 1 mg/L to about 20 mg/L, about 1 mg/L to about 15 mg/L, about 1 mg/L to about 10 mg/L, about 1 mg/L to about 5 mg/L, about 2 mg/L to about 40 mg/L, 2 mg/L to about 30 mg/L, 2 mg/L to about 25 mg/L, 2 mg/L to about 20 mg/L, about 2 mg/L to about 15 mg/L, about 2 mg/L to about 10 mg/L, about 2 mg/L to about 5 mg/L, about 5 mg/L to about 40 mg/L, 5 mg/L to about 30 mg/L, 5 mg/L to about 25 mg/L, 5 mg/L to about 20 mg/L, about 5 mg/L to about 15 mg/L, about 5 mg/L to about 10 mg/L, about 10 mg/L to about 40 mg/L, 10 mg/L to about 30 mg/L, 10 mg/L to about 25 mg/L, 10 mg/L to about 20 mg/L, or about 10 mg/L to about 15 mg/L, or at about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 35 mg/L, or about 40 mg/L, including all ranges and values derivable therebetween.

In specific embodiments, the first bud induction medium and/or the second (or extended) bud induction medium may comprise an auxin, wherein the auxin is 2,4-dichlorophenoxy-acetic acid (2,4-D). In some embodiments, the concentration of 2,4-D in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all values and ranges derivable therebetween. In further embodiments, the concentration of 2,4-D in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In some embodiments, the first bud induction medium and/or the second (or extended) bud induction medium may comprise an auxin, wherein the auxin is 2,4,5-trichloro-phenoxy acetic acid (2,4,5-T). In certain embodiments, the concentration of 2,4,5-T in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all values and ranges derivable therebetween. In further embodiments, the concentration of 2,4,5-T in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In some embodiments, the first bud induction medium and/or the second (or extended) bud induction medium may comprise an auxin, wherein the auxin is 4-amino-3,5,6-trichloro-picolinic acid (picloram). In further embodiments, the concentration of picloram in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. In specific embodiments, the concentration of picloram in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In certain embodiments, the first bud induction medium and/or the second (or extended) bud induction medium may comprise an auxin, wherein the auxin is indole-3-acetic acid (IAA). In some embodiments, the concentration of IAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In further embodiments, the concentration of IAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

In specific embodiments, the first bud induction medium and/or the second (or extended) bud induction medium may comprise an auxin, wherein the auxin is indole-3-butyric acid (IBA). In some embodiments, the concentration of IBA in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. In further embodiments, the concentration of IBA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

In particular embodiments, the first bud induction medium and/or the second (or extended) bud induction medium may comprise an auxin, wherein the auxin is naphthalene acetic acid (NAA). In further embodiments, the concentration of NAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all values and ranges derivable therebetween. In specific embodiments, the concentration of NAA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all values and ranges derivable therebetween.

In some embodiments, the first bud induction medium and/or the second (or extended) bud induction medium may comprise an auxin, wherein the auxin is 2,3,5-triiodobenzoic acid (TIBA). In further embodiments, the concentration of TIBA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all values and ranges derivable therebetween. In additional embodiments, the concentration of TIBA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all values and ranges derivable therebetween.

In particular embodiments, the first bud induction medium and/or the second (or extended) bud induction medium may comprise an auxin, wherein the auxin is phenylacetic acid (PAA). In further embodiments, the concentration of PAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of PAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all values and ranges derivable therebetween.

In certain embodiments, the first bud induction medium and/or the second (or extended) bud induction medium may comprise an auxin, wherein the auxin is 3,6-dichloro-2-methoxy-benzoic acid (dicamba). In further embodiments, the concentration of dicamba in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. In additional embodiments, the concentration of dicamba in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all values and ranges derivable therebetween.

According to some embodiments, the first bud induction medium comprises a first auxin and a first cytokinin, wherein the first auxin is 2,4-dichlorophenoxy-acetic acid (2,4-D) and the first cytokinin is 6-benzylaminopurine (BAP). According to these embodiments, the concentration of 2,4-D in the first bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all values and ranges derivable therebetween. According to these embodiments, the concentration of 6-benzylaminopurine (BAP) in the first bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween.

According to certain embodiments, the second (or extended) bud induction medium comprises a second auxin and a second cytokinin, wherein the second auxin is 4-amino-3,5,6-trichloro-picolinic acid (picloram) and the second cytokinin is thidiazuron (TDZ). According to these embodiments, the concentration of picloram in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween. According to these embodiments, the concentration of TDZ in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all values and ranges derivable therebetween.

According to some embodiments, methods described herein comprise having a lower density of visible or total monocot seed embryo explant(s) in a bud (or a first bud) induction medium per container or plate, which may be achieved by transferring the cultured monocot seed embryo explant(s) from a given number of co-culture plate(s) or container(s) to a relatively greater number of bud (or first bud) induction plate(s) or container(s). As described herein, transformation of male corn lines, and possibly other monocot germplasms, may often be difficult or less efficient as compared to female corn lines or other monocot germplasms. Compared to bud induction plates for female line corn embryo explants, bud induction plates for male line corn embryo explants can exhibit frequent contamination and tissue necrosis. According to some embodiments, a lower density of visible or total explants per bud (or first bud) induction plate or container may improve transformation, shoot, and/or regeneration frequency of genetically modified plants of monocot or corn embryo explants, or certain monocot or corn lines that are more resistant to efficient transformation and/or regeneration of genetically modified plants, such as certain male germplasm corn lines. According to some embodiments, monocot seed embryo explants may be transferred from one or more co-culture plate(s) or container(s) to a relatively smaller or fewer number of bud induction plate(s) or container(s), such as a ratio of 2:1 or 1:1 in terms of the number of co-culture plate(s) or container(s) to the number of bud induction plate(s) or container(s), but this may result in overcrowding of the number of explants per bud induction plate.

According to some embodiments, monocot seed embryo explants may be transferred from one or more co-culture induction plate(s) or container(s) to a relatively greater or higher number of bud induction plate(s) or container(s), such as a ratio of 1:2, 1:3, 1:4, 1:5, 1:6, 1:7, 1:8, 1:9, 1:10, 1:11, 1:12, 1:13, 1:14, or 1:15 in terms of the number of co-culture plate(s) or container(s) to the number of bud induction plate(s) or container(s). According to some embodiments, a given number of visible (or total) monocot seed embryo explants, such as in a range from about 10 to about 2,000, about 20 to about 2,000, about 30 to about 2,000, about 40 to about 2,000, about 50 to about 2,000, about 10 to about 1,500, about 10 to about 1,000, about 10 to about 1,500, about 10 to about 1,000, about 10 to about 750, about 10 to about 500, about 10 to about 600, about 10 to about 500, about 10 to about 400, about 10 to about 300, about 50 to about 200, about 20 to about 2,000, about 20 to about 1,500, about 20 to about 1,000, about 20 to about 1,500, about 20 to about 1,000, about 20 to about 750, about 20 to about 500, about 20 to about 600, about 20 to about 500, about 20 to about 400, about 20 to about 300, about 20 to about 200, about 30 to about 2,000, about 30 to about 1,500, about 30 to about 1,000, about 30 to about 1,500, about 30 to about 1,000, about 30 to about 750, about 30 to about 500, about 30 to about 600, about 30 to about 500, about 30 to about 400, about 30 to about 300, about 30 to about 200, about 40 to about 2,000, about 40 to about 1,500, about 40 to about 1,000, about 40 to about 1,500, about 40 to about 1,000, about 40 to about 750, about 40 to about 500, about 40 to about 600, about 40 to about 500, about 40 to about 400, about 40 to about 300, about 40 to about 200, about 50 to about 2,000, about 50 to about 1,500, about 50 to about 1,000, about 50 to about 1,500, about 50 to about 1,000, about 50 to about 750, about 50 to about 500, about 50 to about 600, about 50 to about 500, about 50 to about 400, about 50 to about 300, about 50 to about 200, including all ranges and values derivable therebetween, may be transferred from a co-culture plate or medium to a relatively greater or higher number of bud induction plate(s) or container(s), such as 2 or more, 3 or more, 4 or more, 5 or more, 6 or more, 7 or more, 8 or more, 9 or more, or 10 or more bud induction plate(s) or container(s).

According to some embodiments, the number of visible (or total) monocot seed embryo explants transferred or added to the bud induction plate(s) or container(s) may be at a density of less than or equal to about 500 seed embryo explants per plate, less than or equal to about 400 seed embryo explants per plate, less than or equal to about 300 seed embryo explants per plate, less than or equal to about 200 seed embryo explants per plate, less than or equal to about 150 seed embryo explants per plate, less than or equal to about 100 seed embryo explants per plate, less than or equal to about 75 seed embryo explants per plate, less than or equal to about 50 seed embryo explants per plate, or less than or equal to about 30 seed embryo explants per plate, or in a range of densities from about 10 to about 300 seed embryo explants per plate, about 10 to about 250 seed embryo explants per plate, about 10 to about 200 seed embryo explants per plate, about 10 to about 150 seed embryo explants per plate, about 10 to about 100 seed embryo explants per plate, about 10 to about 75 seed embryo explants per plate, about 10 to about 50 seed embryo explants per plate, about 10 to about 25 embryo explants per plate, about 25 to about 300 seed embryo explants per plate, about 25 to about 250 seed embryo explants per plate, about 25 to about 200 seed embryo explants per plate, about 25 to about 150 seed embryo explants per plate, about 25 to about 100 seed embryo explants per plate, about 25 to about 75 seed embryo explants per plate, about 25 to about 50 seed embryo explants per plate, about 50 to about 300 seed embryo explants per plate, about 50 to about 250 seed embryo explants per plate, about 50 to about 200 seed embryo explants per plate, about 50 to about 150 seed embryo explants per plate, about 50 to about 100 seed embryo explants per plate, about 50 to about 75 seed embryo explants per plate, or at about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, about 250, about 275, or about 300 embryo explants per plate, including all ranges and values derivable therebetween. For these density values and ranges, the surface area of each bud induction plate is approximately 11.9 square inches (in²) or 76.8 square centimeters (cm²). Thus, all of the above density values and ranges for seed embryo explants per plate can be readily converted into density values and ranges of seed embryo explants per bud induction surface area (for example, a density of 100 seed embryo explants per plate can be divided by the surface area per plate to provide a density of seed embryo explants per bud induction surface area of about 8.4 seed embryo explants/square inch (in²) or about 1.3 seed embryo explants/square centimeter (cm²), and similar conversions can be readily made for other density values and ranges). Density values and ranges of seed embryo explants per bud induction surface area is a more universal definition for density of seed embryo explants in a variety of different bud induction plate(s) or container(s) that may each have different surface areas.

In some embodiments, the number of visible (or total) monocot seed embryo explants transferred or added to the bud induction plate(s) or container(s) may be at a density of less than or equal to about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 embryo explants per square centimeter (cm²) of bud induction surface area, including all ranges and values derivable therebetween. In certain embodiments, the number of visible (or total) monocot seed embryo explants transferred or added to the bud induction plate(s) or container(s) may be at a density in a range from about 0.1 to about 6.0, about 0.1 to about 5.5, about 0.1 to about 5.0, about 0.1 to about 4.5, about 0.1 to about 4.0, about 0.1 to about 3.5, about 0.1 to about 3.0, about 0.1 to about 2.5, about 0.1 to about 2.0, about 0.1 to about 1.5, about 0.1 to about 1.0, about 0.1 to about 0.5, about 0.2 to about 6.0, about 0.2 to about 5.5, about 0.2 to about 5.0, about 0.2 to about 4.5, about 0.2 to about 4.0, about 0.2 to about 3.5, about 0.2 to about 3.0, about 0.2 to about 2.5, about 0.2 to about 2.0, about 0.2 to about 1.5, about 0.2 to about 1.0, about 0.2 to about 0.5, about 0.3 to about 6.0, about 0.3 to about 5.5, about 0.3 to about 5.0, about 0.3 to about 4.5, about 0.3 to about 4.0, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.4 to about 6.0, about 0.4 to about 5.5, about 0.4 to about 5.0, about 0.4 to about 4.5, about 0.4 to about 4.0, about 0.4 to about 3.5, about 0.4 to about 3.0, about 0.4 to about 2.5, about 0.4 to about 2.0, about 0.4 to about 1.5, about 0.4 to about 1.0, about 0.4 to about 0.5, about 0.5 to about 6.0, about 0.5 to about 5.5, about 0.5 to about 5.0, about 0.5 to about 4.5, about 0.5 to about 4.0, about 0.5 to about 3.5, about 0.5 to about 3.0, about 0.5 to about 2.5, about 0.5 to about 2.0, about 0.5 to about 1.5, about 0.5 to about 1.0, about 0.6 to about 6.0, about 0.6 to about 5.5, about 0.6 to about 5.0, about 0.6 to about 4.5, about 0.6 to about 4.0, about 0.6 to about 3.5, about 0.6 to about 3.0, about 0.6 to about 2.5, about 0.6 to about 2.0, about 0.6 to about 1.5, about 0.6 to about 1.0, about 0.7 to about 6.0, about 0.7 to about 5.5, about 0.7 to about 5.0, about 0.7 to about 4.5, about 0.7 to about 4.0, about 0.7 to about 3.5, about 0.7 to about 3.0, about 0.7 to about 2.5, about 0.7 to about 2.0, about 0.7 to about 1.5, about 0.7 to about 1.0, about 0.8 to about 6.0, about 0.8 to about 5.5, about 0.8 to about 5.0, about 0.8 to about 4.5, about 0.8 to about 4.0, about 0.8 to about 3.5, about 0.8 to about 3.0, about 0.8 to about 2.5, about 0.8 to about 2.0, about 0.8 to about 1.5, about 0.8 to about 1.0, about 0.9 to about 6.0, about 0.9 to about 5.5, about 0.9 to about 5.0, about 0.9 to about 4.5, about 0.9 to about 4.0, about 0.9 to about 3.5, about 0.9 to about 3.0, about 0.9 to about 2.5, about 0.9 to about 2.0, about 0.9 to about 1.5, about 0.9 to about 1.0, about 1.0 to about 6.0, about 1.0 to about 5.5, about 1.0 to about 5.0, about 1.0 to about 4.5, about 1.0 to about 4.0, about 1.0 to about 3.5, about 1.0 to about 3.0, about 1.0 to about 2.5, about 1.0 to about 2.0, about 1.0 to about 1.5, about 1.5 to about 6.0, about 1.5 to about 5.5, about 1.5 to about 5.0, about 1.5 to about 4.5, about 1.5 to about 4.0, about 1.5 to about 3.5, about 1.5 to about 3.0, about 1.5 to about 2.5, or about 1.5 to about 2.0, embryo explants per square centimeter (cm²) of bud induction surface area, including all ranges and values derivable therebetween.

According to embodiments of the present disclosure, the population of monocot embryo explants may be cultured in contact with the first bud induction medium for about 2 days to about 14 days, about 4 days to about 12 days, about 5 days to about 10 days, or about 6 days to about 8 days, including all ranges derivable therebetween. In some embodiments, the monocot embryo explants are cultured in contact with the first bud induction medium for about 2 days, about 3 days, about 4 days, about 5 days, about 6 days, about 7 days (or about 1 week), about 8 days, about 9 days, about 10 days, about 11 days, about 12 days, about 13 days, or about 14 days (or about 2 weeks), including all ranges derivable therebetween. In further embodiments, the monocot embryo explants may be cultured in contact with the first bud induction medium at a temperature in a range from about 20° C. to about 30° C., about 22° C. to about 28° C., about 25° C. to about 30° C., about 25° C. to about 29° C., or about 25° C. to about 28° C., including all ranges derivable therebetween. According to specific embodiments, the monocot embryo explants may be cultured in contact with the first bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. According to an aspect of the present disclosure, the monocot embryo explants may be cultured in contact with the first bud induction medium at elevated temperature, which may be in a range from about 30° C. to about 40° C., about 30° C. to about 38° C., about 30° C. to about 36° C., about 30° C. to about 35° C., about 31° C. to about 40° C., about 31° C. to about 38° C., about 31° C. to about 36° C., about 31° C. to about 35° C., about 32° C. to about 40° C., about 32° C. to about 38° C., about 32° C. to about 36° C., about 32° C. to about 35° C., about 33° C. to about 40° C., about 33° C. to about 38° C., about 33° C. to about 36° C., or about 33° C. to about 35° C., including all ranges derivable therebetween. According to further embodiments, the monocot embryo explants may be cultured in contact with the first bud induction medium at an elevated temperature of about 30° C., about 31° C., about 32° C., about 33° C., about 34° C., about 35° C., about 36° C., about 37° C., about 38° C., about 39° C., or about 40° C., including all ranges derivable therebetween. A selection agent may generally be absent from the first bud induction medium, but the first bud induction medium may alternatively comprise a selection agent.

In another aspect, culturing monocot seed embryo explants in contact with the first bud induction medium at an elevated temperature, for example a temperature in a range from about 30° C. to about 40° C., for about one week may improve transformation, as compared to culturing the explants in contact with the first bud induction medium at a lower temperature, for example at a temperature in a range from about 20° C. to about 30° C., during the first bud induction step.

The first bud induction step may also be carried out under a variety of lighting conditions. While some degree of lighting may generally be used, all or part of the first bud induction step may alternatively be performed in the dark. According to some embodiments, the first bud induction step may be carried out with an average or set light intensity of Photosynthetic Active Radiation (PAR) in a range from about 0 μE/m²·s to about 200 μE/m²·s, 20 μE/m²·s to about 200 μE/m²·s, 20 μE/m²·s to about 180 μE/m²·s, 30 μE/m²·s to about 180 μE/m²·s, 50 μE/m²·s to about 180 μE/m²·s, 50 μE/m²·s to about 150 μE/m²·s, 60 μE/m²·s to about 150 μE/m²·s, 70 μE/m²·s to about 140 μE/m²·s, 80 μE/m²·s to about 130 μE/m²·s, or 90 μE/m²·s to about 120 μE/m²·s. According to further embodiments, the first bud induction step may be carried out with an average or set light intensity of Photosynthetic Active Radiation (PAR) at about 0 μE/m²·s, about 10 μE/m²·s, about 20 μE/m²·s, about 30 μE/m²·s, about 40 μE/m²·s, about 50 μE/m²·s, about 60 μE/m²·s, about 70 μE/m²·s, about 80 μE/m²·s, about 90 μE/m²·s, about 100 μE/m²·s, about 110 μE/m²·s, about 120 μE/m²·s, about 130 μE/m²·s, about 140 μE/m²·s, about 150 μE/m²·s, about 160 μE/m²·s, about 170 μE/m²·s, about 180 μE/m²·s, about 190 μE/m²·s, or about 200 μE/m²·s. In specific embodiments, different amounts of light and dark cycles may be used during the first bud induction step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the first bud induction step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

According to embodiments of the present disclosure, the monocot embryo explants of the population may be cultured in contact with the second (or extended) bud induction medium for about 4 days to about 28 days, about 4 days to about 25 days, about 4 days to about 21 days, about 5 days to about 25 days, about 5 days to about 23 days, about 7 days to about 21 days, about 5 days to about 15 days, about 7 days to about 14 days, about 12 days to about 23 days, or about 14 days to about 21 days, including all ranges derivable therebetween. In some embodiments, the monocot embryo explants may be cultured in contact with the second (or extended) bud induction medium for about 4 days, about 5 days, about 6 days, about 7 days (or about 1 week), about 8 days, about 9 days, about 10 days, about 11 days, about 12 days, about 13 days, about 14 days (or about 2 weeks), about 15 days, about 16 days, about 17 days, about 18 days, about 19 days, about 20 days, about 21 days (or about 3 weeks), about 22 days, about 23 days, about 24 days, about 25 days, about 26 days, about 27 days, or about 28 days (or about 4 weeks), including all ranges derivable therebetween. In further embodiments, the monocot seed embryo explants may be cultured in contact with the second (or extended) bud induction medium at a temperature in a range from about 20° C. to about 32° C., about 20° C. to about 30° C., about 22° C. to about 28° C., about 25° C. to about 30° C., about 25° C. to about 29° C., about 26° C. to about 29° C., about 25° C. to about 28° C., or about 27° C. to about 28° C., including all ranges derivable therebetween. According to certain embodiments, the monocot embryo explants may be cultured in contact with the second (or extended) bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. In a particular embodiment, the monocot seed embryo explant may be cultured in contact with a first bud induction medium for a time period in a range from about 2 days to about 14 days at a temperature in a range from about 20° C. to about 30° C. or at an elevated temperature in a range from about 30° C. to about 40° C., and then subsequently cultured in contact with a second (or extended) bud induction medium for a time period in a range from about 4 days to about 28 days at a temperature in a range from about 20° C. to about 32° C. The second (or extended) bud induction medium may also comprise a selection agent. In specific embodiments, culturing the explants in contact with the second bud induction medium may improve transformation compared to only culturing the explants in contact with the first bud induction medium.

The second (or extended) bud induction step may also be carried out under a variety of lighting conditions. Some degree of lighting may generally be used during the second (or extended) bud induction step. According to some embodiments, the second (or extended) bud induction step may be carried out with an average or set light intensity of Photosynthetic Active Radiation (PAR) in a range from about 30 μE/m²·s to about 200 μE/m²·s, 30 μE/m²·s to about 180 μE/m²·s, 50 μE/m²·s to about 180 μE/m²·s, 50 μE/m²·s to about 150 μE/m²·s, 60 μE/m²·s to about 150 μE/m²·s, 70 μE/m²·s to about 140 μE/m²·s, 80 μE/m²·s to about 130 μE/m²·s, or 90 μE/m²·s to about 120 μE/m²·s. According to further embodiments, the second (or extended) bud induction step may be carried out with an average or set light intensity of Photosynthetic Active Radiation (PAR) at about 10 μE/m²·s, about 20 μE/m²·s, about 30 μE/m²·s, about 40 μE/m²·s, about 50 μE/m²·s, about 60 μE/m²·s, about 70 μE/m²·s, about 80 μE/m²·s, about 90 μE/m²·s, about 100 μE/m²·s, about 110 μE/m²·s, about 120 μE/m²·s, about 130 μE/m²·s, about 140 μE/m²·s, about 150 μE/m²·s, about 160 μE/m²·s, about 170 μE/m²·s, about 180 μE/m²·s, about 190 μE/m²·s, or about 200 μE/m²·s. In certain embodiments, different amounts of light and dark cycles may be used during the second (or extended) bud induction step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the second (or extended) bud induction step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

According to some embodiments, methods described herein comprise having a lower density of visible or total monocot seed embryo explant(s) in a second (or extended) bud induction medium per container or plate, which may be achieved by transferring the cultured monocot seed embryo explant(s) from a given number of bud induction plate(s) or container(s) to a relatively greater number of extended bud induction plate(s) or container(s). As described herein, transformation of male corn lines, and possibly other monocot germplasms, may often be difficult or less efficient as compared to female corn lines or other monocot germplasms. Compared to extended bud induction plates for female line corn embryo explants, extended bud induction plates for male line corn embryo explants can exhibit frequent contamination and tissue necrosis. According to some embodiments, a lower density of visible or total explants per extended bud induction plate or container may improve transformation, shoot and/or regeneration frequency of genetically modified plants of monocot or corn embryo explants, or certain monocot or corn lines that are more resistant to efficient transformation and/or regeneration of genetically modified plants, such as certain male germplasm corn lines. According to some embodiments, monocot seed embryo explants may be transferred from one or more bud induction plate(s) or container(s) to a relatively smaller or fewer number of extended bud induction plate(s) or container(s), such as a ratio of 2:1 or 1:1 in terms of the number of bud induction plate(s) or container(s) to the number of extended bud induction plate(s) or container(s), but this may result in overcrowding of the number of explants per extended bud induction plate.

According to some embodiments, monocot seed embryo explants may be transferred from one or more bud induction plate(s) or container(s) to a relatively greater or higher number of extended bud induction plate(s) or container(s), such as a ratio of 1:2, 1:3, 1:4, 1:5, 1:6, 1:7, 1:8, 1:9, or 1:10 in terms of the number of bud induction plate(s) or container(s) to the number of extended bud induction plate(s) or container(s). According to some embodiments, a given number of visible (or total) monocot seed embryo explants, such as in a range from 10 to about 2,000, about 20 to about 2,000, about 30 to about 2,000, about 40 to about 2,000, about 50 to about 2,000, about 10 to about 1,500, about 10 to about 1,000, about 10 to about 1,500, about 10 to about 1,000, about 10 to about 750, about 10 to about 500, about 10 to about 600, about 10 to about 500, about 10 to about 400, about 10 to about 300, about 50 to about 200, about 20 to about 2,000, about 20 to about 1,500, about 20 to about 1,000, about 20 to about 1,500, about 20 to about 1,000, about 20 to about 750, about 20 to about 500, about 20 to about 600, about 20 to about 500, about 20 to about 400, about 20 to about 300, about 20 to about 200, about 30 to about 2,000, about 30 to about 1,500, about 30 to about 1,000, about 30 to about 1,500, about 30 to about 1,000, about 30 to about 750, about 30 to about 500, about 30 to about 600, about 30 to about 500, about 30 to about 400, about 30 to about 300, about 30 to about 200, about 40 to about 2,000, about 40 to about 1,500, about 40 to about 1,000, about 40 to about 1,500, about 40 to about 1,000, about 40 to about 750, about 40 to about 500, about 40 to about 600, about 40 to about 500, about 40 to about 400, about 40 to about 300, about 40 to about 200, about 50 to about 2,000, about 50 to about 1,500, about 50 to about 1,000, about 50 to about 1,500, about 50 to about 1,000, about 50 to about 750, about 50 to about 500, about 50 to about 600, about 50 to about 500, about 50 to about 400, about 50 to about 300, about 50 to about 200, may be transferred from a bud induction plate or medium to a relatively greater or higher number of extended bud induction plate(s) or container(s), such as 2 or more, 3 or more, 4 or more, 5 or more, 6 or more, 7 or more, 8 or more, 9 or more, or 10 or more extended bud induction plate(s) or container(s).

According to some embodiments, the number of visible (or total) monocot seed embryo explants transferred or added to extended bud induction plate(s) or container(s) may be at a density of less than or equal to about 300 seed embryo explants per plate, less than or equal to about 200 seed embryo explants per plate, less than or equal to about 150 seed embryo explants per plate, less than or equal to about 100 seed embryo explants per plate, less than or equal to about 75 seed embryo explants per plate, less than or equal to about 50 seed embryo explants per plate, less than or equal to about 30 seed embryo explants per plate, or in a range of densities from about 10 to about 300 seed embryo explants per plate, about 10 to about 250 seed embryo explants per plate, about 10 to about 200 seed embryo explants per plate, about 10 to about 150 seed embryo explants per plate, about 10 to about 100 seed embryo explants per plate, about 10 to about 75 seed embryo explants per plate, about 10 to about 50 seed embryo explants per plate, about 25 to about 300 seed embryo explants per plate, about 25 to about 250 seed embryo explants per plate, about 25 to about 200 seed embryo explants per plate, about 25 to about 150 seed embryo explants per plate, about 25 to about 100 seed embryo explants per plate, about 25 to about 75 seed embryo explants per plate, about 25 to about 50 seed embryo explants per plate, about 50 to about 300 seed embryo explants per plate, about 50 to about 250 seed embryo explants per plate, about 50 to about 200 seed embryo explants per plate, about 50 to about 150 seed embryo explants per plate, about 50 to about 100 seed embryo explants per plate, about 50 to about 75 seed embryo explants per plate, or at about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, about 250, about 275, or about 300, including all ranges and values derivable therebetween. For these density values and ranges, the surface area of each extended bud induction plate is approximately 11.9 square inches (in²) or 76.8 square centimeters (cm²). Thus, all of the above density values and ranges for seed embryo explants per plate can be readily converted into density values and ranges of seed embryo explants per extended bud induction surface area (for example, a density of 100 seed embryo explants per plate can be divided by the surface area per plate to provide a density of seed embryo explants per extended bud induction surface area of about 8.4 seed embryo explants/square inch (in²) or about 1.3 seed embryo explants/square centimeter (cm²), and similar conversions can be readily made for other density values and ranges). Density values and ranges of seed embryo explants per extended bud induction surface area is a more universal definition for density of seed embryo explants in a variety of different extended bud induction plate(s) or container(s) that may each have different surface areas.

In some embodiments, the number of visible (or total) monocot seed embryo explants transferred or added to the extended bud induction plate(s) or container(s) may be at a density of less than or equal to about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 embryo explants per square centimeter (cm²) of extended bud induction surface area, including all ranges and values derivable therebetween. In certain embodiments, the number of visible (or total) monocot seed embryo explants transferred or added to the extended bud induction plate(s) or container(s) may be at a density in a range from about 0.1 to about 6.0, about 0.1 to about 5.5, about 0.1 to about 5.0, about 0.1 to about 4.5, about 0.1 to about 4.0, about 0.1 to about 3.5, about 0.1 to about 3.0, about 0.1 to about 2.5, about 0.1 to about 2.0, about 0.1 to about 1.5, about 0.1 to about 1.0, about 0.1 to about 0.5, about 0.2 to about 6.0, about 0.2 to about 5.5, about 0.2 to about 5.0, about 0.2 to about 4.5, about 0.2 to about 4.0, about 0.2 to about 3.5, about 0.2 to about 3.0, about 0.2 to about 2.5, about 0.2 to about 2.0, about 0.2 to about 1.5, about 0.2 to about 1.0, about 0.2 to about 0.5, about 0.3 to about 6.0, about 0.3 to about 5.5, about 0.3 to about 5.0, about 0.3 to about 4.5, about 0.3 to about 4.0, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.4 to about 6.0, about 0.4 to about 5.5, about 0.4 to about 5.0, about 0.4 to about 4.5, about 0.4 to about 4.0, about 0.4 to about 3.5, about 0.4 to about 3.0, about 0.4 to about 2.5, about 0.4 to about 2.0, about 0.4 to about 1.5, about 0.4 to about 1.0, about 0.4 to about 0.5, about 0.5 to about 6.0, about 0.5 to about 5.5, about 0.5 to about 5.0, about 0.5 to about 4.5, about 0.5 to about 4.0, about 0.5 to about 3.5, about 0.5 to about 3.0, about 0.5 to about 2.5, about 0.5 to about 2.0, about 0.5 to about 1.5, about 0.5 to about 1.0, about 0.6 to about 6.0, about 0.6 to about 5.5, about 0.6 to about 5.0, about 0.6 to about 4.5, about 0.6 to about 4.0, about 0.6 to about 3.5, about 0.6 to about 3.0, about 0.6 to about 2.5, about 0.6 to about 2.0, about 0.6 to about 1.5, about 0.6 to about 1.0, about 0.7 to about 6.0, about 0.7 to about 5.5, about 0.7 to about 5.0, about 0.7 to about 4.5, about 0.7 to about 4.0, about 0.7 to about 3.5, about 0.7 to about 3.0, about 0.7 to about 2.5, about 0.7 to about 2.0, about 0.7 to about 1.5, about 0.7 to about 1.0, about 0.8 to about 6.0, about 0.8 to about 5.5, about 0.8 to about 5.0, about 0.8 to about 4.5, about 0.8 to about 4.0, about 0.8 to about 3.5, about 0.8 to about 3.0, about 0.8 to about 2.5, about 0.8 to about 2.0, about 0.8 to about 1.5, about 0.8 to about 1.0, about 0.9 to about 6.0, about 0.9 to about 5.5, about 0.9 to about 5.0, about 0.9 to about 4.5, about 0.9 to about 4.0, about 0.9 to about 3.5, about 0.9 to about 3.0, about 0.9 to about 2.5, about 0.9 to about 2.0, about 0.9 to about 1.5, about 0.9 to about 1.0, about 1.0 to about 6.0, about 1.0 to about 5.5, about 1.0 to about 5.0, about 1.0 to about 4.5, about 1.0 to about 4.0, about 1.0 to about 3.5, about 1.0 to about 3.0, about 1.0 to about 2.5, about 1.0 to about 2.0, about 1.0 to about 1.5, about 1.5 to about 6.0, about 1.5 to about 5.5, about 1.5 to about 5.0, about 1.5 to about 4.5, about 1.5 to about 4.0, about 1.5 to about 3.5, about 1.5 to about 3.0, about 1.5 to about 2.5, or about 1.5 to about 2.0, embryo explants per square centimeter (cm²) of extended bud induction surface area, including all ranges and values derivable therebetween.

Without being bound by theory, the bud induction step(s) may cause differentiation and/or proliferation of cells of the explants to form multiple buds on the explant, which may then be regenerated into a plant. According to some preferred embodiments, the first auxin and cytokinin are different than the second auxin and cytokinin to affect the formation and development of the multiple buds through somewhat different activities and/or modes of action. Without being bound by theory, the first bud induction step may cause differentiation of cells of the explants into multiple buds, whereas the second (or extended) bud induction step may promote proliferation or expansion of the multiple buds to produce a more compact or solid multiple bud explant(s) for further culturing and regeneration into a plant(s). The inclusion of the second (or extended) bud induction step may have the further benefit of reducing chimerism of the resulting genetically modified plants or plant parts. According to certain embodiments, culturing the monocot embryo explants in a first bud induction medium followed by a second (or extended) bud induction medium may reduce chimerism in regenerated plants, as compared to culturing the monocot seed embryo explants in the first bud induction medium but without culturing in the second (or extended) bud induction medium prior to regeneration.

### E. Regeneration of Transformed or Edited Plants

In another aspect of the present disclosure, a plurality of genetically modified plants or plant parts is regenerated from the population of cultured embryo explants in contact with a regeneration medium. According to present embodiments, a regeneration medium may comprise a variety of standard culture media or solution ingredients or components, such as for example, basal salts, macronutrients, micronutrients, sugars, antibiotics and/or vitamins. The regeneration medium may not comprise an auxin or a cytokinin, which may be particularly applicable for regeneration of or from monocot embryo explants, although an auxin and/or a cytokinin may alternatively be present in the regeneration medium, which may be particularly applicable, in some embodiments, to regeneration of or from dicot embryo explants depending on the dicot plant species. The regeneration medium may typically comprise at least one selection agent, which may correspond to a selectable marker present in the heterologous polynucleotide molecule. The regeneration medium may be a solid, semi-solid or liquid medium, although a regeneration media may typically be a solid medium. A solid medium may comprise a gelling or polymeric agent or ingredient, such as agarose or similar, that can solidify and form the solid medium. As used herein, the term “regeneration” refers to the process of growing a plant or part thereof from one or more plant cells or tissues of an explant or any progeny generation of a cell thereof, and the term “regeneration medium” refers to a plant tissue culture medium formulated for regeneration of a plant from an explant. In some embodiments, regeneration or a regeneration step may refer to one or more regeneration step(s) that may involve culturing an explant or cultured explant in contact with two or more regeneration media. These two or more regeneration media may be the same or different regeneration medium/media. Explants of the population may, in some embodiments, be subcultured or transferred from a first regeneration medium to a second regeneration medium, and possibly to a third regeneration medium, and so on.

According to some embodiments, methods described herein comprise having a lower density of visible (or total) monocot seed embryo explant(s) in a regeneration medium per container or plate, which may be achieved by transferring the cultured monocot seed embryo explant(s) from a given number of extended bud induction plate(s) or container(s) to a greater number of regeneration plate(s) or container(s). As described herein, transformation of male corn lines, and possibly other monocot germplasms, may often be difficult or less efficient as compared to female corn lines or other monocot germplasms. Compared to regeneration plates for female line corn embryo explants, regeneration plates for male line corn embryo explants may exhibit frequent contamination and tissue necrosis. According to some embodiments, a lower density of visible or total explants per regeneration plate or container may improve transformation, shoot development and/or regeneration frequency of genetically modified plants of monocot or corn embryo explants, or certain monocot or corn lines that are more resistant to efficient transformation and/or regeneration of genetically modified plants, such as certain male germplasm corn lines.

According to some embodiments, monocot seed embryo explants may be transferred from one or more extended bud induction plate(s) or container(s) to a relatively fewer number of regeneration plate(s) or container(s), such as a ratio of 3:1 or 2:1 in terms of the number of extended bud induction plate(s) or container(s) to the number of regeneration plate(s) or container(s), but this may result in overcrowding of the number of explants per regeneration plate. Transferring explants to solid regeneration media following extended bud induction, at a ratio of 1 regeneration plate for every 3 extended bud induction plates, may result in an increase in contamination and tissue necrosis during regeneration, leading to loss of transformed tissues and regenerated plants.

According to some embodiments, monocot seed embryo explants may be transferred from one or more extended bud induction plate(s) or container(s) to a greater or higher number of regeneration plate(s) or container(s), such as a ratio of 1:1, 1:2, 1:3, 1:4, 1:5, 1:6, 1:7, 1:8, 1:9, or 1:10 in terms of the number of extended bud induction plate(s) or container(s) to the number of regeneration plate(s) or container(s). According to some embodiments, a given number of visible (or total) monocot seed embryo explants, such as in a range from about 10 to about 400, about 10 to about 300, about 10 to about 250, about 10 to about 200, about 10 to about 175, about 10 to about 150, about 10 to about 125, about 10 to about 100, about 10 to about 75, about 10 to about 50, or about 10 to about 25, including all ranges and values derivable therebetween, may be transferred from an extended bud induction plate or medium to a regeneration plate(s) or container(s), such as 1 or more, 2 or more, 3 or more, 4 or more, 5 or more, 6 or more, 7 or more, 8 or more, 9 or more, or 10 or more regeneration plate(s) or container(s). According to some embodiments, the number of visible (or total) monocot seed embryo explants transferred or added to regeneration plate(s) or container(s) may be at a density of less than or equal to about 300 seed embryo explants per plate, less than or equal to about 200 seed embryo explants per plate, less than or equal to about 150 seed embryo explants per plate, less than or equal to about 100 seed embryo explants per plate, or less than or equal to about 75 seed embryo explants per plate, less than or equal to about 50 seed embryo explants per plate, less than or equal to about 30 seed embryo explants per plate, or in a range of densities from about 10 to about 300 seed embryo explants per plate, about 10 to about 250 seed embryo explants per plate, about 10 to about 200 seed embryo explants per plate, about 10 to about 150 seed embryo explants per plate, about 10 to about 100 seed embryo explants per plate, about 10 to about 75 seed embryo explants per plate, about 10 to about 50 seed embryo explants per plate, about 25 to about 300 seed embryo explants per plate, about 25 to about 250 seed embryo explants per plate, about 25 to about 200 seed embryo explants per plate, about 25 to about 150 seed embryo explants per plate, about 25 to about 100 seed embryo explants per plate, about 25 to about 75 seed embryo explants per plate, about 25 to about 50 seed embryo explants per plate, about 50 to about 300 seed embryo explants per plate, about 50 to about 250 seed embryo explants per plate, about 50 to about 200 seed embryo explants per plate, about 50 to about 150 seed embryo explants per plate, about 50 to about 100 seed embryo explants per plate, about 50 to about 75 seed embryo explants per plate, or at about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, about 250, about 275, or about 300, including all ranges and values derivable therebetween. For these density values and ranges, the surface area of each regeneration plate is approximately 11.9 square inches (in²) or 76.8 square centimeters (cm²). Thus, all of the above density values and ranges for seed embryo explants per plate can be readily converted into density values and ranges of seed embryo explants per regeneration surface area (for example, a density of 100 seed embryo explants per plate can be divided by the surface area per plate to provide a density of seed embryo explants per regeneration surface area of about 8.4 seed embryo explants/square inch (in²) or about 1.3 seed embryo explants/square centimeter (cm²), and similar conversions can be readily made for other density values and ranges). Density values and ranges of seed embryo explants per regeneration surface area is a more universal definition for density of seed embryo explants in a variety of different regeneration plate(s) or container(s) that may each have different surface areas.

In some embodiments, the number of visible (or total) monocot seed embryo explants transferred or added to the regeneration plate(s) or container(s) may be at a density of less than or equal to about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 embryo explants per square centimeter (cm²) of regeneration surface area, including all ranges and values derivable therebetween. In certain embodiments, the number of visible (or total) monocot seed embryo explants transferred or added to the regeneration plate(s) or container(s) may be at a density in a range from about 0.1 to about 6.0, about 0.1 to about 5.5, about 0.1 to about 5.0, about 0.1 to about 4.5, about 0.1 to about 4.0, about 0.1 to about 3.5, about 0.1 to about 3.0, about 0.1 to about 2.5, about 0.1 to about 2.0, about 0.1 to about 1.5, about 0.1 to about 1.0, about 0.1 to about 0.5, about 0.2 to about 6.0, about 0.2 to about 5.5, about 0.2 to about 5.0, about 0.2 to about 4.5, about 0.2 to about 4.0, about 0.2 to about 3.5, about 0.2 to about 3.0, about 0.2 to about 2.5, about 0.2 to about 2.0, about 0.2 to about 1.5, about 0.2 to about 1.0, about 0.2 to about 0.5, about 0.3 to about 6.0, about 0.3 to about 5.5, about 0.3 to about 5.0, about 0.3 to about 4.5, about 0.3 to about 4.0, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.4 to about 6.0, about 0.4 to about 5.5, about 0.4 to about 5.0, about 0.4 to about 4.5, about 0.4 to about 4.0, about 0.4 to about 3.5, about 0.4 to about 3.0, about 0.4 to about 2.5, about 0.4 to about 2.0, about 0.4 to about 1.5, about 0.4 to about 1.0, about 0.4 to about 0.5, about 0.5 to about 6.0, about 0.5 to about 5.5, about 0.5 to about 5.0, about 0.5 to about 4.5, about 0.5 to about 4.0, about 0.5 to about 3.5, about 0.5 to about 3.0, about 0.5 to about 2.5, about 0.5 to about 2.0, about 0.5 to about 1.5, about 0.5 to about 1.0, about 0.6 to about 6.0, about 0.6 to about 5.5, about 0.6 to about 5.0, about 0.6 to about 4.5, about 0.6 to about 4.0, about 0.6 to about 3.5, about 0.6 to about 3.0, about 0.6 to about 2.5, about 0.6 to about 2.0, about 0.6 to about 1.5, about 0.6 to about 1.0, about 0.7 to about 6.0, about 0.7 to about 5.5, about 0.7 to about 5.0, about 0.7 to about 4.5, about 0.7 to about 4.0, about 0.7 to about 3.5, about 0.7 to about 3.0, about 0.7 to about 2.5, about 0.7 to about 2.0, about 0.7 to about 1.5, about 0.7 to about 1.0, about 0.8 to about 6.0, about 0.8 to about 5.5, about 0.8 to about 5.0, about 0.8 to about 4.5, about 0.8 to about 4.0, about 0.8 to about 3.5, about 0.8 to about 3.0, about 0.8 to about 2.5, about 0.8 to about 2.0, about 0.8 to about 1.5, about 0.8 to about 1.0, about 0.9 to about 6.0, about 0.9 to about 5.5, about 0.9 to about 5.0, about 0.9 to about 4.5, about 0.9 to about 4.0, about 0.9 to about 3.5, about 0.9 to about 3.0, about 0.9 to about 2.5, about 0.9 to about 2.0, about 0.9 to about 1.5, about 0.9 to about 1.0, about 1.0 to about 6.0, about 1.0 to about 5.5, about 1.0 to about 5.0, about 1.0 to about 4.5, about 1.0 to about 4.0, about 1.0 to about 3.5, about 1.0 to about 3.0, about 1.0 to about 2.5, about 1.0 to about 2.0, about 1.0 to about 1.5, about 1.5 to about 6.0, about 1.5 to about 5.5, about 1.5 to about 5.0, about 1.5 to about 4.5, about 1.5 to about 4.0, about 1.5 to about 3.5, about 1.5 to about 3.0, about 1.5 to about 2.5, or about 1.5 to about 2.0, embryo explants per square centimeter (cm²) of regeneration surface area, including all ranges and values derivable therebetween.

According to some embodiments, methods described herein comprise transferring monocot seed embryo explant(s) from a first regeneration medium in a first set of plate(s) or container(s) to a second regeneration medium in a second set of plate(s) or container(s). As described herein, transformation of male corn lines, and possibly other monocot germplasms, may often be difficult or less efficient as compared to female corn lines or other monocot germplasms. According to some embodiments, a second transfer during regeneration to a second regeneration medium and plate or container may improve transformation, shoot development and/or regeneration frequency of genetically modified plants of monocot or corn embryo explants, or certain monocot or corn lines that are more resistant to efficient transformation and/or regeneration of genetically modified plants, such as certain male germplasm corn lines. According to these embodiments, two regeneration transfers with fresh media and container(s) may reduce the potential loss of transformed tissue and plants, which may be due to reduced contamination and/or tissue necrosis as compared to culturing the monocot embryo explants in the same regeneration medium. According to some embodiments, the monocot embryo explants may be cultured in the first regeneration medium in a range of from about 1 week to about 4 weeks, or about 1 week to about 3 weeks, or for about 1 week, about 2 weeks, about 3 weeks, or about 4 weeks.

According to some embodiments, after a first transfer of explants from one or more extended bud induction plate(s) or container(s) to one or more first regeneration plate(s) or container(s) as described herein, a second transfer of explants, which may be green or greening explants and/or small shoots, from the first regeneration plate(s) or container(s) may be transferred to a one or more second regeneration plate(s) or container(s). The second regeneration container(s) may be, for example, one or more Vivi® tray(s) (Vivi®, The Netherlands) containing a regeneration medium for shoot development and rooting. Following the regeneration step, green plants can be selected and transferred to soil or plugs for further plant development. The selected green or greening plants may be putative transgenic plants and/or may have visible roots and/or no or little sign of chimerism. Because the monocot embryo explants can be selectively transferred in the second regeneration transfer, the explants may be transferred from a first regeneration medium/media and first set of regeneration plate(s) or container(s) to a second regeneration medium/media and second set of regeneration plate(s) or container(s), wherein the number of plate(s) or container(s) containing explants in the first set of regeneration plate(s) or container(s) is the same, higher or much higher than the number of plate(s) or container(s) in the second set of regeneration plate(s) or container(s), which may be a ratio of 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 5:4, 5:3, 5:2, 4:3, 3:2, 2:1, or 1:1, respectively.

According to some embodiments, the number of visible (or total) monocot seed embryo explants transferred or added to the second regeneration plate(s) or container(s) may be at a density of less than or equal to about 300 seed embryo explants per container, less than or equal to about 200 seed embryo explants per container, less than or equal to about 150 seed embryo explants per container, less than or equal to about 100 seed embryo explants per container, or less than or equal to about 75 seed embryo explants per container, or in a range of densities from about 10 to about 300 seed embryo explants per container, about 10 to about 250 seed embryo explants per container, about 10 to about 200 seed embryo explants per container, about 10 to about 150 seed embryo explants per container, about 10 to about 100 seed embryo explants per container, about 10 to about 75 seed embryo explants per container, about 10 to about 50 seed embryo explants per container, about 25 to about 300 seed embryo explants per container, about 25 to about 250 seed embryo explants per container, about 25 to about 200 seed embryo explants per container, about 25 to about 150 seed embryo explants per container, about 25 to about 100 seed embryo explants per container, about 25 to about 75 seed embryo explants per container, about 25 to about 50 seed embryo explants per container, about 50 to about 300 seed embryo explants per container, about 50 to about 250 seed embryo explants per container, about 50 to about 200 seed embryo explants per container, about 50 to about 150 seed embryo explants per container, about 50 to about 100 seed embryo explants per container, about 50 to about 75 seed embryo explants per container, or at about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 125, about 150, about 175, about 200, about 225, about 250, about 275, or about 300, including all ranges and values derivable therebetween. For these density values and ranges, the surface area of a ViVi® tray container may be approximately 68.25 square inches (in²) or 440.3 square centimeters (cm²). Thus, all of the above density values and ranges for seed embryo explants per container can be readily converted into density values and ranges of seed embryo explants per second regeneration surface area (for example, a density of 100 seed embryo explants per second regeneration container can be divided by the surface area per container to provide a density of seed embryo explants per second regeneration surface area of about 1.465 seed embryo explants/square inch (in²) or about 0.227 seed embryo explants/square centimeter (cm²), and similar conversions can be readily made for other density values and ranges). Density values and ranges of seed embryo explants per second regeneration surface area is a more universal definition for density of seed embryo explants in a variety of different regeneration plate(s) or container(s) that may each have different surface areas.

In some embodiments, the number of visible (or total) monocot seed embryo explants transferred or added to the second regeneration plate(s) or container(s) may be at a density of less than or equal to about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 embryo explants per square centimeter (cm²) of second regeneration surface area, including all ranges and values derivable therebetween. In certain embodiments, the number of visible (or total) monocot seed embryo explants transferred or added to the second regeneration plate(s) or container(s) may be at a density in a range from about 0.1 to about 6.0, about 0.1 to about 5.5, about 0.1 to about 5.0, about 0.1 to about 4.5, about 0.1 to about 4.0, about 0.1 to about 3.5, about 0.1 to about 3.0, about 0.1 to about 2.5, about 0.1 to about 2.0, about 0.1 to about 1.5, about 0.1 to about 1.0, about 0.1 to about 0.5, about 0.2 to about 6.0, about 0.2 to about 5.5, about 0.2 to about 5.0, about 0.2 to about 4.5, about 0.2 to about 4.0, about 0.2 to about 3.5, about 0.2 to about 3.0, about 0.2 to about 2.5, about 0.2 to about 2.0, about 0.2 to about 1.5, about 0.2 to about 1.0, about 0.2 to about 0.5, about 0.3 to about 6.0, about 0.3 to about 5.5, about 0.3 to about 5.0, about 0.3 to about 4.5, about 0.3 to about 4.0, about 0.3 to about 3.5, about 0.3 to about 3.0, about 0.3 to about 2.5, about 0.3 to about 2.0, about 0.3 to about 1.5, about 0.3 to about 1.0, about 0.3 to about 0.5, about 0.4 to about 6.0, about 0.4 to about 5.5, about 0.4 to about 5.0, about 0.4 to about 4.5, about 0.4 to about 4.0, about 0.4 to about 3.5, about 0.4 to about 3.0, about 0.4 to about 2.5, about 0.4 to about 2.0, about 0.4 to about 1.5, about 0.4 to about 1.0, about 0.4 to about 0.5, about 0.5 to about 6.0, about 0.5 to about 5.5, about 0.5 to about 5.0, about 0.5 to about 4.5, about 0.5 to about 4.0, about 0.5 to about 3.5, about 0.5 to about 3.0, about 0.5 to about 2.5, about 0.5 to about 2.0, about 0.5 to about 1.5, about 0.5 to about 1.0, about 0.6 to about 6.0, about 0.6 to about 5.5, about 0.6 to about 5.0, about 0.6 to about 4.5, about 0.6 to about 4.0, about 0.6 to about 3.5, about 0.6 to about 3.0, about 0.6 to about 2.5, about 0.6 to about 2.0, about 0.6 to about 1.5, about 0.6 to about 1.0, about 0.7 to about 6.0, about 0.7 to about 5.5, about 0.7 to about 5.0, about 0.7 to about 4.5, about 0.7 to about 4.0, about 0.7 to about 3.5, about 0.7 to about 3.0, about 0.7 to about 2.5, about 0.7 to about 2.0, about 0.7 to about 1.5, about 0.7 to about 1.0, about 0.8 to about 6.0, about 0.8 to about 5.5, about 0.8 to about 5.0, about 0.8 to about 4.5, about 0.8 to about 4.0, about 0.8 to about 3.5, about 0.8 to about 3.0, about 0.8 to about 2.5, about 0.8 to about 2.0, about 0.8 to about 1.5, about 0.8 to about 1.0, about 0.9 to about 6.0, about 0.9 to about 5.5, about 0.9 to about 5.0, about 0.9 to about 4.5, about 0.9 to about 4.0, about 0.9 to about 3.5, about 0.9 to about 3.0, about 0.9 to about 2.5, about 0.9 to about 2.0, about 0.9 to about 1.5, about 0.9 to about 1.0, about 1.0 to about 6.0, about 1.0 to about 5.5, about 1.0 to about 5.0, about 1.0 to about 4.5, about 1.0 to about 4.0, about 1.0 to about 3.5, about 1.0 to about 3.0, about 1.0 to about 2.5, about 1.0 to about 2.0, about 1.0 to about 1.5, about 1.5 to about 6.0, about 1.5 to about 5.5, about 1.5 to about 5.0, about 1.5 to about 4.5, about 1.5 to about 4.0, about 1.5 to about 3.5, about 1.5 to about 3.0, about 1.5 to about 2.5, or about 1.5 to about 2.0, embryo explants per square centimeter (cm²) of second regeneration surface area, including all ranges and values derivable therebetween.

According to many embodiments, the regeneration medium comprises a low salt concentration, which may be more particularly applicable to monocot embryo explants. As used herein, “low salt concentration” refers to a medium comprising total salt concentration that is less than or equal to about 2800 mg/L. As used herein, a “salt” has a commonly understood meaning in the field of chemistry and refers to an ionic chemical compound, or a dissolved chemical compound if present in a solution, comprising at least one cation (or base) and at least one anion (or acid). The regeneration medium may comprise, in some embodiments, a total salt concentration of less than or equal to about 3000 mg/L, about 2800 mg/L, about 2700 mg/L, about 2600 mg/L, about 2500 mg/L, about 2400 mg/L, about 2300 mg/L, about 2200 mg/L, about 2100 mg/L, or about 2000 mg/L. In further embodiments, the regeneration medium may comprise a salt concentration of about 1200 mg/L to about 3000 mg/L, about 1200 mg/L to about 2800 mg/L, about 1300 mg/L to about 2700 mg/L, about 1400 mg/L to about 2600 mg/L, about 1500 mg/L to about 2500 mg/L, about 1600 mg/L to about 2400 mg/L, about 1700 mg/L to about 2400 mg/L, about 1800 mg/L to about 2400 mg/L, about 1900 mg/L to about 2400 mg/L, about 2000 mg/L to about 2400 mg/L, about 2100 mg/L to about 2400 mg/L, about 2200 mg/L to about 2400 mg/L, or about 2300 mg/L, including all ranges derivable therebetween. The total nitrogen concentration of the regeneration medium may, in some embodiments, be in a range from about 0.5 mM to about 20 mM, about 0.5 mM to about 10 mM, about 1 mM to about 20 mM, about 5 mM to about 20 mM, about 1 mM to about 15 mM, about 5 mM to about 15 mM, about 1 mM to about 10 mM, about 1 mM to about 7.5 mM, about 2.5 mM to about 7.5 mM, about 5 mM to about 10 mM, about 10 mM to about 15 mM, about 10 mM to about 20 mM, or about 15 mM to about 20 mM, including all ranges derivable therebetween. As used herein, the term “total nitrogen concentration” refers to the total concentration of nitrogen containing ions, such as nitrate and ammonium ions.

A regeneration medium for use according to the methods described herein may be described, in some embodiments, in terms of its nitrate, ammonium, potassium, or sulfate ion concentration. The nitrate ion concentration in the regeneration medium, which may be more particularly applicable to monocot embryo explants, may be, for example, about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween. The ammonium ion concentration in the regeneration medium, which may be more particularly applicable to monocot embryo explants, may be, for example, about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The potassium ion concentration in the regeneration medium, which may be more particularly applicable to monocot embryo explants, may be, for example, about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The sulfate ion concentration in the regeneration medium, which may be more particularly applicable to monocot embryo explants, may be, for example, about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween.

The regeneration medium may, in some embodiments, be described by its ammonium nitrate, calcium chloride, calcium nitrate, or potassium sulfate concentration. The concentration of ammonium nitrate in the regeneration medium, which may be more particularly applicable to monocot embryo explants, may be, for example, in a range from about 100 mg/L to about 1000 mg/L, about 100 mg/L to about 750 mg/L, about 100 mg/L to about 500 mg/L, about 100 mg/L to about 250 mg/L, or about 250 mg/L to about 500 mg/L, including all ranges derivable therebetween. The concentration of calcium chloride in the regeneration medium, which may be more particularly applicable to monocot embryo explants, may be, for example, less than or equal to about 100 mg/L, greater than or equal to about 50 mg/L, about 50 mg/L to about 100 mg/L, or about 50 mg/L to about 75 mg/L, including all ranges derivable therebetween. The concentration of calcium nitrate in the regeneration medium, which may be more particularly applicable to monocot embryo explants may be, for example, less than or equal to about 500 mg/L, about 100 mg/L to about 500 mg/L, about 100 mg/L to about 300 mg/L, about 300 mg/L to about 400 mg/L, or about 100 mg/L to about 200 mg/L, including all ranges derivable therebetween. The concentration of potassium sulfate in the regeneration medium, which may be more particularly applicable to monocot embryo explants, may be, for example, greater than about 500 mg/L, about 500 mg/L to about 750 mg/L, about 500 mg/L to about 1000 mg/L, about 500 mg/L to about 1500 mg/L, about 500 mg/L to about 2000 mg/L, about 750 mg/L to about 1000 mg/L, or about 1000 mg/L, including all ranges derivable therebetween.

In particular embodiments, the regeneration medium may comprise one or more cytokinins. Non-limiting examples of cytokinins that may be used in the regeneration medium include 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin). The concentration of the cytokinin in the regeneration medium may be, in some embodiments, about 0.1 mg/L to about 50 mg/L, about 0.1 mg/L to about 45 mg/L, about 0.1 mg/L to about 40 mg/L, about 0.1 mg/L to about 35 mg/L, about 0.1 mg/L to about 30 mg/L, about 0.1 mg/L to about 25 mg/L, about 0.1 mg/L to about 20 mg/L, about 0.1 mg/L to about 15 mg/L, about 0.1 mg/L to about 10 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 5 mg/L, about 0.5 mg/L to about 4 mg/L, about 0.5 mg/L to about 3 mg/L, about 1 mg/L to about 5 mg/L, about 1 mg/L to about 4 mg/L, about 1 mg/L to about 3 mg/L, about 5 mg/L to about 40 mg/L, about 5 mg/L to about 30 mg/L, about 10 mg/L to about 30 mg/L, or about 20 mg/L to about 30 mg/L, or about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all values and ranges derivable therebetween. The inclusion of one or more cytokinins in the regeneration medium may, in particular embodiments, improve viability, regenerability and/or transformation/editing frequency following regeneration. In some embodiments, the inclusion of one or more cytokinins in the regeneration medium improves the viability, regenerability, and/or transformation/editing frequency of dicot embryo explants, such as soybean, cotton, or canola embryo explants, following regeneration.

In some embodiments, thidiazuron (TDZ) may be included in the regeneration medium at a concentration in a range from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.25 mg/L to about 1.75 mg/L, about 0.5 mg/L to about 1.5 mg/L, about 0.1 mg/L to about 5 mg/L, about 0.5 mg/L to about 4 mg/L, about 0.5 mg/L to about 3 mg/L, about 0.5 mg/L to about 2 mg/L, about 0.5 mg/L to about 1.5 mg/L, about 1 mg/L to about 5 mg/L, about 1 mg/L to about 4 mg/L, about 1 mg/L to about 3 mg/L, or about 0.1 mg/L, about 0.25 mg/L, about 0.5 mg/L, about 0.75 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween. The inclusion of thidiazuron (TDZ) in the regeneration medium may improve the viability, regenerability, and/or transformation/editing frequency following regeneration.

In some embodiments, a cytokinin other than thidiazuron (TDZ), such as 6-benzylaminopurine (BAP), kinetin, zeatin, diphenyl urea (DPU), 6-(gamma,gamma-dimethylallylamino)purine (2iP), and 6-(3-hydroxybenzylamino)purine (meta-topolin), may be included in the regeneration medium at a concentration adjusted and set according to its relative activity. In some embodiments, 6-benzylaminopurine (BAP), kinetin, zeatin, and meta-topolin may be present in the regeneration medium at a concentration of about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L about 0.1 mg/L to about 5 mg/L, about 0.1 mg/L to about 4 mg/L, about 0.1 mg/L to about 3 mg/L, about 0.1 mg/L to about 2 mg/L, about 1 mg/L to about 10 mg/L, about 2 mg/L to about 10 mg/L, about 2 mg/L to about 8 mg/L, about 2.5 mg/L to about 7.5 mg/L, about 3 mg/L to about 10 mg/L, about 4 mg/L to about 6 mg/L, about 1.0 mg/L, about 1.25 mg/L, about 1.5 mg/L, about 1.75 mg/L, about 2.0 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween, which may improve the viability, regenerability, and/or transformation/editing frequency of embryo explants, such as dicot embryo explants or soybean, cotton, or canola embryo explants, following regeneration.

In some embodiments, 6-(gamma,gamma-dimethylallylamino)purine (2iP) may be present in the regeneration medium at a concentration in a range from about 5 mg/L to about 50 mg/L, about 10 mg/L to about 50 mg/L, about 10 mg/L to about 40 mg/L, about 15 mg/L to about 40 mg/L, about 15 mg/L to about 35 mg/L about 15 mg/L to about 30 mg/L, about 20 mg/L to about 30 mg/L, about 22.5 mg/L to about 30 mg/L, about 22.5 mg/L to about 27.5 mg/L, or of about 5 mg/L, about 7.5 mg/L, about 10 mg/L, about 15 mg/L, about 20 mg/L, about 25 mg/L, about 30 mg/L, about 35 mg/L, about 40 mg/L, about 45 mg/L, or about 50 mg/L, including all ranges and values derivable therebetween, which may improve the viability, regenerability and/or transformation/editing frequency of embryo explants, such as dicot embryo explants or soybean, cotton or canola embryo explants, following regeneration.

In certain embodiments, embryo explants of the population are regenerated in contact with the regeneration medium at a temperature in a range from about 15° C. to about 40° C., about 20° C. to about 40° C., about 25° C. to about 40° C., about 30° C. to about 40° C., about 22° C. to about 38° C., about 34° C. to about 36° C., about 20° C. to about 38° C., about 20° C. to about 35° C., about 20° C. to about 32° C., about 20° C. to about 30° C., about 25° C. to about 40° C., about 25° C. to about 35° C., about 25° C. to about 32° C., about 25° C. to about 31° C., about 25° C. to about 30° C., 25° C. to about 29° C., about 27° C. to about 29° C., or about 27° C. to about 28° C., or at about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C., including all ranges and values derivable therebetween. In one embodiment, the embryo explants are monocot embryo explants and the embryo explants of the population are regenerated at about 20° C. to about 32° C., 25° C. to about 29° C., or about 27° C. to about 28° C., including all ranges and values derivable therebetween. In another embodiment, the embryo explants are dicot embryo explants and the embryo explants of the population are regenerated at a temperature in a range from about 15° C. to about 40° C., about 20° C. to about 40° C., about 25° C. to about 40° C., about 30° C. to about 40° C., about 32° C. to about 38° C., about 34° C. to about 36° C., about 20° C. to about 38° C., about 20° C. to about 35° C., about 20° C. to about 32° C., about 20° C. to about 30° C., about 25° C. to about 40° C., about 25° C. to about 35° C., about 25° C. to about 32° C., about 25° C. to about 31° C., about 25° C. to about 30° C., 25° C. to about 29° C., about 27° C. to about 29° C., or about 27° C. to about 28° C., including all ranges and values derivable therebetween. In still yet another embodiment, the embryo explants are cotton embryo explants, and the cotton embryo explants are regenerated in contact with the first regeneration medium at a temperature in a range from about 25° C. to about 40° C., about 30° C. to about 40° C., about 32° C. to about 38° C., or about 34° C. to about 36° C., including all ranges and values derivable therebetween.

The embryo explants may be regenerated, in some embodiments, in a regeneration medium (or in a first regeneration medium) for a time period in a range from about 5 days to about 70 days, about 10 days to about 70 days, about 14 days to about 70 days, about 14 days to about 60 days, about 14 days to about 50 days, about 20 days to about 50 days, about 28 days to about 42 days, about 30 days to about 40 days, about 32 days to about 38 days, about 20 days to about 40 days, about 20 days to about 36 days, about 22 days to about 34 days, about 24 days to about 32 days, about 21 days to about 63 days, about 28 days to about 56 days, about 35 days to about 49 days, about 20 days to about 70 days, about 30 days to about 70 days, about 35 days to about 65 days, about 40 days to about 60 days, or about 45 days to about 55 days, or for about 10 days, about 20 days, about 21 days, about 28 days, about 30 days, about 35 days, about 40 days, about 42 days, about 49 days, about 56 days, or about 63 days, or about 70 days, including all ranges and values derivable therebetween. In certain embodiments, the embryo explants are monocot embryo explants, and the monocot embryo explants are regenerated for about 20 days to about 50 days or about 28 days to about 42 days, including all ranges and values derivable therebetween. In some embodiments, the embryo explants are soybean embryo explants, and the explants are regenerated in contact with a regeneration medium (or a first regeneration medium) for about 5 days to about 70 days, about 10 days to about 70 days, about 14 days to about 70 days, about 20 days to about 70 days, about 30 days to about 70 days, about 35 days to about 65 days, about 40 days to about 60 days, or about 45 days to about 55 days, about 21 days to about 63 days, about 28 days to about 56 days, or about 42 days or about 49 days, including all ranges an values derivable therebetween. In some embodiments, the embryo explants are cotton embryo explants, and the cotton embryo explants are regenerated in contact with a regeneration medium (or a first regeneration medium) for about 14 days to about 70 days, about 14 days to about 50 days, about 20 days to about 50 days, about 28 days to about 42 days, about 30 days to about 40 days, about 32 days to about 38 days, about 20 days to about 40 days, about 20 days to about 36 days, about 22 days to about 34 days, about 24 days to about 32 days, about 21 days to about 63 days, about 28 days to about 56 days, about 35 days to about 49 days, or about 28 days or about 35 days, including all ranges and values derivable therebetween.

In some embodiments, the embryo explants are cotton embryo explants, and the cotton embryo explants are regenerated in contact with a regeneration medium (or a first regeneration medium) at a higher temperature for a first regeneration period before transferring the cotton embryo explants to a different environment at a lower temperature for a second regeneration period. According to these embodiments, the first regeneration period may be shorter (much shorter) than the second regeneration period. According to these embodiments, the higher temperature for regeneration of cotton embryo explants during the first regeneration period may be in a range from about 30° C. to about 40° C., about 32° C. to about 38° C., or about 34° C. to about 36° C., or at about 35° C., and/or the lower temperature for regeneration of cotton embryo explants during the second regeneration period may be in a range from about 20° C. to about 33° C., about 20° C. to about 30° C., about 23° C. to about 33° C., about 25° C. to about 31° C., or about 27° C. to about 29° C., or at about 28° C. According to these embodiments, the first regeneration period for regeneration of the cotton embryo explants may be in a range from about 1 hour to about 14 days, about 6 hours to about 14 days, about 1 day to about 14 days, about 1 day to about 12 days, about 1 day to about 10 days, about 1 day to about 8 days, about 1 day to about 6 days, about 1 day to about 5 days, about 1 day to about 4 days, or about 2 days to about 3 days, or for about 1 day, about 2 days, about 3 days, about 4 days, about 5 days or about 6 days, and/or the second regeneration period for regeneration of the cotton embryo explants may be in a range from about 1 week to about 8 weeks, about 2 weeks to about 7 weeks, about 2 weeks to about 6 weeks, about 3 weeks to about 7 weeks, or about 4 weeks to about 6 weeks, or for about 1 week, about 2 weeks, about 3 weeks, about 4 weeks, about 5 weeks, about 6 weeks, about 7 weeks or about 8 weeks. In some embodiments, the cotton embryo explants may be transferred from a first regeneration medium to a second regeneration medium, and the cotton embryo explants may be in contact with the second regeneration medium for an extended regeneration period. In certain embodiments, a selected portion of cotton embryo explants may be selected for transfer to the second and/or third regeneration mediums. The portion of cotton embryo explants selected for transfer to the second and/or third regeneration medium may, in some embodiments, be selected for transfer based on a desired phenotype. In particular embodiments, the selected portion of embryo explants may have green, symmetrical spade-shaped leaves and new growth. According to some embodiments, the second regeneration medium may be the same or different than the first regeneration medium and may be described as defined herein for a first regeneration medium (even if different than the first regeneration medium). According to some embodiments, the second regeneration medium may comprise a cytokinin and/or an auxin and/or may or may not comprise a selection agent.

In other embodiments, the embryo explants are canola embryo explants, and the canola embryo explants are regenerated in contact with a regeneration medium (or a first regeneration medium) for about 14 days to about 70 days, about 14 days to about 50 days, about 20 days to about 50 days, about 14 days to about 42 days, about 20 days to about 40 days, about 20 days to about 36 days, for about 21 days to about 36 days, about 22 days to about 34 days, about 24 days to about 32 days, about 2 weeks to about 7 weeks, about 2 weeks to about 6 weeks, about 3 weeks to about 5 weeks, or for about 28 days, including all ranges and values derivable therebetween. After the canola embryo explants are regenerated in contact with the regeneration medium (or the first regeneration medium), the canola embryo explants may be further developed or regenerated for shoot elongation by transferring the canola embryo explants or shoots to a shoot elongation medium (or a first shoot elongation medium), which may be present in a larger container, for a first elongation period, and the canola embryo explants or shoots may be transferred from a first shoot elongation medium to a second shoot elongation medium for a second elongation period for further development or regeneration and shoot elongation. The regeneration medium (or the first regeneration medium) for the canola embryo explants may comprise a cytokinin and/or an auxin. The first shoot elongation medium and/or the second shoot elongation medium for the canola embryo explants or shoots may not comprise a cytokinin and/or an auxin. The first shoot elongation medium and/or the second shoot elongation medium for the canola embryo explants or shoots may or may not comprise a selection agent. According to many of these embodiments, the canola embryo explants and/or shoots may be in contact with each of the first elongation medium and/or the second elongation medium at a temperature in a range from about 20° C. to about 33° C., about 20° C. to about 30° C., about 23° C. to about 33° C., about 25° C. to about 31° C., or about 27° C. to about 29° C., or at a temperature of about 28° C. According to many of these embodiments, the first elongation period and/or the second elongation period for further development or regeneration and shoot elongation of canola embryo explants or shoots may each be in a range from about 1 week to about 8 weeks, about 1 week to about 7 weeks, about 1 week to about 6 weeks, about 1 week to about 5 weeks, about 1 week to about 4 weeks, about 1 week to about 3 weeks, or about 2 weeks to about 3 weeks, or for about 1 week, about 2 weeks, about 3 weeks, about 4 weeks, about 5 weeks, about 6 weeks, about 7 weeks or about 8 weeks.

An elongation medium for embryo explants may comprise a variety of standard culture media or solution ingredients or components, such as for example, basal salts, macronutrients, micronutrients, sugars, antibiotics, selection agents, and/or vitamins. In some embodiments, an elongation medium, such as a first elongation medium and/or a second elongation medium, may not comprise a cytokinin or an auxin. In some embodiments, however, the elongation medium, such as a first elongation medium and/or a second elongation medium, may comprise a cytokinin and/or an auxin. In some embodiments, an elongation medium, such as a first elongation medium and/or a second elongation medium, may or may not comprise a selection agent.

In some embodiments, the embryo explants may be transferred from a first regeneration medium to a second regeneration medium and/or from a second regeneration medium to a third regeneration medium. In some embodiments, the embryo explants are dicot embryo explants, and the dicot embryo explants are transferred to a second regeneration medium following culture in contact with a first regeneration medium and/or transferred to a third regeneration medium following culture in contact with a second regeneration medium. The regeneration medium (or the first regeneration medium) and/or any second regeneration medium and/or third regeneration medium may be the same or different and/or may comprise a selection agent. According to some embodiments, the embryo explants may be in contact with a second regeneration medium and/or a third regeneration medium for a respective extended regeneration period, such as a respective first extended regeneration period and a second extended regeneration period, which may each be in a range from about 1 week to about 8 weeks, about 2 weeks to about 7 weeks, about 2 weeks to about 6 weeks, about 3 weeks to about 7 weeks, or about 4 weeks to about 6 weeks, or for about 1 week, about 2 weeks, about 3 weeks, about 4 weeks, about 5 weeks, about 6 weeks, about 7 weeks or about 8 weeks. A second regeneration medium and/or third regeneration medium, in certain embodiments, may not comprise any auxins. A second regeneration medium and/or third regeneration medium may comprise, in some embodiments, one or more auxins. The inclusion of an auxin in the second regeneration medium may, in certain embodiments, promote root formation. Non-limiting examples of auxins that may be included in a regeneration medium, such as a first regeneration medium, second regeneration medium, and/or a third regeneration medium, and/or an elongation medium, such as a first elongation medium and/or a second elongation medium, are 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). In some embodiments, a regeneration medium comprises indole-3-acetic acid (IAA). Non-limiting examples of cytokinins that may be included in a regeneration medium, such as a first regeneration medium, second regeneration medium, and/or a third regeneration medium, and/or an elongation medium, such as a first elongation medium and/or a second elongation medium, are 6-benzylaminopurine (BAP), thidiazuron (TDZ), kinetin, zeatin, diphenyl urea (DPU), 6-(gamma,gamma-dimethylallylamino)purine (2iP), and 6-(3-hydroxybenzylamino)purine (meta-topolin).

In some particular embodiments, an auxin may be present in a regeneration medium, such as a second regeneration medium and/or a third regeneration medium, and/or an elongation medium, such as a first elongation medium and/or a second elongation medium, at a concentration in a range from about 0.1 mg/L to about 15 mg/L, about 0.1 mg/L to about 12.5 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, or about 0.1 mg/L to about 1 mg/L, or at about 0.1 mg/L to about 0.5 mg/L, about 0.1 mg/L, about 0.2 mg/L, about 0.3 mg/L, about 0.4 mg/L, about 0.5, mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, or about 15 mg/L, including all ranges and values derivable therebetween.

In some embodiments, an auxin present in a regeneration medium and/or an elongation medium is 2,4-dichlorophenoxy-acetic acid (2,4-D), 4-amino-3,5,6-trichloro-picolinic acid (picloram), indole-3-acetic acid (IAA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), or 3,6-dichloro-2-methoxy-benzoic acid (dicamba) and/or at a concentration in a range from about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, or about 0.1 mg/L to about 1 mg/L, or at about 0.1 mg/L to about 0.5 mg/L, or of about 0.1 mg/L, about 0.2 mg/L, about 0.3 mg/L, about 0.4 mg/L, about 0.5, mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween. In some embodiments, the auxin present in a regeneration medium and/or an elongation medium is indole-3-butyric acid (IBA) and/or is present in the regeneration medium and/or an elongation medium at a concentration in a range from about 0.1 mg/L to about 5 mg/L, about 0.1 mg/L to about 4 mg/L, about 0.1 mg/L to about 3 mg/L, about 0.1 mg/L to about 2 mg/L, or about 0.1 mg/L to about 1 mg/L, or at about 0.1 mg/L to about 0.5 mg/L, or of about 0.1 mg/L, about 0.2 mg/L, about 0.3 mg/L, about 0.4 mg/L, about 0.5, mg/L, about 0.6 mg/L, about 0.7 mg/L, about 0.8 mg/L, about 0.9 mg/L, about 1 mg/L, about 2 mg/L, about 3 mg/L, about 4 mg/L, or about 5 mg/L, including all ranges and values derivable therebetween. In some embodiments, the auxin present in a regeneration medium and/or an elongation medium is phenylacetic acid (PAA) and/or is present in the regeneration medium and/or an elongation medium at a concentration in a range from about 0.1 mg/L to about 15 mg/L, about 0.1 mg/L to about 12.5 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, or about 0.1 mg/L to about 1 mg/L, or at about 0.1 mg/L to about 0.5 mg/L, or of about 0.1 mg/L, about 0.2 mg/L, about 0.3 mg/L, about 0.4 mg/L, about 0.5, mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, or about 10 mg/L, including all ranges and values derivable therebetween.

In some embodiments, one or more cytokinins may be present in any second regeneration medium, any third regeneration medium, and/or any elongation medium, such as a first elongation medium and/or a second elongation medium, each at a concentration or within a range of concentrations as described herein for a regeneration medium (or first regeneration medium). In some embodiments, one or more cytokinins may be present in any elongation medium, such as a first elongation medium and/or a second elongation medium, at a lower concentration than present in a regeneration medium. The concentration of a cytokinin in an elongation medium may be within a range from about 0.1 mg/L to about 50 mg/L, about 0.1 mg/L to about 45 mg/L, about 0.1 mg/L to about 40 mg/L, about 0.1 mg/L to about 35 mg/L, about 0.1 mg/L to about 30 mg/L, about 0.1 mg/L to about 25 mg/L, about 0.1 mg/L to about 20 mg/L, about 0.1 mg/L to about 15 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 1 mg/L to about 50 mg/L, about 1 mg/L to about 25 mg/L, about 1 mg/L to about 20 mg/L, about 1 mg/L to about 15 mg/L, about 1 mg/L to about 10 mg/L, about 1 mg/L to about 5 mg/L, about 1 mg/L to about 4 mg/L, about 1 mg/L to about 3 mg/L, about 1 mg/L to about 2 mg/L, about 2 mg/L to about 50 mg/L, about 5 mg/L to about 50 mg/L, about 5 mg/L to about 25 mg/L, about 10 mg/L to about 25 mg/L, or about 10 mg/L to about 20 mg/L, or at about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 12.5 mg/L, about 15 mg/L, about 20 mg/L, about 25 mg/L, about 30 mg/L, about 35 mg/L, about 40 mg/L, about 45 mg/L, or about 50 mg/L, including all ranges and values derivable therebetween.

In some embodiments, the cytokinin present in any elongation medium, such as a first elongation medium and/or a second elongation medium, is 6-benzylaminopurine (BAP), thidiazuron (TDZ), kinetin, zeatin, or 6-(3-hydroxybenzylamino)purine (meta-topolin) and is present in the elongation medium at a concentration within a range from about 0.1 mg/L to about 15 mg/L, about 0.1 mg/L to about 12.5 mg/L, about 0.1 mg/L to about 10 mg/L, about 0.1 mg/L to about 9 mg/L, about 0.1 mg/L to about 8 mg/L, about 0.1 mg/L to about 7 mg/L, about 0.1 mg/L to about 6 mg/L, about 0.1 mg/L to about 5 mg/L, about 0.1 mg/L to about 4 mg/L, about 0.1 mg/L to about 3 mg/L, about 0.1 mg/L to about 2 mg/L, about 0.1 mg/L to about 1 mg/L, about 0.5 mg/L to about 5 mg/L, about 1 mg/L to about 5 mg/L, about 1 mg/L to about 4 mg/L, or about 1 mg/L to about 3 mg/L, or at about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 12.5 mg/L, or about 15 mg/L, including all ranges and values derivable therebetween.

In some embodiments, the cytokinin present in any elongation medium, such as a first elongation medium and/or a second elongation medium, is 6-(gamma,gamma-dimethylallylamino)purine (2iP) and is present in the elongation medium at a concentration within a range from about 0.1 mg/L to about 50 mg/L, about 0.1 mg/L to about 45 mg/L, about 0.1 mg/L to about 40 mg/L, about 0.1 mg/L to about 35 mg/L, about 0.1 mg/L to about 30 mg/L, about 0.1 mg/L to about 25 mg/L, about 0.1 mg/L to about 20 mg/L, about 0.1 mg/L to about 15 mg/L, about 0.1 mg/L to about 10 mg/L, about 1 mg/L to about 50 mg/L, about 1 mg/L to about 25 mg/L, about 1 mg/L to about 20 mg/L, about 1 mg/L to about 15 mg/L, about 1 mg/L to about 10 mg/L, about 2 mg/L to about 50 mg/L, about 5 mg/L to about 50 mg/L, about 5 mg/L to about 25 mg/L, about 10 mg/L to about 25 mg/L, or about 10 mg/L to about 20 mg/L, or at about 10 mg/L, about 12.5 mg/L, about 15 mg/L, about 20 mg/L, about 25 mg/L, about 30 mg/L, about 35 mg/L, about 40 mg/L, about 45 mg/L, or about 50 mg/L, including all ranges and values derivable therebetween.

In specific embodiments, regenerating or growing a genetically modified plants or plant parts on regeneration medium comprising a low salt concentration may improve transformation and/or regeneration compared to genetically modified plants or plant parts regenerated on a regeneration medium comprising a higher salt concentration. In addition, regenerating or growing genetically modified plants or plant parts on medium comprising a low salt concentration may increase rooting frequency compared to genetically modified plants or plant parts regenerated on regeneration medium comprising higher salt concentration.

The regeneration and/or shoot elongation step may also be carried out under a variety of lighting conditions. Some degree of lighting may generally be used during regeneration step. According to some embodiments, the regeneration step may be carried out with an average or set light intensity of Photosynthetic Active Radiation (PAR) in a range from about 30 μE/m²·s to about 250 μE/m²·s, about 30 μE/m²·s to about 225 μE/m²·s, about 30 μE/m²·s to about 200 μE/m²·s, about 40 μE/m²·s to about 200 μE/m²·s, about 50 μE/m²·s to about 200 μE/m²·s, 50 μE/m²·s to about 180 μE/m²·s, 60 μE/m²·s to about 180 μE/m²·s, 70 μE/m²·s to about 180 μE/m²·s, 80 μE/m²·s to about 180 μE/m²·s, 90 μE/m²·s to about 180 μE/m²·s, 100 μE/m²·s to about 170 μE/m²·s, 110 μE/m²·s to about 160 μE/m²·s, or about 120 μE/m²·s to about 150 μE/m²·s. In further embodiments, the regeneration step may be carried out with an average or set light intensity of Photosynthetic Active Radiation (PAR) at about 20 μE/m²·s, about 30 μE/m²·s, about 40 μE/m²·s, about 50 μE/m²·s, about 60 μE/m²·s, about 70 μE/m²·s, about 80 μE/m²·s, about 90 μE/m²·s, about 100 μE/m²·s, about 110 μE/m²·s, about 120 μE/m²·s, about 130 μE/m²·s, about 140 μE/m²·s, about 150 μE/m²·s, about 160 μE/m²·s, about 170 μE/m²·s, about 180 μE/m²·s, about 190 μE/m²·s, about 200 μE/m²·s, about 210 μE/m²·s, about 220 μE/m²·s, about 230 μE/m²·s, about 240 μE/m²·s, or about 250 μE/m²·s. In specific embodiments, different amounts of light and dark cycles may be used during the regeneration step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the regeneration and/or shoot elongation step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

In certain embodiments, the embryo explants and the plurality of genetically modified plants or plant parts are cultured and regenerated without producing a callus tissue culture.

In specific embodiments, the plurality of genetically modified plants or plant parts are non-chimeric or have reduced chimerism. As used herein, the term “chimeric” or “chimerism” refer to a plant, plant tissue, explant, or the like, which is composed of two or more genetically different types of tissues or cells with respect to a genetic modification.

After any regeneration, shoot elongation and/or rooting step(s), the cultured or regenerated embryo explants, shoots, plantlets and/or plants, as the case may be, are transferred to a plug or soil for growth and development of the plant. Seeds may then be collected from regenerated plants and planted to produce progeny plants, which may then be crossed to itself or other plants or lines to produce another generation of progeny plants, and so on. According to embodiments of the present disclosure, cultured or regenerated embryo explants, shoots, plantlets, and/or plants, and any progeny thereof, may be genetically modified or selected for being genetically modified. Any medium described herein as containing a cytokinin and/or auxin may contain two or more cytokinins and/or two or more auxins, respectively, at the concentrations or within the concentration ranges described herein.

After regeneration, the cultured or regenerated embryo explants, shoots, plantlets and/or plants can be transferred to a plug or soil for growth and development of the plant. Seeds may then be collected from regenerated plants and planted to produce progeny plants, which may then be crossed to itself or other plants or lines to produce another generation of progeny plants, and so on. According to embodiments of the present disclosure, cultured or regenerated embryo explants, shoots, plantlets and/or plants, and any progeny thereof, may be genetically modified or selected for being genetically modified. Any medium described herein as containing a cytokinin and/or auxin may contain two or more cytokinins and/or two or more auxins, respectively, at the concentrations or within the concentration ranges described herein.

### F. Genotype Identification

In another aspect of the present disclosure, a genotype of an explant, cultured explant, or genetically modified plant or plant part is identified. As used herein, the term “genotype” refers to the particular genetic constitution or identity of an explant, cultured explant, plant or plant part, or when used in reference to a specific genomic locus or gene, the particular genetic constitution of the specific genomic locus or gene (e.g., wild-type, mutant, etc.) of the explant, cultured explant, plant or plant part. In some embodiments, a genotype of at least one of the explants of the population described herein is identified. In further embodiments, a genotype of a plurality of embryo explants, cultured explants, or genetically modified plants or plant parts is identified. The genotype of one or more embryo explants, cultured explants, or genetically modified plants or plant parts may be identified, in specific embodiments, at any step during the methods provided by the present disclosure. The genotype may be identified, for example, prior to and/or following explant preparation or isolation, explant culture, Rhizobiales inoculation, transformation or editing, bud induction (or first bud induction), second bud induction (or extended bud induction), shoot generation, and/or regeneration as provided by the present disclosure. A genotype may be identified, for example, by identifying one or more genetic markers, each of which comprises a polynucleotide sequence that is, either alone or in combination, characteristic of the genotype. Non-limiting of the types of genetic markers which may be used according to the present disclosure include single nucleotide polymorphisms (SNPs), mutations, sequence differences or variations, and inserted and/or deleted sequences (indels). As used herein, the phrase “characteristic of a genotype” refers to a genetic marker which is associated with a particular genotype. A genotype may be “associated with” or “correlated with” a culturing characteristic or a phenotype, for example, when the genotype has an effect on the culturing characteristic or phenotype. The association or correlation between a genotype and a culturing characteristic or a phenotype may be shown using any method known in the art. In specific embodiments, an association or correlation between a genotype and a culturing characteristic may be shown by genotyping, phenotypic observation, and/or statistical analysis. A genetic marker which is characteristic of a genotype is associated with, present within, or indicative of the particular genotype, but may or may not also be characteristic of one or more other genotypes. In some embodiments, a genetic marker may be “exclusively characteristic” of a genotype, if the genetic marker is characteristic of only the one genotype (either among all known genotypes characterized for the marker or at least among the population of explants, plants or plant parts that are or were being genetically modified in a given treatment or experiment). As used herein, the term “germplasm” refers to a population of plants, plant parts, seeds, and/or explants, wherein the individual members of the population have the same or substantially the same genotype or genetic background. A germplasm comprises identifiable genomic features that are correlated with true breeding characteristics. Germplasm includes varieties of plants that have one or more distinct features or combinations of features for example features that relate to growth rates, morphology, genetics and the like. One of ordinary skill in the art will appreciate that U.S. Pat. Nos. 9,485,940 and 11,206,791 describe exemplify germplasm descriptions for distinct corn and soybean germplasm, respectively (each patent is herein incorporated by reference in its entirety). A germplasm may be identified at the genomic level and distinguished from other germplasms included in the same bulk transformation protocol. Plant germplasms may be useful, for example, in crop breeding, research, conservation, and/or product development and are maintained for these purposes. The term “genetic background” as used herein refers to the genotype of a plant, germplasm, etc., which may be with or without, or apart from, one or more genetic modifications. In specific embodiments, the individual members of a plant germplasm, may have a genetic background or genotype which is at least about 99.99%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90% identical. In specific embodiments, the individual members of a plant germplasm, may have a genetic background or genotype which is at least 99.99%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, or 90% identical.

Compositions as disclosed herein can include at least 2, 3, 4, 5, 10, 15, or 20 distinct germplasm and at least 1, 2, 3, 4, 5, 10, 15, or 20 nucleic acid sequences for transforming, transiently or stably, the germplasm. One of ordinary skill in the art will appreciate that the mixture and therefore, the compositions described herein can be described as a laboratory sample comprising tissue from plants that represent at least two germplasms, and at least one recombinant nucleic acid sequence. Additionally, compositions are also provided that include a mixture of at least two germplasms wherein the at least two germplasms are transformed with at least one recombinant nucleic acid sequence. One of ordinary skill in the art will appreciate that the transformed germplasm can be a plant cell, if protoplast is used as the source of germplasm, plant tissue, if explants are used as the source of germplasm, leaf tissue, if mature plant tissues are used and the like. In another embodiment, compositions described herein include trays, greenhouses, and fields comprising a mixture of plants that are characterized by having at least 2, 3, 4, 5, 10, 15, or 20 distinct germplasm and at least 1, 2, 3, 4, 5, 10, 15, or 20 nucleic acid sequences. Such compositions are useful for, among other things, identifying optimum combinations of germplasm and nucleic acid sequences. Moreover, such compositions are useful for identifying optimum combinations of germplasm and nucleic acid sequences based upon growth, storage, handling, weather tolerance, and other factors useful for producing robust agricultural products.

One of ordinary skill in the art will appreciate that the description provided herein can also be used to identify growth, storage and handling conditions that are important for creating superior plant tissue for use in transformation. A variety of bar codes, such as single nucleotide polymorphisms or other small unique DNA sequences can be introduced into a single germplasm variety, thus creating a population of distinct germplasms. Each of those distinct germplasms can be differentially treated, for example, each of the distinct germplasm can be grown, stored, handled, and processed using a variety of conditions. The population of germplasm can subsequently be placed in a composition that also includes a heterologous nucleic acid sequence and the resulting transformants can then be characterized.

In some embodiments, the present disclosure provides compositions and methods for collective genetic transformation or modification of a population of embryo explants, wherein the population of embryo explants includes variants of the same plant genotype or germplasm, or two or more genotypes or germplasms. Variants of the same plant genotype or germplasm may comprise, for example, substantially the same genotype or genetic background but may further comprise at least one genotypic difference or genetic modification. In certain embodiments, variants of the same plant genotype or germplasm may comprise substantially the same genotype or genetic background and may further comprise about 1 to about 20, about 1 to about 15, about 1 to about 10, about 1 to about 5, about 1, about 2, about 3, about 4, about 5, about 6, about 7, about 8, about 9, about 10, about 11, about 12, about 13, about 14, about 15, about 16, about 17, about 18, about 19, or about 20 genotypic differences. In particular embodiments, variants of the same plant genotype or germplasm may comprise, for example, at least about 10, about 15, about 20, about 25, about 50, about 100, or about 150 genotypic identities and may further comprise about 1 to about 20, about 1 to about 15, about 1 to about 10, about 1 to about 5, about 1, about 2, about 3, about 4, about 5, about 6, about 7, about 8, about 9, about 10, about 11, about 12, about 13, about 14, about 15, about 16, about 17, about 18, about 19, or about 20 genotypic differences. These genotypic identities and differences may be detected, for example, using genetic markers as described herein. Variant plant genotypes or germplasms, in some embodiments, may have the same genotype at least at about 10, about 15, about 25, about 50, about 100, or about 150 genomic loci and may have a different genotype at about 1 to about 20, about 1 to about 15, about 1 to about 10, about 1 to about 5, about 1, about 2, about 3, about 4, about 5, about 6, about 7, about 8, about 9, about 10, about 11, about 12, about 13, about 14, about 15, about 16, about 17, about 18, about 19, or about 20 genomic loci. In some embodiments, variants of the same plant genotype or germplasm may have genotypes or genetic backgrounds which are at least about 99.99%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, or about 90% identical. In particular embodiments, variants of the same plant genotype or germplasm may have genotypes or genetic backgrounds which are at least 99.99%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, or 90% identical. The genotypic differences between variants of the same plant germplasm may be identified for example, by identifying one or more genetic markers, as provided by the present disclosure.

The present disclosure further provides, in particular embodiments, methods for collective genetic transformation or modification comprising: growing one or more germplasm(s) and/or one or more variant(s) of the same germplasm at different geographical locations and/or under different growing conditions; preparing a population of embryo explants from the one or more germplasm(s) and/or one or more variant(s) of the same germplasm grown at different locations and/or under different growing conditions; and collectively introducing a heterologous polynucleotide molecule, a ribonucleoprotein, and/or a site-specific nuclease into one or more explants of the population.

The selection of genetic markers for identifying the genotype of one or more germplasm(s) or germplasm variant(s) present in a population of embryo explants, cultured explants, and/or plant or plant parts may include, in some embodiments, genetic fingerprinting of the individual germplasms of the population. Methods of genetic fingerprinting are known in the art. The data obtained from fingerprinting may then be used to select genetic markers, which when used alone and/or in combination, are able to identify the individual germplasms or germplasm variants. Genetic markers for use according to the present embodiments may, in certain embodiments, have a distinct genotype for a subset of the germplasms of the population; have a high minor (rare) allele frequency within the population and the crop germplasm at large; produce some redundancy in identification when used in combination; and/or are not cost prohibitive when used in combination with the total number of genetic markers in the assay.

Genetic markers, which have a distinct genotype or sequence for a subset of germplasms or for a subset of germplasm variants of the population, allow any germplasm or variant thereof lacking the genotype or sequence at the genomic locus of the genetic marker to be eliminated from consideration for identification. If the population comprises inbred lines, the chosen genotypes are preferably homozygous. Homozygosity at a genetic marker locus indicates that all individual explants from a single germplasm in the population have the same genotype. This allows for any candidate germplasm lacking the genotype to be unambiguously eliminated from consideration for identification of the explant. Heterozygosity at a genetic marker locus is acceptable when the germplasm is an F₁ hybrid, as all individuals from this F₁ hybrid germplasm carry the same heterozygous genotype. This is a distinguishing feature of the hybrid germplasm in the population. If the population comprises segregating populations, a larger number of genetic markers may be required for identification. In an F2 segregating population, individuals of a single germplasm may have different genotypes at a given genetic marker locus due to segregation, therefore that genetic marker locus cannot exclude membership to the germplasm. To identify the origin (original parental cross) of a segregating population, genetic markers are chosen which are “fixed” for a specific parent or origin. Therefore, the presence of an alternate genotype at that genetic marker can eliminate candidate origin. Additional genetic markers are required to determine which segregant population each progeny belongs to within an origin. Thus, a larger number of genetic markers is required to derive both origin and genotypic segregant within the origin of a segregating population.

Genetic markers, which have a high minor allele frequency both among the germplasms of the population and the crop germplasm at large, allow any germplasm not comprising the minor allele at that genetic marker locus to be eliminated from consideration for identification. As used herein, “minor allele frequency” refers to the allelic frequency of the rarest allele (for a biallelic marker) in a population. For a biallelic marker, the frequency of the major allele (most common allele)+the frequency of the minor allele (rare allele)=1, and therefore the presence of the minor allele at a genetic marker locus can be used to eliminate all carriers of the major allele from consideration for identification. If the minor allele frequency is high among the germplasms of the population, such that the minor allele is present in several lines, any genotype at that genetic marker locus (major or minor) can be used to eliminate several candidate germplasms from consideration for identification at once. If the minor allele frequency is high among the crop germplasms at large, the genetic marker may be useful for detecting external contamination.

Genetic markers, which in combination with other markers that have some level of redundancy, allow for the unambiguous identification of each germplasm in the population, even in the presence of a low to moderate amount of missing data during genotyping. It is possible that not every genetic marker locus produces quality data during genotyping, which results in “missing data.” Missing data may occur due to low sample quality, a low amount of DNA or RNA in a sample, reaction failure, low reliability of a particular marker assay, reagent batches, and/or ambiguous reads. Building redundancy into the genetic marker combination pool allows for identification of a germplasm in a population, even if data is missing, through the combination of the remaining genetic markers. The amount of redundancy to build into a particular assay depends on the expected rate of missing data.

The selection of genetic markers which are not cost prohibitive, when used in combination with the total number of genetic markers in the assay, ensures that bulk transformation or editing is a preferred compared to single germplasm transformation or editing.

The combination of genetic markers utilized for deconvolution and identification of individual germplasms may be chosen, for example, according to a few main criteria, such as (1) markers that have high informativeness, specifically those that have a high minor (rare) allele frequency within the bulk, such that the presence of a minor allele in the population is present at a level high enough to allow for elimination of several germplasms from consideration for identification; (2) markers with consistent high call rate (low missing data) and high data quality; and (3) markers, which in combination, produce some redundancy in identification, such that if a sample lacks data for some marker loci, robust identification is possible based on unambiguous data from the total combination of genetic markers, or as otherwise provided herein. In addition, markers may be chosen to cover a wide range of SNPs on different chromosomes to increase coverage of the genome.

Minor allele frequency refers to the allelic frequency of the rarest allele (for a biallelic marker) in a population. For a biallelic marker, the frequency of the major allele (most common allele)+the frequency of the minor allele (rare allele)=1. In most cases, high minor allele frequency for genetic marker selection is >30%. The presence of the minor allele at a genetic marker locus can be used to eliminate all carriers of the major allele. It is beneficial for minor allele frequency to be high among the bulk germplasms. When the minor allele is present in several lines, presence of either allele at that genetic marker locus (major or minor) can be used to eliminate several potential germplasms from consideration for identification at once.

The second criterion above suggests selection of markers of consistently high quality, such as those with high call rate during sequencing. Consistent, high call rates during sequencing indicates a low amount of missing data in the resulting dataset. Missing data (calls or reads) can decrease the ability to deconvolute germplasm in bulk and decrease the confidence of identification of lines with higher levels of missing data. While missing data may occur due to multiple variables, choosing high quality markers with good call rates can help decrease the likelihood of low reliability markers contributing to missing data.

The third criterion above suggests selection of genetic markers, which in combination with other genetic markers, have some level of redundancy. This allows for unambiguous identification of the germplasm, even in the presence of a low to moderate amount of missing data during genotyping. During deconvolution, it is possible that not every genetic marker locus produces quality data, resulting in “missing data.” Missing data may be due to low sample quality, a low amount of DNA in a sample, reaction failure, low reliability of a particular marker assay, reagent batches, and/or ambiguous reads. Building redundancy into the genetic marker combination pool can allow for identification of a germplasm even if data is missing through the combination of the remaining genetic markers. The amount of redundancy to build into an assay depends on the expected rate of missing data.

In some embodiments, the present disclosure provides methods of identifying a genotype of one or more explant(s), cultured explant(s), and/or genetically modified plant(s) or plant part(s). In further embodiments, identifying the genotype comprises detecting at least 1, at least 2, at least 3, at least 4, at least 5, at least 6, at least 7, at least 8, at least 9, at least 10, at least 15, at least 20, or at least 25 genetic markers in one or more sample(s) derived from at least 1, 2, 3, 4, 5, 6, 7, 8, 9, 10, 15, 20, 25, 30, 35, 40, 50, 75, 100, 150, 200, 250, 300, 350, 400, 450, 500, 550, 600, 650, 700, 750, 800, 850, 900, 950, 1000, 1500, 2000, 2500, 3000, 3500, 4000, 4500, or 5000 explant(s), cultured explant(s), and/or modified plant(s) or plant part(s), wherein each of the genetic markers comprises a polynucleotide sequence that is characteristic of the genotype. The sample used to detect the genetic marker(s), in some embodiments, may comprise a nucleic acid or polynucleotide molecule from or derived from one or more explant(s), cultured explant(s), and/or genetically modified plant(s) or plant part(s). The nucleic acid or polynucleotide molecule, in certain embodiments, may be a genomic DNA molecule or a fragment thereof or a mRNA molecule or a fragment thereof or may be derived from or amplified from a genomic DNA molecule or a fragment thereof or a mRNA molecule or a fragment thereof. Any method known in the art may be used to isolate, amplify, or produce a genomic DNA molecule or a fragment thereof or a mRNA molecule or a fragment thereof for use in the methods provided by the present disclosure. Methods of amplification, such as polymerase chain reaction (PCR), reverse transcription PCR (RT-PCR), etc., are known in the art. In particular embodiments, the genotype of the one or more explant(s), cultured explant(s), and/or genetically modified plant(s) or plant part(s) is identified before or after the heterologous polynucleotide molecule is introduced into the at least two embryo explants of the population; before or after co-culturing the at least two embryo explants of the population; before or after culturing the at least two embryo explants of the population in contact with the first bud induction medium; before or after culturing the at least two embryo explants of the population in contact with the second bud induction medium; or before or after regenerating or growing the plurality of genetically modified plants or plant parts from the at least two embryo explants of the population or any progeny generation of a cell thereof. In specific embodiments, each of the genetic markers comprises a polynucleotide sequence that is exclusively characteristic of the genotype. In particular embodiments, identifying the genotype comprises performing genetic sequencing on one or more sample(s) from or derived from at least 1, 2, 3, 4, 5, 6, 7, 8, 9, 10, 15, 20, 25, 30, 35, 40, 50, 75, 100, 150, 200, 250, 300, 350, 400, 450, 500, 550, 600, 650, 700, 750, 800, 850, 900, 950, 1000, 1500, 2000, 2500, 3000, 3500, 4000, 4500, or 5000 explant(s), cultured explant(s), and/or modified plant(s) or plant part(s), and detecting at least 1, at least 2, at least 3, at least 4, at least 5, at least 6, at least 7, at least 8, at least 9, at least 10, at least 15, at least 20, or at least 25 genetic markers in the sample, wherein each of the genetic markers comprises a polynucleotide sequence that is characteristic of the genotype. In certain embodiments, identifying the genotype comprises contacting one or more sample(s) from or derived from at least 1, 2, 3, 4, 5, 6, 7, 8, 9, 10, 15, 20, 25, 30, 35, 40, 50, 75, 100, 150, 200, 250, 300, 350, 400, 450, 500, 550, 600, 650, 700, 750, 800, 850, 900, 950, 1000, 1500, 2000, 2500, 3000, 3500, 4000, 4500, or 5000 explant(s), cultured explant(s), and/or modified plant(s) or plant part(s) with at least 1, at least 2, at least 3, at least 4, at least 5, at least 6, at least 7, at least 8, at least 9, at least 10, at least 15, or at least 20 polynucleotide or DNA probe(s), wherein each of the polynucleotide or DNA probe(s) is specific for one genetic marker; subjecting the sample and the polynucleotide or DNA probe(s) to stringent hybridization conditions; and detecting the hybridization of the polynucleotide or DNA probe(s) to at least 1, at least 2, at least 3, at least 4, at least 5, at least 6, at least 7, at least 8, at least 9, at least 10, at least 15, or at least 20 genetic markers in the sample. Polynucleotide or DNA probes according to the present disclosure may have complete sequence identity with the target sequence, although probes differing from the target sequence that retain the ability to hybridize preferentially to target sequence may be designed by conventional methods. In order for a polynucleotide or nucleic acid molecule to serve as probe it need only be sufficiently complementary in sequence to be able to form a stable double-stranded structure under the particular solvent and salt concentrations employed. Any conventional nucleic acid hybridization or amplification method can be used to identify the presence of a genetic marker in a sample. Probes are generally at least about 11 nucleotides, at least about 18 nucleotides, at least about 24 nucleotides, or at least about 30 nucleotides or more in length. Such probes hybridize specifically to a target polynucleotide sequence under stringent hybridization conditions. Stringent hybridization conditions are known in the art and described in, for example, M R Green and J Sambrook, Molecular cloning: a laboratory manual, 4^{th} Edition, Cold Spring Harbor Laboratory Press, Cold Spring Harbor, N.Y. (2012), incorporated herein by reference. As used herein, two polynucleotide or nucleic acid molecules are capable of specifically hybridizing to one another if the two molecules are capable of forming an anti-parallel, double-stranded nucleic acid structure. A polynucleotide or nucleic acid molecule is the “complement” of another polynucleotide or nucleic acid molecule if they exhibit complete complementarity. As used herein, two polynucleotide or nucleic acid molecules exhibit “complete complementarity” if when aligned every nucleotide of the first molecule is complementary to every nucleotide of the second molecule. Two polynucleotide or nucleic acid molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

In some embodiments, identifying the genotype comprises identifying a genotype of one or more sample(s) derived from a plurality of embryo explants, cultured explants, genetically modified plants or plant parts, which may comprise detecting the presence or absence of a genetic marker in one or more sample(s) derived from at least two embryo explants, cultured explants, and/or genetically modified plants or plant parts. In further embodiments, identifying the genotype comprises identifying a plurality of genotypes in one or more sample(s) derived from a plurality of embryo explants, cultured explants, genetically modified plants or plant parts. Identifying the genotype, in some embodiments, comprises detecting at least two genetic markers in one or more sample(s) derived from at least two embryo explants, cultured explants, and/or genetically modified plants or plant parts. In further embodiments, the at least two genetic markers comprise a first genetic marker and a second genetic marker, wherein the first genetic marker comprises a first polynucleotide sequence that is characteristic of a first genotype and the second genetic marker comprises a second polynucleotide sequence that is characteristic of a second genotype. In specific embodiments, the first polynucleotide sequence is exclusively characteristic of the first genotype or the second polynucleotide sequence is exclusively characteristic of the second genotype. Identifying a genotype, in particular embodiments, may comprise detecting at least 3, at least 4, at least 5, at least 6, at least 7, at least 8, at least 9, at least 10, at least 15, at least 20, or at least 25 genetic markers in one or more sample(s) derived from at least two embryo explants, cultured explants, or genetically modified plants or plant parts, wherein at least two of the genetic markers comprise a polynucleotide sequence that is characteristic of the first genotype and at least one of the genetic markers comprises a polynucleotide sequence that is characteristic of the second genotype.

In further embodiments, the present disclosure provides methods of identifying a genotype of one or more explant(s), cultured explant(s), and/or genetically modified plant(s) or plant part(s) in a population and associating the genotype with at least one culturing characteristic or phenotype. In some embodiments, the methods of the present disclosure may further comprise identifying a genetic marker or a quantitative trait locus (QTL) associated with the at least one culturing characteristic or phenotype. As used herein, the term “phenotypic characteristic” refers to observable characteristics resulting from the interaction of a genotype with its environment. Non-limiting examples of types of phenotypic characteristics that may be observed according to the present disclosure include culturing characteristics and plant or plant part phenotypes. As used herein, the term “culturing characteristic” refers to an observable characteristic associated with culturing explants, plant parts, and plants. Non-limiting examples of culturing characteristics which may be observed and/or assayed according to the present disclosure include an increase or improvement in, or alternatively, a decrease in explant excision efficiency, regeneration efficiency, genetic modification or transformation efficiency, shoot generation, the ability to regenerate into a genetically modified plant or plant part, and expression of a phenotype conferred by the heterologous polynucleotide molecule. As used herein, a “plant or plant part phenotype” refers to an observable plant trait or plant part trait. Non-limiting examples of plant phenotypes which may be observed and/or assayed include plant height, ear height, brace root color, internode direction, internode length, leaf color, leaf length, leaf width, leaf sheath pubescence, leaf marginal waves, tassel length, anther color, glume color, silk color, silk position, husk opening, husk color, cob diameter, kernel row number, kernel number per row, endosperm type, endosperm color, relative maturity, flower color, hilum color, seed coat color, seed shape, leaf shape, growth habit, and any phenotype conferred by a selectable marker gene, a scoreable marker gene, a screenable marker gene, a gene of interest, a guide RNA molecule, or a site-specific nuclease.

In particular embodiments, the present disclosure provides a method of genetically modifying a population of plant embryo explants, the method comprising: collectively introducing a heterologous polynucleotide molecule into at least two embryo explants of the population, wherein the population comprises embryo explants of at least two different plant genotypes, wherein the embryo explants of the at least two different genotypes comprise embryo explants of a first genotype and embryo explants of a second genotype, and wherein the embryo explants of the first genotype and the embryo explants of the second genotype are present in the population at a random or predetermined ratio. In certain embodiments, the population of embryo explants may be excised from a population of plant seeds, wherein the population of plant seeds comprises plant seeds of at least two different genotypes, wherein the at least two different genotypes comprise plant seeds of a first genotype and plant seeds of a second genotype, and wherein the plant seeds of the first genotype and the plant seeds of the second genotype are present in the population at a random or predetermined ratio. As used herein, the term “predetermined ratio” refers to the ratio of the number of embryo explants or plant seeds of one genotype present in the population compared to the number of embryo explants or plant seeds of another genotype. In some embodiments, the predetermined ratio of embryo explants or plant seeds of different genotypes in a population may reflect their relative proportion in the population. In certain embodiments, the population of embryo explants or plant seeds may comprise at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes, and explants or seeds of each different plant genotype may be present in the population at an equal, random or predetermined ratio. In further embodiments, the predetermined ratio is determined based upon at least one culturing characteristic or phenotype associated with a genotype present in the population. The predetermined ratio of the embryo explants or plant seeds of different genotypes in the population, in certain embodiments, results in an approximately equivalent number of regenerated genetically modified plants or plant parts of each genotype. As used herein, “approximately equivalent” refers to a condition where the number of regenerated genetically modified plants or plant parts of each genotype does not deviate more than about 20% from the average number±standard deviation of regenerated genetically modified plants or plant parts produced per genotype for the population as a whole. In specific embodiments, the embryo explants or plant seeds of a first genotype and embryo explants or plant seeds of a second genotype may be present in the population at a predetermined ratio of about 1:1 to about 20:1, about 1:1 to about 15:1, about 1:1 to about 10:1; about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1.8:1, about 1:1 to about 1.6:1, about 1:1 to about 1.4:1, or about 1:1 to about 1.2:1, including all ranges derivable therebetween. For example, if explants or seeds of a particular genotype are discovered or known to comprise a culturing characteristic conferring increased or improved explant excision efficiency, regeneration efficiency, genetic modification or transformation efficiency, ability to regenerate into a genetically modified plant or plant part, and/or expression of a phenotype conferred by the heterologous polynucleotide molecule, explants or seeds of this genotype may be initially included in the population at a lower predetermined ratio. In contrast, if explants or seeds of a particular genotype are discovered or known to comprise a culturing characteristic conferring decreased explant excision efficiency, regeneration efficiency, genetic modification or transformation efficiency, ability to regenerate into a genetically modified plant or plant part, and/or expression of a phenotype conferred by the heterologous polynucleotide molecule, explants or seeds of this genotype may initially be included in the population at a higher predetermined ratio.

In certain embodiments, the present disclosure provides a method of selecting a genetically modified plant produced by the embodiments described herein, wherein the genetically modified plant comprises at least one genetic modification; and crossing the genetically modified plant with itself or another plant to obtain a progeny plant or seed, wherein the other plant has the same or a different genotype as the genetically modified plant. In some embodiments, selecting the genetically modified plant comprises identifying a genotype of the genetically modified plant and selecting the genetically modified plant comprising the genotype. The genotype of the genetically modified plant may be identified according to any of the embodiments of the present disclosure and/or any methods of identifying a genotype known in the art.

In some embodiments, the present disclosure provides a method of genetically modifying a population of plant embryo explants, the method comprising: collectively introducing a heterologous polynucleotide molecule into at least two embryo explants of the population, wherein the population comprises embryo explants of at least two different plant genotypes; observing a first culturing characteristic or phenotype of a first genotype; and observing a second culturing characteristic or phenotype of a second genotype. In further embodiments, the first culturing characteristic or phenotype and the second culturing characteristic or phenotype are the same. In other embodiments, the first culturing characteristic or phenotype and the second culturing characteristic or phenotype are different. In some embodiments, the first genotype and the second genotype are evaluated by comparing the first culturing characteristic or phenotype and the second culturing characteristic or phenotype. In specific embodiments, the population may comprise at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes, and at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, or at least 500 different plant genotypes, or from 2 to about 750, 2 to about 600, about 10 to about 500, about 15 to about 400, about 20 to about 300, about 25 to about 200, about 10 to about 150, about 10 to about 100, about 10 to about 90, about 10 to about 80, about 10 to about 70, about 10 to about 60, about 10 to about 50, about 10 to about 40, about 10 to about 30, about 10 to about 20, 2 to about 50, 2 to about 40, 2 to about 30, 2 to about 20, 2 to about 15, or 2 to about 10 different plant genotypes may be evaluated according to the embodiments provided in the present disclosure. In further embodiments, at least 1, at least 2, at least 3, at least 4, at least 5, at least 6, at least 7, at least 8, at least 9, or at least 10 different culturing characteristics or phenotypes may be observed or evaluated according to the embodiments provided by the present disclosure.

### G. Selection of Genetically Modified Explants, Plants, and Plant Parts

In another aspect, the present disclosure provides methods of selecting genetically modified explants, plants, and plant parts. In some embodiments, the selecting comprises: identifying a genetic modification present in one or more embryo explants of the population or one or more genetically modified plants or plant parts; and selecting one or more embryo explants of the population or one or more genetically modified plants or plant parts comprising the genetic modification. In further embodiments, the selecting may further comprise identifying at least genetic marker which is characteristic of an identified genotype. The selected embryo explant(s) or the selected genetically modified plant(s) or plant part(s), in some embodiments, may comprise the at least one genetic marker which is characteristic of the identified genotype. In alternative embodiments, the selected embryo explant(s) or the selected genetically modified plant(s) or plant part(s) do not further comprise the at least one genetic marker characteristic of the identified genotype. In certain embodiments, the present disclosure further provides methods comprising: regenerating or growing regenerated genetically modified plant(s) or plant part(s) from one or more selected embryo explants or any generation of a progeny cell thereof; or crossing the one or more selected genetically modified plant(s) with itself or a different plant to obtain one or more progeny plant(s) or seed(s). In some embodiments, the different plant used for crossing may have the same genotype of the selected genetically modified plant or may have a different genotype compared to the selected genetically modified plant. In specific embodiments, a first selected genetically modified plant may be crossed with a selected second genetically modified plant. The first and second selected genetically modified plants may, in some embodiments, comprise the same genetic modification or different genetic modifications.

In particular embodiments, the present disclosure further provides methods comprising: observing a culturing characteristic of at least two selected embryo explants or observing a phenotype of at least two selected genetically modified plants. The methods provided by the present disclosure, in some embodiments, further comprise comparing the culturing characteristic of the at least two selected embryo explants or the phenotype of the at least two selected genetically modified plants to determine which of the selected embryo explants or selected genetically modified plants is superior. The superior selected genetically modified plant may, in certain embodiments, then be crossed with itself or a different plant to obtain a progeny plant or seed. A regenerated genetically modified plant may, in some embodiments, be regenerated or grown from the superior selected explant and the regenerated genetically modified plant may then be crossed with itself or a different plant to obtain a progeny plant or seed.

In some embodiments, the present disclosure provides methods comprising introgressing a chromosomal segment conferring one or more culturing characteristic(s) or one or more phenotype(s) into a plant which lacks the culturing characteristic(s) or phenotype(s). As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion. In specific embodiments, the present disclosure provides methods comprising crossing a genetically modified plant comprising a chromosomal segment conferring one or more culturing characteristic(s) or one or more phenotype(s) with itself or a second plant to produce a progeny plant or seed comprising the chromosomal segment.

### H. Genetically Modified Plants by Genetic Engineering

Various genetic engineering technologies have been developed and may be used by those of skill in the art to introduce transgenic or edited traits into plants. The methods generally involve the delivery of a polynucleotide sequence into a plant cell, which may typically be a heterologous and/or recombinant polynucleotide molecule. A heterologous or recombinant polynucleotide molecule may comprise, for example, at least one transgene, expression cassette, or RNA molecule, such as a guide RNA (gRNA). In specific embodiments, the heterologous or recombinant polynucleotide molecule may be part of a ribonucleoprotein (RNP) or a fragment thereof and/or a guide RNA. Expression of the heterologous or recombinant polynucleotide molecule, may for example, produce a gRNA/site-specific nuclease complex for genome editing. In certain embodiments, traits are introduced into plants by altering or introducing a single genetic locus or transgene into the genome of a plant. Methods of genetic engineering to modify, delete, or insert transgenes, edits, mutations and polynucleotide sequences into the genomic DNA of plants are known in the art. Molecular methods of editing a plant cell genome or endogenous plant gene using a genome editing technique are known in the art. According to present embodiments, a polynucleotide or DNA molecule comprising and/or encoding genome editing tools or machinery, such as a guide RNA, site-specific nuclease, and/or template DNA molecule, may be introduced into a plant cell using the methods described herein.

Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “site-specific nuclease” includes any zinc finger nuclease (ZFN), a transcription activator-like effector nuclease (TALEN), ribonucleoprotein, meganuclease, recombinase, transposase, or other nuclease that can introduce a double-stranded break (DSB) or single-stranded nick into a polynucleotide sequence and/or its complementary DNA strand at or near a target site, such as a target site within the genome of a plant cell. As used herein, a “target region” or a “targeted region” refers to a polynucleotide sequence or region that is flanked by two or more target sites. Without being limiting, in some embodiments, a target region may be subjected to a mutation, deletion, insertion or inversion. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE (transcription activator-like effector)-endonuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

According to some embodiments, a site-specific nuclease may be co-delivered with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease provided herein may be selected from the group consisting of a zinc-finger nuclease (ZFN), a TALE-endonuclease (TALEN), a meganuclease, an RNA-guided endonuclease, a recombinase, a transposase, or any combination thereof. See, e.g., Khandagale et al. (Plant Biotechnol Rep 10:327-343, 2016); and Gaj et al. (Trends Biotechnol. 31(7):397-405, 2013. Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent App. Pub. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs, PvuII functions as a highly specific cleavage domain when coupled to a TALE (see Yank et al., PLoS One 8:e82539, 2013). MutH is capable of introducing strand-specific nicks in DNA (see Gabsalilow et al., Nucleic Acids Research. 41:e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40: W117-122, 2012); Cermak et al. (Nucleic Acids Research 39:e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR-associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site specific nuclease may lead to knockdown of gene expression via repair of the DSB or nick, which may result in a mutation or insertion of a sequence at the site of the DSB or nick through cellular repair mechanisms. Such cellular repair mechanism may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of a plant. According to this approach, mutations, such as deletions, insertions, inversions, and/or substitutions may be introduced at a target site via imperfect repair of the DSB or nick to produce a knock-out or knock-down of a gene. Such mutations may be generated by imperfect repair of the targeted locus even without the use of a donor template molecule. A “knock-out” of a gene may be achieved by inducing a DSB or nick at or near the endogenous locus of the gene that results in non-expression of the protein or expression of a non-functional protein, whereas a “knock-down” of a gene may be achieved in a similar manner by inducing a DSB or nick at or near the endogenous locus of the gene that is repaired imperfectly at a site that does not affect the coding sequence of the gene in a manner that would eliminate the function of the encoded protein. For example, the site of the DSB or nick within the endogenous locus may be in the upstream or 5′ region of the gene (e.g., a promoter and/or enhancer sequence) to affect or reduce its level of expression.

Similarly, such targeted knock-out or knock-down mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out or knock-down mutations of a gene may be achieved by substituting, inserting, deleting or inverting at least a portion of the gene, such as by introducing a frame shift or premature stop codon into the coding sequence of the gene or disrupting a promoter sequence or the sequence of another non-coding regulatory element of the gene. A deletion of a portion of a gene may also be introduced by generating DSBs or nicks at two target sites and causing a deletion of the intervening target region flanked by the target sites.

In further embodiments, genetic modification of a plant may comprise transformation of a plant, plant part, plant tissue or plant cell to insert a polynucleotide or DNA sequence or transgene into the genome of the plant, plant part, plant tissue or plant cell. Methods for transformation of plants that are known in the art and applicable to many crop species include, but are not limited to, electroporation, microprojectile or particle bombardment, microinjection, PEG-mediated transformation, Agrobacterium-mediated transformation, and other modes of direct DNA uptake. Bacteria known to mediate plant cell transformation include a number of species of bacterial genera, species, and strains that may be assigned to the order Rhizobiales other than Agrobacterium, including but not limited to, bacterial species and strains from the taxonomic families Rhizobiaceae (e.g. Rhizobium spp., Sinorhizobium spp.), Phyllobacteriaceae (e.g. Mesorhizobium spp., Phyllobacterium spp.), Brucellaceae (e.g. Ochrobactrum spp.), Bradyrhizobiaceae (e.g. Bradyrhizobium spp.), and Xanthobacteraceae (e.g. Azorhizobium spp.), among others. According to some embodiments, Agrobacterium-mediated transformation is mediated by Agrobacterium tumefaciens. Targets for such transformation have often been undifferentiated callus tissues, although differentiated tissues have also been used for transient and stable plant transformation. As is well known in the art, other methods for plant transformation may be utilized, for instance as described by Miki et al., (1993, “Procedures for Introducing Foreign DNA into Plants,” in Methods in Plant Molecular Biology and Biotechnology, Glick B. R. and Thompson, J. E. Eds., CRC Press, Inc., Boca Raton, pages 67-88).

In specific embodiments, microprojectile bombardment may be employed to deliver a polynucleotide or DNA molecule, vector, sequence, segment, or RNP to at least one cell of plant explants. In this method, particles are coated with a polynucleotide or polynucleotide/protein complex and delivered into cells by a propelling force. Exemplary particles may include those comprised of tungsten, platinum, or gold. For bombardment, explants or other target cells may be arranged on a solid culture medium. The cells to be bombarded are positioned at an appropriate distance below the projectile stopping plate. A polynucleotide may be delivered into plant cells by acceleration using a biolistics particle delivery system, which may propel particles coated with a DNA or polynucleotide molecule through a screen, such as a stainless steel or Nytex screen, and toward the explants positioned on a surface. The screen may disperse the particles so that they are not delivered to the recipient cells in large aggregates. Microprojectile bombardment techniques are widely applicable and may be used to transform a variety of plant species.

Agrobacterium-mediated or Rhizobiales-mediated transformation of explants is another widely applicable system for introducing heterologous and/or recombinant DNA molecules into plant cells. Modern Agrobacterium-mediated transformation vectors are capable of replication in E. coli as well as in Agrobacterium, allowing for convenient manipulations (see, e.g., Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti plasmids can be used for transformation. Agrobacterium-mediated transformation is often the method of choice for many plant species. The use of Agrobacterium-mediated plant integrating vectors to introduce DNA into plant cells is known in the art (see, e.g., Fraley et al., Nat. Biotechnol., 3:629-635, 1985; U.S. Pat. No. 5,563,055).

A number of promoters and expression elements have utility for plant gene expression of any selectable marker, scoreable marker, transgene, or any other gene of agronomic interest. Promoters may include any constitutive promoter, tissue specific promoter, organ specific promoter, inducible promoter, reproductive tissue promoter, developmental stage promoter, viral promoter, or the like. Examples of various types of promoters and expression elements are known in art. Expression elements that may be useful for plant gene expression may include, for example, various promoters, enhancers, leaders, 5′ and 3′ untranslated regions, introns, terminators, and the like, as known in the art. A selectable or screenable marker or gene of interest may also be fused to a transit peptide or other targeting sequence. Transport of proteins produced by transgenes to a subcellular compartment such as the chloroplast, vacuole, peroxisome, glyoxysome, cell wall, nucleus, or mitochondrion or for secretion into the apoplast, may be accomplished by means of operably linking the nucleotide sequence encoding a signal or targeting sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art. See, for example Becker et al. (Plant Mol. Biol., 20:49, 1992); Knox et al. (Plant Mol. Biol., 9:3-17, 1987); Lerner et al. (Plant Physiol., 91:124-129, 1989); Fontes et al. (Plant Cell, 3:483-496, 1991); Matsuoka et al. (Proc. Natl. Acad. Sci. USA, 88:834, 1991); Gould et al. (J. Cell. Biol., 108:1657, 1989); Creissen et al. (Plant J., 2:129, 1991); Kalderon et al. (Cell, 39:499-509, 1984); Steifel et al. (Plant Cell, 2:785-793, 1990).

Examples of constitutive promoters may include, for example, the cauliflower mosaic virus (CaMV) 35S promoter, which confers constitutive, high-level expression in most plant tissues (see, e.g., Odel et al., Nature, 313:810, 1985), including monocots (see, e.g., Dekeyser et al., Plant Cell, 2:591, 1990; Terada and Shimamoto, Mol. Gen. Genet., 220:389, 1990), a tandemly duplicated version of the CaMV 35S promoter, the enhanced 35S promoter (e35S), the nopaline synthase promoter (An et al., Plant Physiol., 88:547, 1988), the octopine synthase promoter (Fromm et al., Plant Cell, 1:977, 1989), and the figwort mosaic virus (FMV) promoter as described in U.S. Pat. No. 5,378,619 and an enhanced version of the FMV promoter (eFMV) where the promoter sequence of FMV is duplicated in tandem, the cauliflower mosaic virus 19S promoter, a sugarcane bacilliform virus promoter, a commelina yellow mottle virus promoter, and other plant DNA virus promoters known to express in plant cells.

With an inducible promoter, the rate of transcription increases in response to an inducing agent or signal. Any inducible promoter can be used according to the embodiments of the present disclosure. A variety of plant gene promoters that are regulated in response to environmental, hormonal, chemical, and/or developmental signals can be used for expression of an operably linked gene in plant cells, including promoters regulated by (1) heat (e.g., Callis et al., Plant Physiol., 88:965, 1988), (2) light (e.g., pea rbcS-3A promoter, Kuhlemeier et al., Plant Cell, 1:471, 1989; maize rbcS promoter, Schaffner and Sheen, Plant Cell, 3:997, 1991; or chlorophyll a/b-binding protein promoter, Simpson et al., EMBO J., 4:2723, 1985), (3) hormones, such as abscisic acid (Marcotte et al., Plant Cell, 1:969, 1989), (4) wounding (e.g., Siebertz et al., Plant Cell, 1:961, 1989), or (5) chemicals such as methyl jasmonate, salicylic acid, or Safener. It may also be advantageous to employ organ-specific or tissue specific promoters known in the art (e.g., Roshal et al., EMBO J., 6:1155, 1987; Schernthaner et al., EMBO J., 7:1249, 1988; Bustos et al., Plant Cell, 1:839, 1989).

Exemplary polynucleotide or DNA molecules which may be introduced to the explants of the population provided herein include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to genes that are not normally present in the cell being transformed, or perhaps not present in the form, structure or location. A polynucleotide may include a DNA molecule or sequence which is already present in the plant cell, is from another plant, is from a different organism, is exogenous or generated externally. A transgene or expression cassette may encode a mRNA and protein or an RNA molecule for suppression, such as a miRNA, siRNA, dsRNA, antisense RNA, inverted repeat RNA, or the like. A polynucleotide may be an exogenous, heterologous and/or recombinant polynucleotide or DNA molecule or sequence.

Many different transgenes or genetic modifications could potentially be introduced into a population of explants according to the present disclosure, which may provide a beneficial agronomic trait of a crop plant having the transgene or modification. Non-limiting examples of particular transgenes or genetic modifications and corresponding phenotypes one may choose to introduce into the population include one or more genes for insect resistance, such as a Bacillus thuringiensis (B.t.) gene, pest tolerance, such as genes for fungal disease control, herbicide tolerance, such as genes conferring glyphosate tolerance, and genes for quality improvements, such as yield, nutritional enhancements, environmental or stress tolerances, or any desirable changes in plant physiology, growth, development, morphology or plant product(s). For example, structural genes would include any gene that confers insect resistance, including, but not limited to, a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In some embodiments, the structural gene can confer tolerance to the herbicide glyphosate as conferred by genes including, but not limited to, Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

A variety of assays are known in the art and may be used to confirm the presence of a genetic modification, exogenous DNA sequence, genetic marker, expression cassette or transgene in transformed, edited, or genetically modified explants, cultured explants, plants or plant parts. Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot hybridization; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot hybridization. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acid or polynucleotide molecules can be isolated using techniques routine in the art. For example, nucleic acids can be isolated using any method including, without limitation, recombinant nucleic acid technology and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blot hybridization, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blot analysis, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### I. Culture Media

A variety of tissue culture media are known that, when supplemented appropriately, support plant tissue growth and development, including formation of mature plants from excised plant tissue. As used herein, the term “tissue culture media” refers to liquid, semi-solid, or solid media used to support plant growth and development in a non-soil environment. These tissue culture media can either be purchased as a commercial preparation or custom prepared and modified by those of skill in the art. Examples of such media include, but are not limited to those described by Murashige and Skoog, (Physiologia Plantarum, 15:473-497, 1962); Chu et al., (Scient. Sin., 18: 659-668, 1975); Linsmaier and Skoog, (Physiologia Plantarum, 18:100-127, 1965); Uchimiya and Murashige, (Plant Physiol. 57:424-429, 1976); Gamborg et al., (Exp. Cell Res., 50:151-158, 1968); Duncan et al., (JAMA, 254:2609-2613, 1985); McCown and Lloyd, (HortSciences, 16:453, 1981); Nitsch and Nitsch (Science, 163:85-87, 1969); and Schenk and Hildebrandt, (Canadian J. Botany, 50:199-204, 1972), or derivations of these media supplemented accordingly. Those of skill in the art are aware that media and media supplements, such as nutrients and plant growth regulators for use in transformation and regeneration are usually optimized for the particular target crop or variety of interest, and for specific explant tissues. Tissue culture media may be supplemented with carbohydrates such as, but not limited to, glucose, sucrose, maltose, mannose, fructose, lactose, galactose, and/or dextrose, or ratios of carbohydrates. Reagents are commercially available and can be purchased from a number of suppliers (see, for example Sigma Chemical Co., St. Louis, MO; and PhytoTechnology Laboratories, Shawnee Mission, KS). These tissue culture media may be used to prepare an inoculation, co-culture, bud induction, second bud induction, or regeneration media, and in particular embodiments may comprise a selection agent.

### J. Selectable and Screenable Markers

In particular embodiments, media for use according to the present disclosure may comprise one or more selection agents and the heterologous polynucleotide molecule for use in the present disclosure may comprise a selectable marker gene, wherein the selectable marker gene provides resistance to the selection agent. As used herein, “selectable marker” or “screenable marker” or “scoreable marker” refers to a nucleic acid sequence whose expression confers a phenotype facilitating identification of cells containing the nucleic acid sequence. Examples of various selectable markers and genes providing resistance to transgenic cells are disclosed in Miki and McHugh (J. Biotechnology, 107:193-232, 2004). Selectable marker genes that may be used include, but are in no way limited to, aroA, EPSPS, aadA, pat, bar, hph (hygromycin B phosphotransferase), DMO (dicamba omonooxygenase), CAT and NPT II. Non-limiting examples of selection agents that may be used according to the present disclosure include glyphosate, glufosinate, phosphinothricin, bromoxynil, bialaphos, dicamba, imidazolinone, sulfonylurea, acetolactate synthase inhibitors, protoporphyrinogen oxidase inhibitors, hydroxyphenyl-pyruvate-dioxygenase inhibitors, antibiotic inhibitors, neomycin, kanamycin, paramomycin, G418, aminoglycosides, spectinomycin, streptomycin, hygromycin B, bleomycin, phleomycin, sulfonamides, gentamycin, streptothricin, chloramphenicol, methotrexate, 2-deoxyglucose, betaine aldehyde, S-aminoethyl L-cysteine, 4-methyltryptophan, D-xylose, D-mannose, and benzyladenine-N-3-glucuronidase. The heterologous polynucleotide molecule for use in the present disclosure may, in some embodiments, comprise two or more selectable marker genes. Selection agents for use in the present disclosure may, in some embodiments, be used alone or as a combination of two or more selection agents. In particular embodiments, the embodiments of the present disclosure may be performed in the absence of any selection agent.

According to embodiments of the present disclosure, the insertion sequence of an exogenous polynucleotide or DNA molecule for transformation or genome editing may comprise a plant selectable marker gene to allow for successful selection for, and production of, transformed or transgenic Ro plants. A plant selectable marker gene or transgene may include any gene conferring tolerance to a corresponding selection agent, such that plant cells transformed with the plant selectable marker transgene may tolerate and withstand the selection pressure imposed by the selection agent. As a result, transformed plant cells of an explant are favored to grow, proliferate, and/or develop under selection. Although a plant selectable marker gene is generally used to confer tolerance to a selection agent, additional screenable or scorable marker gene(s) may also be used in addition to the selectable marker, perhaps also along with a gene of agronomic interest. Such screenable marker genes may include, for example, uidA for β-glucuronidase (GUS; e.g., as described in U.S. Pat. No. 5,599,670, which is hereby incorporated by reference) or gfp for green fluorescent protein and variants thereof (GFP described in U.S. Pat. Nos. 5,491,084 and 6,146,826, all of which are hereby incorporated by reference) or crtB for phytoene synthase (e.g., as described in U.S. Pat. Nos. 8,237,016 and 10,240,165, all of which are hereby incorporated by reference. Additional examples of screenable markers may include secretable markers whose expression causes secretion of a molecule(s) that can be detected as a means for identifying transformed cells.

A plant selectable marker gene may comprise a gene encoding a protein that provides or confers tolerance or resistance to an herbicide, such as glyphosate or glufosinate. Examples of known plant selectable marker genes encoding proteins that confer herbicide resistance or tolerance include, for example, a transcribable DNA molecule encoding 5-enolpyruvylshikimate-3-phosphate synthase (EPSPS for glyphosate tolerance; e.g., as described in U.S. Pat. Nos. 5,627,061; 5,633,435; 6,040,497; and 5,094,945, all of which are hereby incorporated by reference); a transcribable DNA molecule encoding a glyphosate oxidoreductase and a glyphosate-N-acetyl transferase (GOX; e.g., as described in U.S. Pat. No. 5,463,175; GAT described in U.S. Patent Publication No. 20030083480); a transcribable DNA molecule encoding phytoene desaturase (crtI; e.g., as described in Misawa, et al., (Plant Journal, 4:833-840, 1993) and Misawa, et al., (Plant Journal, 6:481-489, 1994) for norflurazon tolerance, incorporated herein by reference); and the bar gene (e.g., as described in DeBlock, et al., EMBO Journal, 6:2513-2519 1987) for glufosinate and bialaphos tolerance, incorporated herein by reference). Additional plant selectable marker genes known in the art may include those encoding proteins that confer resistance or tolerance to an antibiotic, such as streptomycin and/or spectinomycin (e.g., aadA, spec/strep), kanamycin (e.g., nptII), hygromycin B (e.g., aph IV), gentamycin (e.g., aac3 and aacC4), and chloramphenicol (e.g., CAT).

The insertion sequence of an exogenous DNA molecule may further comprise sequences for removal of one or more transgene(s) or expression cassette(s), such as a plant selectable marker transgene, or any portion or sequence thereof, after successful production and/or confirmation of a transformed explants, cultured explants, or plants, especially after the transgene or expression cassette is no longer needed. In some embodiments, this may be accomplished by flanking the transgene sequence to be removed, with known or later developed recombination sites (e.g., LoxP sites, FRT sites, gRNA recognition sites, and the similar) that can be recognized and removed by an endogenous or exogenously provided recombinase enzyme (e.g., Cre, Flp, a RNP, such as a gRNA/site-specific nuclease complex for genome editing, and the similar). The recombinase enzyme may be introduced and expressed in trans, such as by crossing the transformed plant to another plant having the recombinase transgene, to accomplish excision of the transgene. Accordingly, the unwanted sequence element or transgene can be removed once its use or purpose has expired, thus preventing its further expression or transmission in the germ line.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description.

### Examples

Those of skill in the art will appreciate the many advantages of the methods and compositions provided by the present disclosure. The following examples are included to demonstrate the preferred embodiments of the disclosure. It should be appreciated by those of skill in the art that the techniques disclosed in the examples that follow represent techniques discovered by the inventors to function well in the practice of the embodiments of the disclosure, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the disclosure. All references cited herein are incorporated herein by reference to the extent that they supplement, explain, provide a background for, or teach methodology, techniques, or compositions employed herein.

### Example 1

### Preparation of Bulked Corn Seeds of Multiple Germplasms for Genetic Modification

A genetically modified plant, plant part, plant tissue, explant, or plant cell comprises a genetic modification or transgene introduced into the genome. Genetic modifications can produce variable phenotypes in different genetic backgrounds of the same crop. Investigation of these variations or interactions for a given genetic modification in different plant germplasms or genetic backgrounds, which may be described as “Germplasm x Transgene/Edit Interactions,” currently involves the creation of a transgenic event or edit in a single germplasm followed by crossing the event or edit into other germplasms. Prior to the present disclosure, any genetic background or germplasm dependent differences in phenotype for a given transgene, transgenic event, or edit could not be tested, observed, screened, or selected until the transgene, event, or edit has been crossed into multiple germplasms over several generations. This process can be time consuming, costly, and inefficient. As provided in the present disclosure, genetic modification of multiple plant germplasms or lines simultaneously or in parallel greatly improves the efficiency of this screening and/or selection process and allows for faster and more cost-effective evaluation, for example, of any Germplasm x Transgene/Edit Interactions. According to this approach, a heterologous polynucleotide molecule is introduced into a population of embryo explants from multiple germplasms at once, which may be followed by deconvolution, screening, and/or selection of individual germplasm(s) containing the genetic modification.

In Example 1, Experiment 1, corn seeds were harvested from multiple female and male inbred lines, female F₁ segregating populations (each produced by a cross of two different female inbred lines), and male F₁ segregating populations (each produced by a cross of two different male inbred lines). The harvested seeds were sorted and mixed into four seed bulks based on size and shape—round (Bulk 1), flat (Bulk 2), large round (Bulk 3), and large flat (Bulk 4). As used herein, the “female” and “male” lines or germplasms refer to inbred lines that are typically used as the female or male line, respectively, in hybrid crosses. Sorting the seeds into different bulks based on size and shape could allow for different mechanical settings to be used to effectively excise explants from the seeds or to ensure balanced inclusion or distribution of seeds of different sizes and shapes for each germplasm type if excised together. In this experiment, seeds of the different inbred lines and segregating populations (segregants) were combined after sorting based on seed size and shape, and the number of different germplasms present in each of Bulks 1-4 were the same. An additional bulk (Bulk 5) consisted of seeds from twenty different hybrid lines produced by crossing different male and female lines. Each of Bulks 1-5 also included seeds of two corn inbred control lines, which serve as a positive and negative control for transformation, respectively. Seeds of each of Bulks 1-5 were excised separately to generate explants for each Bulk. Table 1 provides a summary of the composition of Bulks 1-5 in Example 1, Experiment 1, with values in Tables 1-3 referencing the numbers of distinct plant germplasm lines per bulk for each germplasm type. Each seed bulk was processed to obtain explants for transformation as provided in Example 2. Table 4 provides numbers of seeds of each germplasm in Bulks 1-4.

TABLE 1
Summary of the compositions of seed Bulks 1-5 for
explant preparation in Example 1, Experiment 1.

| Germplasm Type | Bulk 1 | Bulk 2 | Bulk 3 | Bulk 4 | Bulk 5 |
|---|---|---|---|---|---|
| Number of Female Inbreds | 38 | 38 | 38 | 38 | |
| Number of Male Inbreds | 38 | 38 | 38 | 38 | |
| Number of Inbred Controls | 2 | 2 | 2 | 2 | 2 |
| Number of Hybrids | — | — | — | — | 20 |
| Number of Female F1 Segregants | 250 | 250 | 250 | 250 | — |
| Number of Male F1 Segregants | 250 | 250 | 250 | 250 | — |
| Total Number of Seeds | 139,699 | 112,966 | 172,304 | 128,145 | 335,120 |

In Example 1, Experiment 2, corn seeds were harvested from multiple male inbred lines. The harvested seeds were combined into a single seed bulk. The resulting seed bulk was processed to obtain explants for transformation as provided in Examples 2-4. Table 2 provides a summary of the composition of the bulked seeds from the multiple male germplasm lines.

TABLE 2
Summary of the composition of seeds from multiple male lines
for explant preparation in Example 1, Experiment 2.

| Type | Male Bulk |
|---|---|
| Number of Male Inbreds | 20 |
| Total Number of Seeds | 800,000 |

In Example 1, Experiment 3, corn seeds were harvested from multiple tropical female inbred lines and three non-tropical, female control lines. The harvested seeds were combined into a single seed bulk. The resulting seed bulk was processed to obtain explants for transformation and genome editing as provided in Example 4. Table 3 provides a summary of the composition of the bulked seeds from the tropical and non-tropical control germplasm lines.

TABLE 3
Summary of the composition of seeds from multiple tropical and control
lines for explant preparation in Example 1, Experiment 3.

| Germplasm Type | Tropical Bulk |
|---|---|
| Number of Tropical Inbreds | 24 |
| Number of Non-Tropical | 3 |
| Control Inbreds | |
| Total Number of Seeds | 149,923 |

### Example 2

### Bulk Corn Explant Viability and Shoot Generation Ability Assessment

This example evaluates the efficiency of explant excision and explant production in bulked seeds from multiple corn germplasms. In particular, this example evaluates whether a particular explant production process is germplasm independent, whether different germplasms respond differently to seed explant excision, and whether hybrids and inbred lines respond differently to explant production and culturing. A total of five seed bulks were produced (defined as Bulks 1-5 in Example 1, Experiment 1), and each was subjected to an automated seed explant excision process, which included the steps of seed sanitizing/drying, seed milling, explant enrichment, and purification. Briefly, mature or immature seeds were harvested from corn plants grown in the field or greenhouse, and divided into the five bulks based on seed size and shape. The seeds were then sanitized using various methods including: exposure to liquids, temperatures at or below −20° C., gases, and/or UV light. In addition, the seeds may be dried to obtain desired moisture content for storage. Using mechanical means, explants comprising transformable and regenerable tissue, including embryonic and/or meristematic tissue, were excised from seeds. The explants were enriched and purified through removal of debris and unnecessary seed parts, such as by rinsing and/or floatation. Explants may also be dried to obtain a desired moisture content for storage purposes. A random sampling of approximately 700 explants from each of Bulks 1-5 in Example 1, Experiment 1 was collected for initial genotyping and deconvolution using genetic markers as described in Example 5. This data was used to evaluate the explant excision efficiency of each germplasm in bulk.

Excised explants were then evaluated for their ability to grow and generate shoots on culture media in vitro. Briefly, for Example 2, Experiment 1, approximately 5,400 corn excised explants per bulk were removed from the freezer, thawed for 30 minutes, and surface sterilized with 70% ethanol containing 100 g/L PEG MW 8000 (polyethylene glycol) for 3.5 to 4 minutes with agitation in batches of approximately 500-700 explants per container. The explants were then rinsed immediately for 30 seconds using a rinse station or using 500 mL of sterile water for 5 to 6 repeated rinses. Explants were then collected, enriched, and purified by repeated floatation in sterile water. After each float, floating (viable) explants were collected and removed. The float step was repeated 3 to 6 times until no additional viable explants floated to surface. Collected explants were transferred to about 400 mL of rehydration medium containing ⅖ strength B5 macro salts except ½ strength CaCl₂), 1/10 strength B5 vitamins and micro salts, 2.8 mg/L sequestrene, 1 g/L potassium nitrate, 30 g/L dextrose, 3.9 g/L MES, 0.03 g/L Clearys 3336 WP, pH 5.4 for 1 to 2 hours before being plated on solid regeneration media without selection containing MS salts, B5 vitamins, 30 g/L sucrose, 0.69 g/L proline, 1 g/L NZ amine-A, 2 mg/L glycine, 1 g/L MES, 400 mg/L carbenicillin, 200 mg/L cefotaxime, 100 mg/L timentin, 3.5 g/L low EEO agarose, pH 5.8, at a density of about 100 explants per 600 mL culture vessel. The culture vessels were incubated at 28+/−3° C. with a photoperiod of 16-hour light/8-hour dark, and a light intensity target of 160 PAR (about 140 to about 200 PAR is acceptable) for about two weeks. Samples were collected from growing shoots that were at least 5 cm in size with strong meristems and genotyped using genetic markers as described in Example 5.

For these experiments, each of Bulks 1-4 was separately tested, and the data for Bulks 1-4 were combined for each germplasm (male or female inbred line, male or female F₁ segregant, or control inbred line), as identified in Tables 1 and 4. Specifically, experiments were performed with combined seeds and explants for all female and male inbred lines, male and female F₁ segregating populations, and control inbred lines for each Bulk. For example, seeds and explants for all female and male inbred lines, male and female F₁ segregating populations, and control inbred lines in Bulk 1 were combined and tested together, seeds and explants for all female and male inbred lines, male and female F₁ segregating populations, and control inbred lines in Bulk 2 were combined and tested together, and the same for each of Bulks 3 and 4.

Although the germplasms were combined together in this experiment for each Bulk, the data are presented in Table 4 for each germplasm. The data provided in Table 4 was obtained by genotyping the explants and/or growing shoots and then sorting the data by germplasm. As provided in Tables 1 and 4, the 38 inbred lines belonged to female heterotic groups (each identified as F-1, F-2, etc.), and the 38 inbred lines belonged to male heterotic groups (each identified as M-1, M-2, etc.). In addition, 250 male segregants (M-1×M-2, etc.), 250 female segregants (F-1×F-2, etc.) and two female inbred control lines were included in Bulks 1-4. Control Line 1 is a positive control previously demonstrated to exhibit high transformation and/or editing efficiency, whereas Control Line 2 is a negative control previously demonstrated to exhibit a lower transformation and/or editing frequency.

As shown in Table 4, each germplasm represented from 0.63% to 7.73% of the total number of corn kernels or seeds in Bulks 1-4 (see column 4 in Table 4). A total of 2,713 randomly selected explants from Bulks 1-4 were identified by genotyping after explant production and prior to regeneration to determine the explant excision efficiency of each germplasm.

In Table 4, “# Kernels in Bulks 1-4” (see column 3 of Table 4) is the total number of kernels for a specific germplasm included in Bulks 1-4; “% Kernels in Bulks 1-4” (see column 4 of Table 4) is the “# Kernels” for a specific germplasm in a single bulk divided by the total number of kernels in that Bulk×100, calculated for each of Bulks 1-4 separately then averaged (summed and divided by 4), (i.e., ((# of M-1 Kernels in Bulk 1/Total # Kernels in Bulk 1)×100)+((# of M-1 Kernels in Bulk 2/Total # Kernels in Bulk 2)×100)+((# of M-1 Kernels in Bulk 3/Total # Kernels in Bulk 3)×100)+((# of M-1 Kernels in Bulk 4/Total # Kernels in Bulk 4)×100)/4); “# Explants” (see column 5 of Table 4) is the number of corn explants that were sampled and determined by genotyping to be the specific germplasm out of a total of 2,713 randomly sampled explants from Bulks 1-4; and “% Explants” (see column 6 of Table 4) is defined as the “# of Explants” for a specific germplasm in a single bulk divided by the total number of explants in that Bulk×100, calculated for each of Bulks 1-4 separately then averaged (summed and divided by 4). In column 7 of Table 4, “Relative Explant Presence” is defined as the “% Explants” (see column 6 of Table 4) divided by the “% Kernels in Bulks 1-4” (see column 4 of Table 4) and indicates the relative representation of each germplasm in Bulks 1-4. “# Shoots Generated” represents the number of explants that generated shoots on culture media for each germplasm as determined by genotyping (see column 8 of Table 4). “% Shoots Generated” (see column 9 of Table 4) is defined as the number of explants that generated shoots on culture media and determined to be the specific germplasm by genotyping in a single bulk divided by total # of shoot-generating explants identified by genotyping for all germplasms in that Bulk×100, calculated for each of Bulks 1-4 separately then averaged (summed and divided by 4)×100. “Relative Shoot Presence” for each germplasm in column 10 of Table 4 is defined as the “% Shoots Generated” (see column 9 of Table 4) divided by the “% Kernels in Bulks 1-4” (see column 4 of Table 4) and indicates the relative representation of shoots generated for each germplasm in Bulks 1-4. Some samples were unable to be identified by genotyping due to missing data as described in Example 5 (averaged <3% for the randomly selected explants, and <1% for the shoots), and were excluded. “F” or “M” in columns 1 and 2 of Table 4 indicates a female or a male inbred line, respectively.

TABLE 4
Response of different germplasms in bulked seed preparations to explant production,
culturing, and shoot generation (Example 2, Experiment 1; Bulks 1-4).

| | | 3 | 4 | | | 7 | | | 10 |
|---|---|---|---|---|---|---|---|---|---|
| | 2 | # Kernels | % Kernels | 5 | 6 | Relative | 8 | 9 | Relative |
| 1 | Heterotic | in Bulks | in Bulks | # | % | Explant | # Shoots | % Shoots | Shoot |
| Germplasm | group | 1-4 | 1-4 | Explants | Explants | Presence | Generated | Generated | Presence |
| F-1 | F | 5,766 | 1.01% | 36 | 1.32% | 1.311 | 31 | 0.87% | 0.862 |
| F-2 | F | 5,956 | 1.10% | 13 | 0.48% | 0.437 | 16 | 0.44% | 0.397 |
| F-3 | F | 5,677 | 1.03% | 20 | 0.74% | 0.721 | 58 | 1.62% | 1.578 |
| F-4 | F | 6,357 | 1.21% | 45 | 1.66% | 1.374 | 50 | 1.38% | 1.139 |
| F-5 | F | 5,754 | 0.97% | 9 | 0.33% | 0.338 | 18 | 0.50% | 0.513 |
| F-6 | F | 5,229 | 0.91% | 15 | 0.55% | 0.603 | 34 | 0.93% | 1.021 |
| F-7 | F | 5,813 | 1.12% | 17 | 0.63% | 0.565 | 57 | 1.53% | 1.371 |
| F-8 | F | 5,992 | 1.09% | 15 | 0.57% | 0.52 | 30 | 0.81% | 0.742 |
| F-9 | F | 5,893 | 1.20% | 41 | 1.52% | 1.264 | 86 | 2.34% | 1.944 |
| F-10 | F | 6,093 | 1.23% | 16 | 0.60% | 0.482 | 33 | 0.89% | 0.72 |
| F-11 | F | 6,092 | 1.10% | 18 | 0.67% | 0.609 | 33 | 0.93% | 0.846 |
| F-12 | F | 6,205 | 1.18% | 25 | 0.94% | 0.798 | 32 | 0.86% | 0.731 |
| F-13 | F | 5,772 | 1.03% | 13 | 0.48% | 0.467 | 36 | 0.99% | 0.966 |
| F-14 | F | 6,024 | 1.20% | 15 | 0.56% | 0.464 | 44 | 1.19% | 0.992 |
| F-15 | F | 5,900 | 1.15% | 14 | 0.52% | 0.451 | 25 | 0.68% | 0.591 |
| F-16 | F | 6,159 | 1.15% | 12 | 0.45% | 0.387 | 44 | 1.19% | 1.029 |
| F-17 | F | 6,125 | 1.17% | 22 | 0.81% | 0.693 | 25 | 0.68% | 0.58 |
| F-18 | F | 6,357 | 1.07% | 36 | 1.32% | 1.233 | 58 | 1.62% | 1.512 |
| F-19 | F | 6,085 | 1.12% | 33 | 1.22% | 1.089 | 55 | 1.52% | 1.355 |
| F-20 | F | 6,277 | 1.17% | 14 | 0.52% | 0.439 | 31 | 0.85% | 0.727 |
| F-21 | F | 5,948 | 1.19% | 29 | 1.08% | 0.907 | 37 | 1.00% | 0.844 |
| F-22 | F | 6,182 | 1.12% | 10 | 0.37% | 0.33 | 35 | 0.98% | 0.876 |
| F-23 | F | 6,318 | 1.22% | 15 | 0.56% | 0.461 | 50 | 1.36% | 1.116 |
| F-24 | F | 6,068 | 1.19% | 12 | 0.45% | 0.375 | 34 | 0.92% | 0.776 |
| F-25 | F | 6,187 | 1.11% | 37 | 1.37% | 1.234 | 36 | 1.00% | 0.901 |
| F-26 | F | 5,980 | 1.11% | 27 | 1.00% | 0.901 | 69 | 1.86% | 1.679 |
| F-27 | F | 6,150 | 1.16% | 39 | 1.45% | 1.244 | 59 | 1.60% | 1.37 |
| F-28 | F | 5,298 | 1.06% | 12 | 0.45% | 0.419 | 34 | 0.92% | 0.862 |
| F-29 | F | 6,126 | 1.15% | 23 | 0.86% | 0.745 | 45 | 1.22% | 1.058 |
| F-30 | F | 7,013 | 1.29% | 21 | 0.78% | 0.601 | 58 | 1.59% | 1.227 |
| F-31 | F | 6,422 | 1.12% | 23 | 0.84% | 0.747 | 57 | 1.62% | 1.45 |
| F-32 | F | 5,815 | 1.08% | 34 | 1.25% | 1.158 | 47 | 1.29% | 1.2 |
| F-33 | F | 5,963 | 1.08% | 16 | 0.60% | 0.554 | 44 | 1.20% | 1.113 |
| F-34 | F | 5,423 | 0.97% | 21 | 0.78% | 0.803 | 68 | 1.83% | 1.889 |
| F-35 | F | 5,554 | 1.09% | 30 | 1.12% | 1.029 | 60 | 1.61% | 1.481 |
| F-36 | F | 5,486 | 0.93% | 28 | 1.03% | 1.103 | 60 | 1.68% | 1.801 |
| F-37 | F | 6,003 | 1.19% | 29 | 1.07% | 0.899 | 63 | 1.71% | 1.438 |
| F-38 | F | 5,814 | 1.15% | 29 | 1.08% | 0.932 | 73 | 1.96% | 1.697 |
| M-1 | M | 5,343 | 0.89% | 50 | 1.83% | 2.066 | 20 | 0.57% | 0.639 |
| M-2 | M | 5,654 | 0.96% | 36 | 1.33% | 1.383 | 34 | 0.96% | 1 |
| M-3 | M | 5,995 | 1.09% | 52 | 1.93% | 1.769 | 38 | 1.04% | 0.957 |
| M-4 | M | 5,566 | 0.90% | 17 | 0.61% | 0.683 | 15 | 0.43% | 0.476 |
| M-5 | M | 4,230 | 0.63% | 31 | 1.11% | 1.76 | 25 | 0.75% | 1.186 |
| M-6 | M | 6,293 | 1.05% | 38 | 1.41% | 1.343 | 56 | 1.56% | 1.492 |
| M-7 | M | 6,090 | 0.97% | 43 | 1.56% | 1.602 | 24 | 0.71% | 0.727 |
| M-8 | M | 6,067 | 1.02% | 55 | 2.02% | 1.966 | 29 | 0.82% | 0.803 |
| M-9 | M | 5,801 | 1.05% | 39 | 1.44% | 1.367 | 34 | 0.93% | 0.88 |
| M-10 | M | 5,889 | 0.98% | 29 | 1.07% | 1.095 | 11 | 0.31% | 0.315 |
| M-11 | M | 6,095 | 1.05% | 36 | 1.32% | 1.252 | 24 | 0.67% | 0.631 |
| M-12 | M | 5,215 | 0.93% | 23 | 0.85% | 0.914 | 20 | 0.54% | 0.58 |
| M-13 | M | 5,685 | 1.03% | 33 | 1.24% | 1.202 | 48 | 1.32% | 1.288 |
| M-14 | M | 5,947 | 0.95% | 25 | 0.93% | 0.976 | 42 | 1.24% | 1.305 |
| M-15 | M | 5,662 | 1.06% | 40 | 1.49% | 1.401 | 52 | 1.42% | 1.34 |
| M-16 | M | 5,873 | 0.97% | 39 | 1.42% | 1.458 | 47 | 1.35% | 1.392 |
| M-17 | M | 6,026 | 1.03% | 34 | 1.24% | 1.202 | 21 | 0.59% | 0.575 |
| M-18 | M | 5,544 | 1.05% | 23 | 0.85% | 0.805 | 49 | 1.35% | 1.28 |
| M-19 | M | 5,907 | 1.13% | 28 | 1.03% | 0.918 | 25 | 0.68% | 0.601 |
| M-20 | M | 5,296 | 0.94% | 34 | 1.27% | 1.361 | 30 | 0.83% | 0.886 |
| M-21 | M | 9,175 | 1.74% | 65 | 2.41% | 1.387 | 43 | 1.16% | 0.669 |
| M-22 | M | 5,896 | 1.04% | 26 | 0.97% | 0.934 | 56 | 1.53% | 1.473 |
| M-23 | M | 5,461 | 0.94% | 16 | 0.58% | 0.621 | 38 | 1.07% | 1.141 |
| M-24 | M | 6,028 | 1.04% | 34 | 1.25% | 1.2 | 29 | 0.81% | 0.775 |
| M-25 | M | 6,019 | 0.92% | 26 | 0.94% | 1.02 | 22 | 0.63% | 0.685 |
| M-26 | M | 6,028 | 1.07% | 36 | 1.32% | 1.237 | 22 | 0.61% | 0.57 |
| M-27 | M | 5,883 | 1.05% | 49 | 1.80% | 1.709 | 52 | 1.46% | 1.384 |
| M-28 | M | 6,100 | 1.11% | 36 | 1.32% | 1.193 | 36 | 1.00% | 0.904 |
| M-29 | M | 6,034 | 1.09% | 29 | 1.07% | 0.978 | 22 | 0.61% | 0.558 |
| M-30 | M | 5,531 | 0.99% | 36 | 1.33% | 1.342 | 32 | 0.88% | 0.887 |
| M-31 | M | 6,081 | 1.09% | 31 | 1.14% | 1.046 | 29 | 0.83% | 0.762 |
| M-32 | M | 6,121 | 1.11% | 42 | 1.54% | 1.389 | 31 | 0.86% | 0.778 |
| M-33 | M | 6,057 | 1.08% | 46 | 1.70% | 1.573 | 25 | 0.70% | 0.65 |
| M-34 | M | 6,511 | 1.18% | 41 | 1.52% | 1.288 | 28 | 0.79% | 0.674 |
| M-35 | M | 6,116 | 1.14% | 27 | 1.00% | 0.877 | 26 | 0.70% | 0.618 |
| M-36 | M | 5,415 | 1.00% | 43 | 1.57% | 1.575 | 9 | 0.25% | 0.253 |
| M-37 | M | 5,957 | 1.06% | 31 | 1.14% | 1.068 | 22 | 0.61% | 0.576 |
| M-38 | M | 5,610 | 1.04% | 52 | 1.92% | 1.848 | 33 | 0.90% | 0.859 |
| 250 Female | F | 42,703 | 7.73% | 155 | 5.71% | 0.738 | 313 | 8.54% | 1.105 |
| Segregants | | | | | | | | | |
| 250 Male | M | 34,445 | 5.90% | 234 | 8.59% | 1.457 | 253 | 7.07% | 1.2 |
| Segregants | | | | | | | | | |
| Control | F | 13,263 | 2.39% | 79 | 2.92% | 1.222 | 113 | 3.09% | 1.294 |
| Line 1 | | | | | | | | | |
| Control | F | 11,226 | 2.21% | 10 | 0.37% | 0.167 | 25 | 0.68% | 0.308 |
| Line 2 | | | | | | | | | |
| Total | | 553,114 | 100% | 2,713 | 100% | — | 3,628 | 100% | — |

The results in Table 4 show that the “% Kernels in Bulks 1-4” for each germplasm ranged from 0.63% to 7.73% prior to the explant excision/production process. Excised corn seed explants were obtained from all germplasms tested, with each germplasm representing from 0.37% to 8.59% of the total number of explants identified by genotyping. Out of a total of 3,628 explants for all germplasms in Bulks 1-4 that generated shoots on culture media and were subsequently identified by genotyping, each germplasm represented 0.25% to 8.54% of the total number of shoots generated (excluding controls). These results indicate that all germplasms evaluated in Bulks 1-4 can be successfully excised from bulked seeds and that explants from each germplasm in Bulks 1-4 are capable of shoot generation. Variation in shoot generation capacity exists between germplasms after bulk excision as demonstrated by the ranges of values shown in Table 4.

The results in Table 5 show that each hybrid germplasm in Example 2, Experiment 1, Bulk 5, represented from 3.6% to 6.9% of the total number of seeds in Bulk 5 (see column 4 of Table 5). A total of 648 randomly selected explants from the Bulk 5 were sampled and identified by genotyping. The definitions for each of the columns and terminology used in Table 5 are as follows: “# Kernels in Bulk 5” (see column 3 of Table 5) is the number of kernels for a specific germplasm included in Bulk 5; “% Kernels in Bulk 5” (see column 4 of Table 5) is the “# Kernels in Bulk 5” for a specific germplasm divided by the total # of kernels for all germplasms in the Bulk×100; “# Explants” (see column 5 of Table 5) is the number of corn explants that were sampled and determined by genotyping to be the specific germplasm out of a total of 648 randomly sampled explants from Bulk 5; and “% Explants” (see column 6 of Table 5) is defined as the number of explants genotyped and determined to be the specific germplasm divided by the total # of explants identified by genotyping for all germplasms in Bulk 5 (i.e., 648)×100. In column 7 of Table 5, “Relative Explant Presence” is defined as the “% Explants” (see column 6 of Table 5) divided by the “% Kernels in Bulk 5” (see column 4 of Table 5) and indicates the relative representation of each germplasm in Bulk 5. “# Shoots Generated” represents the number of explants that generated shoots on culture media for each germplasm as determined by genotyping (see column 8 of Table 5). “% Shoots Generated” (see column 9 of Table 5) is defined as the number of explants that generated shoots on culture media and determined by genotyping to be the specific germplasm divided by total # of shoot-generating explants identified by genotyping for all germplasms in Bulk 5 (i.e., 784)×100. “Relative Shoot Presence” for each germplasm in column 10 of Table 5 is defined as the “% Shoots Generated” (see column 9 of Table 5) divided by the “% Kernels in Bulk 5” (see column 4 of Table 5) and indicates the relative representation of shoots generated of each germplasm in Bulk 5. Some samples were unable to be identified by genotyping due to missing data as described in Example 5 (<2% for the randomly selected explants, and 0% for the shoots), and were excluded.

TABLE 5
Response of different hybrids in bulked seed preparations to explant production,
culturing, and shoot generation (Example 2, Experiment 1; Bulk 5).

| | | | | | | 7 | | | 10 |
|---|---|---|---|---|---|---|---|---|---|
| | 2 | 3 | 4 | 5 | 6 | Relative | 8 | 9 | Relative |
| 1 | Heterotic | # Kernels | % Kernels | # | % | Explant | # Shoots | % Shoots | Shoot |
| Germplasm | group | in Bulk 5 | in Bulk 5 | Explants | Explants | Presence | Generated | Generated | Presence |
| H-1 | FxM | 14,460 | 4.30% | 32 | 4.90% | 1.144 | 43 | 5.50% | 1.271 |
| H-2 | FxM | 15,690 | 4.70% | 8 | 1.20% | 0.264 | 22 | 2.80% | 0.599 |
| H-3 | FxM | 14,100 | 4.20% | 29 | 4.50% | 1.064 | 32 | 4.10% | 0.970 |
| H-4 | FxM | 13,900 | 4.10% | 27 | 4.20% | 1.005 | 32 | 4.10% | 0.984 |
| H-5 | FxM | 12,990 | 3.90% | 29 | 4.50% | 1.155 | 25 | 3.20% | 0.823 |
| H-6 | FxM | 12,030 | 3.60% | 36 | 5.60% | 1.548 | 20 | 2.60% | 0.711 |
| H-7 | FxM | 15,360 | 4.60% | 48 | 7.40% | 1.616 | 57 | 7.30% | 1.586 |
| H-8 | FxM | 15,450 | 4.60% | 39 | 6.00% | 1.305 | 30 | 3.80% | 0.830 |
| H-9 | FxM | 15,570 | 4.60% | 33 | 5.10% | 1.096 | 69 | 8.80% | 1.894 |
| H-10 | FxM | 23,230 | 6.90% | 33 | 5.10% | 0.735 | 52 | 6.60% | 0.957 |
| H-11 | FxM | 21,930 | 6.50% | 38 | 5.90% | 0.896 | 45 | 5.70% | 0.877 |
| H-12 | FxM | 18,200 | 5.40% | 10 | 1.50% | 0.284 | 25 | 3.20% | 0.587 |
| H-13 | FxM | 20,500 | 6.10% | 36 | 5.60% | 0.908 | 44 | 5.60% | 0.917 |
| H-14 | FxM | 18,790 | 5.60% | 32 | 4.90% | 0.881 | 41 | 5.20% | 0.933 |
| H-15 | FxM | 16,380 | 4.90% | 30 | 4.60% | 0.947 | 36 | 4.60% | 0.939 |
| H-16 | FxM | 15,310 | 4.60% | 20 | 3.10% | 0.676 | 43 | 5.50% | 1.201 |
| H-17 | FxM | 15,330 | 4.60% | 36 | 5.60% | 1.214 | 34 | 4.30% | 0.948 |
| H-18 | FxM | 16,960 | 5.10% | 41 | 6.30% | 1.250 | 42 | 5.40% | 1.059 |
| H-19 | FxM | 17,230 | 5.10% | 45 | 6.90% | 1.351 | 48 | 6.10% | 1.191 |
| H-20 | FxM | 16,640 | 5.00% | 39 | 6.00% | 1.212 | 35 | 4.50% | 0.899 |
| Control | F | 2,770 | 0.80% | 5 | 0.80% | 0.934 | 7 | 0.90% | 1.125 |
| Line 1 | | | | | | | | | |
| Control | F | 2,300 | 0.70% | 2 | 0.30% | 0.450 | 2 | 0.30% | 0.429 |
| Line 2 | | | | | | | | | |
| Total | | 335,120 | 100% | 648 | 100% | — | 784 | 100% | — |

The results in Table 5 show that the “% Kernels in Bulk 5” for each germplasm (excluding controls) ranged from 3.6% to 6.9% before the explant excision/production process. Explants could be excised from each hybrid germplasm tested, with each hybrid germplasm representing from 1.2% to 7.4% of the total number of explants identified by genotyping. Out of a total of 784 explants from all germplasms in Bulk 5 that generated shoots on culture media and were subsequently identified by genotyping, each germplasm represented 2.6% to 8.8% of the total number of shoots generated (excluding controls). These results indicate that all tested hybrid germplasms in Bulk 5 can be excised from bulked seeds and that explants from each germplasm in the bulk are capable of shoot generation. Variations in shoot generation capacity exist between hybrid germplasms after bulk excision as demonstrated by the ranges of values shown in Table 5.

In a separate experiment focused on male corn germplasms, the batch of bulked seeds described in Example 1, Experiment 2 was subjected to automated seed explant excision, culturing, and shoot generation as described above for Example 2, Experiment 1. Except in Example 2, Experiment 2, approximately 9,200 explants were thawed and subsequently surface-sterilized in batches of approximately 500 explants per container (1 g dry weight). After rehydration in Example 2, Experiment 2, explants were plated on regeneration medium without selection at a density of approximately 33 explants per container (1 g dry weight over 16 containers).

The results in Table 6 represent combined batches and 2 genotyping replicates. Each germplasm, denoted as M2-#, represents from 0.14% to 40.14% of the total number of corn kernels in the bulk (see “# Kernels in Bulk,” column 4 of Table 6). A total of 695 (Rep 1) and 699 (Rep 2) randomly selected explants from the bulk were identified by genotyping. In addition, samples from a total of 1,011 (Rep 1) and 1,970 (Rep 2) generated shoots were collected and identified by genotyping. Some samples were unable to be identified by genotyping due to missing data as described in Example 5 (averaged <1% for the randomly selected explants, and <3% for the shoots), and were excluded.

The definitions for each of the columns and terminology used in Table 6 are the same as provided above for Bulk 5 in Table 5, except that the numbers and calculations are for 2 replicates of male germplasms in Example 2, Experiment 2.

TABLE 6
Response of different male germplasms in bulked explant preparation to explant
production, culturing, and shoot generation (Example 2, Experiment 2).

| | 2 | 3 | | | | 7 | 8 | 9 | 10 |
|---|---|---|---|---|---|---|---|---|---|
| | # | % | | 5 | 6 | Relative | # | % | Relative |
| 1 | Kernels | Kernels | 4 | # | % | Explant | Generated | Generated | Shoot |
| Germplasm | in Bulk | in Bulk | Replicate | Explants | Explants | Presence | Shoots | Shoots | Presence |
| M2-1 | 60,000 | 7.50% | 1 | 47 | 6.76% | 0.902 | 87 | 8.61% | 1.1474 |
| | | | 2 | 58 | 8.30% | 1.1063 | 151 | 7.66% | 1.022 |
| M2-2 | 40,000 | 5.00% | 1 | 18 | 2.59% | 0.518 | 16 | 1.58% | 0.3165 |
| | | | 2 | 25 | 3.58% | 0.7153 | 33 | 1.68% | 0.335 |
| M2-3 | 250,000 | 31.25% | 1 | 279 | 40.14% | 1.285 | 395 | 39.07% | 1.2502 |
| | | | 2 | 261 | 37.34% | 1.1948 | 735 | 37.31% | 1.1939 |
| M2-4 | 25,000 | 3.13% | 1 | 34 | 4.89% | 1.565 | 26 | 2.57% | 0.8229 |
| | | | 2 | 31 | 4.43% | 1.4192 | 63 | 3.20% | 1.0234 |
| M2-5 | 4,000 | 0.50% | 1 | 2 | 0.29% | 0.576 | 8 | 0.79% | 1.5826 |
| | | | 2 | 2 | 0.29% | 0.5722 | 14 | 0.71% | 1.4213 |
| M2-6 | 40,000 | 5.00% | 1 | 55 | 7.91% | 1.583 | 74 | 7.32% | 1.4639 |
| | | | 2 | 41 | 5.87% | 1.1731 | 141 | 7.16% | 1.4315 |
| M2-7 | 30,000 | 3.75% | 1 | 35 | 5.04% | 1.343 | 48 | 4.75% | 1.2661 |
| | | | 2 | 38 | 5.44% | 1.4497 | 90 | 4.57% | 1.2183 |
| M2-8 | 8,000 | 1.00% | 1 | 5 | 0.72% | 0.719 | 4 | 0.40% | 0.3956 |
| | | | 2 | 8 | 1.14% | 1.1445 | 13 | 0.66% | 0.6599 |
| M2-9 | 40,000 | 5.00% | 1 | 29 | 4.17% | 0.835 | 30 | 2.97% | 0.5935 |
| | | | 2 | 33 | 4.72% | 0.9442 | 88 | 4.47% | 0.8934 |
| M2-10 | 30,000 | 3.75% | 1 | 36 | 5.18% | 1.381 | 43 | 4.25% | 1.1342 |
| | | | 2 | 29 | 4.15% | 1.1063 | 82 | 4.16% | 1.11 |
| M2-11 | 10,000 | 1.25% | 1 | 8 | 1.15% | 0.921 | 5 | 0.49% | 0.3956 |
| | | | 2 | 7 | 1.00% | 0.8011 | 14 | 0.71% | 0.5685 |
| M2-12 | 20,000 | 2.50% | 1 | 23 | 3.31% | 1.324 | 47 | 4.65% | 1.8595 |
| | | | 2 | 15 | 2.15% | 0.8584 | 72 | 3.65% | 1.4619 |
| M2-13 | 25,000 | 3.13% | 1 | 12 | 1.73% | 0.553 | 28 | 2.77% | 0.8863 |
| | | | 2 | 28 | 4.01% | 1.2818 | 55 | 2.79% | 0.8934 |
| M2-14 | 10,000 | 1.25% | 1 | 18 | 2.59% | 2.072 | 20 | 1.98% | 1.5826 |
| | | | 2 | 10 | 1.43% | 1.1445 | 48 | 2.44% | 1.9492 |
| M2-15 | 100,000 | 12.50% | 1 | 34 | 4.89% | 0.391 | 82 | 8.11% | 0.6489 |
| | | | 2 | 56 | 8.01% | 0.6409 | 161 | 8.17% | 0.6538 |
| M2-16 | 30,000 | 3.75% | 1 | 22 | 3.17% | 0.844 | 29 | 2.87% | 0.7649 |
| | | | 2 | 16 | 2.29% | 0.6104 | 66 | 3.35% | 0.8934 |
| M2-17 | 15,000 | 1.88% | 1 | 18 | 2.59% | 1.381 | 23 | 2.27% | 1.2133 |
| | | | 2 | 19 | 2.72% | 1.4497 | 47 | 2.39% | 1.2724 |
| M2-18 | 25,000 | 3.13% | 1 | 17 | 2.45% | 0.783 | 31 | 3.07% | 0.9812 |
| | | | 2 | 13 | 1.86% | 0.5951 | 55 | 2.79% | 0.8934 |
| M2-19 | 30,000 | 3.75% | 1 | 2 | 0.29% | 0.077 | 9 | 0.89% | 0.2374 |
| | | | 2 | 6 | 0.86% | 0.2289 | 33 | 1.68% | 0.4467 |
| M2-20 | 8,000 | 1.00% | 1 | 1 | 0.14% | 0.144 | 6 | 0.59% | 0.5935 |
| | | | 2 | 3 | 0.43% | 0.4292 | 9 | 0.46% | 0.4569 |
| Total | 800,000 | 100% | 1 | 695 | 100% | — | 1,011 | 100% | — |
| | | | 2 | 699 | 100% | — | 1,970 | 100% | — |

The results of Example 2, Experiment 2 demonstrate that excised corn seed explants were obtained from all germplasms tested in the experiment, with each germplasm representing 0.14% to 40.14% of the total explants identified by genotyping. Out of a total of 1,011 (Rep 1) and 1,970 (Rep 2) explants that generated shoots on culture media and were subsequently identified by genotyping, each germplasm represented 0.4% to 39.07% of the total number of shoots generated and identified. These results indicate that all tested male germplasms in Example 2, Experiment 2 can be successfully excised from bulked seeds and that explants from each bulk are capable of shoot generation. Variation in regenerative capacity exists between germplasms after bulk excision as seen in the ranges of values in Table 6.

In summary, the current bulk corn seed explant production process can be applied to all tested germplasms to produce shoot generating explants. Different germplasms respond differently to the explant production based on the high variation in “Relative Explant Presence” and “% Shoots Generated” in Tables 4-6. These results indicate that many germplasms are capable of bulked explant excision, but the variation may indicate that bulk processing of at least some germplasms may benefit from further process optimization. In addition, these results indicate that processing of explants in bulk does not eliminate shoot generating abilities.

### Example 3

### Shoot Generation Ability after Agrobacterium Inoculation of Bulked Corn Excised Explants

Bulked explants comprising many different corn germplasms were evaluated for their ability to grow and generate shoots following Agrobacterium inoculation in Example 3, Experiment 1 and Example 3, Experiment 2 (Tables 7-9). Briefly, explants from bulked seeds were inoculated with Agrobacterium, placed on regeneration medium for two weeks to allow shoot generation, and sampled for genotyping.

Agrobacterium inoculation for Example 3, Experiments 1 and 2 was performed as follows: briefly, corn excised explants were surface-sterilized and rehydrated as described in Example 2. For Agrobacterium preparation, an aliquot of 250 mL of an Agrobacterium glycerol stock solution was inoculated into 250 mL of LB medium with the appropriate antibiotics for the Agrobacterium strain comprising a recombinant DNA construct containing a β-glucuronidase reporter gene. The mixture was cultured with shaking (200 RPM+/−10) at 27.5+/−2° C. for 16-24 hours or until OD₆₆₀ reached 0.7-1.2. After the Agrobacterium cells were pelleted at 3500 RCF for 25 min at 4+/−3° C. and the supernatant was discarded, the Agrobacterium cells were resuspended in 50 mL inoculation medium (⅖ of B5 macro salts, 1/10 of B5 micro salts and vitamins, 1 g/L potassium nitrate, 30 g/L dextrose and 3.9 g/L MES) to a final OD₆₆₀ of 0.25.

The prepared corn explants were weighed and measured to determine number of explants per gram. In Example 3, Experiment 1, approximately 1,500-2,000 explants per bulk, totaling approximately 8,000 explants were utilized. Batches of approximately 2,000 explants were inoculated in 40-50 mL of the Agrobacterium preparation and subjected to a pressure of 300 psi for 3 minutes. After treatment in the pressure chamber, explants from Experiment 1 were centrifuged for 30 minutes at 4° C. Following inoculation, the Agrobacterium solution was removed, and the inoculated explants were blotted on a sterile paper towel to remove excess inoculation medium.

Inoculated explants were plated on solid regeneration medium without selection containing MS salts, B5 vitamins, 30 g/L sucrose, 0.69 g/L proline, 1 g/L NZ amine-A, 2 mg/L glycine, 1 g/L MES, 400 mg/L carbenicillin, 200 mg/L cefotaxime, 100 mg/L timentin, 3.5 g/L low EEO agarose, pH 5.8, at a density of about 100 explants per 600 mL culture vessel in Experiment 1. The vessels were incubated at 28+/−3° C. with a photoperiod of 16-hour light/8-hour dark, and a light intensity target of 160 PAR (about 140 to about 200 PAR is acceptable) for about two weeks. Samples were collected from regenerated plants of at least 5 cm in size with strong meristems and genotyped using genetic markers as described in Example 5.

In Example 3, Experiment 1; Bulks 1-4, each germplasm generated shoots after inoculation, as shown in Table 7. In Table 7, “# Kernels in Bulks 1-4” (see column 3 of Table 7) is the total number of kernels for a specific germplasm included in Bulks 1-4; “% Kernels in Bulks 1-4” (see column 4 of Table 7) is the “# Kernels” for a specific germplasm in a single bulk divided by the total number of kernels in that Bulk×100, calculated for each of Bulks 1-4 separately then averaged (summed and divided by 4) (i.e., (((# of M-1 Kernels in Bulk 1/Total # Kernels in Bulk 1)×100)+((# of M-1 Kernels in Bulk 2/Total # Kernels in Bulk 2)×100)+((# of M-1 Kernels in Bulk 3/Total # Kernels in Bulk 3)×100)+((# of M-1 Kernels in Bulk 4/Total # Kernels in Bulk 4)×100)/4); “# Shoots Generated after Agro” (see column 5 of Table 7) represents the number of explants that generated shoots after Agrobacterium inoculation and were determined by genotyping to be of a specific germplasm from a total of 2,769 shoots generated and identified by genotyping from all germplasms in Bulks 1-4; and “% Shoots Generated after Agro” (see column 6 of Table 7) is defined as the “# of Explants” for a specific germplasm in a single bulk that generated shoots divided by the total number of explants identified by genotyping in that bulk that generated shoots×100, calculated for each of Bulks 1-4 separately then averaged (summed and divided by 4). In column 7 of Table 7, “Relative Shoot Presence after Agro” is defined as the “% Shoots Generated after Agro” (see column 6 of Table 7) divided by the “% Kernels in Bulks 1-4” (see column 4 of Table 7) and indicates the relative representation of the shoot generation of each germplasm in Bulks 1-4 after Agrobacterium inoculation. Some samples were unable to be identified by genotyping due to missing data as described in Example 5 (averaged <4%) and were excluded.

TABLE 7
Response of different germplasms in bulked explant preparations to
Agrobacterium inoculation (Example 3, Experiment 1; Bulks 1-4).

| | | 3 | 4 | 5 | 6 | 7 |
|---|---|---|---|---|---|---|
| | 2 | # Kernels | % Kernels | # Shoots | % Shoots | Relative Shoot |
| 1 | Heterotic | in Bulks | in Bulks | Generated | Generated | Presence After |
| Germplasm | group | 1-4 | 1-4 | After Agro | After Agro | Agro |
| F-1 | F | 5,766 | 1.01% | 27 | 0.96% | 0.95 |
| F-2 | F | 5,956 | 1.10% | 6 | 0.22% | 0.2 |
| F-3 | F | 5,677 | 1.03% | 31 | 1.13% | 1.1 |
| F-4 | F | 6,357 | 1.21% | 29 | 1.07% | 0.88 |
| F-5 | F | 5,754 | 0.97% | 10 | 0.36% | 0.36 |
| F-6 | F | 5,229 | 0.91% | 10 | 0.36% | 0.4 |
| F-7 | F | 5,813 | 1.12% | 38 | 1.40% | 1.25 |
| F-8 | F | 5,992 | 1.09% | 28 | 0.94% | 0.86 |
| F-9 | F | 5,893 | 1.20% | 51 | 1.89% | 1.57 |
| F-10 | F | 6,093 | 1.23% | 21 | 0.78% | 0.63 |
| F-11 | F | 6,092 | 1.10% | 29 | 1.04% | 0.95 |
| F-12 | F | 6,205 | 1.18% | 14 | 0.52% | 0.44 |
| F-13 | F | 5,772 | 1.03% | 14 | 0.50% | 0.48 |
| F-14 | F | 6,024 | 1.20% | 9 | 0.34% | 0.28 |
| F-15 | F | 5,900 | 1.15% | 26 | 0.99% | 0.87 |
| F-16 | F | 6,159 | 1.15% | 12 | 0.45% | 0.39 |
| F-17 | F | 6,125 | 1.17% | 19 | 0.70% | 0.6 |
| F-18 | F | 6,357 | 1.07% | 45 | 1.64% | 1.53 |
| F-19 | F | 6,085 | 1.12% | 62 | 2.22% | 1.98 |
| F-20 | F | 6,277 | 1.17% | 28 | 1.02% | 0.87 |
| F-21 | F | 5,948 | 1.19% | 21 | 0.76% | 0.64 |
| F-22 | F | 6,182 | 1.12% | 21 | 0.76% | 0.68 |
| F-23 | F | 6,318 | 1.22% | 17 | 0.64% | 0.52 |
| F-24 | F | 6,068 | 1.19% | 21 | 0.75% | 0.63 |
| F-25 | F | 6,187 | 1.11% | 38 | 1.35% | 1.22 |
| F-26 | F | 5,980 | 1.11% | 37 | 1.31% | 1.18 |
| F-27 | F | 6,150 | 1.16% | 54 | 1.96% | 1.69 |
| F-28 | F | 5,298 | 1.06% | 20 | 0.74% | 0.69 |
| F-29 | F | 6,126 | 1.15% | 25 | 0.89% | 0.77 |
| F-20 | F | 7,013 | 1.29% | 32 | 1.14% | 0.88 |
| F-31 | F | 6,422 | 1.12% | 35 | 1.27% | 1.14 |
| F-32 | F | 5,815 | 1.08% | 41 | 1.51% | 1.4 |
| F-33 | F | 5,963 | 1.08% | 22 | 0.76% | 0.71 |
| F-34 | F | 5,423 | 0.97% | 65 | 2.31% | 2.39 |
| F-35 | F | 5,554 | 1.09% | 75 | 2.70% | 2.49 |
| F-36 | F | 5,486 | 0.93% | 52 | 1.89% | 2.03 |
| F-37 | F | 6,003 | 1.19% | 47 | 1.78% | 1.49 |
| F-38 | F | 5,814 | 1.15% | 54 | 2.00% | 1.73 |
| M-1 | M | 5,343 | 0.89% | 51 | 1.82% | 2.05 |
| M-2 | M | 5,654 | 0.96% | 14 | 0.50% | 0.52 |
| M-3 | M | 5,995 | 1.09% | 36 | 1.31% | 1.2 |
| M-4 | M | 5,566 | 0.90% | 20 | 0.72% | 0.8 |
| M-5 | M | 4,230 | 0.63% | 11 | 0.39% | 0.62 |
| M-6 | M | 6,293 | 1.05% | 32 | 1.09% | 1.04 |
| M-7 | M | 6,090 | 0.97% | 19 | 0.69% | 0.71 |
| M-8 | M | 6,067 | 1.02% | 22 | 0.80% | 0.78 |
| M-9 | M | 5,801 | 1.05% | 48 | 1.72% | 1.64 |
| M-10 | M | 5,889 | 0.98% | 6 | 0.20% | 0.2 |
| M-11 | M | 6,095 | 1.05% | 27 | 0.98% | 0.93 |
| M-12 | M | 5,215 | 0.93% | 38 | 1.36% | 1.45 |
| M-13 | M | 5,685 | 1.03% | 38 | 1.34% | 1.3 |
| M-14 | M | 5,947 | 0.95% | 24 | 0.83% | 0.88 |
| M-15 | M | 5,662 | 1.06% | 22 | 0.77% | 0.73 |
| M-16 | M | 5,873 | 0.97% | 33 | 1.20% | 1.23 |
| M-17 | M | 6,026 | 1.03% | 22 | 0.80% | 0.78 |
| M-18 | M | 5,544 | 1.05% | 31 | 1.15% | 1.09 |
| M-19 | M | 5,907 | 1.13% | 17 | 0.64% | 0.57 |
| M-20 | M | 5,296 | 0.94% | 37 | 1.28% | 1.37 |
| M-21 | M | 9,175 | 1.74% | 48 | 1.79% | 1.03 |
| M-22 | M | 5,896 | 1.04% | 39 | 1.35% | 1.3 |
| M-23 | M | 5,461 | 0.94% | 25 | 0.90% | 0.96 |
| M-24 | M | 6,028 | 1.04% | 27 | 0.97% | 0.94 |
| M-25 | M | 6,019 | 0.92% | 19 | 0.67% | 0.72 |
| M-26 | M | 6,028 | 1.07% | 26 | 0.95% | 0.89 |
| M-27 | M | 5,883 | 1.05% | 35 | 1.28% | 1.22 |
| M-28 | M | 6,100 | 1.11% | 23 | 0.83% | 0.75 |
| M-29 | M | 6,034 | 1.09% | 30 | 1.09% | 1 |
| M-30 | M | 5,531 | 0.99% | 23 | 0.84% | 0.85 |
| M-31 | M | 6,081 | 1.09% | 26 | 0.95% | 0.87 |
| M-32 | M | 6,121 | 1.11% | 28 | 1.02% | 0.92 |
| M-33 | M | 6,057 | 1.08% | 13 | 0.48% | 0.45 |
| M-34 | M | 6,511 | 1.18% | 25 | 0.90% | 0.77 |
| M-35 | M | 6,116 | 1.14% | 20 | 0.72% | 0.63 |
| M-36 | M | 5,415 | 1.00% | 27 | 0.97% | 0.97 |
| M-37 | M | 5,957 | 1.06% | 23 | 0.83% | 0.78 |
| M-38 | M | 5,610 | 1.04% | 29 | 1.03% | 0.99 |
| 250 Female | F | 42,703 | 7.73% | 225 | 8.10% | 1.05 |
| Segregants | | | | | | |
| 250 Male | M | 34,445 | 5.90% | 216 | 7.76% | 1.32 |
| Segregants | | | | | | |
| Control | F | 13,263 | 2.39% | 103 | 3.75% | 1.57 |
| Line 1 | | | | | | |
| Control | F | 11,226 | 2.21% | 5 | 0.19% | 0.08 |
| Line 2 | | | | | | |
| Total | | 553,114 | 100% | 2,769 | 100% | — |

As shown in Table 7, shoots were generated and identified by genotyping from a total of 2,769 explants after Agrobacterium inoculation, representing every germplasm tested in Bulks 1-4. The “% Shoots Generated after Agro” ranged from 0.2% to 8.1% (excluding controls). These results indicate that all tested germplasms (inbred female, inbred male, and male and female segregants) in Bulks 1-4 were capable of generating shoots after inoculation with Agrobacterium and that such bulked germplasm explants can be transformed via Agrobacterium inoculation. As demonstrated in Table 7, different germplasms respond differently to Agrobacterium inoculation, as indicated by the range of shoot generation frequencies shown (see, e.g., the “% Shoots Generated after Agro”).

As shown in Table 8, all hybrid germplasms tested in Example 3, Experiment 1; Bulk 5, generated shoots after inoculation with Agrobacterium (20/20). The negative control (Control Line 2) failed to generate shoots following inoculation, but the positive control (Control Line 1) generated shoots. The definitions for each of the columns and terminology used in Table 8 are as follows: “# Kernels in Bulk 5” (see column 3 of Table 8) is the number of kernels for a specific germplasm included in Bulk 5; “% Kernels in Bulk 5” (see column 4 of Table 8) is the “# Kernels in Bulk 5” for a specific germplasm divided by the total # of kernels for all germplasms in the Bulk×100; “# Shoots Generated after Agro” (see column 5 of Table 8) represents the number of explants that generated shoots after Agrobacterium inoculation and were determined by genotyping to be of a specific germplasm from a total of 839 genotyped and regenerated shoots from all germplasms in Bulk 5; “% Shoots Generated after Agro” (see column 6 of Table 8) represents the # of explants that generated shoots for a specific germplasm divided by the total # of explants sampled and identified for all germplasms in Bulk 5 (i.e., 839)×100. In column 7 of Table 8, “Relative Shoot Presence after Agro” is defined as the “% Shoots Generated after Agro” (see column 6 of Table 8) divided by “% Kernels in Bulk 5” (see column 4 of Table 8) and indicates the relative representation of the shoot generation of each germplasm in Bulk 5 after Agrobacterium inoculation. Some samples were unable to be identified by genotyping due to missing data as described in Example 5 (<5%) and were excluded.

TABLE 8
Response of different hybrids in bulked explant preparations to
Agrobacterium inoculation (Example 3, Experiment 1; Bulk 5).

| | | | | 5 | 6 | 7 |
|---|---|---|---|---|---|---|
| | 2 | 3 | 4 | # Shoots | % Shoots | Relative Shoot |
| 1 | Heterotic | # Kernels | % Kernels | Generated | Generated | Presence After |
| Germplasm | group | in Bulk 5 | in Bulk 5 | After Agro | After Agro | Agro |
| H-1 | FxM | 14,460 | 4.30% | 41 | 4.90% | 1.133 |
| H-2 | FxM | 15,690 | 4.70% | 38 | 4.50% | 0.967 |
| H-3 | FxM | 14,100 | 4.20% | 24 | 2.90% | 0.680 |
| H-4 | FxM | 13,900 | 4.10% | 40 | 4.80% | 1.149 |
| H-5 | FxM | 12,990 | 3.90% | 25 | 3.00% | 0.769 |
| H-6 | FxM | 12,030 | 3.60% | 39 | 4.60% | 1.295 |
| H-7 | FxM | 15,360 | 4.60% | 51 | 6.10% | 1.326 |
| H-8 | FxM | 15,450 | 4.60% | 42 | 5.00% | 1.086 |
| H-9 | FxM | 15,570 | 4.60% | 40 | 4.80% | 1.026 |
| H-10 | FxM | 23,230 | 6.90% | 70 | 8.30% | 1.204 |
| H-11 | FxM | 21,930 | 6.50% | 43 | 5.10% | 0.783 |
| H-12 | FxM | 18,200 | 5.40% | 24 | 2.90% | 0.527 |
| H-13 | FxM | 20,500 | 6.10% | 45 | 5.40% | 0.877 |
| H-14 | FxM | 18,790 | 5.60% | 50 | 6.00% | 1.063 |
| H-15 | FxM | 16,380 | 4.90% | 44 | 5.20% | 1.073 |
| H-16 | FxM | 15,310 | 4.60% | 47 | 5.60% | 1.226 |
| H-17 | FxM | 15,330 | 4.60% | 43 | 5.10% | 1.120 |
| H-18 | FxM | 16,960 | 5.10% | 47 | 5.60% | 1.107 |
| H-19 | FxM | 17,230 | 5.10% | 49 | 5.80% | 1.136 |
| H-20 | FxM | 16,640 | 5.00% | 28 | 3.30% | 0.672 |
| Control | F | 2,770 | 0.80% | 9 | 1.10% | 1.298 |
| Line 1 | | | | | | |
| Control | F | 2,300 | 0.70% | 0 | 0.00% | 0.000 |
| Line 2 | | | | | | |
| Total | | 335,120 | 100% | 839 | 100% | — |

As shown in Table 8, a total of 839 hybrid explants generated shoots and were identified by genotyping after Agrobacterium inoculation, representing 21/22 germplasms tested in Bulk 5. Control Line 2 failed to produce shoots after Agrobacterium inoculation. This was expected as the line is known to respond poorly to the Agrobacterium inoculation and shoot generation protocol. The “% Shoots Generated after Agro” ranged from 2.9% to 8.3% (excluding the controls). These results indicate that most tested germplasms, with the exception of the negative control in Bulk 5, are capable of shoot generation after challenge with Agrobacterium and can be transformed via Agrobacterium inoculation. The range of shoot generation frequencies after Agrobacterium inoculation shown in Table 8 (represented by the “% Shoots Generated after Agro”) indicate that different germplasms respond differently to the treatments.

The prepared corn explants for Example 3, Experiment 2 were weighed and measured to determine the number of explants per gram and inoculated as described for Example 3, Experiment 1. Except in Example 3, Experiment 2, approximately 11,000 total explants over 17 replicates were utilized (1-2 g dry weight of explants per replicate) and inoculated in batches of approximately 1,000 explants. After treatment in the pressure chamber (as described for Example 3, Experiment 1), explants from Example 3, Experiment 2 were centrifuged for 30 minutes at 4° C. Shoots were generated from explants in Example 3, Experiment 2 as described in Example 3, Experiment 1. Nearly all male germplasms tested in Example 3, Experiment 2 were also capable of generating shoots after Agrobacterium inoculation. As shown in Table 9, 19/20 male germplasms tested generated shoots after Agrobacterium inoculation. The definitions for each of the columns and terminology used in Table 9 are the same as provided in Table 8 for Example 3, Experiment 1; Bulk 5, except that the numbers and calculations are for male germplasms in Example 3, Experiment 2 out of a total of 719 shoots identified by genotyping. Note that some shoots were unable to be identified by genotyping due to missing data as described in Example 5 (<5%) and were excluded from Table 9.

TABLE 9
Response of different male germplasms in bulked explant preparations
to Agrobacterium inoculation (Example 3, Experiment 2).

| | | | | 5 | 6 | 7 |
|---|---|---|---|---|---|---|
| | 2 | 3 | 4 | # Shoots | % Shoots | Relative Shoot |
| 1 | Heterotic | # Kernels | % Kernels | Generated | Generated | Presence After |
| Germplasm | group | in Bulk | in Bulk | After Agro | After Agro | Agro |
| M2-1 | M | 60,000 | 7.50% | 109 | 15.16% | 2.0213 |
| M2-2 | M | 40,000 | 5.00% | 18 | 2.50% | 0.5007 |
| M2-3 | M | 250,000 | 31.25% | 237 | 32.96% | 1.0548 |
| M2-4 | M | 25,000 | 3.13% | 9 | 1.25% | 0.4006 |
| M2-5 | M | 4,000 | 0.50% | 0 | 0.00% | 0.0000 |
| M2-6 | M | 40,000 | 5.00% | 20 | 2.78% | 0.5563 |
| M2-7 | M | 30,000 | 3.75% | 46 | 6.40% | 1.7061 |
| M2-8 | M | 8,000 | 1.00% | 7 | 0.97% | 0.9736 |
| M2-9 | M | 40,000 | 5.00% | 23 | 3.20% | 0.6398 |
| M2-10 | M | 30,000 | 3.75% | 14 | 1.95% | 0.5192 |
| M2-11 | M | 10,000 | 1.25% | 3 | 0.42% | 0.3338 |
| M2-12 | M | 20,000 | 2.50% | 16 | 2.23% | 0.8901 |
| M2-13 | M | 25,000 | 3.13% | 37 | 5.15% | 1.6467 |
| M2-14 | M | 10,000 | 1.25% | 10 | 1.39% | 1.1127 |
| M2-15 | M | 100,000 | 12.50% | 120 | 16.69% | 1.3352 |
| M2-16 | M | 30,000 | 3.75% | 6 | 0.83% | 0.2225 |
| M2-17 | M | 15,000 | 1.88% | 3 | 0.42% | 0.2225 |
| M2-18 | M | 25,000 | 3.13% | 30 | 4.17% | 1.3352 |
| M2-19 | M | 30,000 | 3.75% | 6 | 0.83% | 0.2225 |
| M2-20 | M | 8,000 | 1.00% | 5 | 0.70% | 0.6954 |
| Total | | 800,000 | 100% | 719 | 100% | — |

As shown in Table 9, a total of 719 explants from the male inbred lines generated shoots and were identified by genotyping after Agrobacterium inoculation, representing 19 out of 20 germplasms tested in Example 3, Experiment 2. The “% Shoots Generated after Agro” ranged from 0% to 30.62%. Some samples could not be positively identified and were excluded. These results indicate that most germplasms tested in Example 3, Experiment 2, with the exception of one male line, were capable of shoot generation after inoculation with Agrobacterium, indicating that bulked explants of male germplasms can be transformed via Agrobacterium. The range of shoot generation frequencies after Agrobacterium inoculation demonstrated in Table 9 (represented by the “% Shoots Generated after Agro”) indicate that different germplasms respond differently to the treatments.

In summary, the results show that the current bulk corn seed explant production process can be used to not only produce viable explants from all tested germplasms, but also to generate shoots from most tested female and male germplasms following Agrobacterium inoculation. These results are positive indicators of the potential for bulked explants to survive Agrobacterium-mediated transformation and subsequent shoot generation as described in Example 4.

### Example 4

### Analysis of Transformation and Editing Frequencies of Bulked Corn Excised Explants

The transformation frequencies of bulked explants comprising many different corn germplasms following Agrobacterium-mediated transformation were evaluated for explants from Example 3, Experiment 1; Bulks 3 & 4 only, Example 3, Experiment 2, and Example 3, Experiment 3 (see Tables 10-14). Explants from Example 3, Experiment 1; Bulks 3 & 4 and Example 3, Experiment 3 were additionally analyzed for the frequency of successful gene edits following introduction of a gene editing construct.

Approximately 200,000 total excised corn seed explants from Example 3, Experiment 1; Bulks 3 & 4 were inoculated with Agrobacterium harboring a gene editing construct in 7 batches of 10,000-40,000 explants each, according to the methods described in Example 3, Experiment 1. Approximately 2,000-2,500 explants were inoculated per inoculation container. After inoculation, the Agrobacterium solution was removed, and the inoculated explants were blotted on a sterile paper towel to remove excess Agrobacterium solution. Explants were then transferred to co-culture Petri plates (100 mm) with a piece of sterile Whatman #1 filter paper (82 mm) wetted with 1.25 mL of the rehydration media (⅖ strength B5 macro salts except foe/2 strength CaCl₂), 1/10 strength of B5 micro salts and vitamins, 1 g/L potassium nitrate, 30 g/L dextrose, 2.8 mg/L sequestrene, 3.9 g/L MES, and 0.03 g/L Clearys 3336 WP, pH 5.4), at a density of approximately 550-600 explants/plate arranged in a single layer. The co-culture plates were incubated at 20+/−1° C. for 6 days, with a photoperiod of 16-hours light/8-hour dark, a light intensity target of 90 PAR (ranging from about 65 to about 115 PAR), and a relative humidity (Rh) of approximately 65+/−10%.

After co-culture, explants were transferred to Petri plates (1 co-culture plate to 5 Petri plates) containing a bud induction medium of MS salts, B5 vitamins, 30 g/L sucrose, 0.69 g/L proline, 1 g/L NZ amine-A, 2 mg/L glycine, 1 g/L MES, 1 mg/L 2, 4-D, 10 mg/L BAP, 400 mg/L carbenicillin, 200 mg/L cefotaxime, 100 mg/L timentin, 3.5 g/L low EEO agarose, pH 5.8, at a density of about 100-150 explants per plate and cultured for 7 days at 33+/−1° C., with a photoperiod of 16-hour light/8-hour dark, and a light intensity target of 90 PAR (ranging from about 65 to about 115 PAR).

The explants were then transferred to Petri plates (1 bud induction plate to 2 extended bud induction plates) containing extended bud induction medium containing MS basal salts, B5 vitamins, 60 g/L sucrose, 0.5 g/L glutamine, 1 g/L NZ Amine-A, 0.69 g/L proline, 2 mg/L glycine, 1.95 g/L MES, 1.25 mg/L cupric sulfate, thidiazuron, 2 mg/L picloram, 400 mg/L carbenicillin, 200 mg/L cefotaxime, 100 mg/L timentin, 25 μM glyphosate, and 3.5 g/L low EEO agarose, pH 5.8, at a target density of about 57 explants/plate and cultured at 28+/−3° C. for 14 days, with a photoperiod of 16-hour light/8-hour dark, and a light intensity target of 150 PAR (ranging from about 140 to about 160 PAR).

Finally, explants were transferred to regeneration medium containing LM Woody Plant Medium salts and vitamins, 0.03 g/L Clearys 3336 WP, 30 g/L sucrose, 0.69 g/L proline, 2 mg/L glycine, 1 g/L MES, 3.5 g/L low EEO agarose, 400 mg/L carbenicillin, 200 mg/L cefotaxime, 100 mg/L timentin and 20 μM glyphosate, pH 5.8 in 9 cm Vivi trays (Vivi, The Netherlands), at a density of approximately 150-200 explants/tray, and cultured at 28+/−3° C. for 6 weeks, with a photoperiod of 16-hour light/8-hour dark, and a light intensity target of 160 PAR (ranging from about 140 to about 200 PAR). Regenerated plants were collected and assayed for genotyping, transformation, and confirmation of gene editing in the bulk explants.

As shown in Table 10, a total of 139 explants identified by genotyping were successfully transformed. The “% Transformants” ranged from 0% to 15.1%, and there were 96 successful edits by the gene editing construct, including 16 edits in the positive control line.

In Table 10, “# Kernels in Bulks 3 & 4” (see column 3 of Table 10) is the number of kernels for a specific germplasm included in Bulks 3 & 4; “% Kernels in Bulks 3 & 4” (see column 4 of Table 10) is the “# Kernels in Bulks 3 & 4” for a specific germplasm divided by the total # of kernels for all germplasms in Bulks 3 & 4×100; “# Transformants” (see column 5 of Table 10) is the number of explants that were transformed for a specific germplasm of a total of 139 explants identified by genotyping from all germplasms in Bulks 3 & 4; and “% Transformants” (see column 6 of Table 10) represents # of explants that were transformed for a specific germplasm divided by the total # of explants that were transformed and identified by genotyping for all germplasms in Bulks 3 & 4 (e.g., 139 for Bulks 3 & 4)×100. In column 7 of Table 10, “Relative Transformation Rate” is defined as the “% Transformants” divided by the “% Kernels in Bulks 3 & 4” (see column 4 of Table 10) and indicates the relative transformation frequency of each germplasm in Bulks 3 & 4 (compared to all lines in the bulk). The number of transformed explants identified by genotyping that contained germplasm edits (“# Edited”) is shown in column 8 of Table 10. “% Edited” represents # of transformed explants that produced edited plants for a specific germplasm divided by the total # of explants (96) that produced edited plants for all germplasms identified by genotyping in Bulks 3 & 4. The “Relative Editing Rate” for each germplasm in column 10 of Table 10 is defined as the “% Edited” (see column 9 of Table 10) divided by the “% Kernels in Bulks 3 & 4” (see column 4 of Table 10) and indicates the relative representation of edited plants of each germplasm in Bulks 3 & 4.

TABLE 10
Response of different germplasms in bulked explant preparations to Agrobacterium-mediated
transformation and editing (Example 4, Experiment 1; Bulks 3 & 4).

| | | 3 | 4 | | | 7 | | | 10 |
|---|---|---|---|---|---|---|---|---|---|
| | 2 | # Kernels | % Kernels | 5 | 6 | Relative | 8 | 9 | Relative |
| 1 | Heterotic | in Bulks | in Bulks | # | % | Transformation | # | % | Editing |
| Germplasm | group | 3 & 4 | 3 & 4 | Transformants | Transformants | Rate | Edited | Edited | Rate |
| F-1 | F | 4,160 | 1.40% | 0 | 0.00% | 0 | 0 | 0.0% | 0.00 |
| F-2 | F | 2,269 | 0.80% | 0 | 0.00% | 0 | 0 | 0.0% | 0.00 |
| F-3 | F | 3,897 | 1.30% | 0 | 0.00% | 0 | 0 | 0.0% | 0.00 |
| F-4 | F | 2,906 | 1.00% | 0 | 0.00% | 0 | 0 | 0.0% | 0.00 |
| F-5 | F | 4,368 | 1.50% | 0 | 0.00% | 0 | 0 | 0.0% | 0.00 |
| F-6 | F | 4,217 | 1.40% | 0 | 0.00% | 0 | 0 | 0.0% | 0.00 |
| F-7 | F | 1,514 | 0.50% | 0 | 0.00% | 0 | 0 | 0.0% | 0.00 |
| F-8 | F | 932 | 0.30% | 0 | 0.00% | 0 | 0 | 0.0% | 0.00 |
| F-9 | F | 1,730 | 0.60% | 0 | 0.00% | 0 | 0 | 0.0% | 0.00 |
| F-10 | F | 1,028 | 0.30% | 0 | 0.00% | 0 | 0 | 0.0% | 0.00 |
| F-11 | F | 3,537 | 1.20% | 0 | 0.00% | 0 | 0 | 0.0% | 0.00 |
| F-12 | F | 1,817 | 0.60% | 0 | 0.00% | 0 | 0 | 0.0% | 0.00 |
| F-13 | F | 3,236 | 1.10% | 0 | 0.00% | 0 | 0 | 0.0% | 0.00 |
| F-14 | F | 1,845 | 0.60% | 0 | 0.00% | 0 | 0 | 0.0% | 0.00 |
| F-15 | F | 2,375 | 0.80% | 0 | 0.00% | 0 | 0 | 0.0% | 0.00 |
| F-16 | F | 2,381 | 0.80% | 0 | 0.00% | 0 | 0 | 0.0% | 0.00 |
| F-17 | F | 2,983 | 1.00% | 0 | 0.00% | 0 | 0 | 0.0% | 0.00 |
| F-18 | F | 5,593 | 1.90% | 1 | 0.70% | 0.39 | 0 | 0.0% | 0.00 |
| F-19 | F | 3,594 | 1.20% | 1 | 0.70% | 0.6 | 0 | 0.0% | 0.00 |
| F-20 | F | 2,693 | 0.90% | 1 | 0.70% | 0.8 | 1 | 1.0% | 1.16 |
| F-21 | F | 2,178 | 0.70% | 1 | 0.70% | 0.99 | 1 | 1.0% | 1.44 |
| F-22 | F | 4,265 | 1.40% | 2 | 1.40% | 1.01 | 1 | 1.0% | 0.73 |
| F-23 | F | 1,902 | 0.60% | 1 | 0.70% | 1.14 | 1 | 1.0% | 1.65 |
| F-24 | F | 1,837 | 0.60% | 1 | 0.70% | 1.18 | 0 | 0.0% | 0.00 |
| F-25 | F | 3,912 | 1.30% | 3 | 2.20% | 1.66 | 3 | 3.1% | 2.40 |
| F-26 | F | 3,405 | 1.10% | 3 | 2.20% | 1.9 | 3 | 3.1% | 2.76 |
| F-27 | F | 2,114 | 0.70% | 2 | 1.40% | 2.04 | 0 | 0.0% | 0.00 |
| F-28 | F | 1,019 | 0.30% | 1 | 0.70% | 2.12 | 1 | 1.0% | 3.07 |
| F-29 | F | 1,908 | 0.60% | 2 | 1.40% | 2.27 | 2 | 2.1% | 3.28 |
| F-30 | F | 3,538 | 1.20% | 5 | 3.60% | 3.05 | 4 | 4.2% | 3.54 |
| F-31 | F | 6,004 | 2.00% | 13 | 9.40% | 4.68 | 6 | 6.3% | 3.13 |
| F-32 | F | 2833 | 0.90% | 7 | 5.00% | 5.34 | 6 | 6.3% | 6.63 |
| F-33 | F | 2,966 | 1.00% | 8 | 5.80% | 5.83 | 0 | 0.0% | 0.00 |
| F-34 | F | 2,577 | 0.90% | 8 | 5.80% | 6.71 | 5 | 5.2% | 6.07 |
| F-35 | F | 1,021 | 0.30% | 4 | 2.90% | 8.47 | 3 | 3.1% | 9.20 |
| F-36 | F | 4,544 | 1.50% | 21 | 15.10% | 9.99 | 12 | 12.5% | 8.26 |
| F-37 | F | 1,936 | 0.60% | 11 | 7.90% | 12.28 | 10 | 10.4% | 16.17 |
| F-38 | F | 1,406 | 0.50% | 10 | 7.20% | 15.37 | 9 | 9.4% | 20.03 |
| M-1 | M | 4,346 | 1.40% | 0 | 0.00% | 0 | 0 | 0.0% | 0.00 |
| M-2 | M | 3,578 | 1.20% | 0 | 0.00% | 0 | 0 | 0.0% | 0.00 |
| M-3 | M | 3,931 | 1.30% | 0 | 0.00% | 0 | 0 | 0.0% | 0.00 |
| M-4 | M | 5,001 | 1.70% | 0 | 0.00% | 0 | 0 | 0.0% | 0.00 |
| M-5 | M | 4,173 | 1.40% | 0 | 0.00% | 0 | 0 | 0.0% | 0.00 |
| M-6 | M | 3,690 | 1.20% | 0 | 0.00% | 0 | 0 | 0.0% | 0.00 |
| M-7 | M | 5,701 | 1.90% | 0 | 0.00% | 0 | 0 | 0.0% | 0.00 |
| M-8 | M | 4,604 | 1.50% | 0 | 0.00% | 0 | 0 | 0.0% | 0.00 |
| M-9 | M | 2,653 | 0.90% | 0 | 0.00% | 0 | 0 | 0.0% | 0.00 |
| M-10 | M | 4,031 | 1.30% | 0 | 0.00% | 0 | 0 | 0.0% | 0.00 |
| M-11 | M | 4,557 | 1.50% | 0 | 0.00% | 0 | 0 | 0.0% | 0.00 |
| M-12 | M | 2,074 | 0.70% | 0 | 0.00% | 0 | 0 | 0.0% | 0.00 |
| M-13 | M | 1,923 | 0.60% | 0 | 0.00% | 0 | 0 | 0.0% | 0.00 |
| M-14 | M | 4,257 | 1.40% | 0 | 0.00% | 0 | 0 | 0.0% | 0.00 |
| M-15 | M | 1,441 | 0.50% | 0 | 0.00% | 0 | 0 | 0.0% | 0.00 |
| M-16 | M | 5,390 | 1.80% | 0 | 0.00% | 0 | 0 | 0.0% | 0.00 |
| M-17 | M | 5,220 | 1.70% | 0 | 0.00% | 0 | 0 | 0.0% | 0.00 |
| M-18 | M | 1,794 | 0.60% | 0 | 0.00% | 0 | 0 | 0.0% | 0.00 |
| M-19 | M | 2,069 | 0.70% | 0 | 0.00% | 0 | 0 | 0.0% | 0.00 |
| M-20 | M | 2,916 | 1.00% | 0 | 0.00% | 0 | 0 | 0.0% | 0.00 |
| M-21 | M | 3,256 | 1.10% | 0 | 0.00% | 0 | 0 | 0.0% | 0.00 |
| M-22 | M | 2,049 | 0.70% | 0 | 0.00% | 0 | 0 | 0.0% | 0.00 |
| M-23 | M | 4,811 | 1.60% | 0 | 0.00% | 0 | 0 | 0.0% | 0.00 |
| M-24 | M | 5,082 | 1.70% | 0 | 0.00% | 0 | 0 | 0.0% | 0.00 |
| M-25 | M | 4,884 | 1.60% | 0 | 0.00% | 0 | 0 | 0.0% | 0.00 |
| M-26 | M | 4,303 | 1.40% | 0 | 0.00% | 0 | 0 | 0.0% | 0.00 |
| M-27 | M | 3,661 | 1.20% | 0 | 0.00% | 0 | 0 | 0.0% | 0.00 |
| M-28 | M | 4,326 | 1.40% | 0 | 0.00% | 0 | 0 | 0.0% | 0.00 |
| M-29 | M | 4,569 | 1.50% | 0 | 0.00% | 0 | 0 | 0.0% | 0.00 |
| M-30 | M | 2,428 | 0.80% | 0 | 0.00% | 0 | 0 | 0.0% | 0.00 |
| M-31 | M | 3,534 | 1.20% | 0 | 0.00% | 0 | 0 | 0.0% | 0.00 |
| M-32 | M | 3,810 | 1.30% | 0 | 0.00% | 0 | 0 | 0.0% | 0.00 |
| M-33 | M | 3,713 | 1.20% | 0 | 0.00% | 0 | 0 | 0.0% | 0.00 |
| M-34 | M | 3,421 | 1.10% | 0 | 0.00% | 0 | 0 | 0.0% | 0.00 |
| M-35 | M | 2,269 | 0.80% | 0 | 0.00% | 0 | 0 | 0.0% | 0.00 |
| M-36 | M | 3,724 | 1.20% | 0 | 0.00% | 0 | 0 | 0.0% | 0.00 |
| M-37 | M | 3,749 | 1.20% | 0 | 0.00% | 0 | 0 | 0.0% | 0.00 |
| M-38 | M | 2,265 | 0.80% | 1 | 0.70% | 0.95 | 1 | 1.0% | 1.38 |
| 250 Female | F | 21,676 | 7.20% | 13 | 9.40% | 1.3 | 11 | 11.5% | 1.59 |
| Segregants | | | | | | | | | |
| 250 Male | M | 22,811 | 7.60% | 1 | 0.70% | 0.09 | 0 | 0.0% | 0.00 |
| Segregants | | | | | | | | | |
| Control | F | 6,793 | 2.30% | 18 | 12.90% | 5.73 | 16 | 16.7% | 7.37 |
| Line 1 | | | | | | | | | |
| Control | F | 3,526 | 1.20% | 0 | 0.00% | 0 | 0 | 0.0% | 0.00 |
| Line 2 | | | | | | | | | |
| Total | | 300,449 | 100% | 139 | 100% | — | 96 | 100% | — |

As shown in Table 10, a total of 139 explants identified by genotyping were successfully transformed. The “% Transformants” ranged from 0% to 15.1%. There were 96 successful edits by the gene editing construct, representing 17 unique female inbred lines, 10 F×F unique F₁ segregating lines, and 1 unique male inbred. The “% Edited” ranged from 0% to 16.7%. These results indicate that bulked germplasms can be successfully transformed and edited, but different germplasms in the bulk have varying transformation and editing frequencies. This suggests that further process optimization may be helpful to improve efficiency for bulked germplasms. Alternatively, the bulked germplasm transformation/gene editing method may be used to screen or select for germplasms with relatively high transformation or gene editing frequencies.

In Example 4, Experiment 2, approximately 265,000 total explants (19 replicates of approximately 14,000) from the male bulk germplasms of Example 3, Experiment 2, were inoculated with Agrobacterium harboring a recombinant DNA construct containing a β-glucuronidase reporter gene as described in Example 3, Experiment 2. After the inoculation, the Agrobacterium suspension was removed, and the inoculated explants were blotted on a sterile paper towel to remove excess Agrobacterium solution. Explants were then transferred to co-culture plates as described for Example 4, Experiment 1 at a density of fewer than 700 explants per plate.

After co-culture, explants were transferred to Petri plates containing bud induction media as described for Example 4, Experiment 1. The explants were then transferred to extended bud induction medium containing MS basal salts, B5 vitamins, 60 g/L sucrose, 0.5 g/L glutamine, 1 g/L NZ Amine-A, 0.69 g/L proline, 2 mg/L glycine, 1.95 g/L MES, 1.25 mg/L cupric sulfate, thidiazuron, 2 mg/L picloram, 400 mg/L carbenicillin, 200 mg/L cefotaxime, 100 mg/L timentin, 25 μM glyphosate, and 3.5 g/L low EEO agarose, pH 5.8, at a target density of about 70 explants/plate and cultured at 28+/−3° C. for 14 days, with a photoperiod of 16-hour light/8-hour dark, and a light intensity target of 150 PAR (ranging from about 140 to about 160 PAR).

Next, the explants were transferred to 40 ml plates with regeneration medium containing LM Woody Plant Medium salts and vitamins, 0.03 g/L Clearys 3336 WP, 30 g/L sucrose, 0.69 g/L proline, 2 mg/L glycine, 1 g/L MES, 3.5 g/L low EEO agarose, 400 mg/L carbenicillin, 200 mg/L cefotaxime, 100 mg/L timentin and 20 mM glyphosate, pH 5.8, at a density of approximately 70 explants per plate, and cultured at 28+/−3° C., with a photoperiod of 16-hour light/8-hour dark, and a light intensity target of 160 PAR (ranging from about 140 to about 200 PAR) for 10-14 days, depending on plant development.

Finally, green explants were transferred to regeneration medium containing LM Woody Plant Medium salts and vitamins, 0.03 g/L Clearys 3336 WP, 30 g/L sucrose, 0.69 g/L proline, 2 mg/L glycine, 1 g/L MES, 3.5 g/L low EEO agarose, 400 mg/L carbenicillin, 200 mg/L cefotaxime, 100 mg/L timentin and 20 mM glyphosate, pH 5.8 in 9 cm Vivi trays (Vivi, The Netherlands), at a target density of about 100-200 explants/tray, which was dependent on number of green explants, and cultured at 28+/−3° C. for 4 weeks, with a photoperiod of 16-hour light/8-hour dark, and a light intensity target of 160 PAR (ranging from about 140 to about 200 PAR). Regenerated plants were collected and assayed for genotyping and transformation.

The results of Example 4, Experiment 2 are summarized in Table 11. The definitions for each of the columns and terminology used in Table 11 are the same as provided above for Bulks 3 & 4 in Table 10, except that the numbers and calculations are for male germplasms in Example 4, Experiment 2 out of a total of 131 transformants identified by genotyping. Editing was not performed in Example 4, Experiment 2.

TABLE 11
Response of different male germplasms in bulked explant preparations to
Agrobacterium-mediated transformation (Example 4, Experiment 2).

| | | | | | | 7 |
|---|---|---|---|---|---|---|
| | 2 | 3 | 4 | 5 | 6 | Relative |
| 1 | Heterotic | # Kernels | % Kernels | # | % | Transformation |
| Germplasm | group | in Bulk | in Bulk | Transformants | Transformants | Rate |
| M2-1 | M | 60,000 | 7.50% | 0 | 0.00% | 0.0000 |
| M2-2 | M | 40,000 | 5.00% | 0 | 0.00% | 0.0000 |
| M2-3 | M | 250,000 | 31.25% | 58 | 44.27% | 1.4168 |
| M2-4 | M | 25,000 | 3.13% | 1 | 0.76% | 0.2443 |
| M2-5 | M | 4,000 | 0.50% | 0 | 0.00% | 0.0000 |
| M2-6 | M | 40,000 | 5.00% | 51 | 38.93% | 7.7863 |
| M2-7 | M | 30,000 | 3.75% | 1 | 0.76% | 0.2036 |
| M2-8 | M | 8,000 | 1.00% | 0 | 0.00% | 0.0000 |
| M2-9 | M | 40,000 | 5.00% | 0 | 0.00% | 0.0000 |
| M2-10 | M | 30,000 | 3.75% | 0 | 0.00% | 0.0000 |
| M2-11 | M | 10,000 | 1.25% | 0 | 0.00% | 0.0000 |
| M2-12 | M | 20,000 | 2.50% | 6 | 4.58% | 1.8321 |
| M2-13 | M | 25,000 | 3.13% | 2 | 1.53% | 0.4885 |
| M2-14 | M | 10,000 | 1.25% | 0 | 0.00% | 0.0000 |
| M2-15 | M | 100,000 | 12.50% | 11 | 8.40% | 0.6718 |
| M2-16 | M | 30,000 | 3.75% | 0 | 0.00% | 0.0000 |
| M2-17 | M | 15,000 | 1.88% | 0 | 0.00% | 0.0000 |
| M2-18 | M | 25,000 | 3.13% | 1 | 0.76% | 0.2443 |
| M2-19 | M | 30,000 | 3.75% | 0 | 0.00% | 0.0000 |
| M2-20 | M | 8,000 | 1.00% | 0 | 0.00% | 0.0000 |
| Total | | 800,000 | 100% | 131 | 100% | — |

As shown in Table 11, a total of 131 explants identified by genotyping were successfully transformed, representing 8 out of 20 germplasms tested in Example 4, Experiment 2. The “% Transformants” ranged from 0% to 44.27%. These results indicate that many of the bulked germplasms can be successfully transformed, but different germplasms in the bulk exhibit varying transformation frequencies.

In Example 4, Experiment 3, approximately 23,000 explants from Example 3, Experiment 3 were inoculated in two batches of approximately 11,675 visible explants (25 g dry weight) each with Agrobacterium containing a separate gene editing construct according to the methods described in Example 3, Experiment 3. Except that approximately 2,000-2,500 explants were inoculated per inoculation container. Co-culture, bud induction, extended bud induction, and regeneration were performed as described for Example 4, Experiment 1. Regenerated plants were sampled and assayed for genotype and confirmation of transformation and gene editing.

As shown in Table 12, a total of 55 explants identified by genotyping were successfully transformed, and 21 of those explants were successfully edited such that >10% of sequencing reads contained the expected edit. Numerous experiments have demonstrated that if >10% of sequencing reads contain the expected edit, then the edit is likely heritable (Heritable Edit) (data not shown). The definitions for each of the columns and terminology used in Table 12 are the same as provided above for Bulks 3 & 4 in Table 10, except that the numbers and calculations are for tropical female germplasms (and non-tropical controls) with heritable edits out of a total of 55 transformants. An additional transformant with a Heritable Edit was unable to be identified by genotyping and was excluded. Regional Group indicates whether the line is Non-Tropical (NT) or Tropical (TR).

TABLE 12
Response of different germplasms in bulked explant preparations to Agrobacterium-mediated
transformation and editing (Heritable Edits) (Example 4, Experiment 3).

| | | | | | | | | | 10 |
|---|---|---|---|---|---|---|---|---|---|
| | | | | | | 7 | | | Relative |
| | 2 | 3 | 4 | 5 | 6 | Relative | 8 | 9 | Editing |
| 1 | Regional | # Kernels | % Kernels | # | % | Transformation | # Edited | % Edited | Rate |
| Germplasm | group | in Bulk | in Bulk | Transformants | Transformants | Rate | (Heritable) | (Heritable) | (Heritable) |
| F3-1 | NT | 5,828 | 3.9% | 27 | 49.1% | 12.63 | 14 | 66.7% | 17.15 |
| F3-2 | NT | 6,033 | 4.0% | 17 | 30.9% | 7.68 | 5 | 23.8% | 5.92 |
| F3-3 | NT | 7,084 | 4.7% | 2 | 3.6% | 0.77 | 0 | 0.0% | 0.00 |
| F3-4 | TR | 7,050 | 4.7% | 3 | 5.5% | 1.16 | 1 | 4.8% | 1.01 |
| F3-5 | TR | 7,986 | 5.3% | 3 | 5.5% | 1.02 | 0 | 0.0% | 0.00 |
| F3-6 | TR | 5,085 | 3.4% | 1 | 1.8% | 0.54 | 1 | 4.8% | 1.40 |
| F3-7 | TR | 7,923 | 5.3% | 1 | 1.8% | 0.34 | 0 | 0.0% | 0.00 |
| F3-8 | TR | 6,684 | 4.5% | 1 | 1.8% | 0.41 | 0 | 0.0% | 0.00 |
| F3-9 | TR | 7,146 | 4.8% | 0 | 0.0% | 0.00 | 0 | 0.0% | 0.00 |
| F3-10 | TR | 2,216 | 1.5% | 0 | 0.0% | 0.00 | 0 | 0.0% | 0.00 |
| F3-11 | TR | 4,983 | 3.3% | 0 | 0.0% | 0.00 | 0 | 0.0% | 0.00 |
| F3-12 | TR | 5,441 | 3.6% | 0 | 0.0% | 0.00 | 0 | 0.0% | 0.00 |
| F3-13 | TR | 1,902 | 1.3% | 0 | 0.0% | 0.00 | 0 | 0.0% | 0.00 |
| F3-14 | TR | 6,459 | 4.3% | 0 | 0.0% | 0.00 | 0 | 0.0% | 0.00 |
| F3-15 | TR | 6,716 | 4.5% | 0 | 0.0% | 0.00 | 0 | 0.0% | 0.00 |
| F3-16 | TR | 2,661 | 1.8% | 0 | 0.0% | 0.00 | 0 | 0.0% | 0.00 |
| F3-17 | TR | 6,212 | 4.1% | 0 | 0.0% | 0.00 | 0 | 0.0% | 0.00 |
| F3-18 | TR | 6,274 | 4.2% | 0 | 0.0% | 0.00 | 0 | 0.0% | 0.00 |
| F3-19 | TR | 1,085 | 0.7% | 0 | 0.0% | 0.00 | 0 | 0.0% | 0.00 |
| F3-20 | TR | 7,147 | 4.8% | 0 | 0.0% | 0.00 | 0 | 0.0% | 0.00 |
| F3-21 | TR | 2,119 | 1.4% | 0 | 0.0% | 0.00 | 0 | 0.0% | 0.00 |
| F3-22 | TR | 5,010 | 3.3% | 0 | 0.0% | 0.00 | 0 | 0.0% | 0.00 |
| F3-23 | TR | 3,132 | 2.1% | 0 | 0.0% | 0.00 | 0 | 0.0% | 0.00 |
| F3-24 | TR | 5,578 | 3.7% | 0 | 0.0% | 0.00 | 0 | 0.0% | 0.00 |
| F3-25 | TR | 5,139 | 3.4% | 0 | 0.0% | 0.00 | 0 | 0.0% | 0.00 |
| F3-26 | TR | 7,409 | 4.9% | 0 | 0.0% | 0.00 | 0 | 0.0% | 0.00 |
| F3-27 | TR | 9,621 | 6.4% | 0 | 0.0% | 0.00 | 0 | 0.0% | 0.00 |
| Total | | 149,923 | 100% | 55 | 100% | — | 21 | 100% | — |

As shown in Table 12, a total of 55 explants identified by genotyping were successfully transformed, representing 8/27 germplasms tested. The “% Transformants” ranged from 0% to 5.5% for the tropical female germplasms. The highest “% Transformants” (49.1%) was obtained from the non-tropical control line of F3-1. The overall transformation frequency for Example 4, Experiment 3 was estimated to be 0.29%, assuming 80% regeneration of the explants. Twenty-one successful heritable edits by the gene editing construct were identified, representing four different germplasms, 19 of which came from the non-tropical control lines F3-1 and F3-2. The “% Edited (Heritable)” ranged from 0% to 4.8% for tropical female germplasms. The highest “% Edited (Heritable)” (66.7%) was obtained from the non-topical control line of F3-1.

As shown in Table 13, of the 55 explants identified by genotyping as transformed in Example 4, Experiment 3, an additional 13 of those explants were successfully edited such that 1-10% of sequencing reads contained the expected editing. These edits may not be heritable (Non-Heritable Edit). The definitions for each of the columns and terminology used in Table 13 are the same as provided above for Bulks 3 & 4 in Table 10, except that the numbers and calculations are for tropical female germplasms (and non-tropical controls) with Non-Heritable Edits out of a total of 55 transformants. Regional Group indicates whether the line is Non-Tropical (NT) or Tropical (TR).

TABLE 13
Response of different germplasms in bulked explant preparations to Agrobacterium-mediated
transformation and editing (Non-Heritable Edits) (Example 4, Experiment 3).

| | | | | | | | | | 10 |
|---|---|---|---|---|---|---|---|---|---|
| | | | | | | 7 | 8 | 9 | Relative |
| | 2 | 3 | 4 | 5 | 6 | Relative | # Edited | % Edited | Editing Rate |
| 1 | Regional | # Kernels | % Kernels | # | % | Transformation | (Non- | (Non- | (Non- |
| Germplasm | group | in Bulk | in Bulk | Transformants | Transformants | Rate | Heritable) | Heritable) | Heritable) |
| F3-1 | NT | 5,828 | 3.9% | 27 | 49.1% | 12.60 | 5 | 38.5% | 9.87 |
| F3-2 | NT | 6,033 | 4.0% | 17 | 30.9% | 7.72 | 5 | 38.5% | 9.63 |
| F3-3 | NT | 7,084 | 4.7% | 2 | 3.6% | 0.77 | 0 | 0.0% | 0.00 |
| F3-4 | TR | 7,050 | 4.7% | 3 | 5.5% | 1.17 | 1 | 7.7% | 1.64 |
| F3-5 | TR | 7,986 | 5.3% | 3 | 5.5% | 1.04 | 2 | 15.4% | 2.91 |
| F3-6 | TR | 5,085 | 3.4% | 1 | 1.8% | 0.53 | 0 | 0.0% | 0.00 |
| F3-7 | TR | 7,923 | 5.3% | 1 | 1.8% | 0.34 | 0 | 0.0% | 0.00 |
| F3-8 | TR | 6,684 | 4.5% | 1 | 1.8% | 0.40 | 0 | 0.0% | 0.00 |
| F3-9 | TR | 7,146 | 4.8% | 0 | 0.0% | 0.00 | 0 | 0.0% | 0.00 |
| F3-10 | TR | 2,216 | 1.5% | 0 | 0.0% | 0.00 | 0 | 0.0% | 0.00 |
| F3-11 | TR | 4,983 | 3.3% | 0 | 0.0% | 0.00 | 0 | 0.0% | 0.00 |
| F3-12 | TR | 5,441 | 3.6% | 0 | 0.0% | 0.00 | 0 | 0.0% | 0.00 |
| F3-13 | TR | 1,902 | 1.3% | 0 | 0.0% | 0.00 | 0 | 0.0% | 0.00 |
| F3-14 | TR | 6,459 | 4.3% | 0 | 0.0% | 0.00 | 0 | 0.0% | 0.00 |
| F3-15 | TR | 6,716 | 4.5% | 0 | 0.0% | 0.00 | 0 | 0.0% | 0.00 |
| F3-16 | TR | 2,661 | 1.8% | 0 | 0.0% | 0.00 | 0 | 0.0% | 0.00 |
| F3-17 | TR | 6,212 | 4.1% | 0 | 0.0% | 0.00 | 0 | 0.0% | 0.00 |
| F3-18 | TR | 6,274 | 4.2% | 0 | 0.0% | 0.00 | 0 | 0.0% | 0.00 |
| F3-19 | TR | 1,085 | 0.7% | 0 | 0.0% | 0.00 | 0 | 0.0% | 0.00 |
| F3-20 | TR | 7,147 | 4.8% | 0 | 0.0% | 0.00 | 0 | 0.0% | 0.00 |
| F3-21 | TR | 2,119 | 1.4% | 0 | 0.0% | 0.00 | 0 | 0.0% | 0.00 |
| F3-22 | TR | 5,010 | 3.3% | 0 | 0.0% | 0.00 | 0 | 0.0% | 0.00 |
| F3-23 | TR | 3,132 | 2.1% | 0 | 0.0% | 0.00 | 0 | 0.0% | 0.00 |
| F3-24 | TR | 5,578 | 3.7% | 0 | 0.0% | 0.00 | 0 | 0.0% | 0.00 |
| F3-25 | TR | 5,139 | 3.4% | 0 | 0.0% | 0.00 | 0 | 0.0% | 0.00 |
| F3-26 | TR | 7,409 | 4.9% | 0 | 0.0% | 0.00 | 0 | 0.0% | 0.00 |
| F3-27 | TR | 9,621 | 6.4% | 0 | 0.0% | 0.00 | 0 | 0.0% | 0.00 |
| Total | | 149,923 | 100% | 55 | 100% | — | 13 | 100% | — |

As shown in Table 13, 13 additional successful Non-Heritable Edits by the gene editing construct were identified, representing 4 different germplasms, 10 of the Non-Heritable Edits came from non-tropical control lines F3-1 and F3-2. The “% Edited (Non-Heritable)” ranged from 0% to 15.4% for tropical female germplasms. The highest “% Edited (Non-Heritable)” (38.5%) was obtained from the non-topical control lines of F3-1 and F3-2. Collectively, 34 out of 55 transformants representing 5 lines contained either Non-Heritable or Heritable Edits, 5 such edits represented 3 different tropical lines in the bulk while 29 edits were in non-tropical controls. An additional Heritable Edit was unable to be identified by genotyping and was excluded.

Overall, different germplasms responded to Agrobacterium-mediated transformation and gene editing differently. Preliminary results seem to indicate that (1) in Example 4, Experiment 1, female inbred lines demonstrated better transformability compared to male inbred lines using the same transformation protocol; (2) several female inbred lines in Example 4, Experiment 1 performed similarly or better than the control line; and (3) the F₁ segregating populations performed slightly better than the inbred lines, indicating heterosis (data not shown). Without being bound by theory, female inbred lines may have performed better than male inbred lines in Example 4, Experiment 1 because the transformation protocol used was developed for female line transformation. In Example 4, Experiment 3, non-tropical lines demonstrated better transformability compared to tropical lines using the same transformation protocol, which may again be due to the transformation protocol being developed for non-tropical germplasms. Thus, the transformation protocol may be further optimized or improved for tropical and/or male germplasm lines. These results indicate that bulk corn explant transformation may be used as a tool to increase efficiency and cost efficacy through the transformation of multiple germplasms at once. These results further demonstrate that the present disclosure provides methods for determining the relative transformation, editing, shoot generation, and/or regeneration frequencies of different germplasms with a given transformation protocol and for optimizing and improving transformation and/or editing protocols for use with different bulk explant preparations.

### Example 5

### Identification of Genotype Using Genetic Markers

Genotyping was performed to deconvolute and identify explants, cultured explants, and/or genetically modified plants or plant parts by germplasm in Examples 1-4. Genotyping was performed at different stages, including explant isolation, shoot generation, Agrobacterium inoculation, transformation, and/or editing. Deconvolution of bulk populations can be performed using a relatively small number of genetic markers to identify combinations of unique polynucleotide sequences bearing different SNPs (Single Nucleotide Polymorphisms) and/or indels that are characteristic of individual germplasms. The combination of genetic markers utilized for deconvolution and identification of individual germplasms was chosen according to specific criteria.

In order to select genetic markers for deconvolution, each germplasm of the bulk was fingerprinted to identify SNPs and/or indels for genetic marker selection. Using the data obtained from fingerprinting, genetic markers were preferentially selected according to the following criteria: (1) genetic markers that have a distinct genotype for a subset of the germplasms of the bulk, such that a germplasm lacking the genotype at the marker locus can be eliminated from consideration for identification; (2) genetic markers that have a high minor (rare) allele frequency within the bulk and the crop germplasm at large, such that the presence of a minor allele in the population is present at a level high enough to allow for elimination of several germplasms from consideration for identification; (3) genetic markers, which in combination, produce some redundancy in identification, such that if a sample lacks data for some marker loci, robust identification is possible based on unambiguous data from the total combination of genetic markers; and (4) genetic markers which are not cost prohibitive when used in combination with the total number of genetic markers in the assay.

More specifically, criteria 1 suggests selection of genetic markers with a distinct genotype for a subset of the germplasms of the bulk. If bulk germplasms are inbred lines, the chosen genotypes are preferably homozygous. Homozygosity at a genetic marker locus is preferred as it indicates that all individual explants from a single germplasm in the bulk have the same genotype. This allows for unambiguous elimination of candidate germplasms, which lack the genotype from identification of the explant. Heterozygosity at a genetic marker locus is acceptable if the germplasm is an F₁ hybrid, as all individuals from this germplasm carry the same heterozygous genotype. Thus, this is a distinguishing feature of the hybrid germplasm in the bulk. If the bulk contains segregating populations, a larger number of markers may be required for deconvolution. In an F₂ segregating population, individuals within a single germplasm may have different genotypes at a single marker locus due to segregation. Therefore, that genetic marker locus cannot exclude membership to the germplasm. To identify origin (original parental cross) of a segregating population, genetic markers are chosen which are “fixed” for a specific origin or parent. Thus, the presence of an alternate genotype at that genetic marker locus can eliminate candidate origin. Additional genetic markers are required to determine which segregant population each progeny belongs to within an origin. Thus, a larger number of genetic markers is required to derive both origin and genotypic segregant within the origin of a segregating population.

Criteria 2 suggests selection of genetic markers with high minor allele frequency both among bulk germplasms and the crop germplasm at large. Minor allele frequency refers to the allelic frequency of the rarest allele (for a biallelic marker) in a population. For a biallelic marker, the frequency of the major allele (most common allele)+the frequency of the minor allele (rare allele)=1. In most cases, high minor allele frequency for genetic marker selection was >30%. The presence of the minor allele at a genetic marker locus can be used to eliminate all carriers of the major allele. It is beneficial for minor allele frequency to be high among the bulk germplasms. When the minor allele is present in several lines, any genotype at that genetic marker locus (major or minor) can be used to eliminate several potential germplasms from consideration for identification at once. It is beneficial for the minor allele frequency to be high in the crop germplasm at large, so that external contamination with variant genotypes can be detected.

Criteria 3 suggests selection of genetic markers, which in combination with other genetic markers, have some level of redundancy. This allows for unambiguous identification of each germplasm, even in the presence of a low to moderate amount of missing data during genotyping. During deconvolution, it is possible that not every genetic marker locus produces quality data, resulting in “missing data.” Missing data may be due to low sample quality, a low amount of DNA in a sample, reaction failure, low reliability of a particular marker assay, reagent batches, and/or ambiguous reads. Building redundancy into the genetic marker combination pool allows for identification of a germplasm even if data is missing through the combination of the remaining genetic markers. The amount of redundancy to build into an assay depends on the expected rate of missing data.

Finally, criteria 4 suggests selecting genetic markers which are not cost prohibitive when used in combination with the total number of genetic markers in the assay, such that bulk transformation or editing is a preferred compared to single germplasm transformation or editing.

Based on the above criteria, Experiments 1-3 relied on a few genetic markers (26, 21 (24 chosen, 21 used), and 23, respectively) to deconvolute and identify the germplasms of each bulk through genotyping. These methods resulted in a majority of germplasms being identified with relatively few germplasms not being identified due to missing data.

### Example 6

### Preparation of Bulked Soybean Seeds of Multiple Germplasms for Transformation and Editing Experiments

As described herein, genetic modifications, such as transgenes or genomic edits, can produce variable phenotypes in different genetic backgrounds of the same crop. Investigation of these variations or interactions for a given genetic modification in different plant germplasms or genetic backgrounds, currently involves the creation of a transgenic event or edit in a single germplasm followed by crossing the event or edit into other germplasms. As provided in the present disclosure, genetic modification of multiple plant germplasms or lines simultaneously or in parallel greatly improves the efficiency of this screening and/or selection process and allows for faster and more cost-effective evaluation, for example, of any Germplasm x Transgene/Edit Interactions. According to this approach, a genetic modification or a heterologous polynucleotide molecule can be introduced into a population of embryo explants from multiple germplasms at once, such as explants combined into one or more containers, which may be followed by deconvolution, screening, and/or selection of individual germplasm(s) containing the genetic modification. In some embodiments, deconvolution, screening, and/or selection of individual germplasm(s) may be based on determination of genotype and/or on phenotypic observations.

In this example, seeds from 104 different soybean germplasms (5,000 seeds/germplasm), including germplasms from each of the maturity groups (00-7) and spanning a diverse set of haplotypes, were combined into a bulk for explant excision and isolation. Table 14 shows the number of germplasms and the total number of seeds included (5,000 seeds/germplasm) from each maturity group.

TABLE 14
Summary of the seed composition of the soybean bulk
from combined germplasms of maturity groups 00-7.

| Maturity Group | # of Germplasms | Total # Seeds |
|---|---|---|
| 00 | 4 | 20,000 |
| 0 | 9 | 45,000 |
| 1 | 15 | 75,000 |
| 2 | 18 | 90,000 |
| 3 | 26 | 130,000 |
| 4 | 22 | 110,000 |
| 5 | 6 | 30,000 |
| 6 | 2 | 10,000 |
| 7 | 2 | 10,000 |
| Totals | 104 | 520,000 |

The batch of bulked seeds, as described in Table 14, was then subjected to an automated seed explant excision process, which includes one or more steps of seed sanitization, seed drying, seed milling, explant enrichment, and explant purification. Briefly, soybean seeds of the bulk were harvested and sanitized using methods known to those of ordinary skill in the art. In certain embodiments, soybean seed may be dried to obtain a desired moisture content for storage. Explants comprising transformable and regenerable tissue, including embryonic and/or meristematic tissue, were excised from the bulked seeds by milling. The explants were then enriched and purified through removal of debris and unnecessary seed parts. In particular embodiments, explants may be dried to obtain a desired moisture content for storage purposes.

The bulk excised explants were transformed using Agrobacterium-mediated transformation as follows and then used as described in subsequent Examples 7-9. Approximately 72,000 explants from the bulk described in Table 1 were inoculated in two transformation initiations of about 36,000 explants each (Initiation 1 and Initiation 2). Briefly, explants stored at −20° C. were retrieved from the freezer and thawed in a sterile laminar flow hood. The explants were then transferred to enough rehydration medium (200 g/L Polyethylene Glycol (PEG), 2 mL Antilife Fungicide with 85% Daconil), to thoroughly cover the explants, mixed well, and then placed on a shaker at 70 rpm for about 1 hour. Following rehydration, explants were transferred to a fishnet in a rinse station and rinsed for 4 minutes with running water from a showerhead. After removal of excess water, rehydrated and rinsed explants were distributed to PLANTCON™ containers for plant tissue culture at a concentration of up to about 35 g of explants per container.

For Agrobacterium preparation, a 100 μL aliquot of an Agrobacterium glycerol stock solution was inoculated into 250 mL of LB medium with the appropriate antibiotics. The Agrobacterium strain utilized comprised a binary vector selected from the group consisting of pM204, pM205 or pM206, each of which comprises three cassettes between the T-DNA borders, including an aadA selectable marker, a Cpf1 cassette, and a gRNA cassette for performing genome editing at a target site. The gRNA cassette of the pM206 vector encodes three gRNAs (gRNAs 1, 2, and 3), whereas the gRNA cassettes of the pM204 and pM205 vectors each encode two gRNAs (gRNAs 1 and 2 for pM204; gRNAs 1 and 3 for pM205). In these experiments, pM206 was used to transform and edit the bulk soybean seed explants, pM204, pM205, and pM206 were used to transform and edit Standard Transformation Line 1, and pM204 and pM205 were used to transform and edit Standard Transformation Line 2. Standard Transformation Lines 1 and 2 are standard soybean lines shown to be efficient for transformation and editing. Two separate transformation initiations were carried out with the Standard Transformation Line 2 using the pM205 vector. Approximately 5,600 explants from each of the standard transformation lines were transformed with each construct. Two equal portions of the mixed bulk soybean seed explant preparation described in Table 14 (Bulks 1 and 2), each containing about 36,000 explants, were transformed with the pM206 construct. The mixture was cultured on a shaker at 200 rpm at 27.5±2° C. for 16-18 hours or until OD₆₆₀ reached 0.6-1.2. After the Agrobacterium cells were pelleted at 3500 RCF for 25 minutes at 4±3° C. and the supernatant was discarded, the Agrobacterium cells were resuspended in inoculation medium (⅖ of B5 macro salts, 1/10 of B5 micro salts and vitamins, 1 g/L potassium nitrate, 30 g/L dextrose, 3.9 g/L 2-(N-morpholino) ethanesulfonic acid (MES), 52 mg/L Lipoic Acid, and 1 mg/L Thidiazuron (TDZ) to a final OD₆₆₀ reading of 0.35.

The prepared soybean explants (about 1400 explants per PLANTCON™) were inoculated in 50 mL of the Agrobacterium preparation in each PLANTCON™ container, and sonicated at 45 kHz for 20 seconds. Following sonication, soybean explants in Agrobacterium preparation were placed on a shaker at 80 rpm for 30±5 minutes. After the inoculation, the Agrobacterium solution was removed through aspiration, and any remaining liquid in the PLANTCON™ was removed using 1-2 sterile filter papers. Explants were then transferred to co-culture plates with a piece of sterile filter paper wetted with 2 mL of co-culture medium (B5 major and minor salts, 60 mg/L CaCl₂, 2.8 mg/L Sequestrene, 1 g/L Potassium Nitrate, 30 g/L Dextrose, 3.9 g/L MES, 10 mg/L Thiabendazole (TBZ), 50 mg/L Nystatin, B5 vitamins, 1 mg/L Thidiazuron (TDZ), and 52 mg/L Lipoic Acid), at a density of about 100 explants per plate arranged in a single layer. The co-culture plates were incubated at 23±3° C. for 3 to 5 days, with a photoperiod of 16-hours light/8-hours dark and a relative humidity of approximately 45±10%, and a light intensity target of 90 μE (range about 65 μE to about 115 μE).

The explants were then transferred to solid selection and regeneration medium (3.21 g/L Gamborgs B5 Medium, 20 g/L sucrose, 1.29 g/L Calcium Gluconate, 0.03 g/L Clearys 3336 WP, 4 g/L Agargel, 200 mg/L Carbenicillin, 100 mg/L Timentin, 200 mg/mL Cefotaxime, and 150 mg/L Spectinomycin) at a density of about 200 explants per Vivi tray (Vivi, The Netherlands). Explants were cultured at 28±3° C. with a photoperiod of 16-hours light/8-hours dark and a light intensity target of −120 μE (range about 110 μE to about 180 μE). Approximately 7 weeks after inoculation, regenerated soybean plants were transferred to soil plug trays and plants were grown in the greenhouse or growth environment with intermittent misting and sub-irrigation with fertigated water (˜1300 electrical conductivity (EC) ±150 EC). Light conditions were maintained at 16 hours of sunlight/day supplemented with grow lights. Edits or events were sampled at about 3 weeks post plugging.

Overall transformation plugging frequency is shown in Table 15. As demonstrated in Table 15, the adjusted plugging frequency (defined as (the total number of plantlets produced divided by the total number of inoculated explants minus number of contaminated explants)×100) was similar between the bulked explants and the two standard transformation germplasms (about 5%), indicating that most, if not all, of the germplasms in the bulk preparation are transformable using the methods described herein. A total of 3,612 regenerated plants were transferred to soil plugs from the 72,000 inoculated explants from Bulk 1 and Bulk 2 combined.

TABLE 15
Overall transformation plugging frequency of soybean
bulks compared to standard transformation lines.

| | | # Explants | Adjusted_Plug_Freq |
|---|---|---|---|
| Vector | Input | Inoculated | (%) |
| pM206 | Bulk 1 | 36,000 | 5.6 |
| pM206 | Bulk 2 | 36,000 | 6.3 |
| pM206 | Standard | 5,600 | 4.5 |
| | Transformation Line 1 | | |
| pM204 | Standard | 5,600 | 4.8 |
| | Transformation Line 1 | | |
| pM204 | Standard | 5,600 | 4.6 |
| | Transformation Line 2 | | |
| pM205 | Standard | 5,600 | 4.6 |
| | Transformation Line 1 | | |
| pM205 | Standard | 5,600 | 4.5 |
| | Transformation Line 2 | | |
| pM205 | Standard | 5,600 | 4.8 |
| | Transformation Line 2 | | |

Leaf samples from 2,016 out of 3,612 plugged plants were collected and assayed for genotype, transgene copy number, and confirmation of gene editing as described in Examples 7-9.

### Example 7

### Selection and Use of Genetic Markers for Identification of Genotypes in Bulk Transformation

Genotyping was performed on the samples from the plugged Ro plants. Deconvolution of bulk populations can be performed using a relatively small number of genetic markers to identify combinations of unique polynucleotide sequences bearing different single nucleotide polymorphisms (SNPs) that are characteristic of individual germplasms present within the bulk. Fifty Taqman™ markers across 20 soybean chromosomes were preferentially selected from known fingerprints for individual lines and used in the deconvolution of the soybean bulk transformation and editing experiments described herein. The selection of the marker pool for the bulk deconvolution in this Example began with a set of 25 background markers. The initial pool of 25 background markers was chosen and optimized across samples from the varied germplasms and was shown in most cases to uniquely identify each sample in the bulk. In the initial pool of 25 markers, 4 lines were indistinguishable based on the markers, 9 had only 1 distinguishable marker, and 14 had only 2 distinguishable markers. In most cases, two distinguishable markers is enough to distinguish between two different lines. Thus, to be able to differentiate most or all of the lines present in the bulks, 25 additional markers were selected using the same criteria to complement the first set and extend coverage. Methods of selection using this combined pool of markers resulted in a majority of germplasms being identified, with relatively few germplasms not being identified due to missing data, and in some cases the indistinguishability of markers.

The combination of genetic markers utilized for deconvolution and identification of individual germplasms may be chosen according to the criteria described in the present specification.

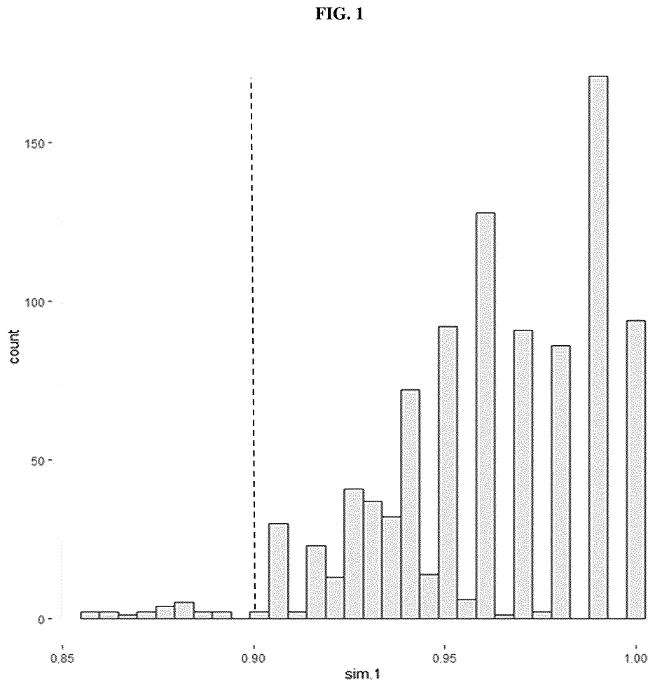

Using these markers, 92% of the 2,016 sampled events (1,859 events) were assigned line identities via Taqman™ genotyping and deconvolution following transformation. These events correspond to individual regenerated plants. Events were removed from further analysis if: >90% similarity threshold to a genotype in the bulk was not achieved (˜400 events removed), T-DNA insertion copy number analysis failed or produced zero reads (˜50 events removed), and/or amplicon sequencing for edits failed or produced zero reads. This resulted in 1,593 events being subjected to further analysis as described in Example 8. Table 16 and FIG. 1 show the results of genotyping using these markers. In FIG. 1, the x-axis shows the similarity score when compared to the markers assigned to each germplasm identity in the bulk, and the y-axis shows the number of samples assigned to a specific similarity score. The black dotted line depicts the threshold of similarity used to assign germplasm identity (0.90 or 90% similarity). Some genetically similar lines could not be distinguished based on the returned data in this example. Lines achieving 90% or higher similarity to a known germplasm identity by marker genotyping were assigned germplasm identities. In Table 16, “Sample Class” denotes different categoric qualifiers used to determine quality of identification and marker selection. “# of Samples (%)” denotes the number and (%) of samples (out of 2,016) that fell in each category. “Heterozygosity <5%” represents the number of samples passing QC checks, and “high” heterozygosity in reads of a sample indicates possible contamination, marker deterioration or other error during experimentation. Those samples with heterozygosity >5% were removed from analysis. As shown in the last two rows of Table 16, collectively 92.2% of samples achieved the threshold (90% similarity) for identity assignation.

TABLE 16
Results of genotyping by marker analysis on bulked
soybean lines following transformation.

| Sample Class | # of Samples (%) |
|---|---|
| Events with >45 markers returned | 1934 (95.9%) |
| Heterozygosity <5% | 1932 (95.8%) |
| No line assignment (data quality issues) | 70 (3.5%) |
| Top hit match <85% (dropped) | 38 (1.9%) |
| Top hit match 85-90% (dropped) | 49 (2.4%) |
| Top hit match 90-95% | 599 (29.7%) |
| Top hit match >95% | 1260 (62.5%) |

As shown in Table 16 and FIG. 1, lines having 90% or greater similarity to an identity by marker genotyping were assigned to the identity. As noted above, lines with <90% similarity to an identity were dropped, as were those with data quality issues. As shown in Table 16, >60% of samples were successfully assigned to a line with >95% similarity, demonstrating a high likelihood of the ability to deconvolute bulked transformants following transformation, and to identify the germplasm identity of plant samples using genotyping. Overall, greater than 95% of samples produced results from at least 45 out of the 50 markers utilized and greater than 95% of samples had less than 5% heterozygous reads, indicating the successful selection of distinct, quality markers for returning data capable of supporting high confidence deconvolution, selection, and identification for use in bulk modification experiments.

### Example 8

### Editing Frequencies of Bulked Soybean Excised Explants

The editing vector pM206 described in Example 6 was designed to edit an endogenous target gene in soybean and comprises three gRNAs known to have different cut frequencies that target different sites in the intended genomic region of the target gene: a low frequency cutter, a high frequency cutter, and a medium frequency cutter. 2,016 events were sampled from the total of 3,612 plugged plants as described in Example 6.

As described in Example 7, 1,859 events were assigned a line identity based on Taqman™ data. Of these 1,859 events, 1,593 events returned data from Taqman™, AmpSeq, and MQC analysis. These 1,593 events were utilized for subsequent analysis. The presence of edited alleles of the target gene in these 1,593 of the events with assigned identities was assessed using amplicon sequencing of the target sites. Amplicon sequencing consisted of the generation of one or more unique PCR products across the genomic region of interest, followed by sequencing analysis using techniques known in the art (e.g., Next-Gen Sequencing). Each of these platforms or assays were overlapping in terms of sequence but were performed as a quality control to validate edits. Sequence data from each sample was then mapped to a reference sequence to identify consensus differences. Edits were called if they represented >10% frequency in the mapped reads. 94% of the events sequenced contained edits (1,505 out of 1,593), representing 101 out of the 104 lines comprised in the original bulk of explants. This demonstrates that different soy lines can be bulked together for transformation, identified using markers, and that the bulk transformation or editing approach can be used to produce edited events across many different soybean germplasms.

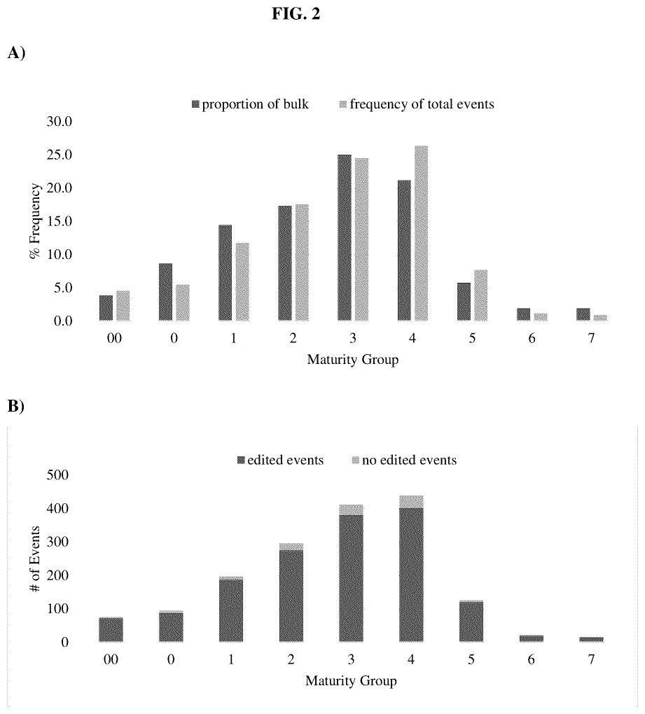

Further analysis was completed to compare transformation and editing frequencies among the different maturity groups comprised in the bulk (FIG. 2). The FIG. 2A compares the frequency of transformation events in each maturity group to the proportion of the bulk consisting of that maturity group, and the FIG. 2B shows the total number of edited versus non-edited events by maturity group. The results demonstrate that no significant bias in transformation or editing frequency was found by maturity group. The number of transformation events per maturity group corresponded to the proportion of that maturity group in the bulk preparation (FIG. 2A), indicating that a wide range of germplasms can be used in the methods provided by the present disclosure for bulk transformation and/or editing purposes. Additional analysis showed no obvious bias in editing frequency among the lines of the bulk, with over 100 lines having edits in >75% of events (FIG. 2B). In addition, relatively “higher” and “lower” performers, in terms of the number of transformation events produced, were similarly present in most maturity groups (data not shown).

The performance of the gRNAs in the bulk was also assessed and compared to the performance of those gRNAs in a standard soybean line used and shown to be efficient for transformation and editing (Table 17). All three gRNAs from the pM206 vector worked in this bulk transformation experiment with cut rates similar to those observed in a standard soybean line (Standard Line 1). In Table 17, Percentage (%) denotes the cut rate by each gRNA in each experiment. (#) denotes the total number of edits at each gRNA site as some events had edits at multiple gRNA sites.

TABLE 17
Performance of gRNAs in soybean bulk editing experiments.

| | Low Frequency | High Frequency | Medium Frequency |
|---|---|---|---|
| | Cutter | Cutter | Cutter |
| Bulk 1 | 6% (891) | 91% (889) | 59% (889) |
| Bulk 2 | 8% (896) | 93% (896) | 60% (895) |
| Bulk Total | 7% (1787) | 92% (1787) | 59% (1784) |
| Standard | 10% (160) | 96% (160) | 75% (160) |
| Line 1 | | | |

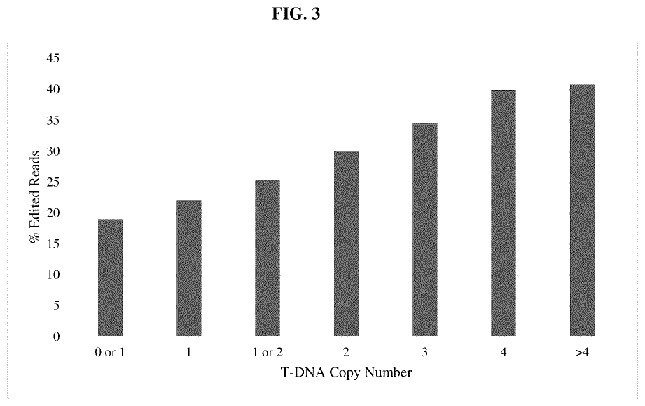

Edited events were assessed for their associated T-DNA copy number by a Taqman™ PCR assay for the 3′UTR of the Cpf1 cassette present in the T-DNA of the pM206 vector construct (FIG. 3). In summary, the events were filtered for those which contained a hit, and >90% of events were confirmed to contain a hit. Then each event was separated into a group based on copy number (0 or 1, 1, 2, 3, etc.). The total number of wild-type reads (u0) per MQC class was then counted using the AmpSeq data, which tracks the number of edited and wild-type reads per sample. Any edits that were below 10% (%=# edit reads/# total u0 reads in sample*100) were removed, leaving edits that were considered to be heritable edits remaining. Duplicate edit types within each MQC copy number class were also removed, however, the same edit could be represented in different MQC copy number classes. In FIG. 3, the x-axis represents the T-DNA copy number detected in each event and the y-axis represents the proportion of edited reads detected. Heritable edits were considered to be those edits returning >10% mapped reads. Following removal of events not classified as heritable and removal of duplicate edit types within each copy number class, resulting in only unique edits by T-DNA copy number, 1,220 events were identified. The data demonstrate that the proportion of heritable edit reads at the target sites increased as the T-DNA copy number increased, when editing was performed in bulk. This data is consistent with other experiments using a single standard transformation germplasm, indicating consistency of editing characteristics between bulk and single transformation germplasm. The number of unique edits by T-DNA copy number is higher than unique edits by target site, as shown in Example 9 below, as the same edit could be represented in multiple T-DNA copy number classes.

Taken together, the assignment of 92% of regenerated plants or events to particular germplasm identities, the targeted editing of 94% of identified lines (101 out of 104 lines in the bulk), and the consistent proportionality of increased heritable edits with increasing T-DNA copy number indicate the high level of success of transforming and editing many different soybean lines in bulk. The data obtained surprisingly does not deviate much from that expected with standard transformation lines. These examples demonstration that the bulk transformation and/or editing methods provided by the present disclosure can be used in many applications involving transformation or editing of multiple lines or germplasms.

### Example 9

### Characterization of Diversity of Generated Edits: Application of Bulk Editing

The use of the 3 gRNAs with different cut frequencies (low, medium, high) resulted in the rapid generation of many edits across the different lines and germplasms represented in the bulk, allowing for the analysis of edit type generation among the different lines and germplasms comprised within the bulk. As described in Example 8, 1,505 out of 1,593 regenerated plants or events sampled were edited events. Amplicon sequencing edits provided identification of the properties of each edit, for example the size and position of deletion(s) and/or the presence of insertions or substitutions. It was found that 840 of the 1,505 edits were present in only a single event (Table 18). In some embodiments, these edits are referred to as unique edits. Some edits were present in multiple edited events. In Table 18, “# of Unique Edits by Site” indicates the number of unique edits generated by a specific gRNA. “gRNA Cut Rate” represents the cut rate of each gRNA across both Bulks 1 and 2. “Unique Lines Represented” denotes the number of lines containing a unique edit by each gRNA, however, as a note some lines may contain edits by multiple gRNAs. Edits spanning multiple target sites, which may be indicative of a larger deletions, were assigned to a single target site to ensure that each edit was only counted once.

TABLE 18
Generation of unique edits by gRNAs in bulk
soybean transformation and editing.

| | # of Unique Edits | gRNA Cut | Unique Lines |
|---|---|---|---|
| gRNA (site) | by Site | Rate | Represented |
| Low frequency cutter | 80 | 7.0% | 63 |
| high frequency cutter | 501 | 92.3% | 101 |
| medium frequency | 259 | 59.3% | 99 |
| cutter | | | |

Consistent recovery of an edit type occurred most often with the high frequency cutter. Table 19 shows a representation of the top recurring edits and their frequency by gRNA target. In Table 19, “# of Events” denotes the number of events with the same recurrent edit. “Edit” denotes the position and size of the edit, for example, “3d5” denotes a 5 bp deletion starting 3 bases from the 5′ end of the gRNA footprint on the sense strand at the target site. “gRNA” denotes which gRNA target was associated with the particular edit.

TABLE 19
Representative top recurring edits following
bulk soybean transformation and editing.

| # of Events | Edit | gRNA |
|---|---|---|
| 397 | 5d5 | Medium frequency cutter |
| 142 | 3d5 | Medium frequency cutter |
| 90 | 4d6 | Medium frequency cutter |
| 59 | 9d9 | Medium frequency cutter |
| 58 | 6d3 | Medium frequency cutter |
| 678 | 4d7 | High frequency cutter |
| 194 | 3d6 | High frequency cutter |
| 136 | 2d11 | High frequency cutter |
| 114 | 2d8 | High frequency cutter |
| 103 | 6d7 | High frequency cutter |

The results shown in the Examples demonstrate the utility of the bulk transformation and/or editing methods provided by the present disclosure. The methods provided by the present disclosure are able generate diverse edits at scale and/or create the same or similar edit(s) at one or more target site(s) in multiple lines or germplasms. In addition, in certain embodiments, the methods provided by the present disclosure may be further used to guide technology optimization for the performance of specific functions, for example, to produce a specific recurring edit across germplasms. These findings surprisingly support the conclusion that bulk transformation/editing methods can be applied to a very diverse, broad spectrum of soybean germplasms and genetics, ranging in different maturities and from different geographies. The methods of the present disclosure may be further used, in particular embodiments, identify and narrow in on specific genomic regions, haplotypes, and/or causal variants associated with transformation competency and/or rate. This bulk transformation methods provide by the present disclosure may further be applied to screen for other phenotypes, such as susceptibility or resistance to molecules and resulting genetic associations, across germplasms, genotypes and/or transgene(s)/edit(s). Application of the methods described in the present disclosure are not limited only to generating transgenes or edits across multiple germplasms or lines, but may also be used in any process in which some type of generation or selection or screening of one or more transgenes or edits is applied followed by genotyping to identify the genetic background or germplasm of selected plants or progeny. Deconvolution may be based on known markers or other features or traits associated with different plants or progeny of the bulked population.

### Example 10

### Additional Bulk Transformation Experiments with Corn Male Lines

An additional bulk of corn seeds, hereinafter referred to as Experiment 4, harvested from multiple male elite lines and a female control line was performed as described in Example 1. The harvested seeds were from plants of each line grown in the same growing conditions and locations. The harvested seeds were combined into a single seed bulk. The resulting seed bulk was processed and excised to obtain explants for transformation, which was carried out as provided in Examples 2-4. Table 20 provides a summary of the composition of the bulked seeds from the multiple male germplasm lines for this Experiment 4.

TABLE 20
Summary of the composition of bulk seeds from multiple
male lines and a female control line for Experiment 4.

| Type | Number of Lines/Seeds |
|---|---|
| Male Inbred Lines | 36 |
| Female Control Line | 1 |
| Total Number of Seeds | 583,652 |

Bulked seeds for this Experiment 4 were subjected to automated seed explant excision, culturing, and shoot generation as described above in Example 2. After excision, a random sampling of 1,408 explants from the bulk in Experiment 4 were collected for initial genotyping and deconvolution using genetic markers as described in Example 5. This data was used to evaluate the explant excision efficiency of each germplasm in the bulk (see Table 21). As in Example 2, excised explants were also evaluated for their ability to grow and generate shoots on culture media in vitro, except that 8 replicates of shoot generation trials were undertaken in Experiment 4 with an average of about 0.94 g (in a range of about 0.5-2.0 grams) of dry weight explants were thawed and used in the replicate experiments. As described in Example 2, after rehydration, explants were plated on regeneration medium without selection at a density of approximately 33 explants per container.

The results in Table 21 represent combined data and results from all plates and replicates. Each germplasm, denoted as M3-# (where # corresponds to each of the 36 male lines) or F3-1 in the case of the single female control, represents from 0.34% to 6.06% of the total number of corn kernels or seeds in the bulk (see “Number (#) Kernels in Bulk,” column 2 of Table 21). A total of 1,408 randomly selected explants from the bulk were genotyped. In addition, samples from a total of 1,760 generated shoots were collected and genotyped. Some samples were unable to be identified by genotyping due to missing data as described in Example 5 (averaging <10% for the randomly selected explants, and <4% for the shoots) and were excluded, resulting in 1,255 genotyped explants and 1,676 genotyped shoots. The definitions for the terminology used in each of the columns of Table 21 are the same as provided above for Bulk 5 in Table 5.

TABLE 21
Response of different male germplasms in the bulk explant preparation for
explant production, culturing, and shoot generation in Experiment 4.

| | | | | | 7 | | | 10 |
|---|---|---|---|---|---|---|---|---|
| | 2 | 3 | 5 | 6 | Relative | 8 | 9 | Relative |
| 1 | # Kernels | % Kernels | # | % | Explant | # Shoots | % Shoots | Shoot |
| Germplasm | in Bulk | in Bulk | Explants | Explants | Presence | Generated | Generated | Presence |
| M3-1 | 3,395 | 0.58% | 5 | 0.40% | 0.68 | 9 | 0.54% | 0.92 |
| M3-2 | 16,238 | 2.78% | 41 | 3.27% | 1.17 | 43 | 2.57% | 0.92 |
| M3-3 | 12,578 | 2.16% | 18 | 1.43% | 0.67 | 36 | 2.15% | 1.00 |
| M3-4 | 13,608 | 2.33% | 22 | 1.75% | 0.75 | 28 | 1.67% | 0.72 |
| M3-5 | 22,812 | 3.91% | 61 | 4.86% | 1.24 | 62 | 3.70% | 0.95 |
| M3-6 | 19,701 | 3.38% | 59 | 4.70% | 1.39 | 90 | 5.37% | 1.59 |
| M3-7 | 17,875 | 3.06% | 56 | 4.46% | 1.46 | 57 | 3.40% | 1.11 |
| M3-8 | 6,427 | 1.10% | 1 | 0.08% | 0.07 | 9 | 0.54% | 0.49 |
| M3-9 | 23,856 | 4.09% | 60 | 4.78% | 1.17 | 114 | 6.80% | 1.66 |
| M3-10 | 20,368 | 3.49% | 38 | 3.03% | 0.87 | 56 | 3.34% | 0.96 |
| M3-11 | 22,711 | 3.89% | 47 | 3.75% | 0.96 | 74 | 4.42% | 1.13 |
| M3-12 | 25,074 | 4.30% | 65 | 5.18% | 1.21 | 72 | 4.30% | 1.00 |
| M3-13 | 17,602 | 3.02% | 53 | 4.22% | 1.40 | 63 | 3.76% | 1.25 |
| M3-14 | 7,026 | 1.20% | 12 | 0.96% | 0.79 | 24 | 1.43% | 1.19 |
| M3-15 | 17,319 | 2.97% | 36 | 2.87% | 0.97 | 55 | 3.28% | 1.11 |
| M3-16 | 24,342 | 4.17% | 25 | 1.99% | 0.48 | 84 | 5.01% | 1.20 |
| M3-17 | 19,469 | 3.34% | 47 | 3.75% | 1.12 | 86 | 5.13% | 1.54 |
| M3-18 | 27,589 | 4.73% | 100 | 7.97% | 1.69 | 143 | 8.53% | 1.81 |
| M3-19 | 9,941 | 1.70% | 4 | 0.32% | 0.19 | 4 | 0.24% | 0.14 |
| M3-20 | 6,370 | 1.09% | 14 | 1.12% | 1.02 | 31 | 1.85% | 1.69 |
| M3-21 | 13,432 | 2.30% | 35 | 2.79% | 1.21 | 27 | 1.61% | 0.70 |
| M3-22 | 13,366 | 2.29% | 38 | 3.03% | 1.32 | 49 | 2.92% | 1.28 |
| M3-23 | 8,920 | 1.53% | 21 | 1.67% | 1.09 | 24 | 1.43% | 0.94 |
| M3-24 | 17,580 | 3.01% | 48 | 3.82% | 1.27 | 36 | 2.15% | 0.71 |
| M3-25 | 15,085 | 2.58% | 23 | 1.83% | 0.71 | 21 | 1.25% | 0.48 |
| M3-26 | 23,563 | 4.04% | 42 | 3.35% | 0.83 | 49 | 2.92% | 0.72 |
| M3-27 | 1,987 | 0.34% | 4 | 0.32% | 0.94 | 0 | 0.00% | 0.00 |
| M3-28 | 6,043 | 1.04% | 6 | 0.48% | 0.46 | 13 | 0.78% | 0.75 |
| M3-29 | 26,534 | 4.55% | 39 | 3.11% | 0.68 | 40 | 2.39% | 0.52 |
| M3-30 | 11,495 | 1.97% | 13 | 1.04% | 0.53 | 19 | 1.13% | 0.58 |
| M3-31 | 6,230 | 1.07% | 7 | 0.56% | 0.52 | 6 | 0.36% | 0.34 |
| M3-32 | 19,627 | 3.36% | 54 | 4.30% | 1.28 | 45 | 2.68% | 0.80 |
| M3-33 | 12,665 | 2.17% | 25 | 1.99% | 0.92 | 15 | 0.89% | 0.41 |
| M3-34 | 11,949 | 2.05% | 47 | 3.75% | 1.83 | 35 | 2.09% | 1.02 |
| M3-35 | 13,578 | 2.33% | 38 | 3.03% | 1.30 | 32 | 1.91% | 0.82 |
| M3-36 | 11,952 | 2.05% | 12 | 0.96% | 0.47 | 24 | 1.43% | 0.70 |
| F3-1 | 35,345 | 6.06% | 39 | 3.11% | 0.51 | 101 | 6.03% | 1.00 |
| Total | 583,652 | 100% | 1,255 | 100% | — | 1,676 | 100% | — |

These results demonstrate that excised corn seed explants were obtained from all germplasms tested in this Experiment 4, with each germplasm representing between 0.08% to 7.97% of the total explants identified by genotyping. Out of a total of 1,676 explants that generated shoots on culture media and were subsequently identified by genotyping, each germplasm represented between 0.0% to 8.53% of the total number of shoots generated and identified. These results indicate that all tested male germplasms can be successfully excised from bulked seeds and that explants from most bulks were able to generate shoots. However, variation in regenerative capacity exists between germplasms after bulk excision as seen in the range of values in Table 21.

The prepared corn explants in this Experiment 4 were weighed and measured to determine the number of explants per gram and inoculated as described for Example 3, except that 8 replicates of approximately 1.5-6.5 grams dry weight of explants were used and inoculated in batches of approximately 570 explants. After treatment in the pressure chamber (as described for Example 3), the explants in this Experiment 4 were centrifuged for about 30 minutes at 4° C. Shoots were generated from explants as described in Example 3. Nearly all male germplasms tested in Experiment 4 were able to generate shoots after Agrobacterium inoculation. As shown in Table 22, 35 of 36 male germplasms tested generated shoots after Agrobacterium inoculation. The definitions for terminology used in each of the columns in Table 22 are the same as provided for Bulk 5 in Table 8 of Example 3, except that the numbers and calculations are for male germplasms and female control germplasm in Experiment 4 out of a total of 1,173 shoots identified by genotyping. Note that some shoots were unable to be identified by genotyping due to missing data as described in Example 5 (<10%) and were excluded from Table 22.

TABLE 22
Response of different male germplasms in bulk explant preparations
to Agrobacterium inoculation in Experiment 4.

| | | | | 5 | 6 | 7 |
|---|---|---|---|---|---|---|
| | 2 | 3 | 4 | # Shoots | % Shoots | Relative Shoot |
| 1 | Heterotic | # Kernels | % Kernels | Generated | Generated | Presence |
| Germplasm | group | in Bulk | in Bulk | After Agro | After Agro | After Agro |
| M3-1 | M | 3,395 | 0.58% | 3 | 0.26% | 0.44 |
| M3-2 | M | 16,238 | 2.78% | 16 | 1.36% | 0.49 |
| M3-3 | M | 12,578 | 2.16% | 29 | 2.47% | 1.15 |
| M3-4 | M | 13,608 | 2.33% | 14 | 1.19% | 0.51 |
| M3-5 | M | 22,812 | 3.91% | 55 | 4.69% | 1.20 |
| M3-6 | M | 19,701 | 3.38% | 28 | 2.39% | 0.71 |
| M3-7 | M | 17,875 | 3.06% | 7 | 0.60% | 0.19 |
| M3-8 | M | 6,427 | 1.10% | 2 | 0.17% | 0.15 |
| M3-9 | M | 23,856 | 4.09% | 83 | 7.08% | 1.73 |
| M3-10 | M | 20,368 | 3.49% | 38 | 3.24% | 0.93 |
| M3-11 | M | 22,711 | 3.89% | 71 | 6.05% | 1.56 |
| M3-12 | M | 25,074 | 4.30% | 8 | 0.68% | 0.16 |
| M3-13 | M | 17,602 | 3.02% | 54 | 4.60% | 1.53 |
| M3-14 | M | 7,026 | 1.20% | 53 | 4.52% | 3.75 |
| M3-15 | M | 17,319 | 2.97% | 17 | 1.45% | 0.49 |
| M3-16 | M | 24,342 | 4.17% | 139 | 11.85% | 2.84 |
| M3-17 | M | 19,469 | 3.34% | 8 | 0.68% | 0.20 |
| M3-18 | M | 27,589 | 4.73% | 18 | 1.53% | 0.32 |
| M3-19 | M | 9,941 | 1.70% | 1 | 0.09% | 0.05 |
| M3-20 | M | 6,370 | 1.09% | 20 | 1.71% | 1.56 |
| M3-21 | M | 13,432 | 2.30% | 23 | 1.96% | 0.85 |
| M3-22 | M | 13,366 | 2.29% | 37 | 3.15% | 1.38 |
| M3-23 | M | 8,920 | 1.53% | 19 | 1.62% | 1.06 |
| M3-24 | M | 17,580 | 3.01% | 11 | 0.94% | 0.31 |
| M3-25 | M | 15,085 | 2.58% | 22 | 1.88% | 0.73 |
| M3-26 | M | 23,563 | 4.04% | 5 | 0.43% | 0.11 |
| M3-27 | M | 1,987 | 0.34% | 0 | 0.00% | 0.00 |
| M3-28 | M | 6,043 | 1.04% | 2 | 0.17% | 0.16 |
| M3-29 | M | 26,534 | 4.55% | 29 | 2.47% | 0.54 |
| M3-30 | M | 11,495 | 1.97% | 24 | 2.05% | 1.04 |
| M3-31 | M | 6,230 | 1.07% | 3 | 0.26% | 0.24 |
| M3-32 | M | 19,627 | 3.36% | 8 | 0.68% | 0.20 |
| M3-33 | M | 12,665 | 2.17% | 27 | 2.30% | 1.06 |
| M3-34 | M | 11,949 | 2.05% | 3 | 0.26% | 0.12 |
| M3-35 | M | 13,578 | 2.33% | 5 | 0.43% | 0.18 |
| M3-36 | M | 11,952 | 2.05% | 50 | 4.26% | 2.08 |
| F3-1 | F | 35,345 | 6.06% | 241 | 20.55% | 3.39 |
| Total | | 583,652 | 100% | 1,173 | 100% | — |

As shown in Table 22, a total of 1,173 explants from the male inbred lines generated shoots and were identified by genotyping after Agrobacterium inoculation, representing 35 out of 36 male germplasms tested in Experiment 4. The “% Shoots Generated after Agro” ranged from 0% to 11.85% for male germplasms. Some samples could not be positively identified and were excluded. These results indicate that most germplasms tested in this Experiment 4, with the exception of one male line, were able to generate shoot(s) after inoculation with Agrobacterium, indicating that bulked explants of male germplasms can be transformed via Agrobacterium and regenerated into shoots and plants. However, the range of shoot generation frequencies after Agrobacterium inoculation in Table 22 (represented by the “% Shoots Generated after Agro”) indicates that different germplasms respond differently to these treatments.

In Experiment 4, approximately 68,400 total explants from the male bulk germplasms were sterilized and rehydrated as a single preparation and then 15 replicates of approximately 4,560 explants were inoculated with Agrobacterium harboring a recombinant DNA construct containing a β-glucuronidase reporter gene as described in Example 3 with −570 explants being inoculated per 50 mL tube. Replicates 1-9 consisted of a single inoculation group of 4,560 explants (“normal” density) while replicates 10-15 consisted of two inoculation groups of 2,280 explants each (“low” density) to improve efficiency and reduce contamination effects. After inoculation, the Agrobacterium suspension was removed, and the inoculated explants were blotted on a sterile paper towel to remove excess Agrobacterium solution. Explants were then transferred to co-culture plates as described in Example 4 at a density of either about 285 or about 570 explants per plate depending on the replicate (low vs. high density, respectively).

After co-culture, explants were transferred to Petri plates containing bud induction media as described in Example 4 at a target density of either about 57 or about 114 explants per plate depending on the replicate (low vs. high density, respectively). The explants were then transferred to extended bud induction medium as previously described at a target density of either about 19 or about 38 regenerable explants per plate depending on the replicate (low vs. high density, respectively) and cultured as described in Example 4. Next, the explants were transferred to 40 ml plates with regeneration medium as previously described at a density of either about 19 or about 38 regenerable explants per plate depending on the replicate (low vs. high density, respectively), and cultured as described in Example 4. Finally, green explants were transferred to regeneration medium and cultured as described in Example 4 at a target density of about 100 explants/tray. Regenerated plants were collected and assayed for genotyping and transformation.

The results of this Experiment 4 are summarized in Table 23. The definitions for the terminology in each of the columns in Table 23 are the same as provided above for Bulks 3 & 4 in Table 10, except that the numbers and calculations are for male germplasms out of a total of 450 transformants identified by genotyping. Editing was not performed in this Experiment 4.

TABLE 23
Response of different male germplasms in bulk explant preparations
to Agrobacterium-mediated transformation in Experiment 4.

| | | | | | | 7 |
|---|---|---|---|---|---|---|
| | 2 | 3 | 4 | 5 | 6 | Relative |
| 1 | Heterotic | # Kernels | % Kernels | # | % | Transformation |
| Germplasm | group | in Bulk | in Bulk | Transformants | Transformants | Rate |
| M3-1 | M | 3,395 | 0.58% | 0 | 0.00% | 0.00 |
| M3-2 | M | 16,238 | 2.78% | 0 | 0.00% | 0.00 |
| M3-3 | M | 12,578 | 2.16% | 0 | 0.00% | 0.00 |
| M3-4 | M | 13,608 | 2.33% | 0 | 0.00% | 0.00 |
| M3-5 | M | 22,812 | 3.91% | 0 | 0.00% | 0.00 |
| M3-6 | M | 19,701 | 3.38% | 0 | 0.00% | 0.00 |
| M3-7 | M | 17,875 | 3.06% | 0 | 0.00% | 0.00 |
| M3-8 | M | 6,427 | 1.10% | 0 | 0.00% | 0.00 |
| M3-9 | M | 23,856 | 4.09% | 5 | 1.11% | 0.27 |
| M3-10 | M | 20,368 | 3.49% | 0 | 0.00% | 0.00 |
| M3-11 | M | 22,711 | 3.89% | 4 | 0.89% | 0.23 |
| M3-12 | M | 25,074 | 4.30% | 13 | 2.89% | 0.67 |
| M3-13 | M | 17,602 | 3.02% | 2 | 0.44% | 0.15 |
| M3-14 | M | 7,026 | 1.20% | 0 | 0.00% | 0.00 |
| M3-15 | M | 17,319 | 2.97% | 0 | 0.00% | 0.00 |
| M3-16 | M | 24,342 | 4.17% | 2 | 0.44% | 0.11 |
| M3-17 | M | 19,469 | 3.34% | 0 | 0.00% | 0.00 |
| M3-18 | M | 27,589 | 4.73% | 2 | 0.44% | 0.09 |
| M3-19 | M | 9,941 | 1.70% | 0 | 0.00% | 0.00 |
| M3-20 | M | 6,370 | 1.09% | 3 | 0.67% | 0.61 |
| M3-21 | M | 13,432 | 2.30% | 0 | 0.00% | 0.00 |
| M3-22 | M | 13,366 | 2.29% | 10 | 2.22% | 0.97 |
| M3-23 | M | 8,920 | 1.53% | 0 | 0.00% | 0.00 |
| M3-24 | M | 17,580 | 3.01% | 0 | 0.00% | 0.00 |
| M3-25 | M | 15,085 | 2.58% | 0 | 0.00% | 0.00 |
| M3-26 | M | 23,563 | 4.04% | 0 | 0.00% | 0.00 |
| M3-27 | M | 1,987 | 0.34% | 0 | 0.00% | 0.00 |
| M3-28 | M | 6,043 | 1.04% | 0 | 0.00% | 0.00 |
| M3-29 | M | 26,534 | 4.55% | 4 | 0.89% | 0.20 |
| M3-30 | M | 11,495 | 1.97% | 1 | 0.22% | 0.11 |
| M3-31 | M | 6,230 | 1.07% | 0 | 0.00% | 0.00 |
| M3-32 | M | 19,627 | 3.36% | 0 | 0.00% | 0.00 |
| M3-33 | M | 12,665 | 2.17% | 1 | 0.22% | 0.10 |
| M3-34 | M | 11,949 | 2.05% | 4 | 0.89% | 0.43 |
| M3-35 | M | 13,578 | 2.33% | 12 | 2.67% | 1.15 |
| M3-36 | M | 11,952 | 2.05% | 0 | 0.00% | 0.00 |
| F3-1 | F | 35,345 | 6.06% | 387 | 86.00% | 14.20 |
| Total | | 583,652 | 100% | 450 | 100% | — |

As shown in Table 23, a total of 450 explants identified by genotyping were successfully transformed, representing 13 out of 36 male germplasms and the female germplasm control tested in Example 4, Experiment 2. The “% Transformants” ranged from 0% to 2.89% among the male germplasm lines. These results indicate that many of the bulked germplasms can be successfully transformed, but different germplasms in the bulk exhibit varying transformation frequencies.

### Example 11

### Bulk Transformation of Plant Protoplasts

A heterologous polynucleotide molecule, a ribonucleoprotein, or a site-specific nuclease may be collectively introduced into a bulk population of distinct plant protoplasts comprising protoplasts from at least two different plant genotypes. Methods of plant protoplast transformation and the regeneration of genetically modified plants therefrom are known in the art, and any such method may be used according to the embodiments of the present disclosure. For example, Reed and Bargmann, Front Genome Ed 3: 734951, 2021, which is incorporated herein by reference, describes such methods. Non-limiting examples of methods for protoplast transformation include electroporation, microprojectile or particle bombardment, microinjection, PEG-mediated transformation, and other modes of direct DNA uptake. A distinct plant protoplast refers to a plant protoplast comprising genomic features that are identifiable and correlated to true breeding characteristics. Distinct plant protoplasts can be identified at the genomic level and distinguished from other plant protoplasts included within the same bulk transformation pool. Following regeneration of a genetically modified plant from a genetically modified distinct plant protoplast, deconvolution may be performed using methods similar to those described in Examples 1-10 of the present disclosure.

### Example 12

### Bulk Transformation Followed by Callus Related Regeneration

A heterologous polynucleotide molecule, a ribonucleoprotein, or a site-specific nuclease may be collectively introduced into a bulk population of distinct embryo explants comprising explants from at least two different plant genotypes, as described herein. The population of distinct embryo explants may include, for example, any plant part or plant tissue that is capable of being genetically modified and subsequently generated/regenerated into a genetically modified plant or plant part. Following genetic modification, the genetically modified explants may be regenerated using any method known in the art, including but not limited to, callus related regeneration methods. Callus related regeneration methods are known in the art and any such method may be used according to the embodiments of the present disclosure. Callus related regeneration methods are described, for example, in U.S. Pat. Pub. Nos. 2004/0210958, 2005/0289673, 2007/0157346, 2007/0271627, 2008/0118981, 2008/0124727, 2008/0282432, and 2008/0057512, each of which is incorporated herein by reference. Following regeneration of a genetically modified plant from a genetically modified distinct embryo explant, deconvolution may be performed using methods similar to those described in Examples 1-10 of the present disclosure.

What is claimed is:

1. A composition comprising a population of distinct germplasm and a heterologous polynucleotide molecule, wherein the population of distinct germplasm comprises germplasm of at least two different plant genotypes.

2. The composition of claim 1, wherein the population is defined as a population of distinct monocot germplasm or dicot germplasm.

3. The composition of claim 2, wherein the population is defined as a population of distinct corn, wheat, rice, barely, sorghum, turfgrass, soybean, cotton, or canola germplasm.

4. The composition of claim 1, wherein the population is defined as a population of distinct monocot or dicot embryo explants.

5. The composition of claim 4, the population is defined as a population of distinct corn, wheat, rice, barely, sorghum, turfgrass, soybean, cotton, or canola embryo explants.

6. The composition of claim 1, wherein the heterologous polynucleotide molecule comprises an expression cassette and the expression cassette comprises a selectable marker, a screenable marker, a coding sequence of interest, a nucleotide sequence encoding a site-specific nuclease, or a nucleotide sequence encoding a guide RNA molecule.

7. The composition of claim 1, wherein the heterologous polynucleotide molecule comprises a guide RNA molecule.

8. The composition of claim 1, further comprising a Rhizobiales bacterium.

9. The composition of claim 8, wherein the Rhizobiales bacterium is selected from the group consisting of:
a) a Rhizobiaceae, a Phyllobacteriaceae, a Brucellaceae, a Bradyrhizobiaceae, and a Xanthobacteraceae bacterium; or
b) an Agrobacterium, a Rhizobium, a Sinorhizobium, a Mesorhizobium, a Phyllobacterium, an Ochrobactrum, a Bradyrhizobium, and an Azorhizobium bacterium.

10. The composition of claim 8, further comprising an inoculation medium.

11. A composition comprising a field, wherein the field comprises plants having a population of distinct germplasm and, wherein at least one plant of each distinct germplasm comprises a heterologous polynucleotide molecule.

12. The composition of claim 1, wherein the distinct germplasm of the at least two different plant genotypes comprise germplasm of a first genotype and germplasm of a second genotype, and wherein the distinct germplasm of the first genotype and the distinct germplasm of the second genotype are present in the population in a predetermined ratio.

13. The composition of claim 12, wherein the predetermined ratio is determined based upon at least one culturing characteristic associated with the first genotype, associated with the second genotype, or associated with the first genotype and the second genotype.

14. The composition of claim 13, wherein the at least one culturing characteristic is selected from the group consisting of: explant excision efficiency, regeneration efficiency, shoot generation efficiency, genetic modification efficiency, transformation efficiency, and ability to regenerate into a genetically modified plant or plant part.

15. The composition of claim 12, wherein the predetermined ratio of the distinct germplasm of the first genotype and the second genotype comprises an approximately equal number of germplasm of the first genotype and of the second genotype.

16. A composition comprising a population of distinct germplasm and a ribonucleoprotein or a site-specific nuclease, wherein the population of distinct germplasm comprises germplasm of at least two different plant genotypes.

17. The composition of claim 16, wherein the ribonucleoprotein comprises a site-specific nuclease and a guide RNA molecule.

18. The composition of claim 16, wherein the population is defined as a population of distinct monocot or dicot germplasm.

19. The composition of claim 17, wherein the population is defined as a population of distinct corn, wheat, rice, barely, sorghum, turfgrass, soybean, cotton, or canola germplasm.

20. The composition of claim 16, wherein the population is defined as a population of distinct monocot or dicot embryo explants.

21. The composition of claim 20, wherein the population is defined as a population of distinct corn, wheat, rice, barely, sorghum, turfgrass, soybean, cotton, or canola embryo explants.

22. The composition of claim 16, further comprising a Rhizobiales bacterium.

23. The composition of claim 22, wherein the Rhizobiales bacterium is selected from the group consisting of:
a) a Rhizobiaceae, a Phyllobacteriaceae, a Brucellaceae, a Bradyrhizobiaceae, and a Xanthobacteraceae bacterium; or
b) an Agrobacterium, a Rhizobium, a Sinorhizobium, a Mesorhizobium, a Phyllobacterium, an Ochrobactrum, a Bradyrhizobium, and an Azorhizobium bacterium.

24. The composition of claim 16, further comprising an inoculation medium.

25. The composition of claim 16, wherein the distinct germplasm of the at least two different plant genotypes comprise germplasm of a first genotype and germplasm of a second genotype, and wherein the distinct germplasm of the first genotype and the distinct germplasm of the second genotype are present in the population in a predetermined ratio.

26. The composition of claim 25, wherein the predetermined ratio is determined based upon at least one culturing characteristic associated with the first genotype, associated with the second genotype, or associated with the first genotype and the second genotype.

27. The composition of claim 26, wherein the at least one culturing characteristic is selected from the group consisting of: explant excision efficiency, regeneration efficiency, shoot generation efficiency, genetic modification efficiency, transformation efficiency, and ability to regenerate into a genetically modified plant or plant part.

28. The composition of claim 25, wherein the predetermined ratio of the distinct germplasm of the first genotype and the second genotype comprises an approximately equal number of germplasm of the first genotype and of the second genotype.

29. A method of producing a population of distinct genetically modified germplasm, comprising:
a) contacting a population of distinct germplasm with at least one heterologous polynucleotide molecule, wherein the population of distinct germplasm comprises germplasm of at least two different plant genotypes;
b) genetically modifying the population of distinct germplasm to be genetically modified; and
c) collecting the population of distinct genetically modified germplasm.

30. The method of claim 29, wherein the population of distinct genetically modified germplasm comprises germplasm of at least two different plant genotypes.

31. The method of claim 30, further comprising identifying a genotype of at least one germplasm of the population of distinct genetically modified germplasm.

32. The method of claim 31, wherein said identifying the genotype comprises detecting at least one genetic marker in the at least germplasm, wherein the at least one genetic marker comprises a polynucleotide sequence that is characteristic of the genotype.

33. The method of claim 32, wherein said polynucleotide sequence is exclusively characteristic of the genotype.

34. The method of claim 31, wherein said identifying the genotype comprises detecting at least 2, at least 3, at least 4, at least 5, at least 6, at least 7, at least 8, at least 9, at least 10, at least 15, at least 20, or at least 25 genetic markers in the at least one germplasm, wherein each of said genetic markers comprises a polynucleotide sequence that is characteristic of the genotype.

35. The method of claim 34, wherein each of said genetic markers comprises a polynucleotide sequence that is exclusively characteristic of the genotype.

36. The method of claim 31, wherein said identifying the genotype comprises identifying at least one genotype of a plurality of germplasms of the population of distinct genetically modified germplasm.

37. The method of claim 36, wherein said identifying the genotype comprises identifying a plurality of genotypes of the plurality of germplasms of the population of distinct genetically modified germplasms.

38. The method of claim 29, further comprising identifying at least one genetic modification present in at least one germplasm of the population of distinct genetically modified germplasm.

39. The method of claim 38, further comprising identifying at least one genetic modification present in a plurality of germplasms of the population of distinct genetically modified germplasm.
